(19) United States
(12) Patent Application Publication
(10) Pub. No.: US 2025/0122267 A1
(43) Pub. Date: Apr. 17, 2025

(54) Gene Therapy Methods for Age-Related Diseases and Conditions

(71) Applicant: President and Fellows of Harvard College, Cambridge, MA (US)

(72) Inventors: Noah Davidsohn, Brookline, MA (US); George M. Church, Brookline, MA (US)

(21) Appl. No.: 18/942,898

(22) Filed: Nov. 11, 2024

Related U.S. Application Data
(63) Continuation of application No. 16/302,865, filed on Nov. 19, 2018.
(60) Provisional application No. 62/421,665, filed on Nov. 14, 2016.
(60) Provisional application No. 62/339,182, filed on May 20, 2016.

(51) Int. Cl. C07K 14/71; A61K 48/00; A61P 3/04; A61P 9/04; C07K 14/50; C12N 15/86

(52) U.S. Cl. CPC C07K 14/71; A61P 3/04; A61P 9/04; C07K 14/50; C12N 15/86; A61K 48/00; C07K 2319/30; C12N 2750/14132; C12N 2750/14143; C12N 2830/48

(57) ABSTRACT

Methods of gene therapy are provided for treating or preventing age-related diseases or conditions by regulating one or more functional proteins associated with age-related diseases or conditions.

### RELATED APPLICATIONS

This application is a continuation application which claims priority to U.S. patent application Ser. No. 16/302,865, filed Nov. 19, 2018; which is a National Stage Application under 35 U.S.C. 371 of co-pending PCT application PCT/US17/33815 designating the United States and filed May 22, 2017; which claims the benefit of U.S. Provisional Application No. 62/421,665 filed on Nov. 14, 2016 and to U.S. Provisional Application No. 62/339,182 filed on May 20, 2016 each of which are hereby incorporated by reference in their entireties.

### STATEMENT OF GOVERNMENT INTERESTS

This invention was made with Government Support under HG005550 awarded by the National Institutes of Health. The Government has certain rights in this invention.

### SEQUENCE LISTING

The instant application contains a Sequence Listing which has been submitted electronically in XML format and is hereby incorporated by reference in its entirety. Said XML copy, created on Nov. 8, 2024, is named “Sequence_Listing_010498.01623_ST26” and is 246 KB in size.

### BACKGROUND

Aging is the gradual loss of function and deterioration at the cellular, tissue, and organ level, leading to increased susceptibility to disease and external stressors, and eventually death. All organisms age, but the effects of aging can be slowed or minimized or manipulated. Numerous experiments have shown the ability to increase maximal lifespan as well as healthspan with decreased susceptibility to other age related pathologies. Aging interventions tested to date have included environmental manipulation such as calorie restriction (CR), small molecule drugs such as rapamycin, and genetic manipulations accomplished through the creation of transgenic animals such as the Ames and Snell dwarf mice. While these experiments have led to a greater understanding of the mechanisms involved in aging, they are not amenable to translation to aging human and pet populations. Calorie restriction requires strict adherence to dietary constraints and evidence to date suggests this is not a likely avenue for treatment. Rapamycin has immune modulation effects that can increase vulnerability to certain pathogens. And creating transgenic animals does not apply to all the existing living organisms. AAV delivery of hTERT into a cancer resistant genetic background mouse is described in Bernardes de Jesus B. et al., (2012) EMBO Mol Med. 4 (8):691-704.

Gene therapy methods are known. For example GLYBERA is a human gene therapy from uniQure that treats lipoprotein lipase deficiency (LPLD) by adding a working copy of the involved gene through intramuscular injections of AAV (adeno-associated virus). SPK-RPE65 is from Spark Therapeutics and is a human gene therapy that treats a rare blinding condition from a non-functioning RPE65 gene.

### SUMMARY

The present disclosure provides gene therapy methods, such as combinational gene therapy methods to provide or regulate one or more endogenous proteins. The disclosure provides a method of treating or preventing age-related diseases and conditions or the aging phenotype using gene therapy. The disclosure provides the identification of genes related to certain diseases and conditions that may be associated with aging. The disclosure provides for the regulation of such genes either by increasing a protein related to a gene or decreasing a protein related to a gene. The disclosure provides for increasing a functional protein related to a gene by introducing a nucleic acid encoding the functional protein which is expressed within a cell. The expression results in the increased amount of the functional protein that can either be intracellular or be secreted providing a therapeutic effect or prophylactic effect. The disclosure provides for the inhibition of a gene thereby decreasing the functional protein associated with the gene by introducing a nucleic acid encoding an inhibitory RNA which when expressed binds to the gene or the messenger RNA to inhibit expression of the functional protein. The disclosure provides for the inhibition of a functional protein thereby decreasing the activity of the functional protein by introducing a nucleic acid encoding a protein inhibitor, such as a soluble receptor protein, which when expressed, binds to the functional protein. The disclosure provides for gene therapy methods using genetic constructs targeting cells in an animal and the delivery of such genetic constructs using vectors, such as viral vectors. The disclosure provides for gene therapy methods using genetic constructs targeting cells in an animal and the delivery of such genetic constructs using methods such as liposomes, synthetic or naturally occurring polymers, electroporation, coated or non-coated nano-particle delivery, bolistic particle delivery, laser mediate transfection (optoporation or phototransfection). See, e.g., Kim, T. K. et al., (2010) Analytical and Bioanalytical Chemistry. 397 (8):3173-3178. The disclosure provides for gene therapy methods using genetic constructs targeting cells in an animal and the delivery of such genetic constructs wherein the genetic construct has been processed from the original DNA into miRNA, shRNA, RNAi, or mRNA (where the mRNA consists of a 5′ Cap and a 3′ poly A or equivalent). According to additional aspects, the RNA is targeted to the ribosome for translation using a 5′ Cap analogue known to those of skill in the art or 3′ poly A analogue known to those of skill in the art. For the gene therapy methods described herein, the disclosure provides for the use of a gene or gene product or DNA encoding the gene or mRNA corresponding to the gene or processed pri-mRNA or miRNA corresponding to the gene in the methods of gene therapy described herein insofar as the gene or gene product or DNA encoding the gene or mRNA corresponding to the gene or processed pri-mRNA or miRNA corresponding to the gene are altered or regulated to provide a cellular effect in the therapeutic or prophylactic methods described herein.

The regulation or providing of certain proteins associated with age-related diseases and conditions provide prophylactic methods or therapeutic methods to address age-related diseases and conditions. The regulation or providing of certain proteins or genes or gene products or DNA encoding the gene or mRNA corresponding to the gene or processed pri-mRNA or miRNA corresponding to the gene associated with age-related diseases and conditions provide methods of rejuvenating organisms including humans and other mammals. The disclosure provides gene therapy methods where one or more or a plurality of nucleic acids, such as genes, are delivered to one or more target cells in an animal. The disclosure provides the delivery to a cell of a plurality of nucleic acids including a single promoter driving their expression using a single vector. The one or more or plurality of nucleic acids are expressed to produce one or more corresponding proteins and the one or more proteins alter a condition of the organism. The disclosure provides for combination therapy where different cell types are targeted by the one or more or plurality of nucleic acids in the animal.

The disclosure provides for combination therapy where one or more cellular processes within a cell are targeted by the one or more or plurality of nucleic acids. The disclosure provides for gene therapy using a viral vector such as a parvoviral virion. The disclosure provides for gene therapy using a viral vector such as an adeno-associated virus (“AAV”). The adeno-associated virus will insert an exogenous gene into a cell and the protein encoded by the exogenous gene will be expressed. In this manner, the protein, whether a functional protein, an inhibitory RNA or an inhibitory protein, will alter the cell and/or the organism harboring the cell.

The disclosure provides for the slowing, inhibiting, forestalling or reversing of age-related diseases or conditions. Exemplary age-related or other diseases or conditions include one or more of cardiovascular diseases, diabetes, atherosclerosis, obesity, cancer, infection, and neurological disorders. The disclosure provides long-term gene therapy treatments to treat and/or prevent age-related or other diseases or conditions. The methods include reversing age-related diseases and conditions and correcting these pathological states resulting in an increased healthspan (years of good quality of life) and lifespan.

The disclosure provides a method for identifying a gene or set of genes to be regulated which prevent or treat one or more diseases or conditions, such as diseases or conditions associated with aging. The gene or set of genes are identified as being related to age-related diseases or conditions. The genes are determined to be associated or non-associated with a particular tissue type so that appropriate regulation of the gene using desired methods can be determined. In addition, genes associated with a particular tissue type may benefit from regulation using particular vectors that deliver to a particular tissue type cell a nucleic acid, inhibitory RNA or inhibitory protein to regulate the amount or activity of the protein within the particular tissue type cell. Tissue specific promoters may be used to express the nucleic acid.

Exemplary nucleic acids encoding particular functional proteins, inhibitory RNA or inhibitory proteins are provided at Appendix A, the sequences of which are provided in Appendix A or are readily known or available in the literature. Likewise, sequences for the functional proteins, inhibitory RNA or inhibitory proteins are known to those of skill in the art or can be derived from the nucleic acid sequences. Appendix B includes the DNA and amino acid sequences for the mouse versions of genes as well as the pri-miRNA DNA constructs that target multiple RNA species.

Functional proteins as described herein can be the full length proteins or proteins which vary from the full length proteins but retain the activity in whole or in part of the full length protein.

Further features and advantages of certain embodiments of the present invention will become more fully apparent in the following description of embodiments and drawings thereof, and from the claims.

### BRIEF DESCRIPTION OF THE FIGURES

FIG. 1 are representative echocardiograms after 7 weeks post AAC surgery.

FIG. 2 is a graph of data demonstrating TGFb1 knockdown versus dosage.

FIG. 3 is a graph of data showing percent fibrosis and related images.

FIG. 4 are images showing WGA staining for control heart and treated heart sections.

FIG. 5 is a graph of data demonstrating change in heart parameters in a control versus STGFbR2-FC administration.

FIG. 6 are representative trichrome staining images.

FIG. 7 is a graph of percent survival versus time in days.

FIG. 8A is a graph of weight loss versus time in days for FGF21. FIG. 8B is a graph of weight loss versus time in days for various FGF21 gene therapies.

FIG. 9 is a graph of weight loss versus time in days for GDF15, adiponectin, ZAG, and Nrf2.

FIG. 10 is a vector map of viral construct including ITRs promoter sTGFbR2-Fc and 3′UTR.

FIG. 11 is a vector map of viral construct including ITRs promoter Nrf2 and 3′UTR.

FIG. 12 is a vector map of a possible construction of 7 Pri-miRNAs and the order they are concatenated. There is also a red mark for where the mismatches are planned in the “shRNA” part for proper processing of the miRNA. Figure discloses the nucleotide sequence as SEQ ID NO: 137 and the amino acid sequences as SEQ ID NOS 138-145, respectively, in order of appearance.

FIG. 13 is a vector map of a possible construction of 6 Pri-miRNAs and the order they are concatenated. There is also a red mark for where the mismatches are planned in the “shRNA” part for proper processing of the miRNA. Figure discloses the nucleotide sequence as SEQ ID NO: 146 and the amino acid sequences as SEQ ID NOS 147-151, respectively, in order of appearance.

FIG. 14 depicts a vector map of sTGFbR2-Fc.

FIG. 15 depicts data of binding of soluble TGFb receptor 2 to TGFb1 in dog serum.

FIG. 16 is a graph of food intake of a control mice versus mice treated with FGF21.

FIG. 17 depicts data of control mice versus mice treated with FGF21.

FIG. 18 depicts glucose level data.

FIG. 19 depicts fractional shortening data.

FIG. 20 depicts data of percent survival after gene therapy.

FIGS. 21A-D depicts data directed to increased healthspan.

FIG. 22 depicts kidneys of control mice versus mice treated with gene therapy as described herein.

### DETAILED DESCRIPTION

The present disclosure provides gene therapy methods where one or more or a plurality of nucleic acids encoding a functional protein, an inhibitory RNA or an inhibitory protein are provided to cells within a subject. The one or more nucleic acids are administered by one or more vectors or combined into a single viral vector, such as an AAV, to treat or prevent diseases or conditions associated with aging and age-related physiological decline.

As used in this specification and the appended claims, the singular forms “a”, “an” and “the” include plural referents unless the context clearly indicates otherwise. Thus, for example, reference to “a protein” includes more than one protein, and reference to “an excipient” includes more than one excipient.

It is further to be understood that use of “or” means “and/or” unless stated otherwise. Similarly, “comprise,” “comprises,” “comprising” “include,” “includes,” and “including” are interchangeable and not intended to be limiting. Also, where descriptions of various embodiments use the term “comprising,” those skilled in the art would understand that in some specific instances, an embodiment can be alternatively described using language “consisting essentially of” or “consisting of.”

The foregoing general description, including the drawings, and the following detailed description are exemplary and explanatory only and are not restrictive of this disclosure.

The section headings used herein are for organizational purposes only and not to be construed as limiting the subject matter described.

### Definitions

In reference to the present disclosure, the technical and scientific terms used in the descriptions herein will have the meanings commonly understood by one of ordinary skill in the art, unless specifically defined otherwise. Accordingly, the following terms are intended to have the following meanings:

“Gene” as used herein refers to a nucleic acid region, also referred to as a transcribed region, which expresses a polynucleotide, such as an RNA. The transcribed polynucleotide can have a sequence encoding a polypeptide, such as a functional protein, which can be translated into the encoded polypeptide when placed under the control of an appropriate regulatory region. A gene may comprise several operably linked fragments, such as a promoter, a 5′leader sequence, a coding sequence and a 3′ nontranslated sequence, such as a polyadenylation site. A chimeric or recombinant gene is a gene not normally found in nature, such as a gene in which, for example, the promoter is not associated in nature with part or all of the transcribed DNA region. “Expression of a gene” refers to the process wherein a gene is transcribed into an RNA and/or translated into a functional protein.

“Gene delivery” or “gene transfer” refers to methods for introduction of recombinant or foreign DNA into host cells. The transferred DNA can remain non-integrated or preferably integrates into the genome of the host cell. Gene delivery can take place for example by transduction, using viral vectors, or by transformation of cells, using known methods, such as electroporation, cell bombardment.

“Transgene” refers to a gene that has been introduced into a host cell. The transgene may comprise sequences that are native to the cell, sequences that do not occur naturally in the cell, or combinations thereof. A transgene may contain sequences coding for one or more proteins that may be operably linked to appropriate regulatory sequences for expression of the coding sequences in the cell.

“Transduction” refers to the delivery of a nucleic acid molecule into a recipient host cell, such as by a gene delivery vector, such as rAAV. For example, transduction of a target cell by a rAAV virion leads to transfer of the rAAV vector contained in that virion into the transduced cell. “Host cell” or “target cell” refers to the cell into which the nucleic acid delivery takes place.

“Functional protein” includes variants, mutations, homologues, and functional fragments of the full length proteins. One of skill will readily be able to construct proteins homologous to the full length proteins which retain the activity, in whole or in part, of the full length protein.

“Vector” refers generally to nucleic acid constructs suitable for cloning and expression of nucleotide sequences. One example of a vector is a viral vector. The term vector may also sometimes refer to transport vehicles comprising the vector, such as viruses or virions, which are able to transfer the vector into and between host cells.

“AAV vector” or “rAAV vector” refers to a recombinant vector derived from an adeno-associated virus serotype, such as AAV1, AAV2, AAV3, AAV4, AAV5, AAV6, AAV7, AAV8, AAV9, AAV10, AAV11, AAV12, AAV2.5, AAvDJ, AAVrh10.XX and others. rAAV vectors can have one or preferably all wild type AAV genes deleted, but still comprise functional ITR nucleic acid sequences. Functional ITR sequences are necessary for the replication, rescue and packaging of AAV virions. The ITR sequences may be wild type sequences or substantially identical sequences (as defined below) or may be altered by for example in insertion, mutation, deletion or substitution of nucleotides, as long as they remain functional.

“Therapeutically effective amount” refers to an amount effective, at dosages and for periods of time necessary, to achieve the desired therapeutic result, such as results directed at age-related diseases or conditions. A therapeutically effective amount of a parvoviral virion or pharmaceutical composition may vary according to factors such as the disease state, age, sex, and weight of the individual, and the ability of the parvoviral virion or pharmaceutical composition to elicit a desired response in the individual. Dosage regimens may be adjusted to provide the optimum therapeutic response. A therapeutically effective amount is also typically one in which any toxic or detrimental effects of the parvoviral virion or pharmaceutical composition are outweighed by the therapeutically beneficial effects.

“Prophylactically effective amount” refers to an amount effective, at dosages and for periods of time necessary, to achieve the desired prophylactic result, such as preventing or inhibiting various age-related diseases or conditions, A prophylactic dose may be used in subjects prior to or at an earlier stage of disease, and a prophylactically effective amount may be more or less than a therapeutically effective amount in some cases.

“Nucleic acid” includes any molecule composed of or comprising monomeric nucleotides. The term “nucleotide sequence” may be used interchangeably with “nucleic acid” herein. A nucleic acid may be an oligonucleotide or a polynucleotide. A nucleic acid may be a DNA or an RNA. A nucleic acid may be a gene. A nucleic acid may be chemically modified or artificial. Artificial nucleic acids include peptide nucleic acid (PNA), Morpholino and locked nucleic acid (LNA), as well as glycol nucleic acid (GNA) and threose nucleic acid (TNA). Each of these is distinguished from naturally-occurring DNA or RNA by changes to the backbone of the molecule. Also, phosphorothioate nucleotides may be used.

“Nucleic acid construct” is herein understood to mean a man-made nucleic acid molecule resulting from the use of recombinant DNA technology. A nucleic acid construct is a nucleic acid molecule, either single- or double-stranded, which has been modified to contain segments of nucleic acids, which are combined and juxtaposed in a manner, which would not otherwise exist in nature. A nucleic acid construct usually is a “vector”, i.e. a nucleic acid molecule which is used to deliver exogenously created DNA into a host cell. One type of nucleic acid construct is an “expression cassette” or “expression vector”. These terms refers to nucleotide sequences that are capable of effecting expression of a gene in host cells or host organisms compatible with such sequences. Expression cassettes or expression vectors typically include at least suitable transcription regulatory sequences and optionally, 3′ transcription termination signals. Additional factors necessary or helpful in effecting expression may also be present, such as expression enhancer elements. A nucleic acid construct can also be a vector in which it directs expression or repression of a protein by operating as RNA instead of DNA. In the case of increasing expression of a target protein this nucleic acid construct can be mRNA or similar in which the cell or more specifically the ribosome would recognize and create many copies of the protein. In the case of repressing expression of a target sequence the RNA can be in the form that acts through preventing the ribosome from creating protein, this can be done through mechanisms of RNAi or shRNA or miRNA or Pri-miRNA. One could also imagine through Boolean logic that if one represses a known repressor of a target sequence one would in turn actually get an increase in the target sequence through repression and one skilled in the art can abstract away from the target protein such that any combination of “inversions” or “imply's” through the delivery of either mRNA (or similar) or shRNA (or similar) can produce the regulation of the target sequence. This can also be done through the vector that provides DNA that must be expressed as in the AAV.

“Operably linked” refers to a linkage of polynucleotide (or polypeptide) elements in a functional relationship. A nucleic acid is “operably linked” when it is placed into a functional relationship with another nucleic acid sequence. For instance, a transcription regulatory sequence is operably linked to a coding sequence if it affects the transcription of the coding sequence. Operably linked means that the DNA sequences being linked are typically contiguous and, where necessary to join two protein encoding regions, contiguous and in reading frame.

“Expression control sequence” refers to a nucleic acid sequence that regulates the expression of a nucleotide sequence to which it is operably linked. An expression control sequence is “operably linked” to a nucleotide sequence when the expression control sequence controls and regulates the transcription and/or the translation of the nucleotide sequence. Thus, an expression control sequence can include promoters, enhancers, internal ribosome entry sites (IRES), transcription terminators, a start codon in front of a protein-encoding gene, splicing signals for introns, 2A peptide sequences (that allow multicistronic expression) and stop codons. The term “expression control sequence” is intended to include, at a minimum, a sequence whose presence is designed to influence expression, and can also include additional advantageous components. For example, leader sequences and fusion partner sequences are expression control sequences. The term can also include the design of the nucleic acid sequence such that undesirable, potential initiation codons in and out of frame, are removed from the sequence. It can also include the design of the nucleic acid sequence such that undesirable potential splice sites are removed. It includes sequences or polyadenylation sequences (pA) which direct the addition of a polyA tail, i.e., a string of adenine residues at the 3′-end of a mRNA, which may be referred to as polyA sequences. It also can be designed to enhance mRNA stability. Expression control sequences which affect the transcription and translation stability, e.g., promoters, as well as sequences which effect the translation, e.g., Kozak sequences, suitable for use in insect cells are well known to those skilled in the art. Expression control sequences can be of such nature as to modulate the nucleotide sequence to which it is operably linked such that lower expression levels or higher expression levels are achieved.

One can also fuse functional domains to already known proteins. Such is the case where a mitochondrial signal is fused to CAT (catalase) such that the catalase is targeted to be shuttled to the mitochondria and perform its function inside or near the mitochondria instead of its natural location. One can also add targeting signals to other proteins to have them targeted to other parts of the cell or even secreted from the cell. In the case of some proteins a better known version can replace the natural sequence for enhanced effect, such as taking the human or mouse secretion signal for TGFbR2 and fusing it to the dog version of the protein.

“Promoter” or “transcription regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter, including e.g. attenuators or enhancers, but also silencers. A “constitutive” promoter is a promoter that is active in most tissues under most physiological and developmental conditions. An “inducible” promoter is a promoter that is physiologically or developmentally regulated, e.g. by the application of a chemical inducer. A “tissue specific” promoter is only active in specific types of tissues or cells. The disclosure provides for the operable linking of nucleic acid constructs to a mammalian cell-compatible expression control sequence, e.g., a promoter. Many such promoters are known in the art (see Sambrook and Russell, 2001, supra). Constitutive promoters that are broadly expressed in many cell types, such as the CMV and hEf1a promoter are disclosed. Variations of the full-length hEf1a are also disclosed which are shorter but still provide effective constitutive expression. Disclosed are promoters that are inducible, tissue-specific, cell-type-specific, or cell cycle-specific. In a disclosed embodiment, the nucleotide sequence encoding the porphobilinogen deaminase is operably linked to a liver-specific promoter. Liver-specific promoters are particularly preferred for use in conjunction the non-erythroid deaminase. Preferably, in a construct of the disclosure an expression control sequence for liver-specific expression are e.g. selected from the group consisting of an α1-anti-trypsin (AAT) promoter, a thyroid hormone-binding globulin promoter, an albumin promoter, a thyroxin-binding globulin (TBG) promoter, an Hepatic Control Region (HCR)-ApoCII hybrid promoter, an HCR-hAAT hybrid promoter, an AAT promoter combined with the mouse albumin gene enhancer (Ealb) element and an apolipoprotein E promoter. Other examples include the E2F promoter for tumour-selective, and, in particular, neurological cell tumour-selective expression (Parr et al., (1997) Nat. Med. 3:1145-9) or the IL-2 promoter for use in mononuclear blood cells (Hagenbaugh et al., (1997) J Exp Med; 185:2101-10).

“3′ UTR” or “3′ non-translated sequence” (also often referred to as 3′ untranslated region, or 3′end) refers to the nucleic acid sequence found downstream of the coding sequence of a gene, which comprises, for example, a transcription termination site and (in most, but not all eukaryotic mRNAs) a polyadenylation signal (such as e.g. AAUAAA or variants thereof). After termination of transcription, the mRNA transcript may be cleaved downstream of the polyadenylation signal and a poly(A) tail may be added, which is involved in the transport of the mRNA to the cytoplasm (where translation takes place).

“Naturally occurring sequence” or “native sequence” as used herein refers to a polynucleotide or amino acid isolated from a naturally occurring source. Included within “native sequence” are recombinant forms of a native polypeptide or polynucleotide which have a sequence identical to the native form.

“Mutant” or “variant” as used herein refers to an amino acid or polynucleotide sequence which has been altered by substitution, insertion, and/or deletion. In some embodiments, a mutant or variant sequence can have increased, decreased, or substantially similar activities or properties in comparison to the parental sequence.

“Percentage of sequence identity” and “percentage homology” are used interchangeably herein to refer to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman, (1981) Adv. Appl. Math. 2:482, by the homology alignment algorithm of Needleman and Wunsch, (1970) J. Mol. Biol. 48:443, by the search for similarity method of Pearson and Lipman, (1988) Proc. Natl. Acad. Sci. USA 85:2444, by computerized implementations of these algorithms (GAP, BESTFIT, FASTA, and TFASTA), or by visual inspection (see generally, Current Protocols in Molecular Biology, F. M. Ausubel et al., eds., Current Protocols, Greene Publishing Associates, Inc. and John Wiley & Sons, Inc., (1995 Supplement)).

Examples of algorithms that are suitable for determining percent sequence identity and sequence similarity are the BLAST and BLAST 2.0 algorithms, which are described in Altschul et al., (1990), J. Mol. Biol. 215:403-410 and Altschul et al., (1977) Nucleic Acids Res. 3389-3402, respectively. Software for performing BLAST analyses is publicly available through the National Center for Biotechnology Information website. This algorithm involves first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff and Henikoff, (1989) Proc. Natl. Acad. Sci. USA 89:10915).

The degree of percent amino acid sequence identity can also be obtained by ClustalW analysis (version W 1.8) by counting the number of identical matches in the alignment and dividing such number of identical matches by the length of the reference sequence, and using the following default ClustalW parameters to achieve slow/accurate pairwise optimal alignments-Gap Open Penalty: 10; Gap Extension Penalty: 0.10; Protein weight matrix: Gonnet series; DNA weight matrix: IUB; Toggle Slow/Fast pairwise alignments=SLOW or FULL Alignment.

“Subject” or “patient” refers to a mammal, such as a non-primate (e.g., cow, pig, horse, cat, dog, rat, etc.) or a primate (e.g., monkey or human). Preferably, the mammal is a domesticated animal, such as a dog, a cat, a mouse, a cow, a sheep, a goat, a horse, a pig, or a human subject. In some embodiments, the human is an adult patient. In some embodiments, the human is a pediatric patient.

### Gene Therapy by Expressing Functional Proteins and Regulating Functional Protein Expression

As summarized above, the present disclosure provides for the regulation of one or more or a plurality of genes or their associated functional proteins in a method of treating or preventing diseases or conditions associated with the targeted genes. In particular the individual targeted gene or one or more of a combination of the targeted genes is associated with age-related diseases or conditions and/or affecting biological lifespan. The genes or gene products targeted by the described gene therapy are involved in diverse cellular roles, such as metabolic activity, insulin-like growth factor activity pathway (i.e., IGF1/GH/mTOR axis), mitochondrial function, inflammatory/fibrosis, autophagy, neural function, genome stability, etc. The gene therapy can be based on, by way of example and not limitation, one or more of a nucleic acid or gene which overexpress a functional protein or a mutant form thereof; expression of a functional protein which regulates another target gene/protein; expression of polynucleotides, such as inhibitory RNA, to regulate expression of a target gene; and expression of gene editing systems that modify in situ the target gene. Such nucleic acids can be a “synthetic nucleotide sequence” which is herein understood to mean that the nucleotide sequence does not occur as such in nature, but rather was designed, engineered and/or constructed by human intervention. The term “synthetic” thus does not necessarily imply that the sequence is exclusively and/or entirely obtained through chemical synthesis. Rather, although parts of the synthetic sequence may at one stage have been obtained through chemical synthesis, molecules comprising a synthetic sequence of the invention will usually be obtained from biological sources such as (cultured, for example recombinant) cells.

In some embodiments, the gene for therapeutic applications and the corresponding expressed gene product are provided in Table 1 which may be administered, for example, by a viral vector system or a Cas9 guide RNA system.

TABLE 1

| | | Expressed | |
|---|---|---|---|
| | | product of the | |
| No. | Gene | Gene | Description of biological effect of Gene expression |
| 1 | ADcy5 | Inhibitory pri- | Decrease ADcy5 in order to decrease cAMP/PKA and |
| | | miRNA/shRNA | increase RAF/MEK/ERK to increase anti-apoptotic effect |
| | | | and cell survival and oxidative resistance. |
| 2 | Adiponectin | over express | Agonist of pParg and AMPK |
| | | protein | |
| 3 | Adra1a (mut) | over express | Constitutively active receptor leads to neurogenesis and |
| | | protein | plasticity and enhanced learning |
| 4 | Agtr1a | Inhibitory pri- | Increases mitochondrial biogenesis and NAMPT and Sirt3 |
| | | miRNA/shRNA | and decreases oxidative damage, especially in kidney and |
| | | | heart |
| 5 | Akt1 | Inhbitory Pri- | Akt1+/− mice showed a decrease of TOR signaling, but |
| | | miRNA/shRNA | phosphorylation of the forkhead transcription factors |
| | | | (FOXO) was not down-regulated, Decreasing Akt1 |
| | | | decreases ribsomal gene expression and lower mitochondrial |
| | | | biomass only need 50% reduction for effect |
| 6 | AMPK | over express | Metabolic involvement and energy expenditure |
| | | protein | |
| 7 | Atg5 | over express | Increases autophagy and increased levels of glutathione |
| | | protein | |
| 8 | BubR1 | over express | BubR1 overabundance exerts a protective effect by |
| | | protein | correcting mitotic checkpoint impairment and microtubule- |
| | | | kinetochore attachment defects. Furthermore, sustained high- |
| | | | level expression of BubR1 extends lifespan and delays age- |
| | | | related deterioration and aneuploidy in several tissues. |
| 9 | MCAT | over express | mitochondrial targeting signal attached to a catalase that |
| | | protein | enables the catalase to act in/near the mitochondria to |
| | | | decrease oxidative damage |
| 10 | Cebpalpha | Inhbitory pri- | Decrease cebpa and increase cebpb |
| | | miRNA/shRNA | |
| 11 | Cebpbeta | over express | acts by turning WAT to BAT by increasing mitochondria |
| | | protein | biogenesis and UCP1 |
| 12 | Cisd2d | over express | Mitochondrial membrane protein, Cisd2 may function as an |
| | | protein | autophagy regulator and may be involved in the Bcl-2- |
| | | | mediated regulation of autophagy and calcium homeostasis |
| 13 | Coq7 | Inhibitory pri- | Loss of Coq7 (or mClk-1) results in decreased ROS and |
| | | miRNA/shRNA | oxidative DNA damage the effect seems to come from the |
| | | | liver. |
| 14 | Ctf1 | Inhibitory pri- | CT-1-null mice shows decreased levels of inflammatory, |
| | | miRNA/shRNA | apoptotic, and senescence pathways, whereas telomere- |
| | | | linked proteins, DNA repair proteins, and antioxidant |
| | | | enzyme activities were increased. |
| 15 | Dgat1 | Inhibitory pri- | Involved in fat synthesis fat such that knocking down |
| | | miRNA/shRNA | expressom can result in less fat and thus less igf1 and |
| | | | increased lifespan |
| 16 | FGF21 | over express | Decreases IGF1 signaling, modulates metabolism, changes |
| | | protein | differentiation of osteoblast and osteoclasts, curbs appetite |
| 17 | GDF15 (hNAG) | over express | Acts through decreasing IGF1/mTOR/insulin signaling . . . |
| | | protein | Reduces weight in mice to prevent them from getting age |
| | | | associated obesity |
| 18 | HAS2 naked mole | over express | Anti-Cancer, Believed to create contact inhibition signals |
| | rat (nmr) | protein | through the body by making the environment more viscous |
| 19 | humanizeFoxP2 | over express | Learning and striatal neuroplasticity. Foxp2(hum/hum) mice |
| | | protein | learn stimulus-response associations faster than their WT |
| | | | littermates |
| 20 | Ikbkb | Inhibitory pri- | Acts through NFKB and GnRH development via immune- |
| | | miRNA/shRNA | neuroendocrine integration, and immune inhibition or GnRH |
| | | | restoration in the hypothalamus/brain |
| 21 | Insr | Inhibitory pri- | Fat Specific Knockout. Makes fat smaller and acts on |
| | | miRNA/shRNA | metabolism and insulin resistance |
| 22 | Klotho | over express | Klotho protein functions as a circulating hormone that binds |
| | | protein | to a cell-surface receptor and represses intracellular signals |
| | | | of insulin and insulin-like growth factor 1 (IGF1) as well as |
| | | | other effects |
| 23 | Mt1 | over express | Decreases anti-oxidants and increases resistance to stress in |
| | | protein | cardiac function. Delays onset of age associated phenotypes. |
| 24 | mTOR | Inhibitory pri- | Acts through NFkb. Active mTORC1 enhances processes |
| | | miRNA/shRNA | including glycolysis, protein, lipid and nucleotide |
| | | | biosynthesis, and it inhibits autophagy. By blocking mTOR |
| | | | you get health and lifespan inprovements in mice. |
| 25 | NEU1 | over express | Reduces B amyloid plaques and decreases AD development |
| | | protein | |
| 26 | nf-kb | Inhibitory pri- | Acts through inflammatory responses and immune |
| | | miRNA/shRNA | modulation |
| 27 | NGF | over express | Makes mice smarter; is a neuropeptide primarily involved in |
| | | protein | the regulation of growth, maintenance, proliferation, and |
| | | | survival of certain target neurons. Can increase pain in |
| | | | different areas and is a target for knockdown in neuropathy. |
| 28 | Nrf2 | over express | Expression of antioxidant and other protective proteins that |
| | | protein | protect against oxidative damage triggered by injury and |
| | | | inflammation. |
| 29 | NUDT1 | over express | Overexpression prevents the age-dependent accumulation of |
| | | protein | DNA 8-oxoguanine that occurs in wild-type mice. These |
| | | | lower levels of oxidized guanines are associated with |
| | | | increased longevity and hMTH1-Tg animals live |
| | | | significantly longer than their wild-type littermates |
| 30 | Pappa | Inhibiory pri- | Activates IGF1 so a knockout decreases expression of IGF1 |
| | | miRNA/shRNA | |
| 31 | Par4 SAC domain | over express | Anti-cancer, pro-apoptotic to cancer cells only, works |
| | | protein | through decreasing NFKb, activated by PKA in tumor cells |
| 32 | Pck1 | over express | Basically extra GTP, Activates mitochondrial biogenesis and |
| | | protein | energy production. Creates extra fat in in muscles to account |
| | | | for the high amount of energy needed. Is involved in the |
| | | | citric acid cycle flux |
| 33 | PCSK9 | Inhibitory pri- | Decreases bad cholesterol |
| | | miRNA/shRNA | |
| 34 | PDE4b | Inhibitory pri- | Disruption of PDE4b increases cAMP levels in the brain |
| | | miRNA/shRNA | makes mice smarter and less anxious |
| 35 | Prkar2b | Inhibitory pri- | Turns on UCP1 and is mediated by increasing intracellular |
| | | miRNA/shRNA | cAMP levels through the modulation of adenylyl cyclase |
| | | | (AC) activity |
| 36 | Rps6kb1 (S6K1) | Inhibitory pri- | part of the mTOR complex, Increases AMPK activation |
| | | miRNA/shRNA | when S6k1 is deleted |
| 37 | sIFG1r-fc | over express | Decreases IGF1 signaling |
| | | soluble receptor | |
| | | protein | |
| 38 | Sirt1 | over express | Sirt1 as a negative regulator of nuclear factor-κB (NF- |
| | | protein | κB)15, 17 and as a positive effector of PGC1α and FoxO |
| | | | through increased orexin type 2 receptor (Ox2r) expression. |
| 39 | Sirt6 | over express | Decreases IGF1 signaling and increases mito |
| | | protein | |
| 40 | Slc13a1 | Inhibitory pri- | Increases Sirt1 (by ≈60%), Cat (by ≈48%), Hdac3 (by |
| | | miRNA/shRNA | ≈22%), Trp53 and Cd55 |
| 41 | Slc13a5 (INDY) | Inhibitory pri- | By decreasing INDY you can activate hepatic AMPK, |
| | | miRNA/shRNA | induces PGC-1α, inhibits ACC-2, and reduces SREBP-1c |
| | | | levels. This signaling network promotes hepatic |
| | | | mitochondrial biogenesis, lipid oxidation, and energy |
| | | | expenditure and attenuates hepatic de novo lipogenesis |
| 42 | TERT | over express | Telomerase extends DNA ends and also promotes other anti |
| | | protein | aging effects and possible cell immortalization |
| 43 | TFAM | over express | Mitochondrial biogenesis and decreased ROS |
| | | protein | |
| 44 | TFEB | over express | Increases lysosomal biogenesis It encodes a transcription |
| | | protein | factor that coordinates expression of lysosomal hydrolases, |
| | | | membrane proteins and genes involved in autophagy. |
| 45 | sTGFbR2 | over express | Decreases fibrosis and inflammatory signaling by blocking |
| | (e.g., sTGFbR2-Fc) | soluble receptor | the effects of TGFB1 and has immune modulating effects |
| | | protein | and can rejuvenate aged neurons and skeletal muscle |
| 46 | Txn1 | over express | Acts through AP1 and NFkb by decreasing some parts of |
| | | protein | NFkb signaling and protecting from oxidative DNA damage |
| | | | and other protein redox states |
| 47 | Ubd | Inhibitory pri- | AMPK and UCP1, FAT10/Ubd regulates lifespan through |
| | | miRNA/shRNA | pleiotropic actions on metabolism and inflammation. |
| 48 | UCP1 | over express | Fat only. Increases thermogenesis and energy expenditure |
| | | protein | |
| 49 | BMP2 | over express | Increases Bone mass by increasing Osteoblasts |
| | | protein | |
| 50 | BMP4 | over express | Increases Bone mass by increasing Osteoblasts |
| | | protein | |
| 51 | Sema3a | over express | Increases Bone mass by decreasing osteoclasts and |
| | | protein | increasing osteoblasts |
| 52 | GDF8 | Inhibitory pri- | Increases Muscle mass |
| | | miRNA/shRNA | |
| 53 | Follistatin | over express | Increases Muscle mass |
| | | protein | |

The description in Table 1 identifies exemplary genes and whether the gene is over expressed in the method (“over expressed”) or inhibited (“pri-miRNA/shRNA”). As such, where the description makes reference to “overexpressed” gene, the gene therapy refers to use of a nucleic acid encoding the indicated protein product and where the protein product is overexpressed. Thus, in such descriptions, a reference to an identified gene also refers to the protein encoded by the gene. For example, “klotho” may refer to both the gene and the protein encoded by the gene. In some embodiments, the nucleic acid can encode a protein product, which is a mutated form of the naturally occurring expressed protein. By way of example and not limitation, Adra1a (mut) refers to a nucleic acid sequence encoding a mutated form of the naturally occurring Adra1a protein product, where the expressed mutated form of the receptor protein is constitutively active. Where the description in Table 1 makes reference to “pri-miRNA/shRNA,”the gene therapy refers to use of a nucleic acid which has a sequence for an expressed pri-miRINA/shRNA where the expressed pri-miRINA/shRNA inhibits or ultimately attenuates expression of the gene product of the target gene.

In some embodiments, a nucleic acid for gene therapy can use sequences which are homologous to the gene sequences provided herein or known in the art and are functional as the reference protein, inhibitory RNA or inhibitory protein. Accordingly, the present disclosure contemplates the nucleic acid sequences described herein and those that are at least 80%, at least 85%, at least 90%, at least 95%, at least 96%, at least 97%, at least 98% or at least 99% homologous thereto. Likewise, the present disclosure contemplates the amino acid sequences described herein and those that are at least 80%, at least 85%, at least 90%, at least 95%, at least 96%, at least 97%, at least 98% or at least 99% homologous thereto, such that the protein retains function or activity, at least in whole or in part. It is to be understood that one of skill can readily design different nucleic acid sequences than those identified herein or known in the art to encode a known protein based on the degeneracy of the genetic code. Accordingly, it is to be understood that identification of specific nucleic acid sequences herein is not intended to be limiting.

It is to be understood that each gene and thus the corresponding nucleic acid in Table 1 can be separately used in the gene therapy method to produce the desired physiological (e.g., therapeutic) effect. In some embodiments, a combination of the nucleic acids in Table 1 can be used in the gene therapy method to produce the desired therapeutic effect. As such, the present disclosure encompasses each and every possible combination of the gene and corresponding nucleic acid in Table 1 for use in gene therapy, as described herein. In some embodiments, the gene therapy includes combinations of any 2, 3, 4, 5, 6, 7, 8, 9, 10, 11, 12, 13, 14, 15 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, or all of the nucleic acids in Table 1, where the combination has the intended therapeutic effect, particularly with regards to treating or preventing an age-related disease or condition. It is to be understood that one of skill in the art can readily envision combinations and subcombinations of the nucleic acids for use in a therapeutic method.

According to one aspect, one or more of the genes in Table 1, such as FGF21 or Klotho, can be operably linked to a stabilizing peptide, such as is exemplified by sTGFbR2-FC, so as to increase its half life. One of skill can readily identify suitable fusion peptides, an example of which is FC. Also, the present disclosure contemplates the modification of one or more of the genes listed in Table 1, such as FGF21 and Klotho, so as to increase stability or half life of the protein encoded by the gene.

According to one aspect, one or more of the genes in Table 1, such as FGF21 or Klotho, can be operably linked to a secretion signal, such that the secretion signal is attached to the secreted protein in a manner to enhance expression. One of skill can readily identify suitable secretion signals and methods of adding a secretion signal to a secreted protein for enhanced expression.

In some embodiments identified in Table 2, a method of gene therapy for treating or preventing age related diseases or conditions comprises administering to a subject in need thereof an effective amount of a vector or plurality of vectors expressing the following genes, which express the gene product(s) as described in Table 1 above.

TABLE 2

| Therapy | The unique combination of genes used for a particular therapy and administered by |
|---|---|
| group | viral vector or a Cas9 guide RNA system |
| 1 | GDF15; |
| 2 | TERT and BubR1; |
| 3 | GDF15, TERT and BubR1; |
| 4 | GDF15, TERT, BubR1, Agtra1a, Adcy5, and Coq7; |
| 5 | GDF15, TERT, BubR1, Agtra1a, Adcy5, Coq7, Slc13a1, and Ikbkb; |
| 6 | BubR1, Cis2d, Txn1, FGF21, BubR1, Agtr1a, ikbkb, mTOR, Nudt1, Slc13a5, pappa, |
| | Coq7, Sdcy5, Agtr1a and Ctf1/akt1; |
| 7 | FGF21, Nrf2, sTGFbR2-Fc, HAS2, Nudt1, TERT, BubR1, Par4, Ubd, Dgat1, Ctf1, Coq7 |
| | Adcy5, Agtr1a, and mTOR; |
| 9 | Atg5, Nudt1, Adra1a (mut), NGF, NEU1, humanized foxP2, TFEB, PDE4b, mTOR, |
| | Slc13a5, Slc13a5, Coq7, Akt1, ikbkb and Slc13a1; |
| 10 | klotho, GDF15 (hNAG), sIGF1r-Fc, Mt1, Adra1a(mut), Nrf2, Rps6kb1, PCsk9, Prkar2b, |
| | Dgat, Ctf1, Coq7, papa, and ikbkb. |
| 11 | Atg5, Cebpa, pb, Ctf1, akt1, Pck1, adiponectin, PcsK9, Nrf2, Cisd2, papa, Dgat, Ctf1, |
| | Coq7, and mTOR. |
| 12 | FGF21, GDF15, klotho, Adra1a (mut), Sirt6, Bubr1, Par4, Coq7, Adcy5, Agtr1a, Agtr1a, |
| | ikbkb, mTOR, Slc13a1, papa, Ctf1, Ctf1, and Slc13a5 |
| 13 | FGF21, GDF15, klotho, TERT, sIGF1r-Fc, Bubr1, Par4, Rps6kb1, PCSk9, Adcy5, |
| | Coq7, Agtr1a, ikbkb, mTOR, and Slc13a1. |
| 14 | klotho, Txn1, Nrf2, TFEB, sTGFbr2-Fc, Nudt1, mt1, Atg5, Bubr1, Par4, Ctf1, Coq7 and |
| | ikbkb. |
| 15 | FGF21, sIGF1r-Fc, klotho, sTGFbr2-Fc, GDF15, HAS2, Mt1, Txn1, Nrf2, mCAT, |
| | Adra1a (mut), TFEB, Bubr1, Par4, Atg5, Cisd2, Nudt1, Sirt1, Sirt6, mTOR, slc13a5, |
| | pappa, ikbkb, adcy5, agtr1a and akt1. |
| 16 | TFEB, Atg5, klotho, UCP1, Cebpbeta, miCebpa, adiponectin, Mt1, Txn1, Nrf2, mCAT, |
| | TERT, Bubr1, Par4, TFAM, Cisd2, Nudt1, Neu1, NGF, Sirt6, Dgat, prkar2b, insr, ubd, |
| | Coq7, Ctf1, mTOR, and Slc13a5 |
| 17 | sTGFbR2-FC and Nrf2 |
| 18 | FGF21, TERT, BubR1, Agtra1a, Adcy5, Coq7, Slc13a1, Ikbkb, Klotho, GDF15, CTF1, |
| | mTOR, Slc13a5, Pappa, Pcsk9, and Rps6kb1 |
| 19 | FGF21, GDF15, Klotho, Adra1a (mut), Sirt6, BubR1, Agtra1a, Adcy5, Akt1, MCAT, |
| | Slc13a1, Ikbkb, Ctf1, mTOR, Coq7, and Slc13a5 |
| 20 | Txn1, Sirt6, Mt1, TFEB, Pck1, Adiponectin, Cisd2, Nudt1, Atg5, Ctf1, Ikbkb, and Coq7 |
| 21 | Fgf21, Nrf2, sTGFbR2-FC, Has2, NudT1, TERT, BubR1, Dgat1, Pappa, Ctf1, mTOR, |
| | Coq7, Slc13a5, Agtra1a, Adcy5, and Akt1 |
| 22 | Ctf1, Coq7, Agtra1a, Adcy5, mTOR, Cisd2, MCAT, FGF21, GDF15, Klotho, Slc13a1, |
| | Ikbkb, Txn1, and Sirt6 |
| 23 | Klotho, GDF15, Neu1, Mt1, Adra1a, hFoxP2, PCSK9, Rps6kb1, Ctf1, Ikbkb, Coq7, |
| | Slc13a1, mTOR, and NudT1 |
| 24 | Atg5, Ctf1, Akt1, BubR1, Pck1, Adiponectin, TERT, Nrf2, Cisd2, Dgat1, Pappa, Ctf1, |
| | mTOR, Coq7, and Slc13a5 |
| 25 | FGF21 and BMP2 |
| 26 | FGF21 and BMP4 |
| 27 | FGF21 and Sema3a |
| 28 | FGF21, BMP2, and BMP4 |
| 29 | FGF21, BMP2, and Sema3a |
| 30 | FGF21, BMP4, and Sema3a |
| 31 | FGF21, BMP2, BMP4, and Sema3a |
| 32 | FGF21 and Klotho |
| 33 | FGF21 and sTGFbR2-FC |
| 34 | Klotho and sTGFbR2-FC |
| 35 | FGF21, Klotho, and sTGFbR2-FC |

In the foregoing exemplary embodiments, where there are two or more genes used for the gene therapy, the genes can be contained in separate gene delivery vectors, either individually or where permissible (e.g., based on the capacity of the viral gene therapy vector) in certain combinations, such as based on the intended target tissue, as further described below.

As further described in detail herein, the nucleic acids in Table 1 and corresponding nucleic acid constructs, expression cassettes, expression vectors, expression control sequences, promoters and other elements related to the delivery of the nucleic acid sequence can be constructed as a gene delivery vector, such as a viral vector. The vector is administered to a mammal under conditions that result in expression of the nucleic acid which alters the levels of a functional protein in a manner to provide a preventative or therapeutic effect. Accordingly, the present disclosure also contemplates a vector, particularly a viral vector, more particularly an AAV vector for each and every one of the genes and corresponding nucleic acids listed in Table 1. Details of such viral vectors are described below.

In some embodiments, the nucleic acids of Table 1 can be collectively regulated to produce the desired therapeutic effect. The gene therapy and the corresponding nucleic acid used can be grouped based on the desired effect, including, among others, effects on metabolism, IGF1 or GH signaling, protein synthesis or autophagy, inflammation, fibrosis or immune response, genome stability, cancer, mitochondrial fitness number or function, oxidation or neuronal function.

Table 3 identifies exemplary genes and their grouping based on the effects on the indicated biological function which can be collectively regulated to achieve a desired effect.

TABLE 3

| | | |
|---|---|---|
| | | Reported literature effect on |
| Category | Gene | median lifespan or expected |

Metabolism

| | |
|---|---|
| FGF21 | 36 |
| GDF15 (hNAG) | 35 |
| Slc13a1 | 25 |
| mTOR | 20 |
| Cebpa/Cebpb | 20 |
| Dgat1 | 20 |
| Insr | 18 |
| Ubd | 15 |
| Prkar2b | 14 |
| UCP1 | 10* |
| Pck1 | 10* |
| Sirt1 | 10* |
| Adiponectin | 0* |
| AMPK | 0* |
| PCSK9 | 0* |
| Slc13a5 (INDY) | 0* |

IGF1/GH

| | |
|---|---|
| FGF21 | 36 |
| GDF15 (hNAG) | 35 |
| Pappa | 30 |
| Klotho | 21 |
| mTOR | 20 |
| Sirt6 | 12 |
| sIFG1r-Fc | 8 |
| Akt1 | 8 |
| Rps6kb1 (S6K1) | 0* |

Protein Synthesis/Autophagy

| | | |
|---|---|---|
| 20 | mTOR | 20 |
| 20 | Cisd2 | 20 |
| 17 | Atg5 | 17 |
| 8 | Akt1 | 8 |
| 0* | TFEB | 0* |

Inflammatory/fibrosis/immune

| | |
|---|---|
| Klotho | 21 |
| Ctf1 | 18 |
| Txn1 | 15 |
| Nrf2 | 10* |
| Sirt1 | 10* |
| sTGFbR2-FC | 0* |

Genome Stability/Cancer

| | |
|---|---|
| Coq7 | 23 |
| Ctf1 | 18 |
| NUDT1 | 16 |
| BubR1 | 15 |
| TERT | 15 |
| Par4 SAC domain | 10 |
| HAS2 | 0* |

Mitochondrial/Oxidative

| | |
|---|---|
| Adcy5 | 30 |
| Agtr1a | 26 |
| Sic13a1 | 25 |
| Coq7 | 23 |
| Cisd2 | 20 |
| MCAT | 20 |
| Mt1 | 14 |
| Sirt6 | 12 |
| Pck1 | 10* |
| Nrf2 | 10* |
| TFAM | 0* |

Neurological

| | |
|---|---|
| Ikbkb | 23 |
| NUDT1 | 16 |
| Adra1a (mut) | 10* |
| NGF | 0* |
| NEU1 | 0* |
| humanizeFoxP2 | 0* |
| PDE4b | 0* |

Bone and Muscle

| | |
|---|---|
| BMP2 | 0* |
| BMP4 | 0* |
| Sema3a | 0* |
| Follistatin | 0* |
| GDF8 | 0* |

10* indicates an hypothesized effect.
0* indicates a positive effect on lifespan.

In some embodiments, the gene therapy method is directed to the exemplary combinations of the nucleic acids (i.e. the genes) provided in each of the groups in Tables 2 or 3. In some embodiments, the gene therapy includes use of the combination or subcombination of nucleic acids for FGF21, GDF15 (hNAG), Slc13a1, mTOR, Cebpa/Cebpb, Dgat1, Insr, Ubd, Prkar2b, UCP1, Pck1, Sirt1, Adiponectin, AMPK, PCSK9 and Slc13a5 (INDY), as provided in Table 1, such as for treating or preventing metabolic conditions or diseases associated with aging.

In some embodiments, the gene therapy includes use of the combination or subcombination of nucleic acids for FGF21, GDF15 (hNAG), Pappa, Klotho, mTOR, Sirt6, sIFG1r-Fc, Akt1, and Rps6 kb1 (S6K1), as provided in Table 1, such as for treating or preventing conditions or diseases associated with IGF1/GH activity, particularly with regards to such activity involved in an age related disease or condition.

In some embodiments, the gene therapy includes use of the combination or subcombination of nucleic acids for mTOR, Cisd2, Atg5, Akt1, TFEB, as provided in Table 1, such as for treating or preventing conditions or diseases associated with protein synthesis and autophagy, particularly with regards to such activity involved in an age related disease or condition.

In some embodiments, the gene therapy includes use of the combination or subcombination of nucleic acids for Klotho, FGF21, Ctf1, Txn1, Nrf2, Sirt1 and sTGFbR2-FC, as provided in Table 1, such as for treating or preventing inflammation, fibrosis, immune conditions or diseases particularly with regards to such activity involved in an age related disease or condition. Exemplary gene combinations include FGF21 and Klotho; FGF21 and STGFbR2-FC; Klotho and sTGFbR2-FC or FGF21, Klotho, and sTGFbR2-FC.

In some embodiments, the gene therapy includes use of the combination or subcombination of nucleic acids for Coq7, Ctf1, NUDT1, BubR1, TERT, Par4 SAC domain, and HAS2, as provided in Table 1, such as for treating or preventing DNA damage, genome instability or cancer, particularly with regards to such activity, e.g., DNA damage or genome instability, involved in an age related disease or condition.

In some embodiments, the gene therapy includes use of the combination or subcombination of nucleic acids for Adcy5, Agtr1a, Cisd2, Coq7, mCAT, Mt1, Pck1, Sirt6, Slc13a1, Nrf2, and TFAM, as provided in Table 1, such as for treating or preventing conditions or diseases associated with mitochondrial function or oxidative damage, particularly with regards to such activity involved in an age related disease or condition.

In some embodiments, the gene therapy includes use of the combination or subcombination of nucleic acids for Ikbkb, NUDT1, Adra1a (mut), NGF, NEU1, humanizeFoxP2 and PDE4b, as provided in Table 1, such as for treating or preventing neurological conditions or diseases, such as cognitive impairment or cognitive decline, particularly with regards neurological conditions or diseases associated with aging.

It is also to be understood that the groups of the genes and corresponding nucleic acids used for treating or preventing conditions or diseases in the corresponding class of biological processes can be used in combination with one or more of the other groups of the genes and corresponding nucleic acids to treat more than one class of biological processes, particularly as those biological processes are associated with aging. Accordingly, encompassed within the present disclosure are gene therapy methods using every possible combination of the genes and corresponding nucleic acids listed in Table 1 or identified in the different groups in Table 2 or Table 3 above. Groups of the genes and corresponding nucleic acids used in gene therapy for (i) treating or preventing metabolic conditions or diseases, (ii) IGF1/GH activity associated conditions or diseases, (iii) conditions or diseases associated with protein synthesis and autophagy, (iv) inflammation, fibrosis, immune conditions or diseases, (v) DNA damage, genome instability or cancer, (vi) conditions or diseases associated with mitochondrial function or oxidative damage; and (vii) neurological conditions or diseases, can be used in combination or subcombination to treat multiple classes of diseases or conditions, particularly those multiple diseases or conditions relate to aging. By way of example, the combination or subcombination of the group of genes and corresponding nucleic acids for treating or preventing neurological diseases or conditions (vii) can be used together with the combination or subcombination of the group of genes and corresponding nucleic acids for treating or preventing diseases or conditions associated with mitochondrial function, i.e., group and oxidative damage, i.e., group (iv). Other such exemplary combinations include, by way of example and not limitation, combinations of 2, 3, 4, 5, 6 or all 7 of the foregoing groups (i) to (vii).

In some embodiments, the set of genes and corresponding nucleic acids for gene therapy methods herein can also be selected based on the tissue type targeted for gene therapy. Appropriate gene delivery constructs for expression in a specified tissue can incorporate the relevant nucleic acids for gene therapy. In some embodiments, the tissue specific delivery is based on choice of the appropriate viral vectors and viral packaging systems. The viral vectors can incorporate suitable promoters and other transcription regulators that allow expression of the gene product in the targeted tissue. The viral packaging systems can use the host cell range specificity of the viral components used to package the viral vectors so that the gene therapy vector is delivered to the targeted tissue. In the context of AAV vectors and capsid design, AAV serotypes, either naturally occurring or synthetic derivatives, can be used to manipulate the tropism range for gene therapy applications, as further described in detail below. For example, neuronal targeted genes may use a viral capsid designed to cross the blood-brain barrier, such as AAV9, and a liver targeted genes may use AAV8 which does not cross the blood-brain barrier to the same extent but does accumulate in the liver and muscle.

Other tissue specific methods can be used to limit expression in the tissue of interest, including, among others, use of tissue specific promoters and miRNA binding sites targeting those sequences expressed in the target tissue(s). Table 4 provides exemplary gene therapy nucleic acid as defined herein, the targeted cell or tissue, the AAV serotype or combination of serotypes having the appropriate tropism for the target tissue, exemplary promoters which function in the specific cell or target tissue to regulate expression, the administration route, and the size of the gene: A=adipose tissue, M=muscle tissue, B=brain tissue, L=liver tissue, E=systemic delivery throughout the organism, N=Not brain tissue, and H=cardiac tissue. Table 4 also indicates whether the gene product is an expressed protein or an inhibitor RNA. In some embodiments, the nucleic acid for gene therapy includes an expression inhibitor element which when expressed inhibits or attenuates expression of the gene product in one or more non-target tissue, also referred to as detargeting (see, e.g., Broderick et a., (2011) Gene Ther. 18 (2):1104-1110, incorporated herein by reference). In Table 4, an exemplary inhibitor element is a sequence for a miRNA at the 3′ UTR of the expressed mRNA such that the mRNA (or other transcribed RNA such as pri-miRNA/shRNA) is silenced in the specified non-target tissue, such as the liver. Various miRNAs for use in detargeting expression in non-target tissues include, among others, miRNA-122 for silencing expression in hepatocytes, miRNA-124 for silencing expression in neuronal cells, and miRNA-142 for silencing expression in hematopoietic cells. Other miRNAs known in the art for inhibiting expression in particular cells and tissues can be used in the present gene therapy applications by the person of skill in the art. One or a combination of such silencing miRNA targeting sequences can be used to inhibit or attenuate undesirable expression of the gene therapy construction in one or more non-target cells and tissues, where the non-target tissues can be different from each other.

TABLE 4

| | | Target | | | 3′ miRNA | Expressed | |
|---|---|---|---|---|---|---|---|
| | Gene | Tissue | AAV type | Promoter | silencer | Gene product | Size |
| 1 | UCP1 | A | AAV:2/8 | adipose | prevent | over express | 924 |
| | | | | | liver | protein | |
| | | | | | expression | | |
| 2 | Cebpbeta | A | AAV:2/8 | adipose | prevent | over express | 891 |
| | | | | | liver | protein | |
| | | | | | expression | | |
| 3 | Adiponectin | S, A | AAV:2/8 | adipose | prevent | over express | 800 |
| | | | | | liver | protein | |
| | | | | | expression | | |
| 4 | sIFG1r-fc | S, L | AAV:2/8 | hEf1a | None | over express | 3507 |
| | | | | | | soluble receptor | |
| | | | | | | protein | |
| 5 | sTGFbr2- | S, L | AAV:2/8 | hEf1a | None | over express | 1251 |
| | Fc | | | | | soluble Rec | |
| 6 | FGF21 | S, L | AAV:2/8 | hEf1a | None | over express | 633 |
| | | | | | | protein | |
| 7 | GDF15 | S, L | AAV:2/8 | hEf1a | None | over express | 912 |
| | (hNAG) | | | | | protein | |
| 8 | Klotho | S, L | AAV:2/8 | hEf1a | None | over express | 3045 |
| | | | | | | protein | |
| 9 | HAS2 | S, N | AAV:2/8 | hEf1a | None | over express | 1659 |
| | | | | | | protein | |
| 10 | Mt1 | H | AAV:2/9 | hEf1a | None | over express | 399 |
| | | | | | | protein | |
| 11 | Nrf2 | E | AAV:2/9 | hEf1a | None | over express | 1794 |
| | | | | | | protein | |
| 12 | Par4 SAC | E, B | AAV:2/9 | hEf1a | None | over express | 180 |
| | domain | | | | | protein | |
| | 137-195 | | | | | | |
| 13 | Txn1 | E | AAV:2/9 | hEf1a | None | over express | 318 |
| | | | | | | protein | |
| 14 | mCat | M, H | AAV9 | hEf1a | None | over express | 1671 |
| | | | | | | protein | |
| 15 | Pck1 | M | AAV:2/9 | muscle | prevent | over express | 1869 |
| | | | | specific | liver | protein | |
| | | | | | expression | | |
| 16 | Adra1a | B, H | AAV:2/PHP B | hEf1a | None | over express | 1401 |
| | (mut) | | | | | protein | |
| 17 | BubR1 | E | AAV:2/PHP B | hEf1a | None | over express | 3159 |
| | | | | | | protein | |
| 18 | TERT | E | AAV:2/PHP B | hEf1a | None | over express | 3424 |
| | | | | | | protein | |
| 19 | TFAM | E | AAV:2/PHP B | hEf1a | None | over express | 732 |
| | | | | | | protein | |
| 20 | TFEB | E | AAV:2/PHP B | hEf1a | None | over express | 1605 |
| | | | | | | protein | |
| 21 | Humanized | B | AAV:2/PHP B | Brain | prevent | over express | 2145 |
| | FoxP2 | | | | liver | protein | |
| | | | | | expression | | |
| 22 | NEU1 | B | AAV:2/PHP B | Brain | prevent | over express | 1230 |
| | | | | | liver | protein | |
| | | | | | expression | | |
| 23 | NGF | B | AAV:2/PHP B | Brain | prevent | over express | 1124 |
| | | | | | liver | protein | |
| | | | | | expression | | |
| 24 | Atg5 | E | AAV:2/PHP | hEf1a | None | over express | 828 |
| | | | B or | | | protein | |
| | | | AAV:2/9 | | | | |
| 25 | Cisd2 | E, M, B | AAV:2/PHP | hef1a | None | over express | 408 |
| | | | B or | | | protein | |
| | | | AAV:2/9 | | | | |
| 26 | NUDT1 | E, B | AAV:2/PHP | hEf1a | None | over express | 471 |
| | | | B or | | | protein | |
| | | | AAV:2/9 | | | | |
| 27 | Sirt1 | E, B | AAV:2/PHP | hEf1a | None | over express | 2214 |
| | | | B or | | | protein | |
| | | | AAV:2/9 | | | | |
| 28 | Sirt6 | E | AAV:2/PHP | hEf1a | None | over express | 993 |
| | | | B or | | | protein | |
| | | | AAV:2/9 | | | | |
| 29 | Dgat1 | A | AAV:2/8 | adipose | prevent | Inhibitory pri- | 363 |
| | | | | | liver | miRNA/shRNA | |
| | | | | | expression | | |
| 30 | Prkar2b | A | AAV:2/8 | adipose | prevent | Inhibitory pri- | 363 |
| | | | | | liver | miRNA/shRNA | |
| | | | | | expression | | |
| 31 | Insr | A | AAV:2/8 | adipose | prevent | Inhibitory pri- | 363 |
| | | | | | liver | miRNA/shRNA | |
| | | | | | expression | | |
| 32 | Ubd | A | AAV:2/8 | adipose | prevent | Inhibitory pri- | 363 |
| | | | | | liver | miRNA/shRNA | |
| | | | | | expression | | |
| 33 | Cebpalpha | A | AAV:2/8 | adipose | prevent | Inhibitory pri- | 363 |
| | | | | | liver | miRNA/shRNA | |
| | | | | | expression | | |
| 34 | PCSK9 | L | AAV:2/8 | hEf1a | None | Inhibitory pri- | 363 |
| | | | | | | miRNA/shRNA | |
| 35 | Rps6kb1 | L | AAV:2/8 | hEf1a | None | Inhibitory pri- | 363 |
| | (S6K1) | | | | | miRNA/shRNA | |
| 36 | Slc13a5 | L | AAV:2/8 | hEf1a | None | Inhibitory pri- | 363 |
| | (INDY) | | | | | miRNA/shRNA | |
| 37 | Pappa | E, M | AAV:2/9 | hEf1a | None | Inhibitory pri- | 363 |
| | | | | | | miRNA/shRNA | |
| 38 | Ikbkb | B | AAV:2/PHP B | hEf1a | none | Inhibitory pri- | 363 |
| | | | | | | miRNA/shRNA | |
| 39 | ADcy5 | E | AAV:2/PHP B | hEf1a | None | Inhibitory pri- | 363 |
| | | | | | | miRNA/shRNA | |
| 40 | Agtr1a | E | AAV:2/PHP B | hEf1a | None | Inhibitory pri- | 363 |
| | | | | | | miRNA/shRNA | |
| 41 | Akt1 | E | AAV:2/PHP B | hEf1a | None | Inhibitory pri- | 363 |
| | | | | | | miRNA/shRNA | |
| 42 | Coq7 | E | AAV:2/PHP B | hef1a | None | Inhibitory pri- | 363 |
| | | | | | | miRNA/shRNA | |
| 43 | Ctf1 | E | AAV:2/PHP B | hef1a | None | Inhibitory pri- | 363 |
| | | | | | | miRNA/shRNA | |
| 44 | PDE4b | B | AAV:2/PHP B | hEf1a | none | Inhibitory pri- | 363 |
| | | | | | | miRNA/shRNA | |
| 45 | mTOR | E | AAV:2/PHP | hEf1a | None | Inhibitory pri- | 363 |
| | | | B or | | | miRNA/shRNA | |
| | | | AAV:2/9 | | | | |
| 46 | Slc13a1 | E | AAV:2/PHP | hEf1a | None | Inhibitory pri- | 363 |
| | | | B or | | | miRNA/shRNA | |
| | | | AAV:2/9 | | | | |
| 47 | AMPK | M | AAV:2/PHP | Muscle | Prevent | Over express | 1680 |
| | | | B or | and | Liver | Protein | |
| | | | AAV:2/9 | adipose | | | |
| 48 | Nf-Kb | E | AAV:2/PHP | hEf1a | None | Inhibitory pri- | 363 |
| | | | B or | | | miRNA/shRNA | |
| | | | AAV:2/9 | | | | |
| 49 | BMP2 | L | AAV:2/8 | hEf1a | None | Over express | |
| | | | | | | Protein | |
| 50 | BMP4 | L | AAV:2/8 | hEf1a | None | Over express | |
| | | | | | | Protein | |
| 51 | Sema3a | Bone | AAV:2/8 | hEf1a | None | Over express | |
| | | | | | | Protein | |
| 52 | GDF8 | L | AAV:2/8 | hEf1a | None | Over express | |
| | | | | | | Protein | |
| 53 | Follistatin | L | AAV:2/8 | hEf1a | None | Over express | |
| | | | | | | Protein | |

In view of the description in Table 4, the present disclosure is directed to an exemplary gene therapy vector containing the elements (i.e., gene, promoter, miRNA silencer) specified for each of embodiments 1-53 in Table 4. In some embodiments, the gene therapy vector can be based on vector construct hEf1α-WPRE3-SV40, where Hef1a refers to human elongation factor 1a promoter; WPRE3 is a truncated version of the woodchuck hepatitis posttranscriptional regulatory element; and SV40 is a truncated version of the SV40 polyadenylation site (PMCID:PMC3975461). The present disclosure is directed to recombinant AAV viral particles having the specific AAV serotype and specified vector elements for each of embodiments 1-53 in Table 4. The AAV capsid protein specifying the serotype of the recombinant viral particle can be provided using the appropriate AAV helper viruses. See, e.g., Yuan et al., 2011, Hum Gene Ther. 22 (5):613-24, incorporated herein by reference). The hEf1a can also refer to a truncated version of the hEf1a promoter that is 231 bp long and referenced as SEQ ID NO:18.

In view of the capacity of gene therapy vectors for delivering nucleic acids into target cells, in some embodiments, the viral vector can have two or more nucleic acids for expression of two or more corresponding functional proteins, inhibitory RNA, or inhibitory proteins. Each of the nucleic acids for expressing the different gene expression products can have its own transcription regulatory elements, and if expressing a protein, separate translational regulatory elements, such that separate RNAs are expressed. In some embodiments, a single RNA can be expressed in a cistronic form, where the gene products are expressed from the single RNA. Thus in some embodiments, the gene therapy vector can have polycistronic elements, such as internal ribosome entry sites (IRES) or 2A sequences (PMCID:PMC3084703) for inducing ribosome skipping as they may be required to express the different gene therapy products from the single RNA.

In some embodiments, in gene therapy with a plurality of nucleic acids expressing a plurality of different gene products, two or more gene delivery vectors, particularly viral vectors, are administered to a mammal. Accordingly, in some embodiments, the present disclosure provides for the concurrent or separate administration of 2, 3, 4, 5, 6, 7, 8, 9, 10, 11, 12, 13, 14, 15, 16, 17, 18, 19, 20 or more separate vectors for delivering the relevant genes in methods of gene therapy. Amounts of each separate vector to be administered alone or as a combination of vectors can be determined based upon, among others, the vector design, the nucleic acid sequences to be delivered, efficiency of delivery to target tissue, mode of administration, and the intended therapeutic effect. In various embodiments, the optimal ratio of each viral vector to be administered concurrently can be assessed from the maximum viral dose for each subject and by the effectiveness of each individual vector. A person of skill can determine the proper ratios and doses based on the present disclosure.

An exemplary set of viral vectors with one or more genes for gene therapy are given in Table 2. For gene therapy with a combination of genes expressing the referenced protein and/or inhibitory RNA, a plurality of viruses as described are administered to a mammal in a method of gene therapy. Accordingly, the disclosure provides methods of administering multiple viruses including one or more or multiple genes, inhibitory RNAs or inhibitory proteins, examples and combinations of which are provided below, particularly for treating or preventing diseases or conditions associated with aging.

In Group 1, virus 1 includes AAV8-GFP as a control and virus 2 includes AAV9:GFP as a control.

In Group 2, a single virus including a single gene, GDF15, is administered to a mammal in a method of gene therapy.

In Group 3, virus 1 includes TERT and virus 2 includes BubR1.

In Group 4, virus 1 includes GDF15, virus 2 includes TERT and virus 3 includes BubR1.

In Group 5, virus 1 includes GDF15, virus 2 includes TERT, virus 3 includes FGF21 and virus 4 includes BubR1.

In Group 6, virus 1 includes GDF15, virus 2 includes TERT, virus 3 includes FGF21, virus 4 includes Klotho and virus 5 includes BubR1.

In Group 7, virus 1 includes BubR1, p2A and Par4, virus 2 includes Cis2d, virus 3 includes Txn1, virus 4 includes FGF21, virus 5 includes BubR1, virus 6 includes Agtr1a, ikbkb and mTOR, virus 7 includes Nudt1, virus 8 includes Slc13a5 and pappa, virus 9 includes Coq7, ASdcy5 and Agtr1a and virus 10 includes Ctf1/akt1.

In Group 8, virus 1 includes FGF21, virus 2 includes Nrf2, virus 3 includes sTGFbR2-Fc, virus 4 includes HAS2, virus 5 includes Nudt1, virus 6 includes TERT, virus 7 includes BubR1, p2A and Par4, virus 8 includes Ubd and Dgat1, virus 9 includes Ctf1 and Coq7 and virus 10 includes Adcy5, Agtr1a and mTOR.

In Group 9, virus 1 includes Atg5, virus 2 includes Nudt1, virus 3 includes Adra1a (mut), virus 4 includes NGF, virus 5 includes NEU1, virus 6 includes humanized foxP2, virus 7 includes TFEB, virus 8 includes PDE4b, mTOR, and Slc13a5, virus 9 includes Slc13a5, Coq7 and Akt1 and virus 10 includes ikbkb and Slc13a1.

In Group 10, virus 1 includes klotho, virus 2 includes GDF15 (hNAG), virus 3 includes sIGF1r-Fc, virus 4 includes Mt1, virus 5 includes Adra1a (mut), virus 6 includes Nrf2, virus 7 includes Rps6 kb1 and PCsk9, virus 8 includes Prkar2b and Dgat, virus 9 includes Ctf1 and Coq7 and virus 10 includes pappa and ikbkb.

In Group 11, virus 1 includes Atg5, virus 2 includes Cebpa and Cebpb, virus 3 includes Ctf1 and akt1, virus 4 includes Pck1, virus 5 includes adiponectin, virus 6 includes PcsK9, virus 7 includes Nrf2, virus 8 includes Cisd2, virus 9 includes pappa and Dgat and virus 10 includes Ctf1, Coq7 and mTOR.

In Group 12, virus 1 includes FGF21, virus 2 includes GDF15, virus 3 includes klotho, virus 4 includes Adra1a (mut), virus 5 includes Sirt6, virus 6 includes Bubr1, p2A and Par4, virus 7 includes Coq7, Adcy5 and Agtr1a, virus 8 includes Agtr1a, ikbkb and mTOR, virus 9 includes Slc13a1, pappa and Ctf1 and virus 10 includes Ctf1, Slc13a5 (AAV9).

In Group 13, virus 1 includes FGF21, virus 2 includes GDF15, virus 3 includes klotho, virus 4 includes TERT, virus 5 includes sIGF1r-Fc, virus 6 includes Bubr1, p2A and Par4, virus 7 includes Rps6 kb1 and PCSk9, virus 8 includes Adcy5 and Coq7, virus 9 includes Agtr1a and ikbkb and virus 10 includes mTOR and Slc13a1.

In Group 14, virus 1 includes klotho, virus 2 includes Txn1, virus 3 includes Nrf2, virus 4 includes TFEB, virus 5 includes sTGFbr2-Fc, virus 6 includes Nudt1, virus 7 includes mt1, virus 8 includes Atg5, virus 9 includes Bubr1, p2A and Par4 and virus 10 includes Ctf1, Coq7 and ikbkb.

In Group 15, virus 1 includes FGF21, IRES, and sIGF1r-Fc, virus 2 includes klotho, virus 3 includes sTGFbr2-Fc, IRES and GDF15, virus 4 includes HAS2, p2A, Mt1, and Txn1, virus 5 includes Nrf2, p2A and mCAT, virus 6 includes Adra1a (mut), p2A and TFEB, virus 7 includes Bubr1, p2A and Par4, virus 8 includes Atg5, p2A, Cisd2 and Nudt1, virus 9 includes Sirt1, p2A and Sirt6 and virus 10 includes mTOR, slc13a5, pappa, ikbkb, adcy5, agtr1a and akt1.

In Group 16, virus 1 includes TFEB, p2A and Atg5, virus 2 includes klotho, virus 3 includes UCP1, p2A, Cebpbeta and miCebpa, virus 4 includes adiponectin, IRES, Mt1, p2A and Txn1, virus 5 includes Nrf2, p2A and mCAT, virus 6 includes TERT, virus 7 includes Bubr1, p2A and Par4, virus 8 includes TFAM, p2A, Cisd2 and Nudt1, virus 9 includes Neu1, p2A, NGF and Sirt6 and virus 10 includes Dgat, prkar2b, insr, ubd, Coq7, Ctf1, mTOR and Slc13a5.

In Group 17, the viruses include sTGFbR2-FC and/or Nrf2.

In Group 18, the viruses include FGF21, TERT, BubR1, Agtra1a, Adcy5, Coq7, Slc13a1, Ikbkb, Klotho, GDF15, CTF1, mTOR, Slc13a5, Pappa, Pcsk9, and/or Rps6 kb1.

In Group 19, the viruses include FGF21, GDF15, Klotho, Adra1a (mut), Sirt6, BubR1, Agtra1a, Adcy5, Akt1, MCAT, Slc13a1, Ikbkb, Ctf1, mTOR, Coq7, and/or Slc13a5.

In Group 20, the viruses include Txn1, Sirt6, Mt1, TFEB, Pck1, Adiponectin, Cisd2, Nudt1, Atg5, Ctf1, Ikbkb, and/or Coq7.

In Group 21, the viruses include Fgf21, Nrf2, sTGFbR2-FC, Has2, NudT1, TERT, BubR1, Dgat1, Pappa, Ctf1, mTOR, Coq7, Slc13a5, Agtra1a, Adcy5, and/or Akt1.

In Group 22, the viruses include Ctf1, Coq7, Agtra1a, Adcy5, mTOR, Cisd2, MCAT, FGF21, GDF15, Klotho, Slc13a1, Ikbkb, Txn1, and/or Sirt6.

In Group 23, the viruses include Klotho, GDF15, Neu1, Mt1, Adra1a, hFoxP2, PCSK9, Rps6 kb1, Ctf1, Ikbkb, Coq7, Slc13a1, mTOR, and/or NudT1.

In Group 24, the viruses include Atg5, Ctf1, Akt1, BubR1, Pck1, Adiponectin, TERT, Nrf2, Cisd2, Dgat1, Pappa, Ctf1, mTOR, Coq7, and/or Slc13a5.

In Group 25, the viruses include FGF21 and/or BMP2.

In Group 26, the viruses include FGF21 and/or BMP4.

In Group 27, the viruses include FGF21 and/or Sema3a.

In Group 28, the viruses include FGF21, BMP2, and/or BMP4.

In Group 29, the viruses include FGF21, BMP2, and/or Sema3a.

In Group 30, the viruses include FGF21, BMP4, and/or Sema3a.

In Group 31, the viruses include FGF21, BMP2, BMP4, and/or Sema3a.

In Group 32, the viruses include FGF21 and Klotho.

In Group 33, the viruses include FGF21, sTGFbR2-FC.

In Group 34, the viruses include Klotho and sTGFbR2-FC.

In Group 35, the viruses include FGF21, Klotho and sTGFbR2-Fc.

Gene Therapy with Pri-miRNA/shRNA Against a Target Gene

In the present disclosure, a gene construct expressing a primary miRNA molecule (pri-miRNA) and/or short hairpin (shRNA) are used to inhibit or attenuate expression of a target gene. A single pri-miRNA may contain from one to six miRNA precursors, and is processed to produce miRNA, which is exported from the nucleus to the cytoplasm, where it silences expression of target RNAs. Exemplary hairpin loop structures are composed of about 70 nucleotides each. Each hairpin is flanked by sequences necessary for efficient processing.

The double-stranded RNA (dsRNA) structure of the hairpins in a pri-miRNA is recognized by a nuclear protein known as DiGeorge Syndrome Critical Region 8 (DGCR8 or “Pasha” in invertebrates), named for its association with DiGeorge Syndrome. DGCR8 associates with the enzyme Drosha, a protein that cuts RNA, to form the Microprocessor complex. See Lee, Y. et al., Nature 425 (6956): 415-9 (2003); Gregory R I. et al., (2006) Methods Mol. Biol. 342:33-47. In this complex, DGCR8 orients the catalytic RNase III domain of Drosha to liberate hairpins from pri-miRNAs by cleaving RNA about eleven nucleotides from the hairpin base (one helical dsRNA turn into the stem). See Han, J et al., (2004) Genes & Development 18(24):3016-27; Han, J. et al. (2006) Cell 125 (5): 887-901. The product resulting has a two-nucleotide overhang at its 3′ end; it has 3′ hydroxyl and 5′ phosphate groups. It is often termed as a pre-miRNA (precursor-miRNA). Sequence motifs downstream of the pre-miRNA that are important for efficient processing have been identified. Conrad, T. et al., Cell Reports 9 (2): 542-554; Auyeung, V. et al., (2013) Cell 152 (4): 844-858; Ali, P. S. et al., (2012) FEBS Letters 586 (22): 3986-90.

Pre-miRNAs that are spliced directly out of introns, bypassing the Microprocessor complex, are known as “Mirtrons.” Originally thought to exist only in Drosophila and C. elegans, mirtrons have now been found in mammals. See Berezikov E. et al., (2007) “Mammalian mirtron genes” Mol. Cell 28 (2): 328-36.

As many as 16% of pre-miRNAs may be altered through nuclear RNA editing. See Kawahara Y. et al., (2008) Nucleic Acids Res. 36 (16): 5270-80; Winter J. et al., (2009) Nat. Cell Biol. 11 (3): 228-34; Ohman M. (2007) Biochimie 89 (10): 1171-6. Most commonly, enzymes known as adenosine deaminases acting on RNA (ADARs) catalyze adenosineto inosine (A to I) transitions. RNA editing can halt nuclear processing (for example, of pri-miR-142, leading to degradation by the ribonuclease Tudor-SN) and alter downstream processes including cytoplasmic miRNA processing and target specificity (e.g., by changing the seed region of miR-376 in the central nervous system). See Kawahara Y, et al., (2008) Nucleic Acids Res. 36 (16): 5270-80.

Pre-miRNA hairpins are exported from the nucleus in a process involving the nucleocytoplasmic shuttler Exportin-5. This protein, a member of the karyopherin family, recognizes a two-nucleotide overhang left by the RNase III enzyme Drosha at the 3′ end of the pre-miRNA hairpin. Exportin-5-mediated transport to the cytoplasm is energy-dependent, using GTP bound to the Ran protein. See Murchison E. P. et al., (2004) Curr. Opin. Cell Biol. 16 (3): 223-9.

In the cytoplasm, the pre-miRNA hairpin is cleaved by the RNase III enzyme Dicer. See Lund E. et al., (2006) Cold Spring Harb. Symp. Quant. Biol. 71:59-66. This endoribonuclease interacts with 5′ and 3′ ends of the hairpin. See Park, J. E. et al., (2011) Nature 475 (7355): 201-5, and cuts away the loop joining the 3′ and 5′ arms, yielding an imperfect miRNA:miRNA*duplex about 22 nucleotides in length. See Lund E. et al., (2006) Cold Spring Harb. Symp. Quant. Biol. 71:59-66. Overall hairpin length and loop size influence the efficiency of Dicer processing. The imperfect nature of the miRNA:miRNA* pairing also affects cleavage. See Lund E. et al, (2006) Cold Spring Harb. Symp. Quant. Biol. 71:59-66; Ji X (2008) Current Topics in Microbiology and Immunology 320:99-116. Some of the G-rich pre-miRNAs can potentially adopt the G-quadruplex structure as an alternative to the canonical stem-loop structure. For example, human pre-miRNA 92b adopts a G-quadruplex structure which is resistant to the Dicer mediated cleavage in the cytoplasm. See Mirihana A. et al., (2015) Chem. Biol. 22:262-272. Although either strand of the duplex may potentially act as a functional miRNA, only one strand is usually incorporated into the RNA-induced silencing complex (RISC) where the miRNA and its mRNA target interact.

Gene Therapy with Cas9 Mediated Regulation of Functional Proteins

The present disclosure also provides method of regulating the target genes and their corresponding functional proteins described herein using a Cas9/guide RNA system with a transcriptional regulator. It is to be understood that one of skill will be able to design suitable guide RNA for forming a co-localization complex with a target nucleic acid including a target gene as described herein.

### Cas9 DNA Binding Proteins

RNA guided DNA binding proteins are readily known to those of skill in the art to bind to DNA for various purposes. Such DNA binding proteins may be naturally occurring. DNA binding proteins having nuclease activity are known to those of skill in the art, and include naturally occurring DNA binding proteins having nuclease activity, such as Cas9 proteins present, for example, in Type II CRISPR systems. Such Cas9 proteins and Type II CRISPR systems are well documented in the art. See Makarova et al., Nature Reviews, Microbiology, Vol. 9, June 2011, pp. 467-477 including all supplementary information hereby incorporated by reference in its entirety. Such RNA guided DNA binding proteins may include one or m ore nuclear localization signals attached thereto for facilitating transfer of the RNA guided DNA binding protein into the nuclease.

In general, bacterial and archaeal CRISPR-Cas systems rely on short guide RNAs in complex with Cas proteins to direct degradation of complementary sequences present within invading foreign nucleic acid. See Deltcheva, E. et al., (2011) Nature 471, 602-607; Gasiunas, G. et al., (2012) Proc Natl Acad Sci USA 109, E2579-2586; Jinek, M. et al. (2012) Science 337, 816-821; Sapranauskas, R. et al., (2011) Nucleic Acids Res 39:9275-9282; and Bhaya, D. et al., (2011) Ann Rev Gen 45:273-297. A recent in vitro reconstitution of the S. pyogenes type II CRISPR system demonstrated that crRNA (“CRISPR RNA”) fused to a normally trans-encoded tracrRNA (“trans-activating CRISPR RNA”) is sufficient to direct Cas9 protein to sequence-specifically cleave target DNA sequences matching the crRNA. Expressing a gRNA homologous to a target site results in Cas9 recruitment and degradation of the target DNA. See H. Deveau et al., (2008) J Bact 190, 1390. Additional useful Cas proteins are from S. thermophilis or S. aureus.

Three classes of CRISPR systems are generally known and are referred to as Type I, Type II or Type III). According to one aspect, a particular useful enzyme according to the present disclosure to cleave dsDNA is the single effector enzyme, Cas9, common to Type II. See K. S. Makarova et al., (2011) Nature Rev Microbiol. 9:467; all publications incorporated herein by reference in its entirety.

In S. pyogenes, Cas9 generates a blunt-ended double-stranded break 3 bp upstream of the protospacer-adjacent motif (PAM) via a process mediated by two catalytic domains in the protein: an HNH domain that cleaves the complementary strand of the DNA and a RuvC-like domain that cleaves the non-complementary strand. See Jinek et al., (2012) Science 337, 816-821, hereby incorporated by reference in its entirety. Cas9 proteins are known to exist in many Type II CRISPR systems including the following as identified in the supplementary information to Makarova et al., Nature Reviews, Microbiology, Vol. 9, June 2011, pp. 467-477: Methanococcus maripaludis C7; Corynebacterium diphtheriae; Corynebacterium efficiens YS-314; Corynebacterium glutamicum ATCC 13032 Kitasato; Corynebacterium glutamicum ATCC 13032 Bielefeld; Corynebacterium glutamicum R; Corynebacterium kroppenstedtii DSM 44385; Mycobacterium abscessus ATCC 19977; Nocardia farcinica IFM10152; Rhodococcus erythropolis PR4; Rhodococcus jostii RHA1; Rhodococcus opacus B4 uid36573; Acidothermus cellulolyticus 11B; Arthrobacter chlorophenolicus A6; Kribbella flavida DSM 17836 uid43465; Thermomonospora curvata DSM 43183; Bifidobacterium dentium Bd1; Bifidobacterium longum DJO10A; Slackia heliotrinireducens DSM 20476; Persephonella marina EX H1; Bacteroides fragilis NCTC 9434; Capnocytophaga ochracea DSM 7271; Flavobacterium psychrophilum JIP02 86; Akkermansia muciniphila ATCC BAA 835; Roseiflexus castenholzii DSM 13941; Roseiflexus RS1; Synechocystis PCC6803; Elusimicrobium minutum Pei191; uncultured Termite group 1 bacterium phylotype Rs D17; Fibrobacter succinogenes S85; Bacillus cereus ATCC 10987; Listeria innocua;Lactobacillus casei; Lactobacillus rhamnosus GG; Lactobacillus salivarius UCC118; Streptococcus agalactiae A909; Streptococcus agalactiae NEM316; Streptococcus agalactiae 2603; Streptococcus dysgalactiae equisimilis GGS 124; Streptococcus equi zooepidemicus MGCS10565; Streptococcus gallolyticus UCN34 uid46061; Streptococcus gordonii Challis subst CH1; Streptococcus mutans NN2025 uid46353; Streptococcus mutans; Streptococcus pyogenes M1 GAS; Streptococcus pyogenes MGAS5005; Streptococcus pyogenes MGAS2096; Streptococcus pyogenes MGAS9429; Streptococcus pyogenes MGAS10270; Streptococcus pyogenes MGAS6180; Streptococcus pyogenes MGAS315; Streptococcus pyogenes SSI-1; Streptococcus pyogenes MGAS10750; Streptococcus pyogenes NZ131; Streptococcus thermophiles CNRZ1066; Streptococcus thermophiles LMD-9; Streptococcus thermophiles LMG 18311; Clostridium botulinum A3 Loch Maree; Clostridium botulinum B Eklund 17B; Clostridium botulinum Ba4 657; Clostridium botulinum F Langeland; Clostridium cellulolyticum H10; Finegoldia magna ATCC 29328; Eubacterium rectale ATCC 33656; Mycoplasma gallisepticum; Mycoplasma mobile 163K; Mycoplasma penetrans; Mycoplasma synoviae 53; Streptobacillus moniliformis DSM 12112; Bradyrhizobium BTAil; Nitrobacter hamburgensis X14; Rhodopseudomonas palustris BisB18; Rhodopseudomonas palustris BisB5; Parvibaculum lavamentivorans DS-1; Dinoroseobacter shibae DFL 12; Gluconacetobacter diazotrophicus Pal 5 FAPERJ; Gluconacetobacter diazotrophicus Pal 5 JGI; Azospirillum B510 uid46085; Rhodospirillum rubrum ATCC 11170; Diaphorobacter TPSY uid29975; Verminephrobacter eiseniae EF01-2; Neisseria meningitides 053442; Neisseria meningitides alpha14; Neisseria meningitides Z2491; Desulfovibrio salexigens DSM 2638; Campylobacter jejuni doylei 269 97; Campylobacter jejuni 81116; Campylobacter jejuni; Campylobacter lari RM2100; Helicobacter hepaticus; Wolinella succinogenes; Tolumonas auensis DSM 9187; Pseudoalteromonas atlantica T6c; Shewanella pealeana ATCC 700345; Legionella pneumophila Paris; Actinobacillus succinogenes 130Z; Pasteurella multocida; Francisella tularensis novicida U112; Francisella tularensis holarctica; Francisella tularensis FSC 198; Francisella tularensis; Francisella tularensis WY96-3418; and Treponema denticola ATCC 35405. The Cas9 protein may be referred by one of skill in the art in the literature as Csn1. An exemplary S. pyogenes Cas9 protein sequence is provided in Deltcheva et al., (2011) Nature 471, 602-607, hereby incorporated by reference in its entirety.

Modification to the Cas9 protein is a representative embodiment of the present disclosure. CRISPR systems useful in the present disclosure are described in Barrangou, R. et al., (2012) Ann Rev Food Sci Technol. 3:143 and Wiedenheft, B. et al., (2012) Nature 482, 331, each of which are hereby incorporated by reference in their entireties.

According to certain aspects, the DNA binding protein is altered or otherwise modified to inactivate the nuclease activity. Such alteration or modification includes altering one or more amino acids to inactivate the nuclease activity or the nuclease domain. Such modification includes removing the polypeptide sequence or polypeptide sequences exhibiting nuclease activity, i.e., the nuclease domain, such that the polypeptide sequence or polypeptide sequences exhibiting nuclease activity, i.e. nuclease domain, are absent from the DNA binding protein. Other modifications to inactivate nuclease activity will be readily apparent to one of skill in the art based on the present disclosure. Accordingly, a nuclease-null DNA binding protein includes polypeptide sequences modified to inactivate nuclease activity or removal of a polypeptide sequence or sequences to inactivate nuclease activity. The nuclease-null DNA binding protein retains the ability to bind to DNA even though the nuclease activity has been inactivated. Accordingly, the DNA binding protein includes the polypeptide sequence or sequences required for DNA binding but may lack the one or more or all of the nuclease sequences exhibiting nuclease activity. Accordingly, the DNA binding protein includes the polypeptide sequence or sequences required for DNA binding but may have one or more or all of the nuclease sequences exhibiting nuclease activity inactivated. See Jinek et al., (2012) Science 337, 816-821. A Cas9 protein lacking nuclease activity is referred to as a nuclease-null Cas9 (“Cas9Nuc”) and exhibits reduced or eliminated nuclease activity, or nuclease activity is absent or substantially absent within levels of detection. According to this aspect, nuclease activity for a Cas9Nuc may be undetectable using known assays, i.e. below the level of detection of known assays.

According to one aspect, the Cas9 protein includes the sequence as set forth for naturally occurring Cas9 from S. thermophiles or S. pyogenes and protein sequences having at least 30%, 40%, 50%, 60%, 70%, 80%, 90%, 95%, 98% or 99% homology thereto and being a DNA binding protein, such as an RNA guided DNA binding protein.

An exemplary CRISPR system includes the S. thermophiles Cas9 nuclease (ST1 Cas9) (see Esvelt, K. M. et al., (2013) Nature Methods. 10(11):1116-21, hereby incorporated by reference in its entirety). An exemplary CRISPR system includes the S. pyogenes Cas9 nuclease (Sp. Cas9), an extremely high-affinity (see Sternberg, S. H., et al., (2014) Nature 507, 62-67, hereby incorporated by reference in its entirety), programmable DNA-binding protein isolated from a type II CRISPR-associated system (see Garneau, J. E. et al., (2010) Nature 468, 67-71 and Jinek, M. et al., (2012) Science 337, 816-821, each of which are hereby incorporated by reference in its entirety). Various Cas proteins are known to those of skill in the art and include CasI (Cas3), Cas IA (Cas8a), CasIB (Cas8b), CasIC (Cas8c), CasID (Cas10d), CasIE (Cse1), CasIF (Csy1), CasIU, CasII (Cas9), CasIIA (Csn2), CasIIB (Cas4), CasIIC, CasIII (Cas10), CasIIIA (Csm2), CasIIIB (Cmr5), CasIIIC, CasIIID, CasIV (Csf1), CasIVA, CasIVB, CasV (Cpf1), C2c2, and C2c1 and the like.

In a multitude of CRISPR-based biotechnology applications (see Mali, P. et al., (2013) Nature Methods 10:957-963; Hsu, P. D. et al., (2014) Cell 157, 1262-1278; Chen, B. et al., (2013) Cell 155:1479-1491; Shalem, O. et al., (2014) Science 343, 84-87; Wang, T. et al., (2014) Science 343:80-84; Nissim, L. et al., (2014) Molecular Cell 54:698-710; Ryan, O. W. et al., (2014) eLife 3; Gilbert, L. A. et al., (2014) Cell 159(3):647-61; and Citorik, R. J. et al., (2014) Nature Biotechnol. 32:1141-1145, each of which are hereby incorporated by reference in its entirety), the guide is often presented in a so-called sgRNA (single guide RNA), wherein the two natural Cas9 RNA cofactors (gRNA and tracrRNA) are fused via an engineered loop or linker.

According to one aspect, the Cas9 protein is an enzymatically active Cas9 protein, a Cas9 protein wild-type protein, a Cas9 protein nickase or a nuclease null or nuclease deficient Cas9 protein. Additional exemplary Cas9 proteins include Cas9 proteins attached to, bound to or fused with functional proteins such as transcriptional regulators, such as transcriptional activators or repressors.

According to certain aspects, the Cas9 protein may be delivered directly to a cell by methods known to those of skill in the art, including injection or lipofection, or as translated from its cognate mRNA, or transcribed from its cognate DNA into mRNA (and thereafter translated into protein). Cas9 DNA and mRNA may be themselves introduced into cells through electroporation, transient and stable transfection (including lipofection) and viral transduction or other methods known to those of skill in the art.

### Guide RNA

The present disclosure provides for the use of guide RNA to target a Cas protein to a target gene as described herein. Such guide RNA can be readily designed by those of skill in the art when knowing the particular target nucleic acid. A guide RNA may include one or more of a spacer sequence, a tracr mate sequence and a tracr sequence. The term spacer sequence is understood by those of skill in the art and may include any polynucleotide having sufficient complementarity with a target nucleic acid sequence to hybridize with the target nucleic acid sequence and direct sequence-specific binding of a CRISPR complex to the target sequence. The guide RNA may be formed from a spacer sequence covalently connected to a tracr mate sequence (which may be referred to as a crRNA) and a separate tracr sequence, wherein the tracr mate sequence is hybridized to a portion of the tracr sequence. According to certain aspects, the tracr mate sequence and the tracr sequence are connected or linked such as by covalent bonds by a linker sequence, which construct may be referred to as a fusion of the tracr mate sequence and the tracr sequence. The linker sequence referred to herein is a sequence of nucleotides, referred to herein as a nucleic acid sequence, which connect the tracr mate sequence and the tracr sequence. Accordingly, a guide RNA may be a two component species (i.e., separate crRNA and tracr RNA which hybridize together) or a unimolecular species (i.e., a crRNA-tracr RNA fusion, often termed a sgRNA).

According to certain aspects, the guide RNA is between about 10 to about 500 nucleotides. According to one aspect, the guide RNA is between about 20 to about 100 nucleotides. According to certain aspects, the spacer sequence is between about 10 and about 500 nucleotides in length. According to certain aspects, the tracr mate sequence is between about 10 and about 500 nucleotides in length. In some embodiments, the tracr sequence is between about 10 and about 100 nucleotides in length. In some embodiments, the linker nucleic acid sequence is between about 10 and about 100 nucleotides in length.

In some embodiments, the guide RNA may be delivered directly to a cell as a native species by methods known to those of skill in the art, including injection or lipofection, or as transcribed from its cognate DNA, with the cognate DNA introduced into cells through electroporation, transient and stable transfection (including lipofection) and viral transduction.

### Modifying Transcription of Target Genes Using Cas9

According to one aspect, an engineered Cas9-gRNA system is provided which enables RNA-guided DNA regulation in cells such as human cells by tethering or connecting transcriptional regulation domains to either a nuclease-null Cas9 or to guide RNAs. According to one aspect of the present disclosure, one or more transcriptional regulatory proteins or domains (such terms are used interchangeably) are joined or otherwise connected to a nuclease-deficient Cas9 or one or more guide RNA (gRNA). The transcriptional regulatory domains correspond to targeted loci. Accordingly, aspects of the present disclosure include methods and materials for localizing transcriptional regulatory domains to targeted loci by fusing, connecting or joining such domains to either Cas9N or to the gRNA.

According to one aspect, a mutant Cas9N-fusion protein capable of transcriptional activation is provided. According to one aspect, a VP64 activation domain (see Zhang et al., Nature Biotechnology 29, 149-153 (2011) hereby incorporated by reference in its entirety) is joined, fused, connected or otherwise tethered to the C terminus of mutant Cas9N. According to one method, the transcriptional regulatory domain is provided to the site of target mitochondrial DNA by the mutant Cas9N protein. According to one method, a mutant Cas9N fused to a transcriptional regulatory domain is provided within a cell along with one or more guide RNAs. The mutant Cas9N with the transcriptional regulatory domain fused thereto bind at or near target mitochondrial DNA. The one or more guide RNAs bind at or near target mitochondrial DNA. The transcriptional regulatory domain regulates expression of the target mitochondrial nucleic acid sequence. According to a specific aspect, a mutant Cas9N-VP64 fusion activated transcription of reporter constructs when combined with gRNAs targeting sequences near the promoter, thereby displaying RNA-guided transcriptional activation.

According to one aspect, a gRNA-fusion protein capable of transcriptional activation is provided. According to one aspect, a VP64 activation domain is joined, fused, connected or otherwise tethered to the gRNA. According to one method, the transcriptional regulatory domain is provided to the site of target mitochondrial DNA by the gRNA. According to one method, a gRNA fused to a transcriptional regulatory domain is provided within a cell along with a mutant Cas9N protein. The mutant Cas9N binds at or near target DNA. The one or more guide RNAs with the transcriptional regulatory protein or domain fused thereto bind at or near target DNA. The transcriptional regulatory domain regulates expression of the target gene. According to a specific aspect, a mutant Cas9N protein and a gRNA fused with a transcriptional regulatory domain activated transcription of reporter constructs, thereby displaying RNA-guided transcriptional activation.

Transcriptional regulator proteins or domains which are transcriptional activators include VP16 and VP64 and others readily identifiable by those skilled in the art based on the present disclosure. For example, one skilled in the art would be able to use Cas9-gRNA system (either with intact cutting or dCas9 with or without being fused to VP16, KRAB, HDAC, methyltransferases etc., or being able to recruit similar activators or repressors with a “spy catcher” or MS2 recruitment domain or similar) can be used to increase or decrease expression of the target genes presented herein to be used in combination for therapeutic or prophylactic effect.

### Target Nucleic Acids

Target nucleic acids include any nucleic acid sequence to which a co-localization complex as described herein can be useful to regulate, such as the genes identified herein. Target nucleic acids include nucleic acid sequences capable of being expressed into proteins. For purposes of the present disclosure, a co-localization complex can bind to or otherwise co-localize with the target nucleic acid at or adjacent or near the target nucleic acid and in a manner in which the co-localization complex may have a desired effect on the target nucleic acid. One of skill based on the present disclosure will readily be able to identify or design guide RNAs and Cas9 proteins which co-localize to a target nucleic acid. One of skill will further be able to identify transcriptional regulator proteins or domains which likewise co-localize to a target nucleic acid.

### Cells

Cells according to the present disclosure include any cell into which foreign nucleic acids can be introduced and expressed as described herein. It is to be understood that the basic concepts of the present disclosure described herein are not limited by cell type. Cells according to the present disclosure include eukaryotic cells, mammalian cells, animal cells, human cells and the like. Further, cells include any in which it would be beneficial or desirable to regulate production of a functional protein. Such cells may include those which are deficient in expression of a particular protein leading to a disease or detrimental condition. Such diseases or detrimental conditions are readily known to those of skill in the art. According to the present disclosure, the nucleic acid responsible for expressing the particular protein may be targeted by the methods described herein and a transcriptional activator resulting in upregulation of the target nucleic acid and corresponding expression of the particular protein. In this manner, the methods described herein provide therapeutic treatment. Such cells may include those which overexpress a particular protein or where production of a particular protein is desired to be reduced leading to a disease or detrimental condition. Such diseases or detrimental conditions are readily known to those of skill in the art. According to the present disclosure, the nucleic acid responsible for expressing the particular protein may be targeted by the methods described herein and a transcriptional depressor or repressor resulting in downregulation of the target nucleic acid and corresponding expression of the particular protein. In this manner, the methods described herein provide therapeutic treatment.

### Delivery of Nucleic Acids Regulating Functional Proteins

Foreign nucleic acids, alternatively referred to as heterologous nucleic acids (i.e., those which are not part of a cell's natural nucleic acid composition) may be introduced into a cell using any method known to those skilled in the art for such introduction. Such methods include transfection, transduction, viral transduction, microinjection, lipofection, nucleofection, nanoparticle bombardment, transformation, conjugation and the like. One of skill in the art will readily understand and adapt such methods using readily identifiable literature sources. Foreign nucleic acids may be delivered to a subject by administering to the subject, such as systemically administering to the subject, such as by intravenous administration or injection, intraperitoneal administration or injection, intramuscular administration or injection, intracranial administration or injection, intraocular administration or injection, subcutaneous administration or injection, a nucleic acid or vector including a nucleic acid as described herein.

Gene therapy methods and methods of delivering genes to subjects, for example using adeno-associated viruses, are described in U.S. Pat. No. 6,967,018, WO2014/093622, US2008/0175845, US 2014/0100265, EP2432490, EP2352823, EP2384200, WO2014/127198, WO2005/122723, WO2008/137490, WO2013/142114, WO2006/128190, WO2009/134681, EP2341068, WO2008/027084, WO2009/054994, WO2014059031, U.S. Pat. No. 7,977,049 and WO 2014/059029, each of which are incorporated herein by reference in its entirety.

### Vectors

Vectors are contemplated for use with the methods and constructs described herein. The term “vector” includes a nucleic acid molecule capable of transporting another nucleic acid to which it has been linked. Vectors used to deliver the nucleic acids to cells as described herein include vectors known to those of skill in the art and used for such purposes. Certain exemplary vectors include, among others, plasmids, lentiviruses, and adeno-associated viruses as is known to those of skill in the art. Vectors include, but are not limited to, nucleic acid molecules that are single-stranded, double stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus, e.g. retroviruses, lentiviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” Common expression vectors of utility in recombinant DNA techniques are often in the form of plasmids. Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell).

Methods of non-viral delivery of nucleic acids or native DNA binding protein, native guide RNA or other native species include lipofection, microinjection, biolistics, virosomes, liposomes, immunoliposomes, polycation or lipid:nucleic acid conjugates, naked DNA, artificial virions, and agent-enhanced uptake of DNA. Lipofection is described in, e.g., U.S. Pat. Nos. 5,049,386, 4,946,787; and 4,897,355, incorporated herein by reference. Lipofection reagents are aso available from commercial sources (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g., in vitro or ex vivo administration) or target tissues (e.g. in vivo administration). The term native includes the protein, enzyme or guide RNA species itself and not the nucleic acid encoding the species.

In some embodiments, the gene therapy vectors for use in the methods herein are parvoviral vectors., such as animal parvoviruses, in particular dependoviruses such as infectious human or simian adeno-associated virus (AAV), and the components thereof (e.g., an animal parvovirus genome) for use as vectors for introduction and/or expression of the nucleotide sequences encoding a porphobilinogen deaminase in mammalian cells. Viruses of the Parvoviridae family are small DNA animal viruses. The family Parvoviridae may be divided between two subfamilies: the Parvovirinae, which infect vertebrates, and the Densovirinae, which infect insects. Members of the subfamily Parvovirinae are herein referred to as the parvoviruses and include the genus Dependovirus. As may be deduced from the name of their genus, members of the Dependovirus are unique in that they usually require coinfection with a helper virus such as adenovirus or herpes virus for productive infection in cell culture. The genus Dependovirus includes AAV, which normally infects humans (e.g., serotypes 1, 2, 3A, 3B, 4, 5, and 6) or primates (e.g., serotypes 1 and 4), and related viruses that infect other warm-blooded animals (e.g., bovine, canine, equine, and ovine adeno-associated viruses). Further information on parvoviruses and other members of the Parvoviridae is described in Kenneth 1. Berns, “Parvoviridae: The Viruses and Their Replication,” Chapter 69 in Fields Virology (3d Ed. 1996). For convenience the present invention is further exemplified and described herein by reference to AAV. It is however understood that the invention is not limited to AAV but may equally be applied to other parvoviruses.

The genomic organization of all known AAV serotypes is very similar. The genome of AAV is a linear, single stranded DNA molecule that is less than about 5,000 nucleotides (nt) in length. Inverted terminal repeats (ITRs) flank the unique coding nucleotide sequences for the non-structural replication (Rep) proteins and the structural (VP) proteins. The VP proteins (VP1, -2 and -3) form the capsid. The terminal 145 nt are self-complementary and are organized so that an energetically stable intramolecular duplex forming a T-shaped hairpin may be formed. These hairpin structures function as an origin for viral DNA replication, serving as primers for the cellular DNA polymerase complex. Following wild-type (wt) AAV infection in mammalian cells the Rep genes (i.e., Rep78 and Rep52) are expressed from the P5 promoter and the P19 promoter, respectively and both Rep proteins have a function in the replication of the viral genome. A splicing event in the Rep ORF results in the expression of actually four Rep proteins (i.e., Rep78, Rep68, Rep52 and Rep40). However, it has been shown that the unspliced mRNA, encoding Rep78 and Rep52 proteins, in mammalian cells are sufficient for AAV vector production. Also in insect cells the Rep78 and Rep52 proteins suffice for AAV vector production.

A “recombinant parvoviral” or “AAV vector” or “rAAV vector” herein refers to a vector comprising one or more polynucleotide sequences of interest, genes of interest or “transgenes” that are flanked by at least one parvoviral or AAV inverted terminal repeat sequences (ITRs). Such rAAV vectors can be replicated and packaged into infectious viral particles when present in an insect host cell that is expressing AAV rep and cap gene products (i.e., AAV Rep and Cap proteins). When an rAAV vector is incorporated into a larger nucleic acid construct (e.g. in a chromosome or in another vector such as a plasmid or baculovirus used for cloning or transfection), then the rAAV vector is typically referred to as a “pro-vector” which can be “rescued” by replication and encapsidation in the presence of AAV packaging functions and necessary helper functions. Thus, in a further aspect the invention relates to a nucleic acid construct comprising a nucleotide sequence encoding a porphobilinogen deaminase as herein defined above, wherein the nucleic acid construct is a recombinant parvoviral or AAV vector and thus comprises at least one parvoviral or AAV ITR. Preferably, in the nucleic acid construct the nucleotide sequence encoding the porphobilinogen deaminase is flanked by parvoviral or AAV ITRs on either side.

AAV is able to infect a number of mammalian cells. See, e.g., Tratschin et al., (1985) Mol. Cell Biol. 5:3251-3260) and Grimm et al., (1999) Hum. Gene Ther. 10:2445-2450). However, AAV transduction of human synovial fibroblasts is significantly more efficient than in similar murine cells, (Jennings et al., (2001) Arthritis Res, 3:1), and the cellular tropicity of AAV differs among serotypes. See, e.g., Davidson et al. (2000) Proc. Natl. Acad. Sci. USA, 97:3428-3432), which discuss differences among AAV2, AAV4, and AAV5 with respect to mammalian CNS cell tropism and transduction efficiency; Goncalves, (2005) Virol J. 2(1):43, which discusses approaches to modification of AAV tropism. In some embodiments, for transduction of liver cells rAAV virions with AAV1, AAV8 and AAV5 capsid proteins are preferred (Nathwani et al., (2007) Blood 109(4):1414-1421; Kitajima et al., (2006) Atherosclerosis 186 (1):65-73), of which is rAAV virions with AAV5 capsid proteins may be most preferred.

AAVs are highly prevalent within the human population. See Gao, G., et al., (2004) J Virol. 78 (12):6381-8; and Boutin, S., et al., (2010) Hum Gene Ther. 21 (6):704-12) and are useful as viral vectors. Many serotypes exist, each with different tropism for tissue types, See Zincarelli, C., et al., (2008) Mol Ther. 16 (6):1073-80), which allows specific tissues to be preferentially targeted with appropriate pseudotyping. Some serotypes, such as serotypes 8, 9, and rh10, transduce the mammalian body. See Zincarelli, C., et al., (2008) Mol Ther. 16 (6):1073-80, Inagaki, K., et al., (2006) Mol Ther. 14 (1):45-53; Keeler, A. M., et al., (2012) Mol Ther. 20 (6):1131-8; Gray, S. J. et al., (2011) Mol Ther. 19 (6):1058-69; Okada, H., et al., (2013) Mol Ther Nucleic Acids. 2:e95; and Foust, K. D., et al., (2009) Nat Biotechnol. 27 (1):59-65. AAV9 has been demonstrated to cross the blood-brain barrier. See Foust, K. D., et al., (2009) Nat Biotechnol. 27 (1):59-65; and Rahim, A. A. et al., (2011) FASEB J. 25 (10):3505-18) that is inaccessible to many viral vectors and biologics. Certain AAVs have a payload of 4.7-5.0 kb, including viral inverted terminal repeats (ITRs), which are required in cis for viral packaging). See Wu, Z. et al., (2010) Mol Ther. 18 (1):80-6; and Dong, J. Y. et al., (1996) Hum Gene Ther. 7 (17):2101-12; all publications incorporated herein by reference.

The AAV VP proteins are known to determine the cellular tropicity of the AAV virion. The VP protein-encoding sequences are significantly less conserved than Rep proteins and genes among different AAV scrotypes. The ability of Rep and ITR sequences to cross-complement corresponding sequences of other serotypes allows for the production of pseudotyped rAAV particles comprising the capsid proteins of one serotype (e.g., AAV5) and the Rep and/or ITR sequences of another AAV serotype (e.g., AAV2). Such pseudotyped rAAV particles are a part of the present invention. Herein, a pseudotyped rAAV particle may be referred to as being of the type “x/y”, where “x” indicates the source of ITRs and “y” indicates the serotype of capsid, for example a 2/5 rAAV particle has ITRs from AAV2 and a capsid from AAV5. Modified “AAV” sequences also can be used in the context of the present disclosure, e.g. for the production of rAAV vectors in insect cells. Such modified sequences e.g. include sequences having at least about 70%, at least about 75%, at least about 80%, at least about 85%, at least about 90%, at least about 95%, or more nucleotide and/or amino acid sequence identity (e.g., a sequence having from about 75% to about 99% nucleotide sequence identity) to an AAV1, AAV2, AAV3, AAV4, AAV5, AAV6, AAV7, AAV8, AAV9, AAV 10, AAV11, AAV12, AAV2.5, AAvDJ, AAVrh10.XX ITR, Rep, or VP can be used in place of wild-type AAV ITR, Rep, or VP sequences. Preferred adenoviral vectors are modified to reduce the host response. See, e.g., Russell (2000) J. Gen. Virol. 81:2573-2604; US patent publication no. 20080008690; and Zaldumbide et al. (2008) Gene Therapy 15 (4):239-46; all publications incorporated herein by reference.

### Regulatory Elements and Terminators

Regulatory elements are contemplated for use with the gene therapy vector constructs described herein. The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, Gene Expression Technology: Methods in Enzymology 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a vector may comprise one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer; see, e.g., Boshart et al, (1985) Cell 41:521-530) the SV40 promoter, the dihydrofolate reductase promoter, the β-actin promoter, the phosphoglycerol kinase (PGK) promoter, and the EF1α promoter and Pol II promoters described herein. Also encompassed by the term “regulatory element” are enhancer elements, such as WPRE; CMV enhancers; the R-U5′ segment in LTR of HTLV-I (Takebe, Y. (1988) Mol. Cell. Biol. 8 (1):466-472); SV40 enhancer; and the intron sequence between exons 2 and 3 of rabbit β-globin (O'Hare K. et al., (1981) Proc. Natl. Acad. Sci. USA. 78 (3):1527-31). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.).

Aspects of the methods described herein may make use of terminator sequences. A terminator sequence includes a section of nucleic acid sequence that marks the end of a gene or operon in genomic DNA during transcription. This sequence mediates transcriptional termination by providing signals in the newly synthesized mRNA that trigger processes which release the mRNA from the transcriptional complex. These processes include the direct interaction of the mRNA secondary structure with the complex and/or the indirect activities of recruited termination factors. Release of the transcriptional complex frees RNA polymerase and related transcriptional machinery to begin transcription of new mRNAs. Terminator sequences include those known in the art and identified and described herein.

### Administration, Dosage and Treatment

In various embodiments, the one or more gene delivery vectors, including viral vectors, and packaged viral particles containing the viral vectors, can be in the form of a medicament or a pharmaceutical composition and may be used in the manufacture of a medicament or a pharmaceutical composition. The pharmaceutical composition may include a pharmaceutically acceptable carrier. Preferably, the carrier is suitable for parenteral administration. In particular embodiments, the carrier is suitable for intravenous, intraperitoneal or intramuscular administration. Pharmaceutically acceptable carrier or excipients are described in, for example, Remington: The Science and Practice of Pharmacy, Alfonso R. Gennaro (Editor) Publishing Company (1997). Exemplary pharmaceutical forms can be in combination with sterile saline, dextrose solution, or buffered solution, or other pharmaceutically acceptable sterile fluids. Alternatively, a solid carrier, may be used such as, for example, microcarrier beads.

Pharmaceutical compositions are typically sterile and stable under the conditions of manufacture and storage. Pharmaceutical compositions may be formulated as a solution, microemulsion, liposome, or other ordered structure suitable to delivery of the gene therapy vectors. The carrier may be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. The vectors of the present disclosure may be administered in a time or controlled release formulation, for example in a composition which includes a slow release polymer or other carriers that will protect the compound against rapid release, including implants and microencapsulated delivery systems. Biodegradable, biocompatible polymers may for example be used, such as ethylene vinyl acetate, polyanhydrides, polyglycolic acid, collagen, polyorthoesters, polylactic acid and polylactic, polyglycolic copolymers (PLG).

In some embodiments, the gene therapy vectors, formulated with any acceptable carriers, can be administered parenterally, such as by intravenous, intraperitoneal, subcutaneous, intramuscular administration, limb perfusion or combinations thereof. The administration can be systemic, such that the gene delivery vectors are delivered through the body of the subject. In some embodiments, the gene delivery vectors can be administered directly into the targeted tissue, such as to the heart, liver, synovium, or intrathecally for neural tissues. In some embodiments, the gene delivery vectors can be administered locally, such as by a catheter. The route of administration can be determined by the person of skill in the art, taking into consideration, for example, the nature of target tissue, gene delivery vectors, intended therapeutic effect, and maximum load that can be administered and absorbed by the targeted tissue(s).

Generally, an effective amount, particularly a therapeutically effective amount, of the gene delivery vectors are administered to a subject in need thereof. A “therapeutically effective amount” refers to an amount effective, at dosages and for periods of time necessary, to achieve the desired therapeutic result, such as treatment or amelioration of an age-related condition. An effective or therapeutically effective amount of vector may vary according to factors such as the disease state, age, sex, and weight of the individual, and the ability of the viral vector to elicit a desired response in the individual. Dosage regimens may be adjusted to provide the optimum therapeutic response.

In particular embodiments, a range for therapeutically or prophylactically effective amounts of a nucleic acid, nucleic acid construct, parvoviral virion or pharmaceutical composition may be from 1×10¹¹ and 1×10¹⁴ genome copy (gc)/kg or 1×10¹² and 1×10¹³ genome copy (gc)/kg. It is to be noted that dosage values may vary with the severity of the condition to be alleviated. The dosage may also vary based on the efficacy of the virion employed. For example AAV8 is better at infecting liver as compared to AAV2 and AAV9 is better at infecting brain than AAV8, in these two cases one would need less AAV8 or AAV9 for the case of liver or brain respectively. For any particular subject, specific dosage regimens may be adjusted over time according to the individual need and the professional judgement of the person administering or supervising the administration of the compositions. Dosage ranges set forth herein are exemplary only and do not limit the dosage ranges that may be selected by medical practitioners.

The tissue target may be specific, for example the liver tissue, or it may be a combination of several tissues, for example the muscle and liver tissues. Exemplary tissue targets may include liver, skeletal muscle, heart muscle, adipose deposits, kidney, lung, vascular endothelium, epithelial and/or hematopoietic cells. In some embodiments, the effective dose range for small animals (mice), following intramuscular injection, may be between 1×10¹² and 1×10¹³ genome copy (gc)/kg, and for larger animals (cats or dogs) and for human subjects, between 1×10¹¹ and 1×10¹² gc/kg, or between 1×10¹¹ and 1×10¹⁴ genome copy (gc)/kg.

In various embodiments, the gene delivery vectors can be administered as a bolus or by continuous infusion over time. In some embodiments, several divided doses can be administered over time or the dose may be proportionally reduced or increased as indicated by the exigencies of the therapeutic situation. In some embodiments, the gene delivery vectors can be administered daily, weekly, biweekly or monthly. The duration of treatment can be for at least one week, one month, 2 months, 3 months, 6 months, or 8 month or more. In some embodiments, the duration of treatment can be for up to 1 year or more, 2 years or more, 3 years or more or indefinitely.

In some embodiments, a therapeutically effective amount is administered to the subject to treat a condition or disease associated with aging, e.g., an age related disease or disorder. The application of the invention extends the period of time for which an individual is generally healthy and free of chronic illness and/or the invention ameliorates disorders that appear often in aged and ageing adult population, including one or more of cardiovascular diseases, diabetes, atherosclerosis, obesity, cancer, infection, and neurological disorders. Any well established indicators of ageing progression can be used.

In some embodiments, the gene therapy described herein is used to least one of the following indicators of aging: reducing the incidence of cancer, delaying or ameliorating cardiovascular disease, such as atherosclerosis; delaying and/or ameliorating osteoporosis; improving glucose tolerance or reducing incidence of related diseases, such as diabetes and obesity; improving or reducing the decline in memory function and other cognitive functions; improving or reducing the decline neuromuscular coordination; and improving or reducing the decline in immune function. The amelioration of age-related disorders provided by the gene therapy methods herein can be as a result of reduction of symptoms in an affected subject or a reduction of incidence of the disease or disorder in a population as compared to an untreated population. The gene therapy has the effect of treating and/or preventing various age-related conditions and diseases, as assessed by particular markers and disorders of ageing. In a further aspect, therefore, the invention refers to a gene therapy method or the use of a nucleic acid vector as described above, for use in the treatment or prevention in a subject of at least a disorder or marker of ageing that is selected from the group of reduced cardiovascular function, osteoporosis, arthrosis, glucose intolerance, insulin resistance, loss of memory, loss of neuromuscular coordination, increase in cardiovascular disease, decrease in heart, circulatory or lung function and decrease in longevity, or combinations thereof.

In some embodiments, the gene therapy described herein is used to extend the lifespan for any particular species of subject. Extended lifespan can be an increase in the average lifespan of an individual of that species who reaches adulthood and/or an extension of the maximum lifespan of that species. In some embodiments, extended lifespan can be a 5%, 10%, 15%, 20% or more increase in maximum lifespan and/or a 5%, 10%, 15%, 20% or more increase in average lifespan.

### EXAMPLES

### Example 1: Methods for Regulating TGFβ1

The present disclosure provides a gene therapy method for the long term regulation of TGFβ1 in an animal such as a human or other mammal such as a domesticated animal such as a dog or cat. The disclosure provides a gene therapy method for the long term regulation of TGFβ1 in an animal such as a human or other mammal such as a domesticated animal such as a dog or cat as a method of treating or preventing inflammation, remodeling, or fibrosis. The disclosure provides a gene therapy method for regulating TGFβ1 in an animal such as a human or other mammal such as a domesticated animal such as a dog or cat for treating a heart pathology, such as increased fibrosis. The disclosure provides for the regulation of TGFβ1 by a gene therapy method including the delivery of a nucleic acid to a cell, for example, by using an adeno-associated vector. The disclosure provides for the regulation of TGFβ1 by the delivery of a nucleic acid that produces a soluble circulating protein that binds TGFβ1 thereby inhibiting its ability to activate its endogenous pathway. The soluble circulating protein can be the extracellular domain of TGFβ receptor 2. One skilled in the art can also create a version from the TGFβ receptor 1 or 3 as well. The soluble TGFβ receptor 2 protein has been truncated at the transmembrane domain of the protein as predicted by annotation software and by hydrophobicity of the amino acids.

### Plasmids

The vector AAV vector was created by amplifying the extracellular domain using Forward primer 5′-GCCACCATGGGTCGGGGGCTGC (SEQ ID NO: 118) and reverse primer 5′-GGACAGGGCTTGATTGTGGGCCCTCTGGGGTCGGGACTGCTGGTGGTGTATTCTT CCG (SEQ ID NO: 119). The bold and italicized part of the forward primer is the Kozak sequence. The bold and italicized part of the reverse primer matches the mouse igg domain that was fused C terminally by overlapping PCR.

Forward primer 5′-CGGAAGAATACACCACCAGCAGTCCCGACCCCAGAGGGCCCACAATCAAGCCCTG TCC (SEQ ID NO:1) and reverse primer 5′-TCATTTACCCGGAGTCCGGGAGAAGCTC (SEQ ID NO:2) were used to amplify the igg domain. The bold and italicized part matches the extracellular domain of TGFbR2 for overlapping PCR. The two parts were combined by using equal molar ratios of the amplified sections in a second round of PCR using the forward primer from TGFbR2 amplification and the reverse primer from igg amplification. This created the fusion protein sTGFbR2-Fc(igg2Ae) for a total length of 1251 base pairs. This was ligated into an AAV backbone using unique restriction enzyme site overhangs NotI and NheI.

### AAV Production

The method of AAV production and titer quantification was carried out according to Lock, M. 2010 Human gene therapy; Kwon, O. et al., (2010) J Histochem Cytochem. 58 (8):687-694. Briefly, Hek 293 cells were triple co-transfected at 75% confluency in one 10 layer Nunc™ Cell Factory™ System from Thermo Scientific (Rockford, IL) using PEI transfection reagent following manufacturer's instructions. Cells and supernatant were harvested separately after 72 hours post transfection. The cells were spun down and lysed with 3 freeze-thaw cycles and incubated with Benzonase (E1015-25KU, Sigma). They were then clarified by spinning at 10,500×G for 20 min and the supernatant was added to the rest of the media supernatant. Everything was filtered through a 0.2 uM filter and was then concentrated using lab scale TFF system (EMD Chemicals, Gibbstown, NJ) down to 15 ml. We used a Pellicon XL 100 kDa filter and followed manufactures instructions (EMD Chemicals, Gibbstown, NJ). The concentrated prep was re-clarified by centrifugation at 10,500×g and 15° C. for 20 min and the supernatant was carefully removed to a new tube. Six iodixanol step gradients were formed according to the method of Zolotukhin and colleagues. See Zolotukhin S., (1999) Gene Ther. 6:973-85, with some modifications as follows: Increasingly dense iodixanol (OptiPrep; Sigma-Aldrich, St Louis, MO) solutions in phosphate-buffered saline (PBS) containing 10 mM magnesium chloride and 25 mM potassium were successively underlaid in 39 ml of Quick-Seal centrifuge tubes (Beckman Instruments, Palo Alto, CA). The steps of the gradient were 4 ml of 15%, 9 ml of 25%, 9 ml of 40%, and 5 ml of 54% iodixanol. Fourteen milliliters of the clarified feedstock was then overlaid onto the gradient and the tube was sealed. The tubes were centrifuged for 70 min at 242,000×g in a VTi 50 rotor (Beckman Instruments) at 18° C. and the 40% gradient was collected through an 18-gauge needle inserted horizontally at the 54%/40% interface. The virus containing iodixanol was diafiltered using Amicon 15-Ultra and washed 5 times with final formulation buffer (PBS-35 mM NaCl), and concentrated to ˜1 ml.

### Vector Characterization

DNase I-resistant vector genomes were titered by TaqMan PCR amplification (Applied Biosystems, Foster City, CA), using primers and probes directed against the WPRE3 poly Adenylation signal encoded in the transgene cassette. The purity of gradient fractions and final vector lots were evaluated by sodium dodecyl sulfate-polyacrylamide gel electrophoresis (SDS-PAGE) and the proteins were visualized by SYPRO ruby staining (Invitrogen) and UV excitation.

### Infection

The mice were infected through intra-peritoneal (IP), tail vein (IV) or retro orbital (RO) injection. Briefly, the IP injection location is located by drawing an imaginary line across the abdomen just above the knees. The needle is inserted along this line on the animal's right side and close to the midline. To perform an IP injection, the mouse must be well restrained so that it cannot move during the procedure. Tilt the entire mouse so that the top of the head is facing toward the ground and its hind legs are higher up and so that it's abdomen is facing you. Insert the needle into the abdomen at about a 30-degree angle, the shaft of the needle should enter to a depth of about half a centimeter. Aspirate to be sure that the needle has not penetrated a blood vessel, the intestines, or the urinary bladder. Inject the contents of the syringe and withdraw the needle and return the mouse to its cage. The recommended needle size for IP injections in the mouse is 25-27 gauge. For IV injection into the tail vein, the mouse is restrained and the tail vein is found by holding the tail over a bright light. The vein is made larger by briefly putting the mice under a heat lamp to dilate the blood vessels. Then using a 25 gauge needle the mouse is injected with up to 200 ul. The RO injections are done under anesthesia. The mouse, while under, is prepared by putting slight pressure to bulge the eye and slide the needle behind the eye with only up to 150 ul for an adult mouse.

### Surgery

Aortic constriction (ACC) is induced in adult animal through constriction of ascending aorta. An incision will be made in the chest wall approximately at the third intercostals space. A rodent rib spreader is inserted and the ribs gently spread to allow access to the thoracic cavity. The ascending aorta is then isolated from the pulmonary artery and a sterile 8.0 prolene ligature is passed around it approximately 3 mm from the base of the heart. A blunted 27 gauge needle is placed on top of the aorta and ligation is tied around the needle. The needle is then carefully removed from under the tie. The rib spreader is closed and the lung re-inflated. The ribs, chest musculature, and skin are closed using sterile sutures (5-0 Dexons and 6-0 Prolene sutures for closing muscle and subcutaneous layers and skin, respectively). The surgeon will minimize pneumothorax by expanding the lungs in concert with the placement of the last suture closing the thoracotomy. Sham operated animals undergo similar procedure without constriction of the aorta. The animal will be closely monitored until full recovery from anesthesia. Once the animal has regained consciousness (and is able to protect its airway), the animal will be extubated. Animals will continually be closely monitored until full neurological consciousness is achieved. Suture will be removed by 10-14 days post-surgery. The date, time, and type of the surgical procedure will be noted on a clinical post-operative record, as required. ACC surgical mortality may be 30%.

### Non-Invasive Echocardiography

To serially non-invasively assess cardiac structure and function, animals at designated time points (not to exceed once per week) undergo non-invasive transthoracic echocardiography. For this, animals are brought to a designated procedure room. The animal is lightly anesthetized with 1.5-5% (mice and rat) isoflurane. Sedation will be confirmed by the lack of response to gentle skin pinch. Eye ointment is applied to the anesthetized animals to prevent the eye from drying and causing irritation or ulceration. Hair will be removed from the animal's chest using #40 blade and medical grade depilatory cream for obtaining clear echo images. The animal is gently placed on a platform, and the echocardiogram probe placed on the left chest wall. The heart rate and respiratory rate of the animal is monitored with the physiologic monitor that is connected to the echo machine and the platform on which the animal is placed while the ultrasound imaging is going on. Ultrasound images are generally obtained within 15 minutes and result in no pain to the animal. During the procedure, animals are closely monitored for any signs of distress, and if any are present, the procedure is immediately terminated and the animal returned to its cage.

### Euthanasia

Animals are euthanized by the slow fill method of CO2 administration according to the equipment available in the facility. Typically, animals are euthanized in the home cage out of view from other animals. A regulator is used to ensure the proper flow rate. Animals should lose consciousness rapidly ˜30 sec. At the cessation of breathing (several minutes) animals will undergo a secondary physical method of euthanasia.

### Tissue Harvest

Tissues are immediately harvested after euthanasia. Part of each organ was snap frozen in dry ice for qPCR analysis and sequencing and the other part of each organ was then formalin fixed overnight 24-48 hours depending on size and frozen in OCT buffer for sectioning and analysis.

### Blood Collection

The mice are held by their scruff and a needle is used to puncture the mandibular vein/artery and blood is collected in heparin coated tubes.

### Staining and Sectioning

The mice are sectioned using a microtome and then paraffin embedded. Deparafinization and rehydration takes place by heating the slides to 50° C. and then successive baths of xylene and ethanol and finally DI H₂O. The slides are then incubated in boiling citrate buffer for 10 minutes and are cooled on the bench at room temperature. They are then washed in PBS 5× for 2 min each. The slides are blocked in 3% BSA in PBS at room temperature for 1 hour. Primary antibody was applied in 3% BSA/PBS at a 1:300 dilution overnight at 4° C. The slides are then washed in PBS 5× for 2 min each. The secondary antibody in 3% BSA/PBS is applied for 1 hr at 37° C., dilution 1/100. The slides are then washed in PBS 5× for 2 min each. 50 μl per slide DAPI (or Hoechst final 5 μg/ml) in PBS is applied for 30 minutes in room temperature (in humid chamber). The slides are then washed in PBS 5× for 2 min each. Mount with mounting medium without DAPI and cover.

### Inhibition of Transforming Growth Factor β1 (TGFβ1)

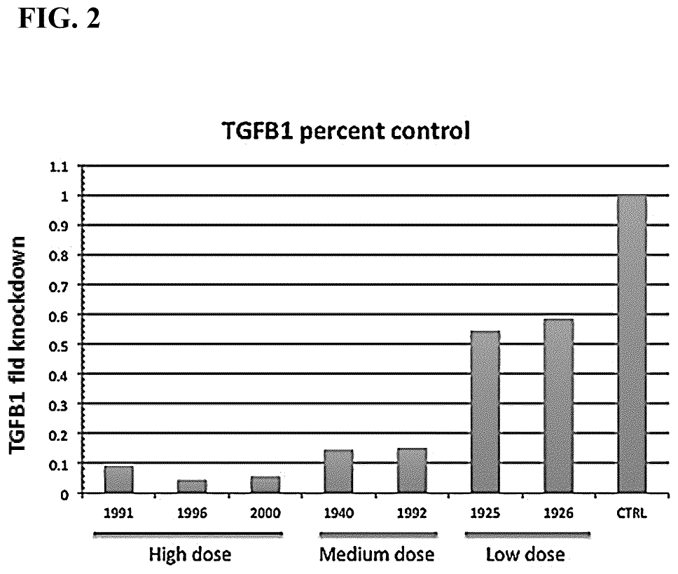

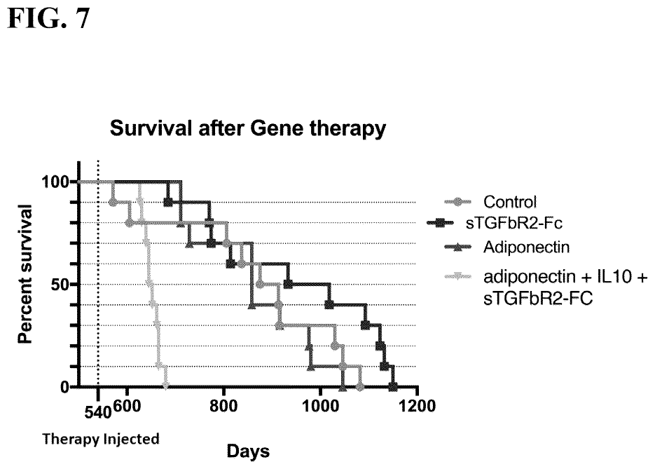

A nucleic acid encoding a soluble receptor protein for Transforming Growth Factor Receptor II (TGFbRII) was delivered using an AAV to cells in a mouse model of HCM/DCM that uses aortic banding to achieve a pressure overload that eventually causes the desired phenotype. The AAV was used for the long-term permanent decrease of TGFβ1, which is beneficial for reducing heart failure and promoting longevity as indicated in the survival curve shown in FIG. 7. The soluble receptor protein binds TGFβ1 in order to reduce signaling of this pathway and alleviate fibrotic tissue induction and inflammatory responses. As indicated in FIG. 2, administration of the AAV with the nucleic acid encoding the soluble receptor protein for Transforming Growth Factor Receptor II (TGFbRII) decreased serum TGFβ1 by up to 95% leading to a reduction in TGFβ1 signaling.

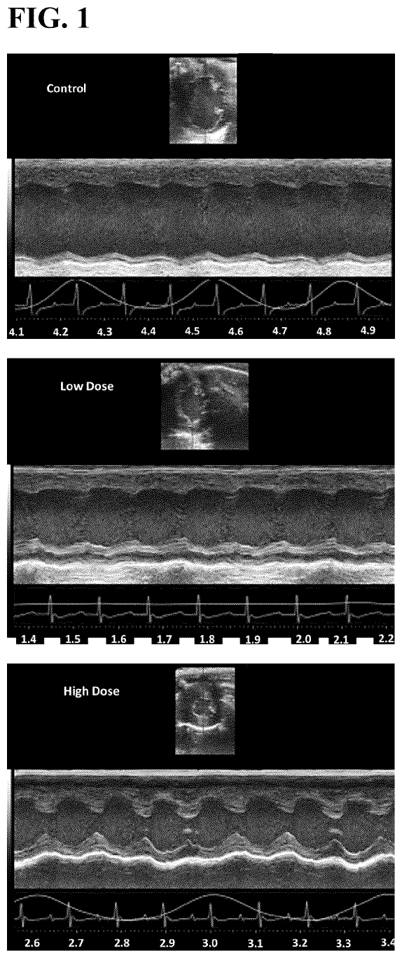

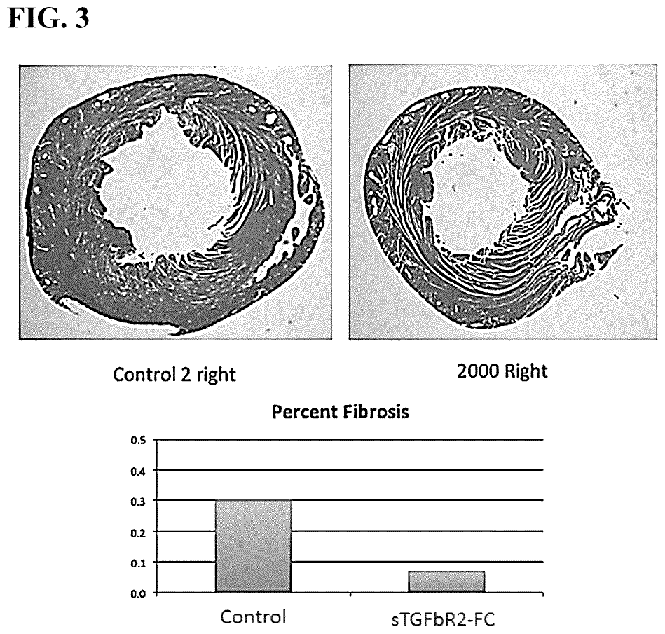

The gene therapy affected fibrotic lesion development. As indicated in FIG. 3, the control samples have approximately 30% fibrosis on the total area of sectioned heart as compared to the AAV treated mouse heart that has approximately 8%. The gene therapy also impacted the functioning of the heart 7 weeks post AAC surgery. As shown in FIG. 1, there is a large difference in the strength of heart contractility and left ventricle volume as seen in the echocardiograms. FIG. 5 demonstrates that control animals have a negative change in several parameters such as heart wall thickness, ejection fraction, fractional shortening, and left ventricle volume. The gene therapy animals have either no or a positive change in these parameters.

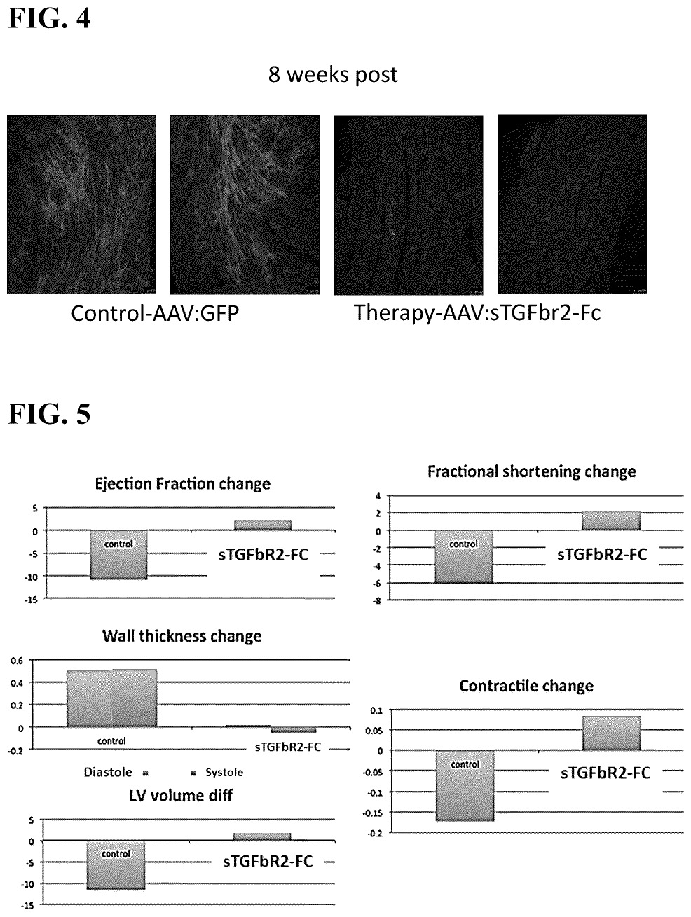

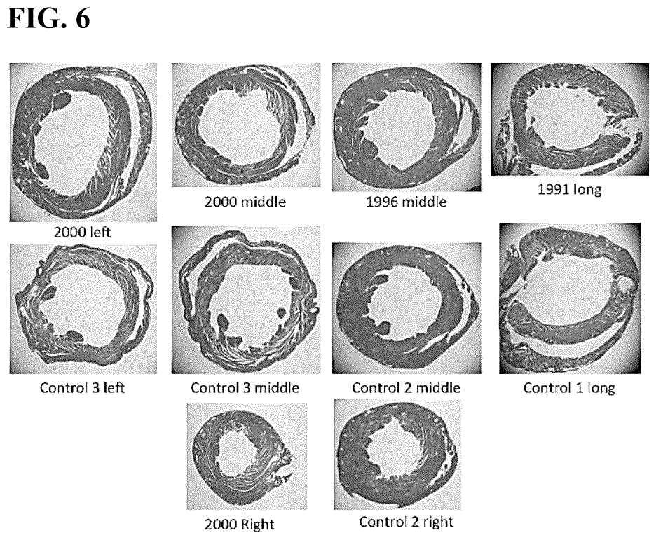

FIG. 4 depicts WGA and DAPI staining for control heart. FIG. 6 depicts representative trichrome staining images.

### Example 2: Combination Gene Therapy

The disclosure provides a gene therapy method for the delivery of a nucleic acid encoding a soluble receptor protein for Transforming Growth Factor Receptor II (TGFbRII) and a nucleic acid encoding for Nrf2 (or nuclear factor (erythroid-derived 2)-like 2 Nfe212). Nrf2 is an antioxidant protein that protects against oxidative damage triggered by injury and inflammation. The disclosure provides a method of using each in combination by directly injecting two viruses each containing one transgene cassette. (i.e. ITR-hEf1α-sTGFbR2-Fc-WPRE3-SV40 pA-ITR AND ITR-hEf1α-Nrf2-WPRE3-SV40 pA-ITR). The disclosure provides combining both transgenes into one vector by use of the viral 2A sequences or IRES whereby two genes are expressed from one promoter. (i.e. ITR-hEf1α-sTGFbR2-Fc-P2A-Nrf2-WPRE3-SV40 pA-ITR or ITR-hEf1α-sTGFbR2-Fc-IRES-Nrf2-WPRE3-SV40 pA-ITR).

### Example 3: Methods for Regulating Adiponectin

The disclosure provides a gene therapy method for regulating Adiponectin in an animal such as a human or other mammal such as a domesticated animal such as a dog or cat. An adeno-associated virus is provided including a constitutive promoter driving the expression of a nucleic acid encoding adiponectin and DsbA-L (GSTK1). The nucleic acid construct may include a 3′UTR including WPRE3 and late SV40 pA. A nucleic acid encoding adiponectin is provided in a first vector and a nucleic acid encoding DsbA-L is provided in a second vector. The first vector and the second vector are self-complimentary AAV vectors. A self-complementary adeno-associated virus (scAAV) is a viral vector engineered from the naturally occurring adeno-associated virus (AAV). The rAAV is termed “self-complementary” because the coding region has been designed to form an intra-molecular double-stranded DNA template. A rate-limiting step for the standard AAV genome involves the second-strand synthesis since the typical AAV genome is a single-stranded DNA template. However, this is not the case for scAAV genomes. Upon infection, rather than waiting for cell mediated synthesis of the second strand, the two complementary halves of scAAV will associate to form one double stranded DNA (dsDNA) unit that is ready for immediate replication and transcription. In gene therapy application utilizing rAAV, the virus transduces the cell with a single stranded DNA (ssDNA) flanked by two Inverted Terminal Repeats (ITRs). These ITRs form hairpins at the end of the sequence to serve as primers to initiate synthesis of the second strand before subsequent steps of infection can begin. The second strand synthesis is considered to be one of several blocks to efficient infection. Additional advantages of scAAV include increased and prolonged transgene expression in vitro and in vivo, as well as higher in vivo DNA stability and more effective circularization.

### Example 4: Method for Treating Obesity

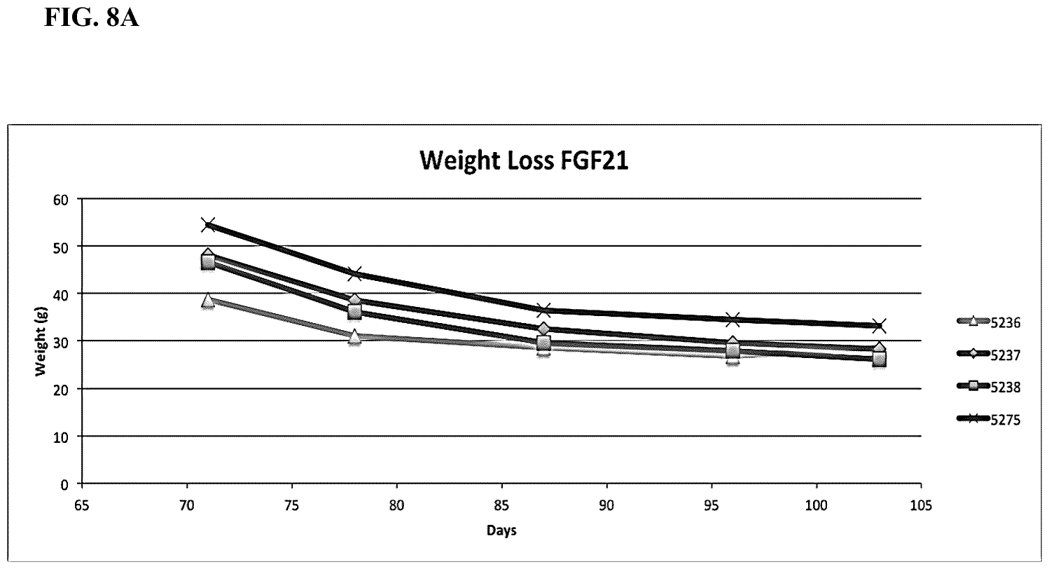

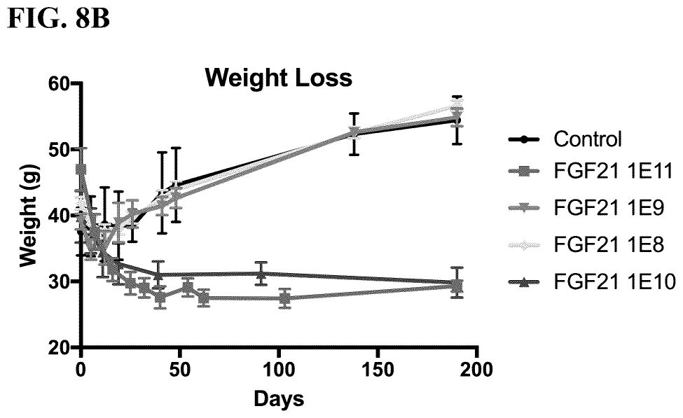

The disclosure provides for a gene therapy method for the delivery of a nucleic acid encoding fibroblast growth factor 21 (FGF21) and a nucleic acid encoding either BMP2, BMP4 or Sema3a or all of them together. FGF21 is known to shift the balance of osteoblasts and osteoclasts toward osteoclastic formation through the inhibition of differentiation of cells into osteoblasts and the promotion of differentiation of cells into osteoclasts. To balance the negative side effect of bone loss from increased FGF21 expression, multiple genes are delivered. FGF21 will cause weight loss as seen in FIGS. 8A and 8B without any apparent toxicity (seen by body condition score and activity monitoring). The mice while maintained on a high fat diet were able to lose up to 40% of their body weight (back to the normal weight for a mouse their age) and seem to have plateaued into the normal range. To combat the bone loss, one or two or all of BMP2, BMP4 or Sema3a are delivered with FGF21 simultaneously or in series as part of a combination therapy to shift the balance back to homeostasis for osteoblasts and osteoclasts.

According to certain aspects, methods are provided for losing weight in an individual or increasing metabolic rate of an individual comprising delivery of a nucleic acid encoding fibroblast growth factor 21 (FGF21) to the individual in a gene therapy method, such as using an AAV or otherwise regulating, (upregulating or downregulating) FGF21.

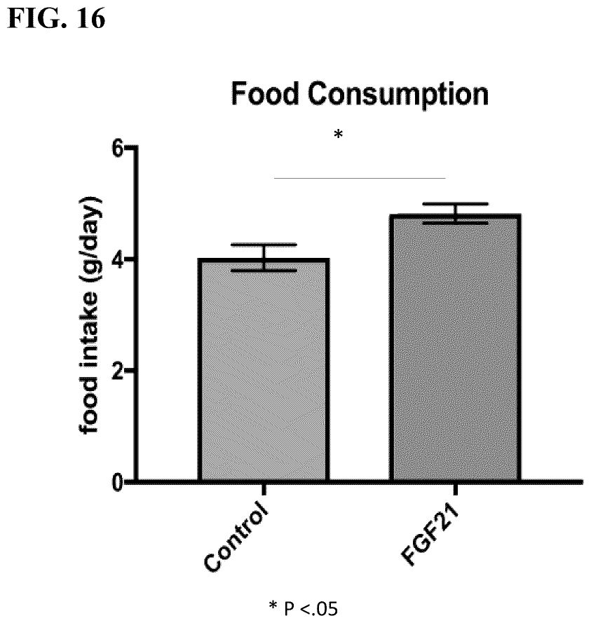

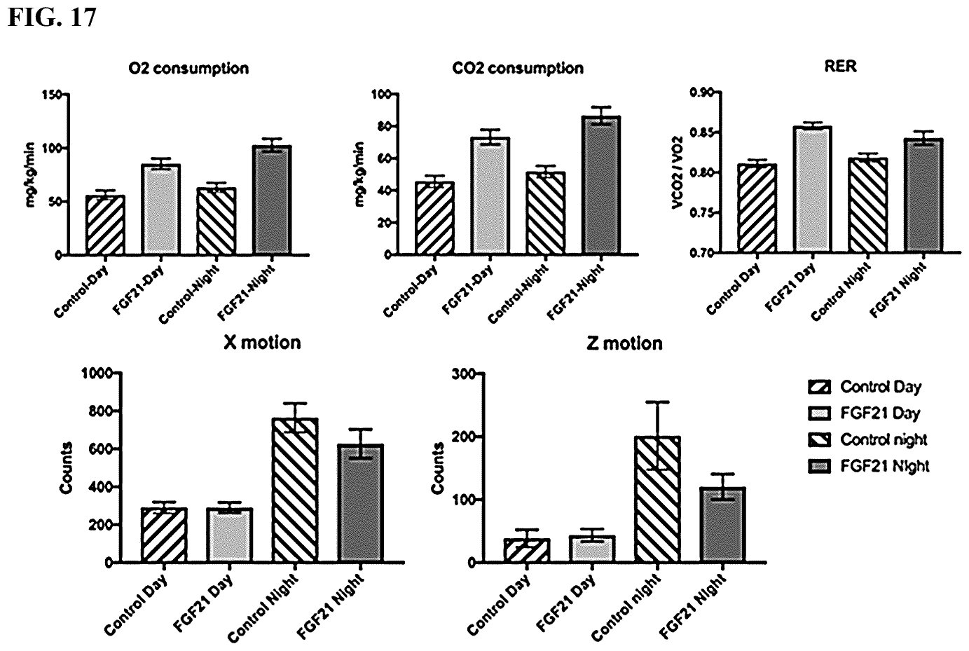

In an experiment conducted, food intake of mice was measured where the mice were provided with FGF21 as a gene therapy and mice that received the therapy consume more high fat food but are still able to maintain a normal lean mouse weight, indicating an altered metabolic state. See FIG. 16. The effect of the gene therapy on respiration rate and activity was determined. Mice treated with FGF21 were placed into the Columbus Instruments CLAMS system to measure their O₂ consumption, CO₂ production and their movement in X, Y, Z plane and the results are shown in FIG. 17. Once the mice were in the system the data was generated automatically. The FGF21 mice that maintain a lean body weight while consuming more food have higher metabolic activities as indicated by the increased O₂ consumptions and CO₂ production as compared to controls. However, as noted by motion sensors, the FGF21 mice show less movement providing another indication that their altered mitochondrial activity and metabolic state and not their behavior is responsible for their ability to maintain a lean body mass while on a high fat diet.

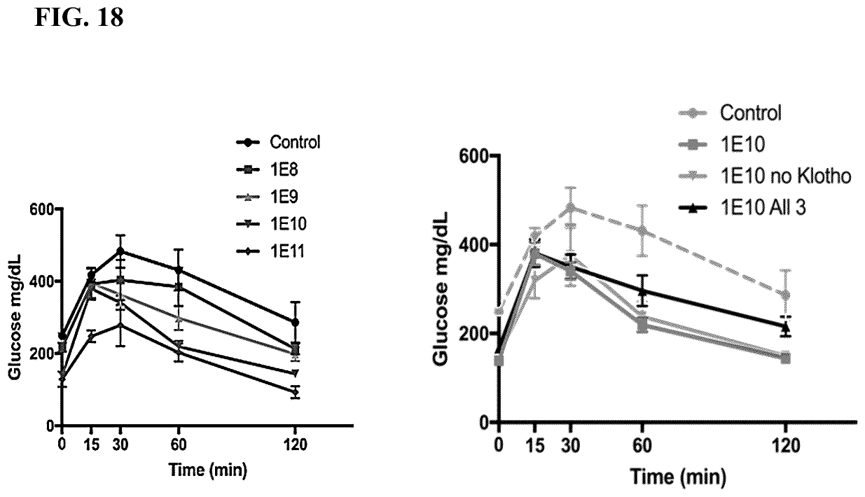

The effect of FGF21 on glucose and insulin sensitivity was tested through the Glucose tolerance test and determined to have a large effect as indicated in FIG. 18. Briefly, mice were fasted overnight anywhere from 6-10 hours. Blood was collected to analyze baseline blood glucose levels. The mice were then given a dose of glucose solution through oral gavage with up to 500 ul of 250 mg/ml glucose in ddH₂O. Then blood was taken and blood glucose was measured in the following increments: 15 min, 30 min, 60 min, and 120 min. The data is shown in FIG. 18 for several different doses of FGF21 in the left hand graph and then for different combinations of other proteins with a constant 1E10 dose for FGF21. FGF21+sTGFbR2-FC, and FGF21+sTGFbR2-FC+Klotho were tested and the results presented in the right hand graph with the differences in glucose indicated.

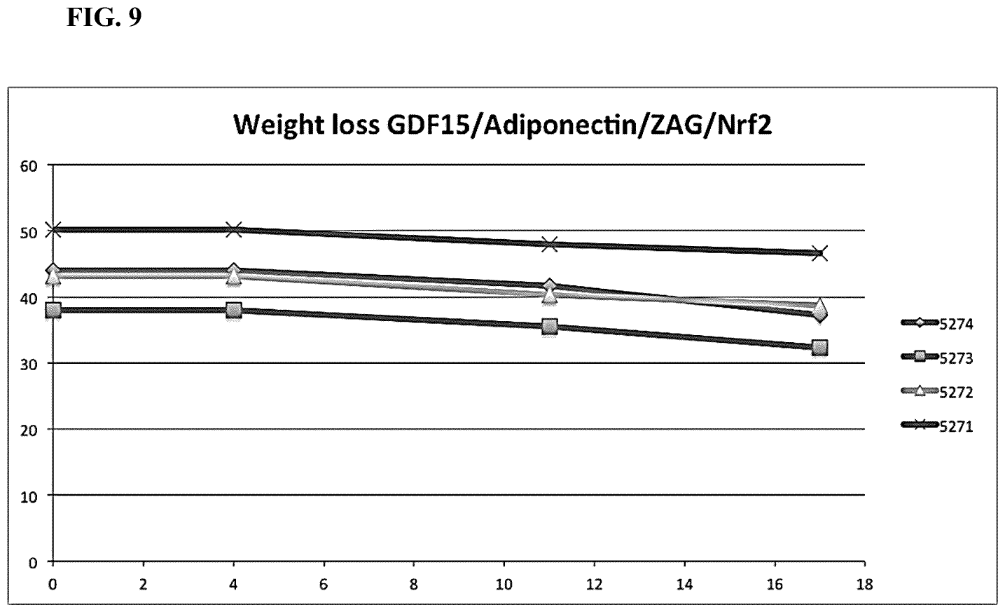

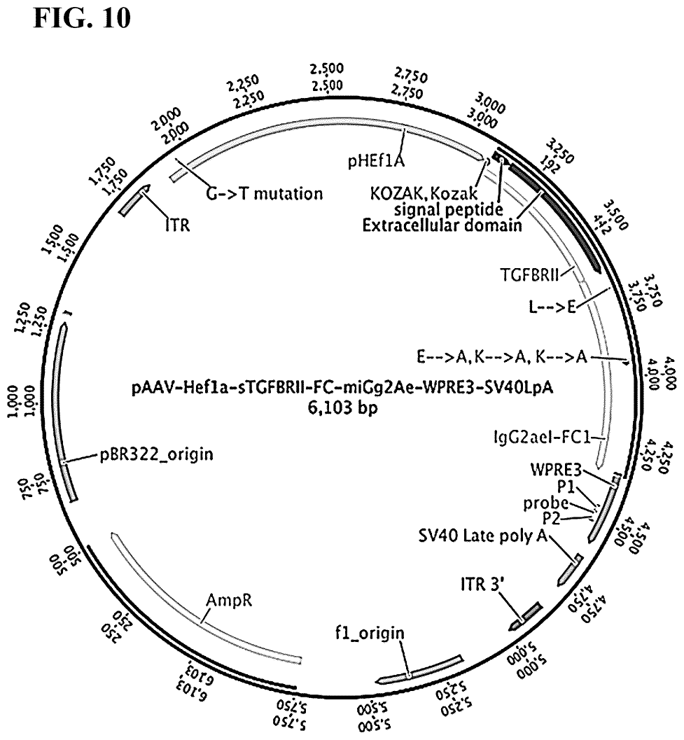

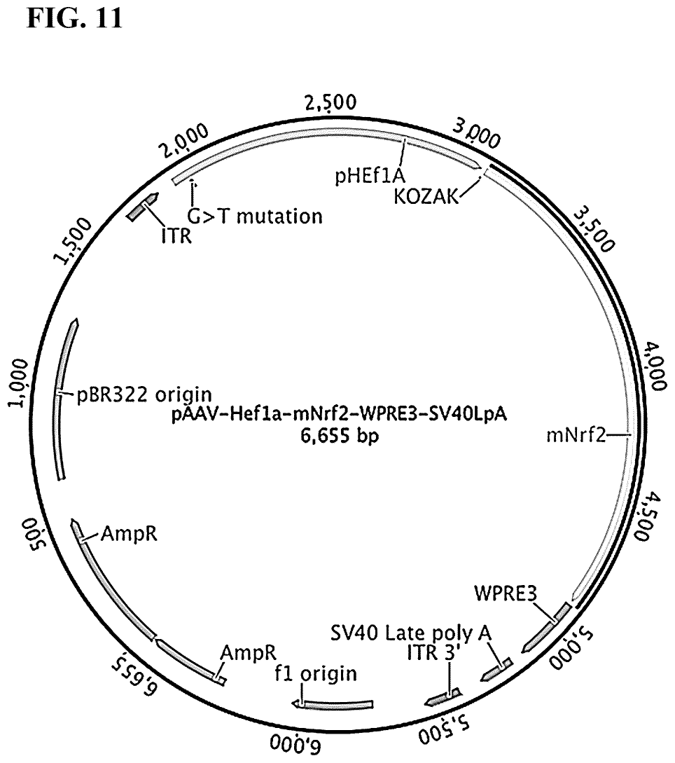

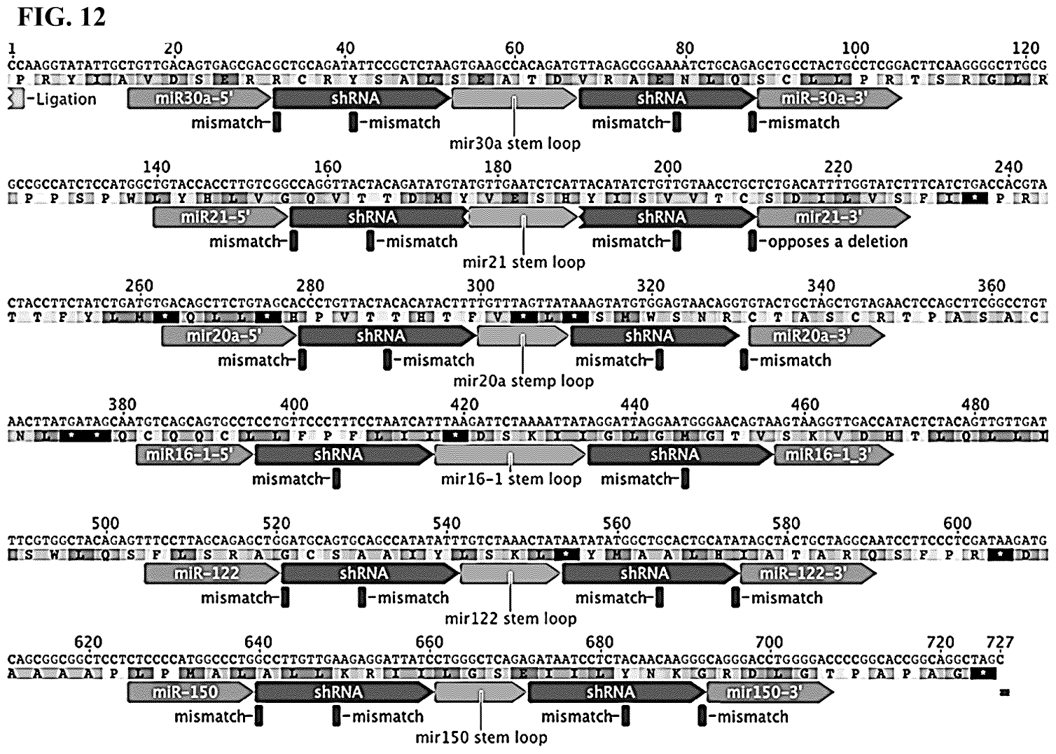

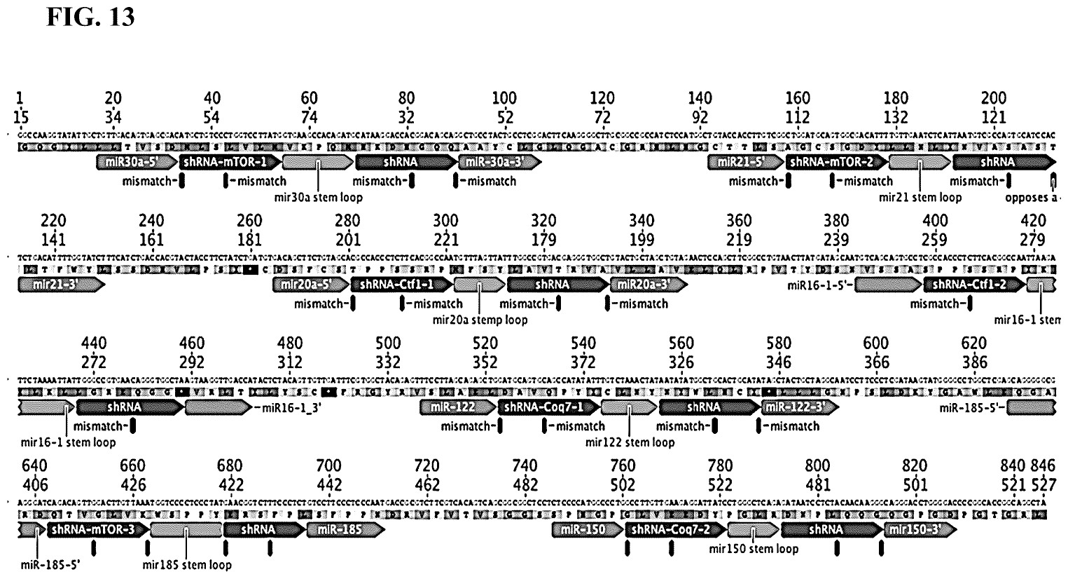

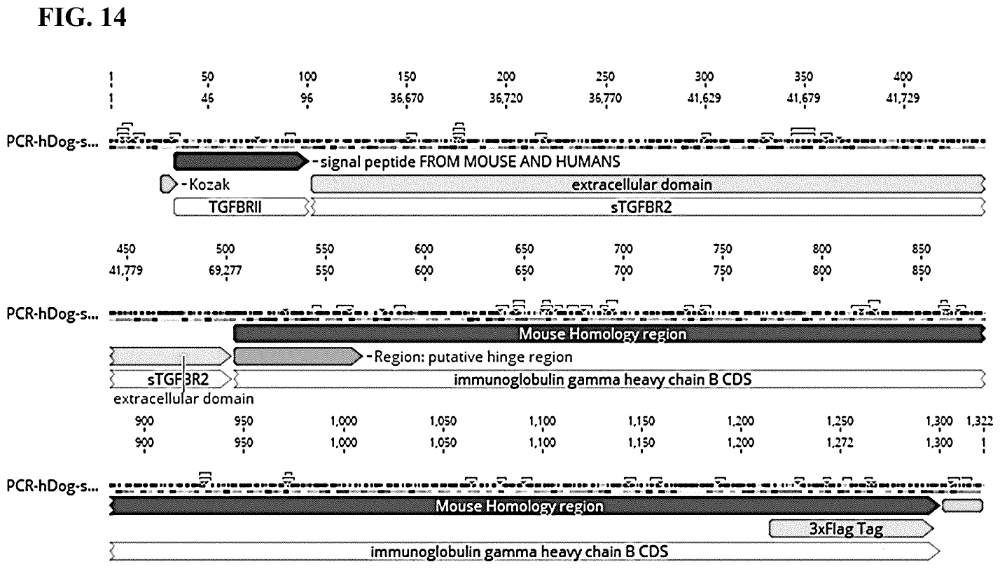

The disclosure provides for a gene therapy method for the delivery of a nucleic acid encoding Growth differentiation factor 15 (GDF15) and a nucleic acid encoding adiponectin, and a nucleic acid encoding ZAG and a nucleic acid encoding Nrf2. Combining delivery of an effective amount of all 4 of these nucleic acids has shown evidence of weight loss without toxicity (seen by body condition score and activity monitoring). These mice have lost up to 15% of their weight and continue in a downward trend as seen in FIG. 9.

### Example 5: Expression Cassette

The disclosure provides an expression cassette contained within an AAV including 14 Pri-miRNA-shRNA sequences. The vector has a first cassette facing up stream and a second cassette facing downstream. The first cassette includes 7 miRNA-shRNA sequences. The second cassette include miRNA-shRNA sequences. The upstream facing cassette and the downstream facing cassette prevent read through between the two cassettes. A schematic of the two cassettes is as follows: ITR_3′UTR-1_7miRNAs_Promoter-1_Promoter-2_7miRNAs_3′UTR-2_ITR. The first cassette faces upstream 3′←5′. <------- and the second cassette faces downstream 5′→3′ (“normal”)------->as indicated in the schematic ITR_←-----------------_--------------->_ITR. ITR-bGHpA-7miRNA-CMV-hEf1α-7miRNA-WPRE3-SV40 pA-ITR.

### Example 6: Modification of Dog Protein Dog-stgfbr2-Fc

The dog protein dog-stgfbr2-fc was modified to include the mouse/human secretion signal. The nucleic acid encoding dog-stgfbr2-fc was modified to replace the following innate secretion signal ATGCACAGTCAAGGGCGGGGTTGCAACAACACAAAACAAAACAAAACTTCCGGA CTTCGACCTGCAGCTGAGAAGAACATCTCGCAAAGCGGCGTT (SEQ ID NO: 120)
with the mouse/human secretion signal as follows: ATGGGTCGGGGGCTGCTCCGGGGCCTGTGGCCGCTGCATATCGTCCTGTGGACGC GCATCGCCAGCACG (SEQ ID NO: 121). The nucleic acid sequence encoding the final protein is as follows.

(SEQ ID NO: 122)
ATGGGTCGGGGGCTGCTCCGGGGCCTGTGGCCGCTGCATATCGTCCTGTG
GACGCGCATCGCCAGCACGAATAATGACATGATGGTCACTGACAGCAATG
GTGTCATCAAATTTCCACAATTGTGTAAATTTTGTGATGTGAGATCTTCC
ACCTGTGACAACCAGAAATCTTGCATGAGCAACTGCAGCATTACATCCAT
CTGTGAGAAGCCACATGAAGTCTGTCTGGCTGTCTGGAGAAAGAATGATG
AGAACATAACACTAGAGACTCTCTGCCATGACCCCAAGGATACCTACCAT
GGAATTGTTCTCGAAGATGCTGCCTCTTCGAAGTGCATTATGAAAGAAAA
GAAGGTGCTGGGGGAGACTTTCTTTATGTGTTCCTGTAGCTCCGACGAGT
GCAACGACTACATCATCTTCTCTGAAGAATATGCCACCAACAACCCTGAC
TTGTTGTTAGTCATATTCCAACCCAAAAGAGAAAATGGAAGAGTTCCTCG
CCCACCTGATTGTCCCAAATGCCCAGCCCCTGAAATGCTGGGAGGGCCTT
CGGTCTTCATCTTTCCCCCGAAACCCAAGGACACCCTCTTGATTGCCCGA
ACACCTGAGGTCACATGTGTGGTGGTGGATCTGGACCCAGAAGACCCTGA
GGTGCAGATCAGCTGGTTCGTGGACGGTAAGCAGATGCAAACAGCCAAGA
CTCAGCCTCGTGAGGAGCAGTTCAATGGCACCTACCGTGTGGTCAGTGTC
CTCCCCATTGGGCACCAGGACTGGCTCAAGGGGAAGCAGTTCACGTGCAA
AGTCAACAACAAAGCCCTCCCATCCCCGATCGAGAGGACCATCTCCAAGG
CCAGAGGGCAAGCCCATCAGCCCAGTGTGTATGTCCTGCCGCCATCCCGG
GAGGAGTTGAGCAAGAACACAGTCAGCTTGACATGCCTGATCAAAGACTT
CTTCCCACCTGACATTGATGTGGAGTGGCAGAGCAATGGACAGCAGGAGC
CTGAGAGCAAGTACCGCACGACCCCGCCCCAGCTGGACGAGGACGGGTCC
TACTTCCTGTACAGCAAGCTCTCTGTGGACAAGAGCCGCTGGCAGCGGGG
AGACACCTTCATATGTGCGGTGATGCATGAAGCTCTACACAACCACTACA
CACAGGAATCCCTCTCCCATTCTCCGGGTAAAGGAGGGAGTGGTGGGTCC
GATTACAAAGATCACGATGGGGACTATAAAGATCACGACATCGACTATAA
GGATGACGATGATAAATGA.

Bold indicates the secretion signal. Bold and italics indicates the canine IGb heavy chain. Non-bolded indicates the canine TGFb receptor 2 extra cellular domain.

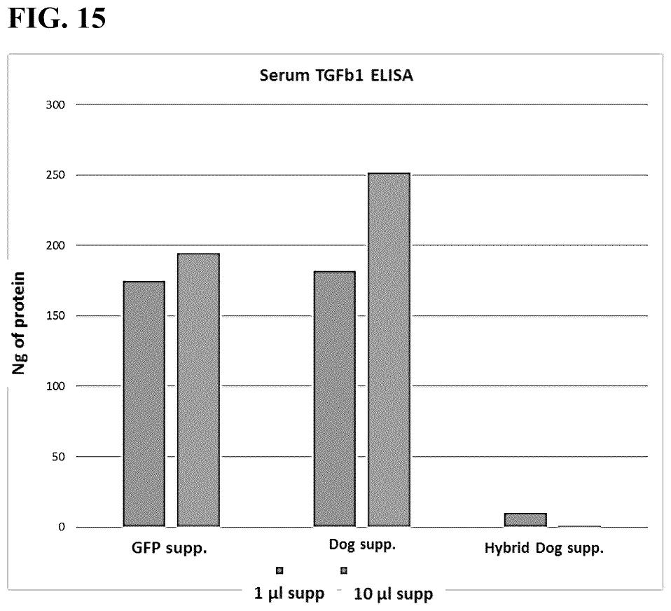

FIG. 15 indicates an in vitro ELISA assay in which it was demonstrated that the hybrid protein performs better than the original canine protein. The ELISA detects TGFbl, except when TGFb1 is bound by soluble TGF receptor 2 and is therefore prevented from binding in the ELISA assay. Briefly, supernatant from 293-Hek cells that were transfected with each construct or sHef1a-EGFP as a control were mixed with dog serum containing natural dog TGFb1 and the cells were assayed for their ability to secrete soluble TGF receptor 2. As indicated in FIG. 15, the natural dog protein did not get produced or secreted as well as the hybrid dog protein including the mouse/human secretion signal. The 293-Hek cells were better able to secrete the hybrid dog protein including the mouse/human secretion signal.

The experiments include the following secretion factors in place of the natural TGFbR2 secretion signal. One of skill will be able to identify additional useful secretion signals in the publicly available information that modulate expression to the desired level based on the present disclosure. Also, a screening mutagenesis can be carried out to find the optimal secretion signal for the particular peptide sequence to be secreted. According to this aspect, the sequence that is being secreted can modulate the efficacy of the secretion signal and the secretion signal can be optimized for a particular gene of interest.

According to one aspect, a vector can include a synthetic intron to increase expression via enhanced transport of the RNA out of the nucleus. Synthetic or natural introns known to those of skill in the art can be used for this purpose.

Chymo Trypsinogen (world wide sebsite unitargeting.com/Resources/Trends07.pdf)

(SEQ ID NO: 123)
ATGGCTTTTCTTTGGTTGCTGAGCTGCTGGGCACTGCTGGGTACTACTTT
TGGA
(SEQ ID NO: 124)
MAFLWLLSCWALLGTTFG
Trypsinogen
(SEQ ID NO: 125)
ATGAACTTGCTTCTCATCCTGACTTTTGTTGCAGCCGCCGTGGCT
HeavyChain
(SEQ ID NO: 126)
ATGGAGTTCGGGCTTTCTTGGGTGTTCTTGGTCGCTTTGTTTCGGGGGGT
CCAGTGT
(SEQ ID NO: 127)
MEFGLSWVFLVALFRGVQC
IL2-ILco1 (PMID: 15619290)
(SEQ ID NO: 128)
ATGAGGATGCAACTTCTCCTCTTGATAGCCCTTTCCTTGGCTCTGGTCAC
CAACAGC
(SEQ ID NO: 129)
MRMQLLLLIALSLALVTNS
IL2-ILco2 (PMID: 15619290)
(SEQ ID NO: 130)
MRRMQLLLLIALSLALVTNS
IL2 (PMID: 15619290)
(SEQ ID NO: 131)
MQLLSCIALILALV
Human serum albumin
(SEQ ID NO: 132)
MKWVTFISLLFLFSSAYS
human azurocidin preprotein
(SEQ ID NO: 133)
MTRLTVLALLAGLLASSRA
Gaussia luciferase
(SEQ ID NO: 134)
MGVKVLFALICIAVAEA

### Example 7: Methods of Preventing Heart Failure

According to certain aspects, gene therapy methods are provided for treating or preventing heart failure. A first group of mice was treated with a 1E11vg/mouse for AAV8:sHef1a-sTGFbR2-FC-WPRE3-SV40 pA and 1E11vg/mouse for AAV9:sHef1a-Nrf2-WPRE3-SV40 pA (double therapy). A second group of mice was treated with 1E11vg/mouse for AAV8:sHef1a-sTGFbR2-FC-WPRE3-SV40 pA (single therapy). Control AAC mice underwent surgery but did not receive the therapy. Mice receiving either the double therapy or the single therapy had higher fractional shortening, higher ejection fraction and lower heart mass compared to a control. According to certain aspects, mice are treated with sTGFbR2-FC, sTGFbR2-FC+FGF21, sTGFbR2-FC+Klotho, or sTGFbR2-FC+FGF21+Klotho in a method of treating or preventing heart failure or renal failure.

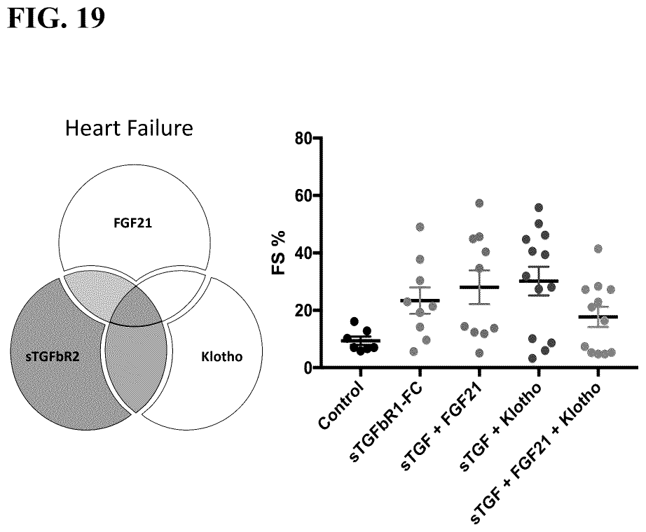

The following combination of genes was assessed as a gene therapy method, such as by using one or more AAVs for treating or preventing heart failure: FGF21, Klotho, and STGFbR2-FC. As indicated at FIG. 19, the Fractional shortening measurements (at 3 months post-surgery, surgery described previously for AAC) indicate that the combinations of genes treat ot prevent heart failure. More mice in the combination groups bifurcate into the compensated regime thereby overcoming the surgical banding placed 3 months prior. All the control mice become decompensated and will likely have died in the coming weeks. Whereas the mice in the other groups that ended up compensating showed no signs that they were going to die. The echocardiograms were all performed on conscience mice and were analyzed with native echo software.

### Example 8: Methods of Promoting Weight Loss

According to certain aspects, gene therapy methods are provided for promoting weight loss, such as for promoting weight loss in obese individuals. Fat mice were obtained from Jackson labs that were on a high fat diet for 3 months. Post arrival from Jackson labs, the mice were maintained on a 45% high fat diet D12451 from research diets form about 1 week. The mice were then injected with different doses of AAV8:sHef1a-FGF21-WPRE3-SV40 pA, as well as one combination of 1E9vg/mouse AAV8:sHef1a-FGF21-WPRE3-SV40 pA, 1E11vg/mouse AAV8:sHef1a-Klotho-WPRE3-SV40 pA, 1E11vg/mouse AAV8:sHef1a-STGFbR2-Fc-WPRE3-SV40 pA. All doses promoted weight loss while 1E11 and 1E10 promoted sustained weight loss.

### Example 9: Methods of Increasing Life Span

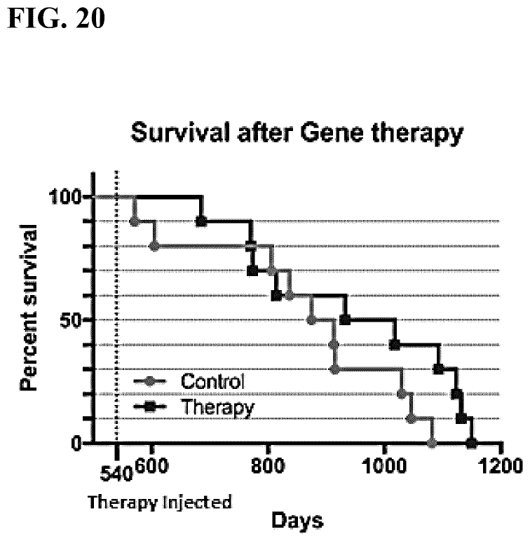

According to certain aspects, gene therapy methods as described herein are provided for increasing life span, health span or survival of an individual. An experiment was conducted using mice treated with sTGFbR2-FC in a gene therapy method described herein. Mice administered the sTGFbR2-FC gene provided an increase in life span as indicated in FIG. 20 with an approximate 10% increase in mean and maximum life span.

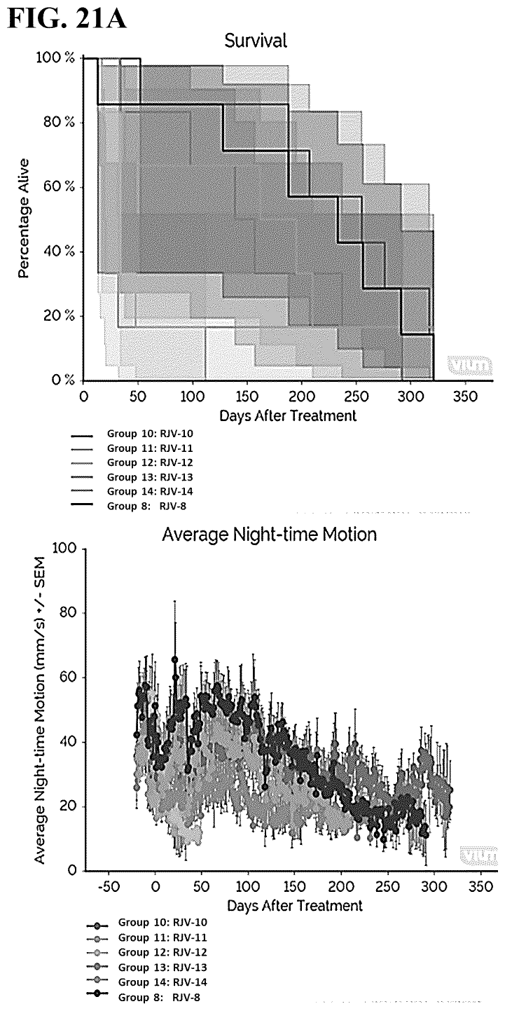

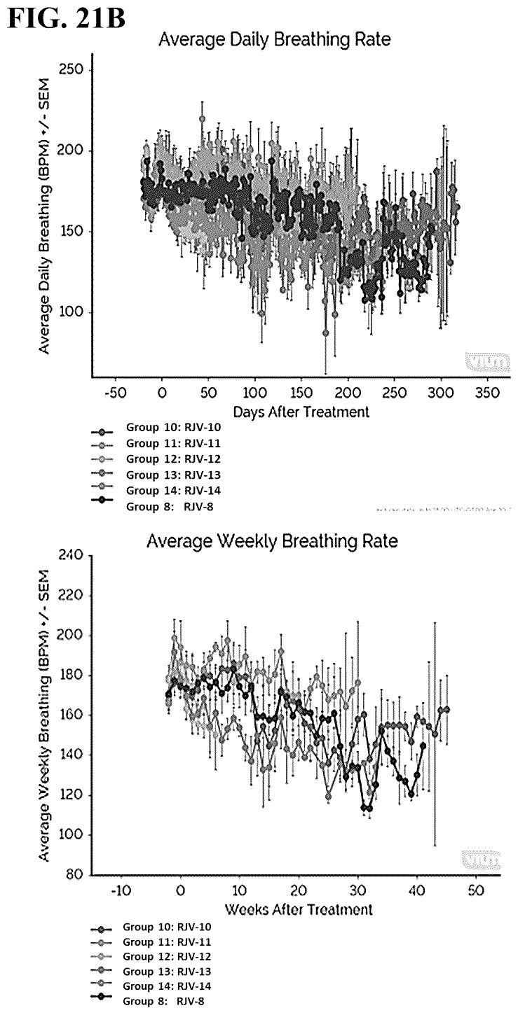

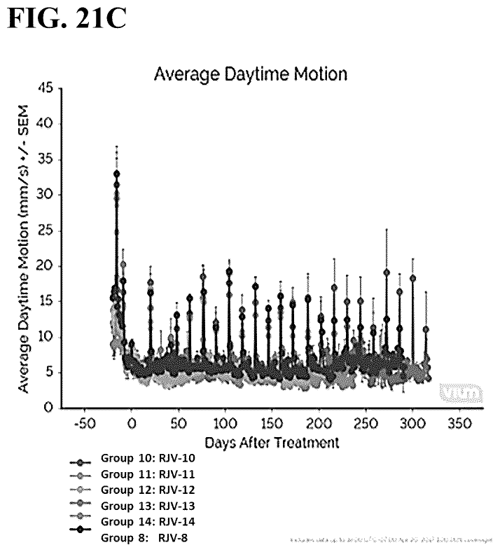

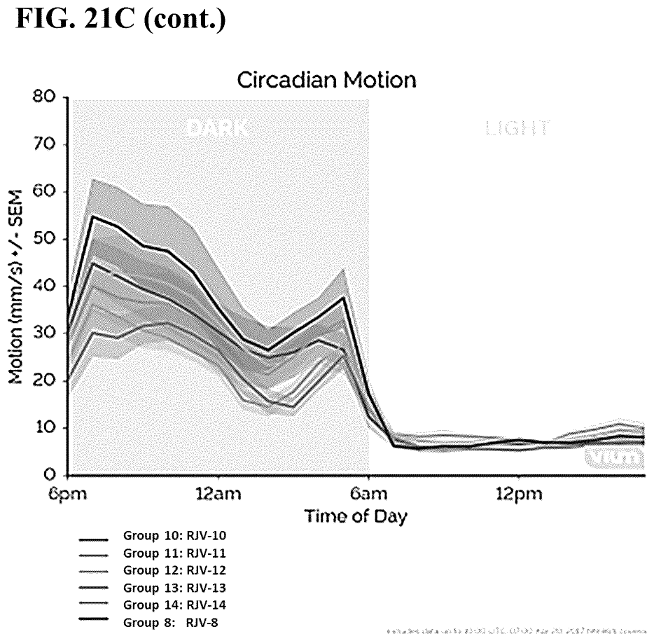

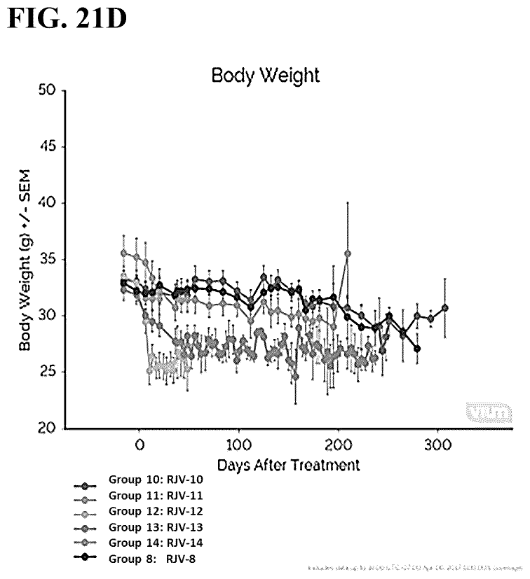

An additional experiment was carried out to determine increase in healthspan as measured by increased activity and respiration into old age using cameras and sensors 24 hours a day 7 days a week that provided image analysis to provide these metrics. The body weight of the mice was measured over time. The results are presented in FIGS. 21A-D. The experiment yielded numerous differences between the various therapy groups for circadian rhythms, breathing rates, lifespan, daily motions, and nighttime motion. The therapy groups and combination of gene therapies are identified in the Table 5 below.

| Group | RJV-1 | RJV-2 | RJV-3 | RJV-4 |
|---|---|---|---|---|
| Virus 1 | MT1 | sTGFbR2-FC | FGF21 | TERT |
| Virus 2 | Sirt6 | NRF2 | | |
| Virus 3 | sIGF1r-FC | | | |
| Virus 4 | Agtra1a-Adcy5-Coq7-(AAV-1) | | | |
| Virus 5 | Slc13a1-Ikbkb-(AAV-19) | | | |
| Virus 6 | Klotho | | | |
| Virus 7 | GDF15 | | | |
| Virus 8 | Ctf1-mTOR-Coq7-(AAV-16) | | | |
| Virus 9 | Slc13a5-Pappa-(AAV-22) | | | |
| Virus 10 | PCSK9-Rps6kb1-(AAV-17) | | | |

| Group | RJV-5 | RJV-6 | RJV-7 | RJV-8 |
|---|---|---|---|---|
| Virus 1 | FGF21 | FGF21 | FGF21 | AAV8:GFP |
| Virus 2 | TERT | TERT | TERT | AAV9:GFP |
| Virus 3 | | BubR1 | BubR1 | |
| Virus 4 | | Agtrala-Adcy5-Coq7-(AAV-1) | Agtra1a-Adcy5-Coq7-(AAV-1) | |
| Virus 5 | | | Slc13a1-Ikbkb-(AAV-19) | |
| Virus 6 | | | | |
| Virus 7 | | | | |
| Virus 8 | | | | |
| Virus 9 | | | | |
| Virus 10 | | | | |

| Group | RJV-9 | RJV-10 | RJV-11 | RJV-12 |
|---|---|---|---|---|
| Virus 1 | Nrf2 | Txn1 | PCSK9-Rps6kb1-(AAV-17) | Ctf1-mTOR-Coq7-(AAV-16) |
| Virus 2 | Txn1 | Sirt6 | Klotho | Agtra1a-Adcy5-mTOR-(AAV-6) |
| Virus 3 | TFAM-p2A-Cisd2-P2A-Nudt1 | Mt1 | Nrf2 | Cisd2 |
| Virus 4 | Klotho | TFEB | Txn1 | MCAT |
| Virus 5 | Sirt6 | Pck1 | HAS2 | FGF21 |
| Virus 6 | Atg5 | Adiponectin | Nudt1 | GDF15 |
| Virus 7 | Agtra1a-Adcy5-Akt1-(AAV-4) | Cisd2 | BubR1 | Klotho |
| Virus 8 | MCAT | Nudt1 | Dgat1-Pappa-(AAV-14) | Slc13a1-Ikbkb-(AAV-19) |
| Virus 9 | Slc13a1-Ikbkb-(AAV-19) | Atg5 | Ctf1-mTOR-Coq7-Slc13a5-(AAV-11) | Txn1 |
| Virus 10 | Ctf1-mTOR-Coq7-Slc13a5- | Ctf1-Ikbkb-Coq7-(AAV-9) | Agtra1a-Adcy5-Akt1-(AAV-4) | Sirt6 |
| | (AAV-11) | | | |

| Group | RJV-13 | RJV-14 |
|---|---|---|
| Virus 1 | MCAT | Txn1 |
| Virus 2 | Klotho | PCSK9-Rps6kb1-(AAV-17) |
| Virus 3 | GDF15 | Atg5 |
| Virus 4 | Neu1 | Ctf1-Akt1-(AAV-7) |
| Virus 5 | Mt1 | Pck1 |
| Virus 6 | hFoxP2 | klotho |
| Virus 7 | PCSK9-Rps6kb1-(AAV-17) | Nrf2 |
| Virus 8 | Ctf1-Ikbkb-Coq7-(AAV-9) | Cisd2 |
| Virus 9 | Slc13a1-mTOR-(AAV-20) | Dgat1-Pappa-(AAV-14) |
| Virus 10 | TFAM-p2A-Cisd2-P2A-Nudt1 | Ctf1-mTOR-Coq7-Slc13a5-(AAV-11) |

### Example 10: Methods of Treating or Preventing Renal Failure

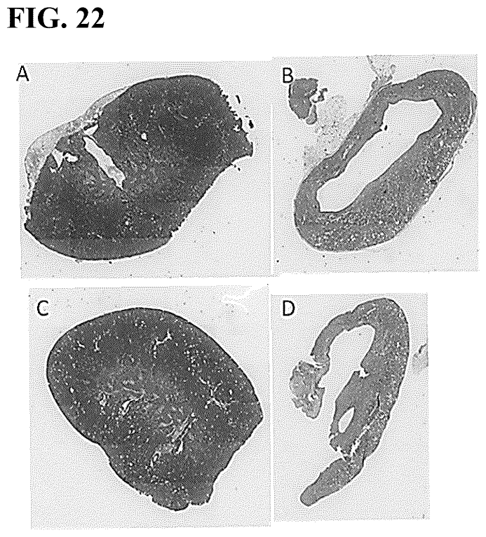

According to certain aspects, gene therapy methods are provided for treating or preventing renal failure. The following combination of genes is assessed as a gene therapy method, such as by using one or more AAVs for treating or preventing heart failure: FGF21, Klotho, and sTGFbR2-FC. FIG. 22 demonstrates a marked difference between the kidneys of control mice and mice treated with sTGFbR2-Fc gene therapy. A and C depict non-surgical contralateral control kidneys. B and D depict UUO kidneys with B depicting improved results compared to D.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

APPENDIX A
SEQUENCES: BOLD = secretion signal when indicated

```
    1. (SEQ ID NO: 3) sTGFBr2 Human DNA
ATGGGTCGGGGGCTGCTCAGGGGCCTGTGGCCGCTGCACATCGTCCTGTGGACGC
GTATCGCCAGCACGATCCCACCGCACGTTCAGAAGTCGGATGTGGAAATGGAGGCCCA
GAAAGATGAAATCATCTGCCCCAGCTGTAATAGGACTGCCCATCCACTGAGACATATTA
ATAACGACATGATAGTCACTGACAACAACGGTGCAGTCAAGTTTCCACAACTGTGTAAA
TTTTGTGATGTGAGATTTTCCACCTGTGACAACCAGAAATCCTGCATGAGCAACTGCAGC
ATCACCTCCATCTGTGAGAAGCCACAGGAAGTCTGTGTGGCTGTATGGAGAAAGAATGA
CGAGAACATAACACTAGAGACAGTTTGCCATGACCCCAAGCTCCCCTACCATGACTTTAT
TCTGGAAGATGCTGCTTCTCCAAAGTGCATTATGAAGGAAAAAAAAAAGCCTGGTGAGA
CTTTCTTCATGTGTTCCTGTAGCTCTGATGAGTGCAATGACAACATCATCTTCTCAGAAGA
ATATAACACCAGCAATCCTGAC
    2. (SEQ ID NO: 4) sTGFBr2 Dog DNA
ATGCACAGTCAAGGGCGGGGTTGCAACAACACAAAACAAAACAAAACTTCCGGACT
TCGACCTGCAGCTGAGAAGAACATCTCGCAAAGCGGCGTTAATAATGACATGATGGT
CACTGACAGCAATGGTGTCATCAAATTTCCACAATTGTGTAAATTTTGTGATGTGAGATC
TTCCACCTGTGACAACCAGAAATCTTGCATGAGCAACTGCAGCATTACATCCATCTGTGA
GAAGCCACATGAAGTCTGTCTGGCTGTCTGGAGAAAGAATGATGAGAACATAACACTAG
AGACTCTCTGCCATGACCCCAAGGATACCTACCATGGAATTGTTCTCGAAGATGCTGCCT
CTTCGAAGTGCATTATGAAAGAAAAGAAGGTGCTGGGGGAGACTTTCTTTATGTGTTCCT
GTAGCTCCGACGAGTGCAACGACTACATCATCTTCTCTGAAGAATATGCCACCAACAACC
CTGACTTGTTGTTAGTCATATTCCAA
    3. (SEQ ID NO: 5) Another version of sTGFBr2 with mouse/human secretion signal
ATGGGTCGGGGGCTGCTCCGGGGCCTGTGGCCGCTGCATATCGTCCTGTGGACGC
GCATCGCCAGCACGAATAATGACATGATGGTCACTGACAGCAATGGTGTCATCAAATTT
CCACAATTGTGTAAATTTTGTGATGTGAGATCTTCCACCTGTGACAACCAGAAATCTTGC
ATGAGCAACTGCAGCATTACATCCATCTGTGAGAAGCCACATGAAGTCTGTCTGGCTGTC
TGGAGAAAGAATGATGAGAACATAACACTAGAGACTCTCTGCCATGACCCCAAGGATAC
CTACCATGGAATTGTTCTCGAAGATGCTGCCTCTTCGAAGTGCATTATGAAAGAAAAGAA
GGTGCTGGGGGAGACTTTCTTTATGTGTTCCTGTAGCTCCGACGAGTGCAACGACTACAT
CATCTTCTCTGAAGAATATGCCACCAACAACCCTGACTTGTTGTTAGTCATATTCCAA
    4. sTGFBr2 Cat DNA
    5. 04 sTGFBr2 Cow DNA
    6. sTGFBr2 Sheep DNA
    7. sTGFBr2 Horse DNA
    8. sTGFBr2 Pig DNA
    9. (SEQ ID NO: 6) sTGFBr2 Mouse DNA
ATGGGTCGGGGGCTGCTCCGGGGCCTGTGGCCGCTGCATATCGTCCTGTGGACGC
GCATCGCCAGCACGATCCCGCCGCACGTTCCCAAGTCGGATGTGGAAATGGAAGCCCA
GAAAGATGCATCCATCCACCTAAGCTGTAATAGGACCATCCATCCACTGAAACATTTTAA
CAGTGATGTCATGGCCAGCGACAATGGCGGTGCGGTCAAGCTTCCACAGCTGTGCAAGT
TTTGCGATGTGAGACTGTCCACTTGCGACAACCAGAAGTCCTGCATGAGCAACTGCAGCA
TCACGGCCATCTGTGAGAAGCCGCATGAAGTCTGCGTGGCCGTGTGGAGGAAGAACGAC
AAGAACATTACTCTGGAGACGGTTTGCCACGACCCCAAGCTCACCTACCACGGCTTCACT
CTGGAAGATGCCGCTTCTCCCAAGTGTGTCATGAAGGAAAAGAAAAGGGCGGGCGAGAC
TTTCTTCATGTGTGCCTGTAACATGGAAGAGTGCAACGATTACATCATCTTTTCGGAAGA
ATACACCACCAGCAGTCCCGAC
  10. (SEQ ID NO: 7) sTGFBr2 Human AA
MGRGLLRGLWPLHIVLWTRIASTIPPHVQKSDVEMEAQKDEIICPSCNRTAHPLRHINND
MIVTDNNGAVKFPQLCKFCDVRFSTCDNQKSCMSNCSITSICEKPQEVCVAVWRKNDENITL
ETVCHDPKLPYHDFILEDAASPKCIMKEKKKPGETFFMCSCSSDECNDNIIFSEEYNTSNPD
  11. (SEQ ID NO: 8) sTGFBr2 Dog AA
MHSQGRGCNNTKQNKTSGLRPAAEKNISQSGVNNDMMVTDSNGVIKFPQLCKFCDVRSS
TCDNQKSCMSNCSITSICEKPHEVCLAVWRKNDENITLETLCHDPKDTYHGIVLEDAASSKCI
MKEKKVLGETFFMCSCSSDECNDYIIFSEEYATNNPD
  12. sTGFBr2 Cat AA
  13. sTGFBr2 Cow AA
  14. sTGFBr2 Sheep AA
  15. sTGFBr2 Horse AA
  16. sTGFBr2 Pig AA
  17. (SEQ ID NO: 9) sTGFBr2 Mouse AA
MGRGLLRGLWPLHIVLWTRIASTIPPHVPKSDVEMEAQKDASIHLSCNRTIHPLKHFNSDV
MASDNGGAVKLPQLCKFCDVRLSTCDNQKSCMSNCSITAICEKPHEVCVAVWRKNDKNITL
ETVCHDPKLTYHGFTLEDAASPKCVMKEKKRAGETFFMCACNMEECNDYIIFSEEYTTSSPD
  18. Fc Human
  19. (SEQ ID NO: 10) Fc Dog
CCCAAAAGAGAAAATGGAAGAGTTCCTCGCCCACCTGATTGTCCCAAATGCCCAGCCCC
TGAAATGCTGGGAGGGCCTTCGGTCTTCATCTTTCCCCCGAAACCCAAGGACACCCTCTT
GATTGCCCGAACACCTGAGGTCACATGTGTGGTGGTGGATCTGGACCCAGAAGACCCTG
AGGTGCAGATCAGCTGGTTCGTGGACGGTAAGCAGATGCAAACAGCCAAGACTCAGCCT
CGTGAGGAGCAGTTCAATGGCACCTACCGTGTGGTCAGTGTCCTCCCCATTGGGCACCAG
GACTGGCTCAAGGGGAAGCAGTTCACGTGCAAAGTCAACAACAAAGCCCTCCCATCCCC
GATCGAGAGGACCATCTCCAAGGCCAGAGGGCAAGCCCATCAGCCCAGTGTGTATGTCC
TGCCGCCATCCCGGGAGGAGTTGAGCAAGAACACAGTCAGCTTGACATGCCTGATCAAA
GACTTCTTCCCACCTGACATTGATGTGGAGTGGCAGAGCAATGGACAGCAGGAGCCTGA
GAGCAAGTACCGCACGACCCCGCCCCAGCTGGACGAGGACGGGTCCTACTTCCTGTACA
GCAAGCTCTCTGTGGACAAGAGCCGCTGGCAGCGGGGAGACACCTTCATATGTGCGGTG
ATGCATGAAGCTCTACACAACCACTACACACAGGAATCCCTCTCCCATTCTCCGGGTAAA
TGA
  20. (SEQ ID NO: 11) Fc Dog AA version:
PKRENGRVPRPPDCPKCPAPEMLGGPSVFIFPPKPICDTLLIARTPEVTCVVVDLDPEDPEVQIS
WFVDGKQMQTAKTQPREEQFNGTYRVVSVLPIGHQDWLKGKQFTCKVNNKALPSPIERTIS
KARGQAHQPSVYVLPPSREELSKNTVSLTCLIKDFFPPDIDVEWQSNGQQEPESKYRTTPPQL
DEDGSYFLYSKLSVDKSRWQRGDTFICAVMHEALHNHYTQESLSHSPGK
  21. Fc Cat
  22. Fc Cow
  23. Fc Sheep
  24. Fc Horse
  25. Fc Pig
  26. (SEQ ID NO: 12) Fc Mouse
CCCAGAGGGCCCACAATCAAGCCCTGTCCTCCATGCAAATGCCCAGCACCTAACCTCGA
GGGTGGACCATCCGTCTTCATCTTCCCTCCAAAGATCAAGGATGTACTCATGATCTCCCT
GAGCCCCATAGTCACATGTGTGGTGGTGGATGTGAGCGAGGATGACCCAGATGTCCAGA
TCAGCTGGTTTGTGAACAACGTGGAAGTACACACAGCTCAGACACAAACCCATAGAGAG
GATTACAACAGTACTCTCCGGGTGGTCAGTGCCCTCCCCATCCAGCACCAGGACTGGATG
AGTGGCAAGGCGTTCGCATGCGCGGTCAACAACAAAGACCTCCCAGCGCCCATCGAGAG
AACCATCTCAAAACCCAAAGGGTCAGTAAGAGCTCCACAGGTATATGTCTTGCCTCCACC
AGAAGAAGAGATGACTAAGAAACAGGTCACTCTGACCTGCATGGTCACAGACTTCATGC
CTGAAGACATTTACGTGGAGTGGACCAACAACGGGAAAACAGAGCTAAACTACAAGAA
CACTGAACCAGTCCTGGACTCTGATGGTTCTTACTTCATGTACAGCAAGCTGAGAGTGGA
AAAGAAGAACTGGGTGGAAAGAAATAGCTACTCCTGTTCAGTGGTCCACGAGGGTCTGC
ACAATCACCACACGACTAAGAGCTTCTCCCGGACTCCGGGTAAATGA
  27. (SEQ ID NO: 13) Fc Mouse AA version:
PRGPTIKPCPPCKCPAPNLEGGPSVFIFPPKIKDVLMISLSPIVTCVVVDVSEDDPDVQISWFVN
NVEVHTAQTQTHREDYNSTLRVVSALPIQHQDWMSGKAFACAVNNKDLPAPIERTISKPKGS
VRAPQVYVLPPPEEEMTKKQVTLTCMVTDFMPEDIYVEWTNNGKTELNYKNTEPVLDSDGS
YFMYSKLRVEKKNWVERNSYSCSVVHEGLHNHHTTKSFSRTPGK
  28. UCP1
  29. AMPK
  30. humanizeFoxP2
  31. NEU1
  32. NGF
  33. BubR1
  34. NAMPT
  35. Nmnat1
  36. (SEQ ID NO: 14) mNrf2 (mXXX indicates Murine for mouse)
ATGATGGACTTGGAGTTGCCACCGCCAGGACTACAGTCCCAGCAGGACATGGATTTGATT
GACATCCTTTGGAGGCAAGACATAGATCTTGGAGTAAGTCGAGAAGTGTTTGACTTTAGT
CAGCGACAGAAGGACTATGAGTTGGAAAAACAGAAAAAACTCGAAAAGGAAAGACAAG
AGCAACTCCAGAAGGAACAGGAGAAGGCCTTTTTCGCTCAGTTTCAACTGGATGAAGAA
ACAGGAGAATTCCTCCCAATTCAGCCGGCCCAGCACATCCAGACAGACACTAGTGGATC
CGCCAGCTACTCCCAGGTTGCCCACATTCCCAAACAAGATGCCTTGTACTTTGAAGACTG
TATGCAGCTTTTGGCAGAGACATTCCCATTTGTTGATGACCATGAGTCGCTTGCCCTGGA
TATCCCCAGCCACGCTGAAAGTTCAGTCTTCACTGCCCCTCATCAGGCCCAGTCCCTCAA
TAGCTCTCTGGAGGCAGCCATGACTGATTTAAGCAGCATAGAGCAGGACATGGAGCAAG
TTTGGCAGGAGCTATTTTCCATTCCCGAATTACAGTGTCTTAATACCGAAAACAAGCAGC
TGGCTGATACTACCGCTGTTCCCAGCCCAGAAGCCACACTGACAGAAATGGACAGCAAT
TACCATTTTTACTCATCGATCTCCTCGCTGGAAAAAGAAGTGGGCAACTGTGGTCCACAT
TTCCTTCATGGTTTTGAGGATTCTTTCAGCAGCATCCTCTCCACTGATGATGCCAGCCAGC
TGACCTCCTTAGACTCAAATCCCACCTTAAACACAGATTTTGGCGATGAATTTTATTCTGC
TTTCATAGCAGAGCCCAGTGACGGTGGCAGCATGCCTTCCTCCGCTGCCATCAGTCAGTC
ACTCTCTGAACTCCTGGACGGGACTATTGAAGGCTGTGACCTGTCACTGTGTAAAGCTTT
CAACCCGAAGCACGCTGAAGGCACAATGGAATTCAATGACTCTGACTCTGGCATTTCACT
GAACACAAGTCCCAGCCGAGCGTCCCCAGAGCACTCCGTGGAGTCTTCCATTTACGGAG
ACCCACCGCCTGGGTTCAGTGACTCGGAAATGGAGGAGCTAGATAGTGCCCCTGGAAGT
GTCAAACAGAACGGCCCTAAAGCACAGCCAGCACATTCTCCTGGAGACACAGTACAGCC
TCTGTCACCAGCTCAAGGGCACAGTGCTCCTATGCGTGAATCCCAATGTGAAAATACAAC
AAAAAAAGAAGTTCCCGTGAGTCCTGGTCATCAAAAAGCCCCATTCACAAAAGACAAAC
ATTCAAGCCGCTTAGAGGCTCATCTCACACGAGATGAGCTTAGGGCAAAAGCTCTCCATA
TTCCATTCCCTGTCGAAAAAATCATTAACCTCCCTGTTGATGACTTCAATGAAATGATGTC
CAAGGAGCAATTCAATGAAGCTCAGCTCGCATTGATCCGAGATATACGCAGGAGAGGTA
AGAATAAAGTCGCCGCCCAGAACTGTAGGAAAAGGAAGCTGGAGAACATTGTCGAGCTG
GAGCAAGACTTGGGCCACTTAAAAGACGAGAGAGAAAAACTACTCAGAGAAAAGGGAG
AAAACGACAGAAACCTCCATCTACTGAAAAGGCGGCTCAGCACCTTGTATCTTGAAGTCT
TCAGCATGTTACGTGATGAGGATGGAAAGCCTTACTCTCCCAGTGAATACTCTCTGCAGC
AAACCAGAGATGGCAATGTGTTCCTTGTTCCCAAAAGCAAGAAGCCAGATACAAAGAAA
AACTAG
  37. Sirt6
  38. TERT
  39. (SEQ ID NO: 15) mTFAM
ATGGCGCTGTTCCGGGGAATGTGGAGCGTGCTAAAAGCACTGGGGCGCACGGGGGTCGA
GATGTGCGCGGGCTGCGGGGGTCGCATCCCCTCGTCTATCAGTCTTGTCTGTATTCCGAA
GTGTTTTTCCAGCATGGGTAGCTATCCAAAGAAACCTATGAGTTCATACCTTCGATTTTCC
ACAGAACAGCTACCCAAATTTAAAGCTAAACACCCAGATGCAAAACTTTCAGAATTGGT
TAGGAAAATTGCAGCCCTGTGGAGGGAGCTACCAGAAGCAGAAAAAAAGGTTTATGAA
GCTGATTTTAAAGCTGAGTGGAAAGCATACAAAGAAGCTGTGAGCAAGTATAAAGAGCA
GCTAACTCCAAGTCAGCTGATGGGTATGGAGAAGGAGGCCCGGCAGAGACGGTTAAAAA
AGAAAGCACTGGTAAAGAGAAGAGAATTAATTTTGCTTGGAAAACCAAAAAGACCTCGT
TCAGCATATAACATTTATGTATCTGAAAGCTTCCAGGAGGCAAAGGATGATTCGGCTCAG
GGAAAATTGAAGCTTGTAAATGAGGCTTGGAAAAATCTGTCTCCTGAGGAAAAGCAGGC
ATATATTCAGCTTGCTAAAGATGATAGGATTCGTTACGACAATGAAATGAAGTCTTGGGA
AGAGCAGATGGCTGAAGTTGGACGAAGTGATCTCATCCGTCGAAGTGTGAAACGATCCG
GAGACATCTCTGAGCATTAA
  40. TFEB
  41. Fat1
  42. (SEQ ID NO: 16) Adiponectin
ATGCTACTGTTGCAAGCTCTCCTGTTCCTCTTAATCCTGCCCAGTCATGCCGAAGATGACG
TTACTACAACTGAAGAGCTAGCTCCTGCTTTGGTCCCTCCACCCAAGGGAACTTGTGCAG
GTTGGATGGCAGGCATCCCAGGACATCCTGGCCACAATGGCACACCAGGCCGTGATGGC
AGAGATGGCACTCCTGGAGAGAAGGGAGAGAAAGGAGATGCAGGTCTTCTTGGTCCTAA
GGGTGAGACAGGAGATGTTGGAATGACAGGAGCTGAAGGGCCACGGGGCTTCCCCGGA
ACCCCTGGCAGGAAAGGAGAGCCTGGAGAAGCCGCTTATGTGTATCGCTCAGCGTTCAG
TGTGGGGCTGGAGACCCGCGTCACTGTTCCCAATGTACCCATTCGCTTTACTAAGATCTT
CTACAACCAACAGAATCATTATGACGGCAGCACTGGCAAGTTCTACTGCAACATTCCGG
GACTCTACTACTTCTCTTACCACATCACGGTGTACATGAAAGATGTGAAGGTGAGCCTCT
TCAAGAAGGACAAGGCCGTTCTCTTCACCTACGACCAGTATCAGGAAAAGAATGTGGAC
CAGGCCTCTGGCTCTGTGCTCCTCCATCTGGAGGTGGGAGACCAAGTCTGGCTCCAGGTG
TATGGGGATGGGGACCACAATGGACTCTATGCAGATAACGTCAACGACTCTACATTTACT
GGCTTTCTTCTCTACCATGATACCAACTGATAA
  42. Klotho (canine) (SEQ ID NO: 135)
ATGGCCACCTGCATTTTACAGATGAGATTCCTAAGGCTGGGGAAGATACTGTTCCACTCC
AGCCCACAAAGCACAGGTGGCAGTGGTGGGACCCGGGGACCTCGAGCTCCGGCACAGCT
GCGAACGCAGCGTGGCACAGATAAGTTAGTTGCTAAGTCAGAGCTCAAGGCTAAAACGG
CCCACCGCGCGCTGGCCGACCACTTCAGGGACTACGCCGAGCTCTGCTTCCGCCACTTCT
GCGGCCAGGTCAAGTACTGGATCACCATCGACAACCCCTACGTGGTGGCCTGGCACGGC
TACGCCACCGGTCGCCTGGCACCCGGAGTCAGAGGCAGCCCGCGGCTCGGGTACCTGGT
GGCGCACAACCTCCTCCTGGCTCACGCCAAAATCTGGCATCTCTACAATACTTCTTTCCG
CCCAACTCAGGGAGGCCAGGTATCCATTGCCCTAAGCTCCCACTGGATCAATCCTCGAAG
AATGACCGACCATAGCATCAAAGAATGTCAAAAATCTCTTGACTTTGTACTAGGCTGGTT
TGCCAAGCCCATATTTATTGATGGTGACTATCCTGAGAGCATGAAGAATAACCTGTCATC
TCTTCTGCCTGTTTTTACTGAATCTGAGAAAAAGTTCATCAAGGGAACAGCTGACTTTTTT
GCTCTTTCTTTTGGACCAACTTTGAGTTTTCAACTCTTGGACCCTCATATGAAGTTCCACC
AATTAGAATCTCCCAGCCTGAGGCAACTCCTTTCTTGGATTGACCTTGAATATAACCACC
CTCAAATATTTATTGTGGAAAATGGCTGGTTTGTCTCAGGGACCACCAAGAGAGATGATG
CCAAATATATGTATTACCTCAAAAAATTCATAATGGAAACCTTAAAAGCCATCAGGCTGG
ATGGGGTGGATGTCATAGGATACACAGCGTGGTCCCTTATGGATGGCTTCGAGTGGCAC
AGAGGCTACAGCATCAGACGTGGACTCTTCTACGTGGACTTTCTAAGCCAGGATAAGAA
ACTGTTGCCAAAGTCTTCAGCCTTGTTCTACCAAAAGCTGATAGAGAAAAATGGCTTCCC
TCCTTTACCTGAAAATCAGCCCCTAGAAGGGACATTTCCCTGTGACTTTGCTTGGGGAAT
TGTTGACAACTACATTCAAGTGGACACCACTCTGTCTCAGTTTACCGACCCGAACGTTTA
CCTGTGGGACGTCCATCACAGCAAGAGGCTGATTAAGGTGGACGGGCTGCGGGCCAAGA
AGAGGAAGCCCTACTGCGTGGACTTTGCCGCCATCGGGCCCCAGGTGGCCCTGCTGCAG
GAGATGCACGTCTCGCATTTTCACTTCTCGCTGGACTGGGCCCTGCTCCTGCCGCTGGGC
AACCAGTCCCGGGTGAACCACGCGGCCCTGCACTACTACGGCTGCGTGGCCAGCGAGCT
CCTGCGCGCCAACATCACCCCGGTGGTGGCGCTCTGGAGACCAGCCGCTGCGCACCAGG
GTCTGCCTGGACCGCTGGCACAGCGCGGTGCCTGGGAGAACCCACGCACCGCCCTGGCG
TTCGCCGAGTACGCGCGCCTGTGCTTCCGCGCCCTGGGCCGCCACGTCAAGGTGTGGATC
ACGCTGCGCGAGCCGCCCACGCGGAACCTGACGCTCCGCGCCGGGCACAACCTGCTGCG
GGCGCACGCGCTGGCCTGGCGCGTGTACGACGAGCAGTTCCGGGGCTCGCAGCAGGGGA
AGGTGTCCATCGCCCTGCAGGCCGACTGGGTGGAGCCCGCCTGCCCCTCCTCCCAGAAGG
ACCGCGAAGTGGCCGAGAGGGTTCTGGAGTTCGACGTCGGCTGGCTGGCCGAGCCCATC
TTCGGCTCCGGGGACTACCCGCGGCTGATGCGCGACTGGCTCACCCGGAGAGACCATTCC
CTCCTGCCCTATTTCACTGACGAAGAGAAGAGGCTAATCCGGGGTTCCTTTGACTTCCTG
GCCTTGAGCCATTACACCACCATCCTCGTGGACTGGGAAAAGGAAGACCCAGTCAAATA
CAATGATTACCTGGAAGTGCAGGAGATGACCGACATCACCTGGCTCAACTCCCCCAGTC
AGGTGGCCGTAGTGCCCTGGGGCCTGCGCAAAGTGCTCAACTGGCTCAAGTTCAAGTAC
GGAGACCTCCCCATGTATATCGTATCCAACGGCATAGATGACGATCCGCGGGCAGCCCA
GGACTCGTTGAGGGTGTATTACATGCAGAACTATGTAAATGAAGCTCTGAAAGCCTACGT
ATTGGATGGTATCAATCTTTGTGGATACTTTGCCTACTCATTTAATGATCGCACAGCTCCG
AAGTTTGGCCTCTATCATTATGCTGCAAACCAGTTTGAGCCCAAACCGTCGGTGAAGCAT
TACAGGAAAATTATTGACAACAATGGCTTCCCAGGCCCTGAAACTTTGGGGCGGTTTTGT
CCAGAGGAATTCACCCTGTGCACCGAATGCAGCTTTTTTCACACCCGAAAGTCTTTACTG
GCTTTCATAGCTTTCCTACTTTTTGCTTTTATTATTTCTCTTTCTCTGATTTTCTACTACTCT
AGGAAAGGCAGAAGAAGTTATAAAGGAGGGAGTGGTGGGTCCGATTACAAAGATCACG
ATGGGGACTATAAAGATCACGACATCGACTATAAGGATGACGATGATAAATGA
  43. FGF21 (canine) (SEQ ID NO: 136)
ATGGGCTGGGCCGAGGCCGGGTTCGAGCACCTGGGACTGTGGGTCCCTGTGCTGGCTGT
GCTTTTGCTGGAAGCCTGCCGGGCACATCCGATCCCTGACTCCAGCCCCCTCCTACAATT
TGGAGGTCAAGTTCGACAGCGGTACCTCTACACCGACGATGCCCAGGAGACAGAGGCCC
ACCTAGAGATCAGGGCCGATGGCACAGTGGTGGGGGCTGCCCGCCAGAGCCCTGAAAGT
CTCCTGGAGCTGAAAGCCCTAAAGCCAGGGGTCATTCAAATCTTGGGAGTCAAAACATC
CAGGTTCCTGTGCCAGGGCCCAGATGGGACACTATATGGCTCGCTCCATTTCGACCCTGT
GGCCTGCAGTTTCCGAGAACTGCTTCTTGAGGATGGGTACAACATCTACCACTCCGAGAC
CCTTGGTCTCCCGCTTCGCCTGCGCCCCCACAACTCCGCATACCGGGACTTGGCACCCCG
CGGGCCTGCCCGCTTCCTGCCACTGCCAGGCCTGCTTCCAGCACCCCCAGAGCCTCCAGG
GATCCTGGCCCCGGAGCCTCCTGACGTGGGCTCCTCGGACCCTCTGAGCATGGTGGGGCC
TTCACAGGGCCGGAGTCCCAGCTATGCTTCCTGATAG
  44. GDF15 (hNAG)
  45. IL4
  46. ZAG
  47. HAS2
  48. Txn1
  49. Cat
  50. Plau
  51. Ucp2
  52. Atg5
  53. NUDT1
  54. Mt1
  55. Adra1a (mut)
  56. Pck1
  57. Serpine1
  58. GH
  59. IFG1
  60. TGFb1
  61. PDE4b
  62. mTOR
  63. nf-kb
  64. PCSK9
  65. bCATm
  66. ADcy5
  67. Coq7
  68. Eps8
  69. Insr
  70. Pappa
  71. Shc1
  72. Agtr1a
  73. Slc13a1
  74. Dgat1
  75. Ikbkb
  76. Kcna3
  77. Myc
  78. Surf1
  79. Ubd
  80. Rps6kb1
  81. Ctf1
  82. Htt
  83. Irs1
  84. Irs2
  85. Mif
  86. Trpv1
  87. Prkar2b
  88. Gsta4
  89. Akt1
  90. Gpx4
  91. Bax
  92. Cebpalpha
  93. Cebpbeta
  94. (SEQ ID NO: 17) heF1a Promoter
TTGGCTCCGGTGCCCGTCAGTGGGCAGAGCGCACATCGCCCACAGTCCCCGAGAAGTTGT
GGGGAGGGGTCGGCAATTGAACCGGTGCCTAGAGAAGGTGGCGCGGGGTAAACTGGGA
AAGTGATGTCGTGTACTGGCTCCGCCTTTTTCCCGAGGGTGGGGGAGAACCGTATATAAG
TGCAGTAGTCGCCGTGAACGTTCTTTTTCGCAACGGGTTTGCCGCCAGAACACAGGTAAG
TGCCGTGTGTGGTTCCCGCGGGCCTGGCCTCTTTACGGGTTATGGCCCTTGCGTGCCTTGA
ATTACTTCCACCTGGCTGCAGTACGTGATTCTTGATCCCGAGCTTCGGGTTGGAAGTGGG
TGGGAGAGTTCGAGGCCTTGCGCTTAAGGAGCCCCTTCGCCTCGTGCTTGAGTTGAGGCC
TGGCCTGGGCGCTGGGGCCGCCGCGTGCGAATCTGGTGGCACCTTCGCGCCTGTCTCGCT
GCTTTCGATAAGTCTCTAGCCATTTAAAATTTTTGATGACCTGCTGCGACGCTTTTTTTCT
GGCAAGATAGTCTTGTAAATGCGGGCCAAGATCTGCACACTGGTATTTCGGTTTTTGGGG
CCGCGGGCGGCGACGGGGCCCGTGCGTCCCAGCGCACATGTTCGGCGAGGCGGGGCCTG
CGAGCGCGGCCACCGAGAATCGGACGGGGGTAGTCTCAAGCTGGCCGGCCTGCTCTGGT
GCCTGGCCTCGCGCCGCCGTGTATCGCCCCGCCCTGGGCGGCAAGGCTGGCCCGGTCGGC
ACCAGTTGCGTGAGCGGAAAGATGGCCGCTTCCCGGCCCTGCTGCAGGGAGCTCAAAAT
GGAGGACGCGGCGCTCGGGAGAGCGGGCGGGTGAGTCACCCACACAAAGGAAAAGGGC
CTTTCCGTCCTCAGCCGTCGCTTCATGTGACTCCACGGAGTACCGGGCGCCGTCCAGGCA
CCTCGATTAGTTCTCGAGCTTTTGGAGTACGTCGTCTTTAGGTTGGGGGGAGGGGTTTTAT
GCGATGGAGTTTCCCCACACTGAGTGGGTGGAGACTGAAGTTAGGCCAGCTTGGCACTT
GATGTAATTCTCCTTGGAATTTGCCCTTTTTGAGTTTGGATCTTGGTTCATTCTCAAGCCT
CAGACAGTGGTTCAAAGTTTTTTTCTTCCATTTCAGGT
  95. (SEQ ID NO: 18) sheF1a Promoter
AGGCTCCGGTGCCCGTCAGTGGGCAGAGCGCACATCGCCCACAGTCCCCGAGAAGTTGG
GGGGAGGGGTCGGCAATTGAACCGGTGCCTAGAGAAGGTGGCGCGGGGTAAACTGGGA
AAGTGATGTCGTGTACTGGCTCCGCCTTTTTCCCGAGGGTGGGGGAGAACCGTATATAAG
TGCAGTAGTCGCCGTGAACGTTCTTTTTCGCAACGGGTTTGCCGCCAGAACACA
  96. (SEQ ID NO: 19) rheF1a Promoter
GGATCTGCGATCGCTCCGGTGCCCGTCAGTGGGCAGAGCGCACATCGCCCACAGTCCCC
GAGAAGTTGGGGGGAGGGGTCGGCAATTGAACGGGTGCCTAGAGAAGGTGGCGCGGGG
TAAACTGGGAAAGTGATGTCGTGTACTGGCTCCGCCTTTTTCCCGAGGGTGGGGGAGAAC
CGTATATAAGTGCAGTAGTCGCCGTGAACGTTCTTTTTCGCAACGGGTTTGCCGCCAGAA
CACAGCTGAAGCTTCGAGGGGCTCGCATCTCTCCTTCACGCGCCCGCCGCCCTACCTGAG
GCCGCCATCCACGCCGGTTGAGTCGCGTTCTGCCGCCTCCCGCCTGTGGTGCCTCCTGAA
CTGCGTCCGCCGTCTAGGTAAGTTTAAAGCTCAGGTCGAGACCGGGCCTTTGTCCGGCGC
TCCCTTGGAGCCTACCTAGACTCAGCCGGCTCTCCACGCTTTGCCTGACCCTGCTTGCTCA
ACTCTACGTCTTTGTTTCGTTTTCTGTTCTGCGCCGTTACAGATCCAAGCTGTGACCGGCG
CCTAC
  97. AAT Promoter
  98. thyroid hormone-binding globulin promoter
  99. albumin promoter
100. thyroxin-binding globulin (TBG) promoter
101. Hepatic Control Region (HCR)-ApoCII hybrid promoter
102. HCR-hAAT hybrid promoter
103. AAT promoter combined with mouse albumin gene enhancer (Ealb) element and an
apolipoprotein E promoter
104. (SEQ ID NO: 20) P2A
GGATCTGGCGCCACCAACTTCTCTCTGCTGAAGCAGGCCGGCGACGTGGAGGAGAACCC
AGGCCCA
105. (SEQ ID NO: 21) T2A
GAGGGCCGCGGCAGCCTGCTGACCTGCGGCGACGTGGAGGAAAACCCCGGCCCC
106. E2A
107. (SEQ ID NO: 22) 105 IRES
GCCCCTCTCCCTCCCCCCCCCCTAACGTTACTGGCCGAAGCCGCTTGGAATAAGGCCGGT
GTGCGTTTGTCTATATGTTATTTTCCACCATATTGCCGTCTTTTGGCAATGTGAGGGCCCG
GAAACCTGGCCCTGTCTTCTTGACGAGCATTCCTAGGGGTCTTTCCCCTCTCGCCAAAGG
AATGCAAGGTCTGTTGAATGTCGTGAAGGAAGCAGTTCCTCTGGAAGCTTCTTGAAGACA
AACAACGTCTGTAGCGACCCTTTGCAGGCAGCGGAACCCCCCACCTGGCGACAGGTGCC
TCTGCGGCCAAAAGCCACGTGTATAAGATACACCTGCAAAGGCGGCACAACCCCAGTGC
CACGTTGTGAGTTGGATAGTTGTGGAAAGAGTCAAATGGCTCTCCTCAAGCGTATTCAAC
AAGGGGCTGAAGGATGCCCAGAAGGTACCCCATTGTATGGGATCTGATCTGGGGCCTCG
GTGCACATGCTTTACATGTGTTTAGTCGAGGTTAAAAAAACGTCTAGGCCCCCCGAACCA
CGGGGACGTGGTTTTCCTTTGAAAAACACGATGATAAGCCACC
108. AAV1 ITR
109. (SEQ ID NO: 23) AAV2 ITR 5′ ITR
CCTGCAGGCAGCTGCGCGCTCGCTCGCTCACTGAGGCCGCCCGGGCGTCGGGCGACCTTT
GGTCGCCCGGCCTCAGTGAGCGAGCGAGCGCGCAGAGAGGGAGTGGCCAACTCCATCAC
TAGGGGTTCCT
110. (SEQ ID NO: 24) AAV2 ITR 3′ ITR
CCTGCAGGCAGCTGCGCGCTCGCTCGCTCACTGAGGCCGCCCGGGCAAAGCCCGGGCGT
CGGGCGACCTTTGGTCGCCCGGCCTCAGTGAGCGAGCGAGCGCGCAGAGAGGGAGTGGC
CAACTCCATCACTAGGGGTTCCT
111. AAV5 ITR
112. AAV6 ITR
113. AAV7 ITR
114. AAV8 ITR
115. AAV9 ITR
116. (SEQ ID NO: 25) AAV-8 capsid
ATGGCTGCCGATGGTTATCTTCCAGATTGGCTCGAGGACAACCTCTCTGAGGGCATTCGC
GAGTGGTGGGCGCTGAAACCTGGAGCCCCGAAGCCCAAAGCCAACCAGCAAAAGCAGG
ACGACGGCCGGGGTCTGGTGCTTCCTGGCTACAAGTACCTCGGACCCTTCAACGGACTCG
ACAAGGGGGAGCCCGTCAACGCGGCGGACGCAGCGGCCCTCGAGCACGACAAGGCCTA
CGACCAGCAGCTGCAGGCGGGTGACAATCCGTACCTGCGGTATAACCACGCCGACGCCG
AGTTTCAGGAGCGTCTGCAAGAAGATACGTCTTTTGGGGGCAACCTCGGGCGAGCAGTC
TTCCAGGCCAAGAAGCGGGTTCTCGAACCTCTCGGTCTGGTTGAGGAAGGCGCTAAGAC
GGCTCCTGGAAAGAAGAGACCGGTAGAGCCATCACCCCAGCGTTCTCCAGACTCCTCTA
CGGGCATCGGCAAGAAAGGCCAACAGCCCGCCAGAAAAAGACTCAATTTTGGTCAGACT
GGCGACTCAGAGTCAGTTCCAGACCCTCAACCTCTCGGAGAACCTCCAGCAGCGCCCTCT
GGTGTGGGACCTAATACAATGGCTGCAGGCGGTGGCGCACCAATGGCAGACAATAACGA
AGGCGCCGACGGAGTGGGTAGTTCCTCGGGAAATTGGCATTGCGATTCCACATGGCTGG
GCGACAGAGTCATCACCACCAGCACCCGAACCTGGGCCCTGCCCACCTACAACAACCAC
CTCTACAAGCAAATCTCCAACGGGACATCGGGAGGAGCCACCAACGACAACACCTACTT
CGGCTACAGCACCCCCTGGGGGTATTTTGACTTTAACAGATTCCACTGCCACTTTTCACC
ACGTGACTGGCAGCGACTCATCAACAACAACTGGGGATTCCGGCCCAAGAGACTCAGCT
TCAAGCTCTTCAACATCCAGGTCAAGGAGGTCACGCAGAATGAAGGCACCAAGACCATC
GCCAATAACCTCACCAGCACCATCCAGGTGTTTACGGACTCGGAGTACCAGCTGCCGTAC
GTTCTCGGCTCTGCCCACCAGGGCTGCCTGCCTCCGTTCCCGGCGGACGTGTTCATGATTC
CCCAGTACGGCTACCTAACACTCAACAACGGTAGTCAGGCCGTGGGACGCTCCTCCTTCT
ACTGCCTGGAATACTTTCCTTCGCAGATGCTGAGAACCGGCAACAACTTCCAGTTTACTT
ACACCTTCGAGGACGTGCCTTTCCACAGCAGCTACGCCCACAGCCAGAGCTTGGACCGG
CTGATGAATCCTCTGATTGACCAGTACCTGTACTACTTGTCTCGGACTCAAACAACAGGA
GGCACGGCAAATACGCAGACTCTGGGCTTCAGCCAAGGTGGGCCTAATACAATGGCCAA
TCAGGCAAAGAACTGGCTGCCAGGACCCTGTTACCGCCAACAACGCGTCTCAACGACAA
CCGGGCAAAACAACAATAGCAACTTTGCCTGGACTGCTGGGACCAAATACCATCTGAAT
GGAAGAAATTCATTGGCTAATCCTGGCATCGCTATGGCAACACACAAAGACGACGAGGA
GCGTTTTTTTCCCAGTAACGGGATCCTGATTTTTGGCAAACAAAATGCTGCCAGAGACAA
TGCGGATTACAGCGATGTCATGCTCACCAGCGAGGAAGAAATCAAAACCACTAACCCTG
TGGCTACAGAGGAATACGGTATCGTGGCAGATAACTTGCAGCAGCAAAACACGGCTCCT
CAAATTGGAACTGTCAACAGCCAGGGGGCCTTACCCGGTATGGTCTGGCAGAACCGGGA
CGTGTACCTGCAGGGTCCCATCTGGGCCAAGATTCCTCACACGGACGGCAACTTCCACCC
GTCTCCGCTGATGGGCGGCTTTGGCCTGAAACATCCTCCGCCTCAGATCCTGATCAAGAA
CACGCCTGTACCTGCGGATCCTCCGACCACCTTCAACCAGTCAAAGCTGAACTCTTTCAT
CACGCAATACAGCACCGGACAGGTCAGCGTGGAAATTGAATGGGAGCTGCAGAAGGAA
AACAGCAAGCGCTGGAACCCCGAGATCCAGTACACCTCCAACTACTACAAATCTACAAG
TGTGGACTTTGCTGTTAATACAGAAGGCGTGTACTCTGAACCCCGCCCCATTGGCACCCG
TTACCTCACCCGTAATCTG
117. AAV-9 capsid
118. (SEQ ID NO: 26) AAV-PHP.b capsid
ATGGCTGCCGATGGTTATCTTCCAGATTGGCTCGAGGACAACCTTAGTGAAGGAATTCGC
GAGTGGTGGGCTTTGAAACCTGGAGCCCCTCAACCCAAGGCAAATCAACAACATCAAGA
CAACGCTCGAGGTCTTGTGCTTCCGGGTTACAAATACCTTGGACCCGGCAACGGACTCGA
CAAGGGGGAGCCGGTCAACGCAGCAGACGCGGCGGCCCTCGAGCACGACAAGGCCTAC
GACCAGCAGCTCAAGGCCGGAGACAACCCGTACCTCAAGTACAACCACGCCGACGCCGA
GTTCCAGGAGCGGCTCAAAGAAGATACGTCTTTTGGGGGCAACCTCGGGCGAGCAGTCT
TCCAGGCCAAAAAGAGGCTTCTTGAACCTCTTGGTCTGGTTGAGGAAGCGGCTAAGACG
GCTCCTGGAAAGAAGAGGCCTGTAGAGCAGTCTCCTCAGGAACCGGACTCCTCCGCGGG
TATTGGCAAATCGGGTGCACAGCCCGCTAAAAAGAGACTCAATTTCGGTCAGACTGGCG
ACACAGAGTCAGTCCCAGACCCTCAACCAATCGGAGAACCTCCCGCAGCCCCCTCAGGT
GTGGGATCTCTTACAATGGCTTCAGGTGGTGGCGCACCAGTGGCAGACAATAACGAAGG
TGCCGATGGAGTGGGTAGTTCCTCGGGAAATTGGCATTGCGATTCCCAATGGCTGGGGG
ACAGAGTCATCACCACCAGCACCCGAACCTGGGCCCTGCCCACCTACAACAATCACCTCT
ACAAGCAAATCTCCAACAGCACATCTGGAGGATCTTCAAATGACAACGCCTACTTCGGCT
ACAGCACCCCCTGGGGGTATTTTGACTTCAACAGATTCCACTGCCACTTCTCACCACGTG
ACTGGCAGCGACTCATCAACAACAACTGGGGATTCCGGCCTAAGCGACTCAACTTCAAG
CTCTTCAACATTCAGGTCAAAGAGGTTACGGACAACAATGGAGTCAAGACCATCGCCAA
TAACCTTACCAGCACGGTCCAGGTCTTCACGGACTCAGACTATCAGCTCCCGTACGTGCT
CGGGTCGGCTCACGAGGGCTGCCTCCCGCCGTTCCCAGCGGACGTTTTCATGATTCCTCA
GTACGGGTATCTGACGCTTAATGATGGAAGCCAGGCCGTGGGTCGTTCGTCCTTTTACTG
CCTGGAATATTTCCCGTCGCAAATGCTAAGAACGGGTAACAACTTCCAGTTCAGCTACGA
GTTTGAGAACGTACCTTTCCATAGCAGCTACGCTCACAGCCAAAGCCTGGACCGACTAAT
GAATCCACTCATCGACCAATACTTGTACTATCTCTCTAGAACTATTAACGGTTCTGGACA
GAATCAACAAACGCTAAAATTCAGTGTGGCCGGACCCAGCAACATGGCTGTCCAGGGAA
GAAACTACATACCTGGACCCAGCTACCGACAACAACGTGTCTCAACCACTGTGACTCAA
AACAACAACAGCGAATTTGCTTGGCCTGGAGCTTCTTCTTGGGCTCTCAATGGACGTAAT
AGCTTGATGAATCCTGGACCTGCTATGGCCAGCCACAAAGAAGGAGAGGACCGTTTCTTT
CCTTTGTCTGGATCTTTAATTTTTGGCAAACAAGGTACCGGCAGAGACAACGTGGATGCG
GACAAAGTCATGATAACCAACGAAGAAGAAATTAAAACTACTAACCCGGTAGCAACGG
AGTCCTATGGACAAGTGGCCACAAACCACCAGAGTGCCCAAACTTTGGCGGTGCCTTTTA
AGGCACAGGCGCAGACCGGTTGGGTTCAAAACCAAGGAATACTTCCGGGTATGGTTTGG
CAGGACAGAGATGTGTACCTGCAAGGACCCATTTGGGCCAAAATTCCTCACACGGACGG
CAACTTTCACCCTTCTCCGCTGATGGGAGGGTTTGGAATGAAGCACCCGCCTCCTCAGAT
CCTCATCAAAAACACACCTGTACCTGCGGATCCTCCAACGGCCTTCAACAAGGACAAGCT
GAACTCTTTCATCACCCAGTATTCTACTGGCCAAGTCAGCGTGGAGATCGAGTGGGAGCT
GCAGAAGGAAAACAGCAAGCGCTGGAACCCGGAGATCCAGTACACTTCCAACTATTACA
AGTCTAATAATGTTGAATTTGCTGTTAATACTGAAGGTGTATATAGTGAACCCCGCCCCA
TTGGCACCAGATACCTGACTCGTAATCTG
119. (SEQ ID NO: 27) WPRE
AATCAACCTCTGGATTACAAAATTTGTGAAAGATTGACTGGTATTCTTAACTATGTTGCT
CCTTTTACGCTATGTGGATACGCTGCTTTAATGCCTTTGTATCATGCTATTGCTTCCCGTA
TGGCTTTCATTTTCTCCTCCTTGTATAAATCCTGGTTGCTGTCTCTTTATGAGGAGTTGTGG
CCCGTTGTCAGGCAACGTGGCGTGGTGTGCACTGTGTTTGCTGACGCAACCCCCACTGGT
TGGGGCATTGCCACCACCTGTCAGCTCCTTTCCGGGACTTTCGCTTTCCCCCTCCCTATTG
CCACGGCGGAACTCATCGCCGCCTGCCTTGCCCGCTGCTGGACAGGGGCTCGGCTGTTGG
GCACTGACAATTCCGTGGTGTTGTCGGGGAAGCTGACGTCCTTTCCATGGCTGCTCGCCT
GTGTTGCCACCTGGATTCTGCGCGGGACGTCCTTCTGCTACGTCCCTTCGGCCCTCAATCC
AGCGGACCTTCCTTCCCGCGGCCTGCTGCCGGCTCTGCGGCCTCTTCCGCGTCTTCGCCTT
CGCCCTCAGACGAGTCGGATCTCCCTTTGGGCCGCCTCCCCGCCTG
120. (SEQ ID NO: 28) WPRE3
AATCAACCTCTGGATTACAAAATTTGTGAAAGATTGACTGGTATTCTTAACTATGTTGCT
CCTTTTACGCTATGTGGATACGCTGCTTTAATGCCTTTGTATCATGCTATTGCTTCCCGTA
TGGCTTTCATTTTCTCCTCCTTGTATAAATCCTGGTTAGTTCTTGCCACGGCGGAACTCAT
CGCCGCCTGCCTTGCCCGCTGCTGGACAGGGGCTCGGCTGTTGGGCACTGACAATTCCGT
GGTGTT
121. (SEQ ID NO: 29) SV40pA
GCTTTATTTGTGAAATTTGTGATGCTATTGCTTTATTTGTAACCATTATAAGCTGCAATAA
ACAAGTTAACAACAACAATTGCATTCATTTTATGTTTCAGGTTCAGGGGGAGATGTGGGA
GGTTTTTTAAAGC
122. (SEQ ID NO: 30) bGHpA
ACGGGTGGCATCCCTGTGACCCCTCCCCAGTGCCTCTCCTGGCCCTGGAAGTTGCCACTC
CAGTGCCCACCAGCCTTGTCCTAATAAAATTAAGTTGCATCATTTTGTCTGACTAGGTGTC
CTTCTATAATATTATGGGGTGGAGGGGGGTGGTATGGAGCAAGGGGCAAGTTGGGAAGA
CAACCTGTAGGGCCTGCGGGGTCTATTGGGAACCAAGCTGGAGTGCAGTGGCACAATCT
TGGCTCACTGCAATCTCCGCCTCCTGGGTTCAAGCGATTCTCCTGCCTCAGCCTCCCGAGT
TGTTGGGATTCCAGGCATGCATGACCAGGCTCAGCTAATTTTTGTTTTTTTGGTAGAGAC
GGGGTTTCACCATATTGGCCAGGCTGGTCTCCAACTCCTAATCTCAGGTGATCTACCCAC
CTTGGCCTCCCAAATTGCTGGGATTACAGGCGTGAACCACTGCTCCCTTCCCTGTCCTTCT
GATTTTGTAGGTAACCACGTGCGGACCGA
123. (SEQ ID NO: 31) rBGpA
TGGCTAATAAAGGAAATTTATTTTCATTGCAATAGTGTGTTGGAATTTTTTGTGTCTCTCA
CTCGGAAGGACATATGGGAGGGCAAATCATTTAAAACATCAGAATGAGTATTTGGTTTA
GAGTTTGGCAACATATGCCCATATGCTGGCTGCCATGAACAAAGGTTGGCTATAAAGAG
GTCATCAGTATATGAAACAGCCCCCTGCTGTCCATTCCTTATTCCATAGAAAAGCCTTGA
CTTGAGGTTAGATTTTTTTTATATTTTGTTTTGTGTTATTTTTTTCTTTAACATCCCTAAAA
TTTTCCTTACATGTTTTACTAGCCAGATTTTTCCTCCTCTCCTGACTACTCCCAGTCATAGC
TGTCCCTCTTCTCTTATGGAGATC
124. (SEQ ID NO: 32) hGHpA
ACGGGTGGCATCCCTGTGACCCCTCCCCAGTGCCTCTCCTGGCCCTGGAAGTTGCCACTC
CAGTGCCCACCAGCCTTGTCCTAATAAAATTAAGTTGCATCATTTTGTCTGACTAGGTGTC
CTTCTATAATATTATGGGGTGGAGGGGGGTGGTATGGAGCAAGGGGCAAGTTGGGAAGA
CAACCTGTAGGGCCTGCGGGGTCTATTGGGAACCAAGCTGGAGTGCAGTGGCACAATCT
TGGCTCACTGCAATCTCCGCCTCCTGGGTTCAAGCGATTCTCCTGCCTCAGCCTCCCGAGT
TGTTGGGATTCCAGGCATGCATGACCAGGCTCAGCTAATTTTTGTTTTTTTGGTAGAGAC
GGGGTTTCACCATATTGGCCAGGCTGGTCTCCAACTCCTAATCTCAGGTGATCTACCCAC
CTTGGCCTCCCAAATTGCTGGGATTACAGGCGTGAACCACTGCTCCCTTCCCTGTCCTT
```

APPENDIX B

| | | Molecule | |
|---|---|---|---|
| Name | SEQ ID | Type | Sequence |
| mAdipoQ | SEQ ID NO: 33 | DNA | ATGCTACTGTTGCAAGCTCTCCTGTTCCTCTTAATCCTGCCC |
| | | | AGTCATGCCGAAGATGACGTTACTACAACTGAAGAGCTAGC |
| | | | TCCTGCTTTGGTCCCTCCACCCAAGGGAACTTGTGCAGGTTG |
| | | | GATGGCAGGCATCCCAGGACATCCTGGCCACAATGGCACAC |
| | | | CAGGCCGTGATGGCAGAGATGGCACTCCTGGAGAGAAGGG |
| | | | AGAGAAAGGAGATGCAGGTCTTCTTGGTCCTAAGGGTGAGA |
| | | | CAGGAGATGTTGGAATGACAGGAGCTGAAGGGCCACGGGG |
| | | | CTTCCCCGGAACCCCTGGCAGGAAAGGAGAGCCTGGAGAA |
| | | | GCCGCTTATGTGTATCGCTCAGCGTTCAGTGTGGGGCTGGA |
| | | | GACCCGCGTCACTGTTCCCAATGTACCCATTCGCTTTACTAA |
| | | | GATCTTCTACAACCAACAGAATCATTATGACGGCAGCACTG |
| | | | GCAAGTTCTACTGCAACATTCCGGGACTCTACTACTTCTCTT |
| | | | ACCACATCACGGTGTACATGAAAGATGTGAAGGTGAGCCTC |
| | | | TTCAAGAAGGACAAGGCCGTTCTCTTCACCTACGACCAGTA |
| | | | TCAGGAAAAGAATGTGGACCAGGCCTCTGGCTCTGTGCTCC |
| | | | TCCATCTGGAGGTGGGAGACCAAGTCTGGCTCCAGGTGTAT |
| | | | GGGGATGGGGACCACAATGGACTCTATGCAGATAACGTCAA |
| | | | CGACTCTACATTTACTGGCTTTCTTCTCTACCATGATACCAA |
| | | | CTGA |
| mAdipoQ | SEQ ID NO: 34 | AA | MLLLQALLFLLILPSHAEDDVTTTEELAPALVPPPKGTCAGWM |
| | | | AGIPGHPGHNGTPGRDGRDGTPGEKGEKGDAGLLGPKGETGD |
| | | | VGMTGAEGPRGFPGTPGRKGEPGEAAYVYRSAFSVGLETRVT |
| | | | VPNVPIRFTKIFYNQQNHYDGSTGKFYCNIPGLYYFSYHITVYM |
| | | | KDVKVSLFKKDKAVLFTYDQYQEKNVDQASGSVLLHLEVGD |
| | | | QVWLQVYGDGDHNGLYADNVNDSTFTGFLLYHDTN |
| mNrf2 | SEQ ID NO: 35 | DNA | ATGATGGACTTGGAGTTGCCACCGCCAGGACTACAGTCCCA |
| | | | GCAGGACATGGATTTGATTGACATCCTTTGGAGGCAAGACA |
| | | | TAGATCTTGGAGTAAGTCGAGAAGTGTTTGACTTTAGTCAG |
| | | | CGACAGAAGGACTATGAGTTGGAAAAACAGAAAAAACTCG |
| | | | AAAAGGAAAGACAAGAGCAACTCCAGAAGGAACAGGAGA |
| | | | AGGCCTTTTTCGCTCAGTTTCAACTGGATGAAGAAACAGGA |
| | | | GAATTCCTCCCAATTCAGCCGGCCCAGCACATCCAGACAGA |
| | | | CACTAGTGGATCCGCCAGCTACTCCCAGGTTGCCCACATTC |
| | | | CCAAACAAGATGCCTTGTACTTTGAAGACTGTATGCAGCTTT |
| | | | TGGCAGAGACATTCCCATTTGTTGATGACCATGAGTCGCTTG |
| | | | CCCTGGATATCCCCAGCCACGCTGAAAGTTCAGTCTTCACT |
| | | | GCCCCTCATCAGGCCCAGTCCCTCAATAGCTCTCTGGAGGC |
| | | | AGCCATGACTGATTTAAGCAGCATAGAGCAGGACATGGAGC |
| | | | AAGTTTGGCAGGAGCTATTTTCCATTCCCGAATTACAGTGTC |
| | | | TTAATACCGAAAACAAGCAGCTGGCTGATACTACCGCTGTT |
| | | | CCCAGCCCAGAAGCCACACTGACAGAAATGGACAGCAATT |
| | | | ACCATTTTTACTCATCGATCTCCTCGCTGGAAAAAGAAGTG |
| | | | GGCAACTGTGGTCCACATTTCCTTCATGGTTTTGAGGATTCT |
| | | | TTCAGCAGCATCCTCTCCACTGATGATGCCAGCCAGCTGAC |
| | | | CTCCTTAGACTCAAATCCCACCTTAAACACAGATTTTGGCGA |
| | | | TGAATTTTATTCTGCTTTCATAGCAGAGCCCAGTGACGGTGG |
| | | | CAGCATGCCTTCCTCCGCTGCCATCAGTCAGTCACTCTCTGA |
| | | | ACTCCTGGACGGGACTATTGAAGGCTGTGACCTGTCACTGT |
| | | | GTAAAGCTTTCAACCCGAAGCACGCTGAAGGCACAATGGAA |
| | | | TTCAATGACTCTGACTCTGGCATTTCACTGAACACAAGTCCC |
| | | | AGCCGAGCGTCCCCAGAGCACTCCGTGGAGTCTTCCATTTA |
| | | | CGGAGACCCACCGCCTGGGTTCAGTGACTCGGAAATGGAGG |
| | | | AGCTAGATAGTGCCCCTGGAAGTGTCAAACAGAACGGCCCT |
| | | | AAAGCACAGCCAGCACATTCTCCTGGAGACACAGTACAGCC |
| | | | TCTGTCACCAGCTCAAGGGCACAGTGCTCCTATGCGTGAAT |
| | | | CCCAATGTGAAAATACAACAAAAAAAGAAGTTCCCGTGAGT |
| | | | CCTGGTCATCAAAAAGCCCCATTCACAAAAGACAAACATTC |
| | | | AAGCCGCTTAGAGGCTCATCTCACACGAGATGAGCTTAGGG |
| | | | CAAAAGCTCTCCATATTCCATTCCCTGTCGAAAAAATCATTA |
| | | | ACCTCCCTGTTGATGACTTCAATGAAATGATGTCCAAGGAG |
| | | | CAATTCAATGAAGCTCAGCTCGCATTGATCCGAGATATACG |
| | | | CAGGAGAGGTAAGAATAAAGTCGCCGCCCAGAACTGTAGG |
| | | | AAAAGGAAGCTGGAGAACATTGTCGAGCTGGAGCAAGACT |
| | | | TGGGCCACTTAAAAGACGAGAGAGAAAAACTACTCAGAGA |
| | | | AAAGGGAGAAAACGACAGAAACCTCCATCTACTGAAAAGG |
| | | | CGGCTCAGCACCTTGTATCTTGAAGTCTTCAGCATGTTACGT |
| | | | GATGAGGATGGAAAGCCTTACTCTCCCAGTGAATACTCTCT |
| | | | GCAGCAAACCAGAGATGGCAATGTGTTCCTTGTTCCCAAAA |
| | | | GCAAGAAGCCAGATACAAAGAAAAACTAG |
| mNrf2 | SEQ ID NO: 36 | AA | MMDLELPPPGLQSQQDMDLIDILWRQDIDLGVSREVFDFSQRQ |
| | | | KDYELEKQKKLEKERQEQLQKEQEKAFFAQFQLDEETGEFLPI |
| | | | QPAQHIQTDTSGSASYSQVAHIPKQDALYFEDCMQLLAETFPF |
| | | | VDDHESLALDIPSHAESSVFTAPHQAQSLNSSLEAAMTDLSSIE |
| | | | QDMEQVWQELFSIPELQCLNTENKQLADTTAVPSPEATLTEMD |
| | | | SNYHFYSSISSLEKEVGNCGPHFLHGFEDSFSSILSTDDASQLTS |
| | | | LDSNPTLNTDFGDEFYSAFIAEPSDGGSMPSSAAISQSLSELLDG |
| | | | TIEGCDLSLCKAFNPKHAEGTMEFNDSDSGISLNTSPSRASPEH |
| | | | SVESSIYGDPPPGFSDSEMEELDSAPGSVKQNGPKAQPAHSPGD |
| | | | TVQPLSPAQGHSAPMRESQCENTTKKEVPVSPGHQKAPFTKD |
| | | | KHSSRLEAHLTRDELRAKALHIPFPVEKIINLPVDDFNEMMSKE |
| | | | QFNEAQLALIRDIRRRGKNKVAAQNCRKRKLENIVELEQDLGH |
| | | | LKDEREKLLREKGENDRNLHLLKRRLSTLYLEVFSMLRDEDG |
| | | | KPYSPSEYSLQQTRDGNVFLVPKSKKPDTKKN |
| sTGFBRII | SEQ ID NO: 37 | DNA | ATGGGTCGGGGGCTGCTCCGGGGCCTGTGGCCGCTGCATAT |
| | | | CGTCCTGTGGACGCGCATCGCCAGCACGATCCCGCCGCACG |
| | | | TTCCCAAGTCGGATGTGGAAATGGAAGCCCAGAAAGATGCA |
| | | | TCCATCCACCTAAGCTGTAATAGGACCATCCATCCACTGAA |
| | | | ACATTTTAACAGTGATGTCATGGCCAGCGACAATGGCGGTG |
| | | | CGGTCAAGCTTCCACAGCTGTGCAAGTTTTGCGATGTGAGA |
| | | | CTGTCCACTTGCGACAACCAGAAGTCCTGCATGAGCAACTG |
| | | | CAGCATCACGGCCATCTGTGAGAAGCCGCATGAAGTCTGCG |
| | | | TGGCCGTGTGGAGGAAGAACGACAAGAACATTACTCTGGA |
| | | | GACGGTTTGCCACGACCCCAAGCTCACCTACCACGGCTTCA |
| | | | CTCTGGAAGATGCCGCTTCTCCCAAGTGTGTCATGAAGGAA |
| | | | AAGAAAAGGGCGGGCGAGACTTTCTTCATGTGTGCCTGTAA |
| | | | CATGGAAGAGTGCAACGATTACATCATCTTTTCGGAAGAAT |
| | | | ACACCACCAGCAGTCCCGACCCCAGAGGGCCCACAATCAA |
| | | | GCCCTGTCCTCCATGCAAATGCCCAGCACCTAACCTCGAGG |
| | | | GTGGACCATCCGTCTTCATCTTCCCTCCAAAGATCAAGGATG |
| | | | TACTCATGATCTCCCTGAGCCCCATAGTCACATGTGTGGTGG |
| | | | TGGATGTGAGCGAGGATGACCCAGATGTCCAGATCAGCTGG |
| | | | TTTGTGAACAACGTGGAAGTACACACAGCTCAGACACAAAC |
| | | | CCATAGAGAGGATTACAACAGTACTCTCCGGGTGGTCAGTG |
| | | | CCCTCCCCATCCAGCACCAGGACTGGATGAGTGGCAAGGCG |
| | | | TTCGCATGCGCGGTCAACAACAAAGACCTCCCAGCGCCCAT |
| | | | CGAGAGAACCATCTCAAAACCCAAAGGGTCAGTAAGAGCT |
| | | | CCACAGGTATATGTCTTGCCTCCACCAGAAGAAGAGATGAC |
| | | | TAAGAAACAGGTCACTCTGACCTGCATGGTCACAGACTTCA |
| | | | TGCCTGAAGACATTTACGTGGAGTGGACCAACAACGGGAAA |
| | | | ACAGAGCTAAACTACAAGAACACTGAACCAGTCCTGGACTC |
| | | | TGATGGTTCTTACTTCATGTACAGCAAGCTGAGAGTGGAAA |
| | | | AGAAGAACTGGGTGGAAAGAAATAGCTACTCCTGTTCAGTG |
| | | | GTCCACGAGGGTCTGCACAATCACCACACGACTAAGAGCTT |
| | | | CTCCCGGACTCCGGGTAAATGA |
| sTGFBRII | SEQ ID NO: 38 | AA | MGRGLLRGLWPLHIVLWTRIASTIPPHVPKSDVEMEAQKDASI |
| | | | HLSCNRTIHPLKHFNSDVMASDNGGAVKLPQLCKFCDVRLST |
| | | | CDNQKSCMSNCSITAICEKPHEVCVAVWRKNDKNITLETVCH |
| | | | DPKLTYHGFTLEDAASPKCVMKEKKRAGETFFMCACNMEEC |
| | | | NDYIIFSEEYTTSSPDPRGPTIKPCPPCKCPAPNLEGGPSVFIFPP |
| | | | KIKDVLMISLSPIVTCVVVDVSEDDPDVQISWFVNNVEVHTAQ |
| | | | TQTHREDYNSTLRVVSALPIQHQDWMSGKAFACAVNNKDLPA |
| | | | PIERTISKPKGSVRAPQVYVLPPPEEEMTKKQVTLTCMVTDFM |
| | | | PEDIYVEWTNNGKTELNYKNTEPVLDSDGSYFMYSKLRVEKK |
| | | | NWVERNSYSCSVVHEGLHNHHTTKSFSRTPGK |
| GDF15 | SEQ ID NO: 39 | DNA | ATGGCCCCGCCCGCGCTCCAGGCCCAGCCTCCAGGCGGCTC |
| | | | TCAACTGAGGTTCCTGCTGTTCCTGCTGCTGTTGCTGCTGCT |
| | | | GCTGTCATGGCCATCGCAGGGGGACGCCCTGGCAATGCCTG |
| | | | AACAGCGACCCTCCGGCCCTGAGTCCCAACTCAACGCCGAC |
| | | | GAGCTACGGGGTCGCTTCCAGGACCTGCTGAGCCGGCTGCA |
| | | | TGCCAACCAGAGCCGAGAGGACTCGAACTCAGAACCAAGT |
| | | | CCTGACCCAGCTGTCCGGATACTCAGTCCAGAGGTGAGATT |
| | | | GGGGTCCCACGGCCAGCTGCTACTCCGCGTCAACCGGGCGT |
| | | | CGCTGAGTCAGGGTCTCCCCGAAGCCTACCGCGTGCACCGA |
| | | | GCGCTGCTCCTGCTGACGCCGACGGCCCGCCCCTGGGACAT |
| | | | CACTAGGCCCCTGAAGCGTGCGCTCAGCCTCCGGGGACCCC |
| | | | GTGCTCCCGCATTACGCCTGCGCCTGACGCCGCCTCCGGAC |
| | | | CTGGCTATGCTGCCCTCTGGCGGCACGCAGCTGGAACTGCG |
| | | | CTTACGGGTAGCCGCCGGCAGGGGGCGCCGAAGCGCGCAT |
| | | | GCGCACCCAAGAGACTCGTGCCCACTGGGTCCAGGGCGCTG |
| | | | CTGTCACTTGGAGACTGTGCAGGCAACTCTTGAAGACTTGG |
| | | | GCTGGAGCGACTGGGTGCTGTCCCCGCGCCAGCTGCAGCTG |
| | | | AGCATGTGCGTGGGCGAGTGTCCCCACCTGTATCGCTCCGC |
| | | | GAACACGCATGCGCAGATCAAAGCACGCCTGCATGGCCTGC |
| | | | AGCCTGACAAGGTGCCTGCCCCGTGCTGTGTCCCCTCCAGC |
| | | | TACACCCCGGTGGTTCTTATGCACAGGACAGACAGTGGTGT |
| | | | GTCACTGCAGACTTATGATGACCTGGTGGCCCGGGGCTGCC |
| | | | ACTGCGCTTGA |
| GDF15 | SEQ ID NO: 40 | AA | MAPPALQAQPPGGSQLRFLLFLLLLLLLLSWPSQGDALAMPEQ |
| | | | RPSGPESQLNADELRGRFQDLLSRLHANQSREDSNSEPSPDPAV |
| | | | RILSPEVRLGSHGQLLLRVNRASLSQGLPEAYRVHRALLLLTPT |
| | | | ARPWDITRPLKRALSLRGPRAPALRLRLTPPPDLAMLPSGGTQ |
| | | | LELRLRVAAGRGRRSAHAHPRDSCPLGPGRCCHLETVQATLE |
| | | | DLGWSDWVLSPRQLQLSMCVGECPHLYRSANTHAQIKARLHG |
| | | | LQPDKVPAPCCVPSSYTPVVLMHRTDSGVSLQTYDDLVARGC |
| | | | HCA |
| hFoxp2 | SEQ ID NO: 41 | DNA | ATGATGCAGGAATCTGCGACAGAGACAATAAGCAACAGTTC |
| | | | AATGAATCAAAATGGAATGAGCACTCTAAGCAGCCAATTAG |
| | | | ATGCTGGCAGCAGAGATGGAAGATCAAGTGGTGACACCAG |
| | | | CTCTGAAGTAAGCACAGTAGAACTGCTGCATCTGCAACAAC |
| | | | AGCAGGCTCTCCAGGCAGCAAGACAACTTCTTTTACAGCAG |
| | | | CAAACAAGTGGATTGAAATCTCCTAAGAGCAGTGATAAACA |
| | | | GAGACCACTGCAGGTGCCTGTGTCAGTGGCCATGATGACTC |
| | | | CCCAGGTGATCACCCCTCAGCAAATGCAGCAGATCCTTCAG |
| | | | CAACAAGTCCTGTCTCCTCAGCAGCTACAAGCCCTTCTCCA |
| | | | ACAACAGCAGGCTGTCATGCTGCAGCAGCAACAACTACAA |
| | | | GAGTTTTACAAGAAACAGCAAGAGCAGTTACATCTTCAGCT |
| | | | TTTGCAGCAGCAGCAGCAACAGCAGCAGCAGCAACAACAG |
| | | | CAGCAACAACAGCAGCAGCAACAACAACAACAACAGCAGC |
| | | | AACAACAGCAGCAGCAGCAGCAACAGCAGCAGCAGCAGCA |
| | | | ACAGCATCCTGGAAAGCAAGCGAAAGAGCAGCAGCAGCAG |
| | | | CAGCAGCAACAGCAATTGGCAGCCCAGCAGCTTGTCTTCCA |
| | | | GCAGCAGCTTCTCCATATGCAACAACTCCAGCAGCAGCAGC |
| | | | ATCTGCTCAGCCTTCAGCGTCAGGGACTCATCTCCATTCCAC |
| | | | CTGGCCAGGCAGCACTTCCTGTCCAATCGCTGCCTCAAGCT |
| | | | GGCTTAAGTCCTGCTGAGATTCAGCAGTTATGGAAAGAAGT |
| | | | GACTGGAGTTCACAGTATGGAAGACAATGGCATTAAACATG |
| | | | GAGGGCTAGACCTCACTACTAACAATTCCTCCTCGACTACC |
| | | | TCCTCCAACACTTCCAAAGCATCACCACCAATAACTCATCA |
| | | | TTCCATAGTGAATGGACAGTCTTCAGTTCTAAGTGCAAGAC |
| | | | GAGACAGCTCGTCACATGAGGAGACTGGGGCCTCTCACACT |
| | | | CTCTATGGCCATGGAGTTTGCAAATGGCCAGGCTGTGAAAG |
| | | | CATTTGTGAAGATTTTGGACAGTTTTTAAAGCACCTTAACAA |
| | | | TGAACACGCATTGGATGACCGAAGCACTGCTCAGTGTCGAG |
| | | | TGCAAATGCAGGTGGTGCAACAGTTAGAAATACAGCTTTCT |
| | | | AAAGAACGCGAACGTCTTCAAGCAATGATGACCCACTTGCA |
| | | | CATGCGACCCTCAGAGCCCAAACCATCTCCCAAACCTCTAA |
| | | | ATCTGGTGTCTAGTGTCACCATGTCGAAGAATATGTTGGAG |
| | | | ACATCCCCACAGAGCTTACCTCAAACCCCTACCACACCAAC |
| | | | GGCCCCAGTCACCCCGATTACCCAGGGACCCTCAGTAATCA |
| | | | CCCCAGCCAGTGTGCCCAATGTGGGAGCCATACGAAGGCGA |
| | | | CATTCAGACAAATACAACATTCCCATGTCATCAGAAATTGC |
| | | | CCCAAACTATGAATTTTATAAAAATGCAGATGTCAGACCTC |
| | | | CATTTACTTATGCAACTCTCATAAGGCAGGCTATCATGGAGT |
| | | | CATCTGACAGGCAGTTAACACTTAATGAAATTTACAGCTGG |
| | | | TTTACACGGACATTTGCTTACTTCAGGCGTAATGCAGCAACT |
| | | | TGGAAGAATGCAGTACGTCATAATCTTAGCCTGCACAAGTG |
| | | | TTTTGTTCGAGTAGAAAATGTTAAAGGAGCAGTATGGACTG |
| | | | TGGATGAAGTAGAATACCAGAAGCGAAGGTCACAAAAGAT |
| | | | AACAGGAAGTCCAACCTTAGTAAAAAATATACCTACCAGTT |
| | | | TAGGCTATGGAGCAGCTCTTAATGCCAGTTTGCAGGCTGCC |
| | | | TTGGCAGAGAGCAGTTTACCTTTGCTAAGTAATCCTGGACT |
| | | | GATAAATAATGCATCCAGTGGCCTACTGCAGGCCGTCCACG |
| | | | AAGACCTCAATGGTTCTCTGGATCACATTGACAGCAATGGA |
| | | | AACAGTAGTCCGGGCTGCTCACCTCAGCCGCACATACATTC |
| | | | AATCCACGTCAAGGAAGAGCCAGTGATTGCAGAGGATGAA |
| | | | GACTGCCCAATGTCCTTAGTGACAACAGCTAATCACAGTCC |
| | | | AGAATTAGAAGACGACAGAGAGATTGAAGAAGAGCCTTTA |
| | | | TCTGAAGATCTGGAATGA |
| hFoxp2 | SEQ ID NO: 42 | AA | MMQESATETISNSSMNQNGMSTLSSQLDAGSRDGRSSGDTSSE |
| | | | VSTVELLHLQQQQALQAARQLLLQQQTSGLKSPKSSDKQRPL |
| | | | QVPVSVAMMTPQVITPQQMQQILQQQVLSPQQLQALLQQQQA |
| | | | VMLQQQQLQEFYKKQQEQLHLQLLQQQQQQQQQQQQQQQQ |
| | | | QQQQQQQQQQQQQQQQQQQQQQQQHPGKQAKEQQQQQQQ |
| | | | QQLAAQQLVFQQQLLHMQQLQQQQHLLSLQRQGLISIPPGQA |
| | | | ALPVQSLPQAGLSPAEIQQLWKEVTGVHSMEDNGIKHGGLDL |
| | | | TTNNSSSTTSSNTSKASPPITHHSIVNGQSSVLSARRDSSSHEET |
| | | | GASHTLYGHGVCKWPGCESICEDFGQFLKHLNNEHALDDRST |
| | | | AQCRVQMQVVQQLEIQLSKERERLQAMMTHLHMRPSEPKPSP |
| | | | KPLNLVSSVTMSKNMLETSPQSLPQTPTTPTAPVTPITQGPSVIT |
| | | | PASVPNVGAIRRRHSDKYNIPMSSEIAPNYEFYKNADVRPPFTY |
| | | | ATLIRQAIMESSDRQLTLNEIYSWFTRTFAYFRRNAATWKNAV |
| | | | RHNLSLHKCFVRVENVKGAVWTVDEVEYQKRRSQKITGSPTL |
| | | | VKNIPTSLGYGAALNASLQAALAESSLPLLSNPGLINNASSGLL |
| | | | QAVHEDLNGSLDHIDSNGNSSPGCSPQPHIHSIHVKEEPVIAED |
| | | | EDCPMSLVTTANHSPELEDDREIEEEPLSEDLE |
| mAtg5 | SEQ ID NO: 43 | DNA | ATGACAGATGACAAAGATGTGCTTCGAGATGTGTGGTTTGG |
| | | | ACGAATTCCAACTTGCTTTACTCTCTATCAGGATGAGATAAC |
| | | | TGAAAGAGAAGCAGAACCATACTATTTGCTTTTGCCAAGAG |
| | | | TCAGCTATTTGACGTTGGTAACTGACAAAGTGAAAAAGCAC |
| | | | TTTCAGAAGGTTATGAGACAAGAAGATGTTAGTGAGATATG |
| | | | GTTTGAATATGAAGGCACACCCCTGAAATGGCATTATCCAA |
| | | | TTGGTTTACTATTTGATCTTCTTGCATCAAGTTCAGCTCTTCC |
| | | | TTGGAACATCACAGTACATTTCAAGAGTTTTCCAGAAAAGG |
| | | | ACCTTCTACACTGTCCATCCAAGGATGCGGTTGAGGCTCACT |
| | | | TTATGTCGTGTATGAAAGAAGCTGATGCTTTAAAGCATAAA |
| | | | AGTCAAGTGATCAACGAAATGCAGAAAAAAGACCACAAGC |
| | | | AGCTCTGGATGGGACTGCAGAATGACAGATTTGACCAGTTT |
| | | | TGGGCCATCAACCGGAAACTCATGGAATATCCTCCAGAAGA |
| | | | AAATGGATTTCGTTATATCCCCTTTAGAATATATCAGACCAC |
| | | | GACGGAGCGGCCTTTCATCCAGAAGCTGTTCCGGCCTGTGG |
| | | | CCGCAGATGGACAGCTGCACACACTTGGAGATCTCCTCAGA |
| | | | GAAGTCTGTCCTTCCGCAGTCGCCCCTGAAGATGGAGAGAA |
| | | | GAGGAGCCAGGTGATGATTCACGGGATAGAGCCAATGCTG |
| | | | GAAACCCCTCTGCAGTGGCTGAGCGAGCATCTGAGCTACCC |
| | | | AGATAACTTTCTTCATATTAGCATTGTCCCCCAGCCAACAGA |
| | | | TTAA |
| mAtg5 | SEQ ID NO: 44 | AA | MTDDKDVLRDVWFGRIPTCFTLYQDEITEREAEPYYLLLPRVS |
| | | | YLTLVTDKVKKHFQKVMRQEDVSEIWFEYEGTPLKWHYPIGL |
| | | | LFDLLASSSALPWNITVHFKSFPEKDLLHCPSKDAVEAHFMSC |
| | | | MKEADALKHKSQVINEMQKKDHKQLWMGLQNDRFDQFWAI |
| | | | NRKLMEYPPEENGFRYIPFRIYQTTTERPFIQKLFRPVAADGQL |
| | | | HTLGDLLREVCPSAVAPEDGEKRSQVMIHGIEPMLETPLQWLS |
| | | | EHLSYPDNFLHISIVPQPTD |
| mBub1b | SEQ ID NO: 45 | DNA | ATGGCGGAGGCGAGTGAAGCCATGTGCCTGGAGGGAGCAG |
| | | | AGTGGGAGCTGAGTAAAGAAAACATACAGCCCTTACGGCA |
| | | | CGGGCGGGTCATGTCCACACTTCAGGGAGCTTTGGCAAAGC |
| | | | AAGAGTCAGCTGGCCACACTGCTCTGCAGCAGCAGAAACG |
| | | | GGCATTTGAATCTGAAATCCGCTTTTACTCTGGAGATGACCC |
| | | | TCTGGATGTGTGGGACAGATATATTAATTGGACAGAACAGA |
| | | | ACTACCCTCAAGGGGGGAAGGAGAGTAACATGTCAGCGTTA |
| | | | GTGGAGAGAGCGATAGAAGCACTCCAAGGAGAGACGCGCT |
| | | | ATTATAATGACCCCCGCTTTCTCAGTCTCTGGATCAAATTGG |
| | | | GACATTTGTGCAATGAACCTTTGGATATGTACAGCTATTTAC |
| | | | AAAGCCAAGGAATTGGCGTTTCCCTTGCCCAGTTCTATATTT |
| | | | CATGGGCTGAAGAATACGAAGCTAGAGAAAATTTCAAGAA |
| | | | AGCGGACATAATATTTCAGGAAGGGATTGAACGCAAGGCTG |
| | | | AGCCCCTGGACAGACTGCAGTCCCAGCACAGACAGTTCCAG |
| | | | TCTCGAGTGTCGCGACAAGCTTTCTTGGCCCTTGGGAATGA |
| | | | AGAGGAGGAGGCTTTGGAGCCTTCTGAACCACAGAGAAGCT |
| | | | CGCTAGCTGAGCTGAAGAGCAGAGGGAAGAAGATGGCCAG |
| | | | AGCGCCCATCAGCCGTGTCGGAAGTGCTCTGAAAGCTCCAG |
| | | | GTCAGAGCAGAGGATTCCTAAATGCAGTTCCCCAGCCAGTA |
| | | | CACGGTAATCGCAGGATCACCGTTTTTGATGAAAATGCCGA |
| | | | TACCGCGTCTAGACCGGAGTTATCTAAGCCTGTAGCCCAGC |
| | | | CATGGATGGCACCCCCTGTGCCCAGGGCCAAAGAGAACGA |
| | | | ACTTCAGCCAGGCCCATGGAGCACAGACAGGCCGGTGGGA |
| | | | CGCAGGCCTCATGACAATCCAGCCTCTGTGACGTCGATACC |
| | | | CAGCGTGCTTCCCAGCTTTACGCCGTACGTGGAAGAGAGCG |
| | | | CCCAGCAGACAGTCATGACACCATGCAAGATTGAGCCTAGT |
| | | | ATCAACCATGTTCTCAGCACCAGGAAGCCAGGAAGAGAAG |
| | | | AAGGAGACCCCTTGCAGAGAGTTCAGAGTCATCAGCAAGGC |
| | | | TGTGAGGAGAAGAAGGAGAAGATGATGTACTGTAAGGAGA |
| | | | AGATCTATGCCGGAGTTGGGGAGTTCTCCTTTGAGGAGATC |
| | | | CGAGCTGAAGTGTTCCGAAAGAAGCTGAAAGAACGAAGGG |
| | | | AAGCCGAGCTGTTGACCAGTGCAAAGAAGAGGGAGGAGAT |
| | | | GCAGAAGCAGATCGAAGAGATGGAGAGGAGGCTGAAGGCA |
| | | | ATGCAGGCTGTTCAGCAAGAAGGAGCTGGTGGCCAGCAAG |
| | | | AAGAGAAGATGCCTACAGAGGACCCAGCCAGATTGCAGAT |
| | | | TGCTTCGGGGCCTCAGGAAATGTCGGGAGTTCCTCTGTCCTG |
| | | | TTCCATCTGTCCACTAAGCTCGAATCCTAGGGAAATTTCACC |
| | | | TGCTGAGAACATTTTGCAAGAACAGCCTGATTCTAAAGGTT |
| | | | CCAGTATGCCTTTCTCCATTTTTGATGAGTCTCTTTCAGACA |
| | | | AAAAAGACAAAAGTCCTGCTACAGGTGGTCCACAGGTTCTC |
| | | | AATGCCCAGAGAAGACCCCTTTCAGTTCTCAAAACTACAGA |
| | | | AGTGGGCACCACAAATGAGGATGTGTCTCCCGATATTTGTG |
| | | | ATGAACTCACAGAACTTGAGCCTCTGAGTGAAGACGCCATC |
| | | | ATCACTGGTTTCAGGAACGTCACTCTCTGTCCCAACCCTGAG |
| | | | GACACTTGTGACTTTGCTAGAGCAGCTCGTTTGGCATCTACT |
| | | | CCTTTCCATGAGATACTGTCCTCGAAGGGCATCGCTGCTGAT |
| | | | CCCGAGGGACTGTTGCAGGAAGAGGATCTGGATGGGAAGG |
| | | | CCGCCGAGGCTCATCACACTGTTCATCACCAGGCCCTCATC |
| | | | ATAAAGAAACTGAGCCCAATTATTGAAGACAGCCGTGAGGC |
| | | | CACCCACTCATCTGGCTTCTCCAGGTCTTCTTCCTCAGCTCC |
| | | | CAGTACATCCTCCATCAAAGGCTTTCAGCTTCTGGAAAAGC |
| | | | TGGAGCTGACTAATGACGGGGCAGAAAATGCTATTCAGTCA |
| | | | CCCTGGTGTTCACAGTATCGCCTACAACTGTTAAAATCCCTA |
| | | | CTAGAGATAAGTGCTTTTGCGGAGTTTTCTGTGGAAGACCG |
| | | | ACCGATGCCTGTGCTGGAAATAGGGAAGGAGATTGAGTTAG |
| | | | GTCCTGAGGATTACGTCATCAAGCAAGAGCACCTAACATGT |
| | | | GACGATTACAGGTTATTCTGGGTGGCACCAAGAAGCTCTGC |
| | | | AGAGCTAACCATGATAAAGGCATCATCTCAGCCTATCCCGT |
| | | | GGGATTTTTATATCAACCTCAAGTTGAAGGAGCGTCTGAAT |
| | | | GAGGACTATGACCAGCTTTGCAGCTGCTGTCAGTACCAAGA |
| | | | TGGCCATGTTGTTTGGTACCAGTATATAAACTGCTCCACCCT |
| | | | TCAGAATCTTCTCCAACACAGCGAATTTGTTACTCATGAAAT |
| | | | AATAGTGTTGATTATTTACAACCTCTTGACAATCGTGGAGA |
| | | | AGCTACACAGAGCTGAAATAGTGCACGGAGACTTGAGTCCA |
| | | | CGGAGTCTGATCCTACGAAACAGAATCCACGACCCCTATGA |
| | | | CTATGTAAATAAGGACGATCACGCTGTGAGGATCATGGACT |
| | | | TCTCCTACAGTGTTGACCTGAGGGTGCAGCTGGATGCGTTTG |
| | | | CCTATAGTGGCTTTCGGACTGCACAGATCCTGGAAGGACAA |
| | | | AAGATCCTGGCTAACTGTTCTTCTCCCTACCATGTAGATCTG |
| | | | TTGGGTATAGCAGACCTAGCGCACTTACTCCTGTTCAAGGA |
| | | | GCACCTCCATGTCTTCTGGGATGGACTCCTCTGGAAACTTAG |
| | | | CCAGAGCACCTCTGAGCTAAAAGACAGTGAATTGTGGAATA |
| | | | AATTCTTTGTGCGGATTCTGAATGCCAGTGACAAGTCCACA |
| | | | GTGTCTGTTCTGGGGGAGCTGGCAGCAGAAATGGGTGGGGC |
| | | | TTTTGATGCCACATTCCATAGCCACCTGAACAGAGCCCTGTG |
| | | | GAAGCTGGGGAAGACAATCAGCCCGGAAGCTTTGCTCACTC |
| | | | AGCAAGACAAGCAGCCAGGCGGCTCCCAGAGCCCTGCCTA |
| | | | A |
| mBub1b | SEQ ID NO: 46 | AA | MAEASEAMCLEGAEWELSKENIQPLRHGRVMSTLQGALAKQE |
| | | | SAGHTALQQQKRAFESEIRFYSGDDPLDVWDRYINWTEQNYP |
| | | | QGGKESNMSALVERATEALQGETRYYNDPRFLSLWIKLGHLCN |
| | | | EPLDMYSYLQSQGIGVSLAQFYISWAEEYEARENFKKADIIFQE |
| | | | GIERKAEPLDRLQSQHRQFQSRVSRQAFLALGNEEEEALEPSEP |
| | | | QRSSLAELKSRGKKMARAPISRVGSALKAPGQSRGFLNAVPQP |
| | | | VHGNRRITVFDENADTASRPELSKPVAQPWMAPPVPRAKENE |
| | | | LQPGPWSTDRPVGRRPHDNPASVTSIPSVLPSFTPYVEESAQQT |
| | | | VMTPCKIEPSINHVLSTRKPGREEGDPLQRVQSHQQGCEEKKE |
| | | | KMMYCKEKIYAGVGEFSFEEIRAEVFRKKLKERREAELLTSAK |
| | | | KREEMQKQIEEMERRLKAMQAVQQEGAGGQQEEKMPTEDPA |
| | | | RLQIASGPQEMSGVPLSCSICPLSSNPREISPAENILQEQPDSKGS |
| | | | SMPFSIFDESLSDKKDKSPATGGPQVLNAQRRPLSVLKTTEVGT |
| | | | TNEDVSPDICDELTELEPLSEDAIITGFRNVTLCPNPEDTCDFAR |
| | | | AARLASTPFHEILSSKGIAADPEGLLQEEDLDGKAAEAHHTVH |
| | | | HQALIIKKLSPIIEDSREATHSSGFSRSSSSAPSTSSIKGFQLLEKL |
| | | | ELTNDGAENAIQSPWCSQYRLQLLKSLLEISAFAEFSVEDRPMP |
| | | | VLEIGKEIELGPEDYVIKQEHLTCDDYRLFWVAPRSSAELTMIK |
| | | | ASSQPIPWDFYINLKLKERLNEDYDQLCSCCQYQDGHVVWYQ |
| | | | YINCSTLQNLLQHSEFVTHEIIVLIIYNLLTIVEKLHRAEIVHGDL |
| | | | SPRSLILRNRIHDPYDYVNKDDHAVRIMDFSYSVDLRVQLDAF |
| | | | AYSGFRTAQILEGQKILANCSSPYHVDLLGIADLAHLLLFKEHL |
| | | | HVFWDGLLWKLSQSTSELKDSELWNKFFVRILNASDKSTVSVL |
| | | | GELAAEMGGAFDATFHSHLNRALWKLGKTISPEALLTQQDKQ |
| | | | PGGSQSPA |
| MCat | SEQ ID NO: 47 | DNA | ATGTCCGTCCTGACGCCGCTGCTGCTGCGGGGCTTGACAGG |
| | | | CTCGGCCCGGCGGCTCCCAGTGCCGCGCGCCAAGATCCATT |
| | | | CGTTGGGGGATCCACCGGTCGCCACCATGTCGGACAGTCGG |
| | | | GACCCAGCCAGCGACCAGATGAAGCAGTGGAAGGAGCAGC |
| | | | GGGCCTCGCAGAGACCTGATGTCCTGACCACCGGAGGCGGG |
| | | | AACCCAATAGGAGATAAACTTAATATCATGACCGCGGGGTC |
| | | | CCGAGGGCCCCTCCTCGTTCAGGATGTGGTTTTCACTGACGA |
| | | | GATGGCACACTTTGACAGAGAGCGGATTCCTGAGAGAGTGG |
| | | | TACACGCAAAAGGAGCAGGTGCTTTTGGATACTTTGAGGTC |
| | | | ACCCACGATATCACCAGATACTCCAAGGCAAAGGTGTTTGA |
| | | | GCATATTGGAAAGAGGACCCCTATTGCCGTTCGATTCTCCA |
| | | | CAGTCGCTGGAGAGTCAGGCTCAGCTGACACAGTTCGTGAC |
| | | | CCTCGGGGGTTTGCAGTGAAATTTTACACTGAAGATGGTAA |
| | | | CTGGGATCTTGTGGGAAACAACACCCCTATTTTCTTCATCAG |
| | | | GGATGCCATATTGTTTCCATCCTTTATCCATAGCCAGAAGAG |
| | | | AAACCCACAGACTCACCTGAAGGATCCTGACATGGTCTGGG |
| | | | ACTTCTGGAGTCTTCGTCCCGAGTCTCTCCATCAGGTTTCTT |
| | | | TCTTGTTCAGTGACCGAGGGATTCCCGATGGTCACCGGCAC |
| | | | ATGAATGGCTATGGATCACACACCTTCAAGTTGGTTAATGC |
| | | | AGATGGAGAGGCAGTCTATTGCAAGTTCCATTACAAGACCG |
| | | | ACCAGGGCATCAAAAACTTGCCTGTTGGAGAGGCAGGAAG |
| | | | GCTTGCTCAGGAAGATCCGGATTATGGCCTCCGAGATCTTTT |
| | | | CAATGCCATCGCCAATGGCAATTACCCGTCCTGGACGTTTTA |
| | | | CATCCAGGTCATGACTTTTAAGGAGGCAGAAACTTTCCCAT |
| | | | TTAATCCATTTGATCTGACCAAGGTTTGGCCTCACAAGGACT |
| | | | ACCCTCTTATACCAGTTGGCAAACTGGTTTTAAACAAAAAT |
| | | | CCAGTTAATTACTTTGCTGAAGTTGAACAGATGGCTTTTGAC |
| | | | CCAAGCAATATGCCCCCTGGCATCGAGCCCAGCCCTGACAA |
| | | | AATGCTTCAGGGCCGCCTTTTTGCCTACCCGGACACTCACCG |
| | | | CCACCGCCTGGGACCCAACTATCTGCAGATACCTGTGAACT |
| | | | GTCCCTACCGCGCTCGAGTGGCCAACTACCAGCGTGATGGC |
| | | | CCCATGTGCATGCATGACAACCAGGGTGGTGCCCCCAACTA |
| | | | TTACCCCAACAGCTTCAGCGCACCAGAGCAGCAGCGCTCAG |
| | | | CCCTGGAGCACAGCGTCCAGTGCGCTGTAGATGTGAAACGC |
| | | | TTCAACAGTGCTAATGAAGACAATGTCACTCAGGTGCGGAC |
| | | | ATTCTACACAAAGGTGTTGAACGAGGAGGAGAGGAAACGC |
| | | | CTGTGTGAGAACATTGCCGGCCACCTGAAGGACGCTCAGCT |
| | | | TTTCATTCAGAAGAAAGCGGTCAAGAATTTCACTGACGTCC |
| | | | ACCCTGACTATGGGGCCCGCATCCAGGCTCTTCTGGACAAG |
| | | | TACAACGCTGAGAAGCCTAAGAACGCAATTCACACCTACAC |
| | | | GCAGGCCGGCTCTCACATGGCTGCGAAGGGAAAAGCTAAC |
| | | | CTGTAA |
| MCat | SEQ ID NO: 48 | AA | MSVLTPLLLRGLTGSARRLPVPRAKIHSLGDPPVATMSDSRDP |
| | | | ASDQMKQWKEQRASQRPDVLTTGGGNPIGDKLNIMTAGSRGP |
| | | | LLVQDVVFTDEMAHFDRERIPERVVHAKGAGAFGYFEVTHDI |
| | | | TRYSKAKVFEHIGKRTPIAVRFSTVAGESGSADTVRDPRGFAV |
| | | | KFYTEDGNWDLVGNNTPIFFIRDAILFPSFIHSQKRNPQTHLKD |
| | | | PDMVWDFWSLRPESLHQVSFLFSDRGIPDGHRHMNGYGSHTF |
| | | | KLVNADGEAVYCKFHYKTDQGIKNLPVGEAGRLAQEDPDYG |
| | | | LRDLFNAIANGNYPSWTFYIQVMTFKEAETFPFNPFDLTKVWP |
| | | | HKDYPLIPVGKLVLNKNPVNYFAEVEQMAFDPSNMPPGIEPSP |
| | | | DKMLQGRLFAYPDTHRHRLGPNYLQIPVNCPYRARVANYQRD |
| | | | GPMCMHDNQGGAPNYYPNSFSAPEQQRSALEHSVQCAVDVK |
| | | | RFNSANEDNVTQVRTFYTKVLNEEERKRLCENIAGHLKDAQLF |
| | | | IQKKAVKNFTDVHPDYGARIQALLDKYNAEKPKNAIHTYTQA |
| | | | GSHMAAKGKANL |
| mCisd2 | SEQ ID NO: 49 | DNA | ATGGTCCTGGACAGCGTGGCCCGCATCGTGAAGGTGCAGCT |
| | | | GCCCGCCTACCTCAAGCAGCTCCCGGTCCCCGACAGCATCA |
| | | | CCGGGTTCGCCCGCCTCACAGTTTCAGACTGGCTCCGCCTAC |
| | | | TGCCCTTCCTCGGGGTACTTGCGCTTCTGGGCTACCTCGCAG |
| | | | TGCGCCCATTCTTCCCAAAGAAGAAGCAACAGAAGGATAGC |
| | | | TTGATCAATCTTAAGATACAAAAGGAAAATCCCAAGGTGGT |
| | | | GAATGAGATAAACATTGAAGATCTGTGTCTCACCAAAGCAG |
| | | | CTTATTGTAGGTGCTGGCGGTCCAAGACGTTTCCTGCCTGTG |
| | | | ATGGATCCCATAATAAGCATAATGAATTGACAGGCGATAAC |
| | | | GTGGGTCCTCTCATCCTGAAGAAGAAAGAAGTATAA |
| mCisd2 | SEQ ID NO: 50 | AA | MVLDSVARIVKVQLPAYLKQLPVPDSITGFARLTVSDWLRLLP |
| | | | FLGVLALLGYLAVRPFFPKKKQQKDSLINLKIQKENPKVVNEIN |
| | | | IEDLCLTKAAYCRCWRSKTFPACDGSHNKHNELTGDNVGPLIL |
| | | | KKKEV |
| mFgf21 | SEQ ID NO: 51 | DNA | ATGGAATGGATGAGATCTAGAGTTGGGACCCTGGGACTGTG |
| | | | GGTCCGACTGCTGCTGGCTGTCTTCCTGCTGGGGGTCTACCA |
| | | | AGCATACCCCATCCCTGACTCCAGCCCCCTCCTCCAGTTTGG |
| | | | GGGTCAAGTCCGGCAGAGGTACCTCTACACAGATGACGACC |
| | | | AAGACACTGAAGCCCACCTGGAGATCAGGGAGGATGGAAC |
| | | | AGTGGTAGGCGCAGCACACCGCAGTCCAGAAAGTCTCCTGG |
| | | | AGCTCAAAGCCTTGAAGCCAGGGGTCATTCAAATCCTGGGT |
| | | | GTCAAAGCCTCTAGGTTTCTTTGCCAACAGCCAGATGGAGC |
| | | | TCTCTATGGATCGCCTCACTTTGATCCTGAGGCCTGCAGCTT |
| | | | CAGAGAACTGCTGCTGGAGGACGGTTACAATGTGTACCAGT |
| | | | CTGAAGCCCATGGCCTGCCCCTGCGTCTGCCTCAGAAGGAC |
| | | | TCCCCAAACCAGGATGCAACATCCTGGGGACCTGTGCGCTT |
| | | | CCTGCCCATGCCAGGCCTGCTCCACGAGCCCCAAGACCAAG |
| | | | CAGGATTCCTGCCCCCAGAGCCCCCAGATGTGGGCTCCTCT |
| | | | GACCCCCTGAGCATGGTAGAGCCTTTACAGGGCCGAAGCCC |
| | | | CAGCTATGCGTCCTGA |
| mFgf21 | SEQ ID NO: 52 | AA | MEWMRSRVGTLGLWVRLLLAVFLLGVYQAYPIPDSSPLLQFG |
| | | | GQVRQRYLYTDDDQDTEAHLEIREDGTVVGAAHRSPESLLEL |
| | | | KALKPGVIQILGVKASRFLCQQPDGALYGSPHFDPEACSFRELL |
| | | | LEDGYNVYQSEAHGLPLRLPQKDSPNQDATSWGPVRFLPMPG |
| | | | LLHEPQDQAGFLPPEPPDVGSSDPLSMVEPLQGRSPSYAS |
| mKlotho | SEQ ID NO: 53 | DNA | ATGCTAGCCCGCGCCCCTCCTCGCCGCCCGCCGCGGCTGGT |
| | | | GCTGCTCCGTTTGCTGTTGCTGCATCTGCTGCTGCTCGCCCT |
| | | | GCGCGCCCGCTGCCTGAGCGCTGAGCCGGGTCAGGGCGCGC |
| | | | AGACCTGGGCTCGCTTCGCGCGCGCTCCTGCCCCAGAGGCC |
| | | | GCTGGCCTCCTCCACGACACCTTCCCCGACGGTTTCCTCTGG |
| | | | GCGGTAGGCAGCGCCGCCTATCAGACCGAGGGCGGCTGGC |
| | | | GACAGCACGGCAAAGGCGCGTCCATCTGGGACACTTTCACC |
| | | | CATCACTCTGGGGCGGCCCCGTCCGACTCCCCGATCGTCGT |
| | | | GGCGCCGTCGGGTGCCCCGTCGCCTCCCCTGTCCTCCACTGG |
| | | | AGATGTGGCCAGCGATAGTTACAACAACGTCTACCGCGACA |
| | | | CAGAGGGGCTGCGCGAACTGGGGGTCACCCACTACCGCTTC |
| | | | TCCATATCGTGGGCGCGGGTGCTCCCCAATGGCACCGCGGG |
| | | | CACTCCCAACCGCGAGGGGCTGCGCTACTACCGGCGGCTGC |
| | | | TGGAGCGGCTGCGGGAGCTGGGCGTGCAGCCGGTGGTTACC |
| | | | CTGTACCATTGGGACCTGCCACAGCGCCTGCAGGACACCTA |
| | | | TGGCGGATGGGCCAATCGCGCCCTGGCCGACCATTTCAGGG |
| | | | ATTATGCCGAGCTCTGCTTCCGCCACTTCGGTGGTCAGGTCA |
| | | | AGTACTGGATCACCATTGACAACCCCTACGTGGTGGCCTGG |
| | | | CACGGGTATGCCACCGGGCGCCTGGCCCCGGGCGTGAGGG |
| | | | GCAGCTCCAGGCTCGGGTACCTGGTTGCCCACAACCTACTT |
| | | | TTGGCTCATGCCAAAGTCTGGCATCTCTACAACACCTCTTTC |
| | | | CGCCCCACACAGGGAGGCCGGGTGTCTATCGCCTTAAGCTC |
| | | | CCATTGGATCAATCCTCGAAGAATGACTGACTATAATATCA |
| | | | GAGAATGCCAGAAGTCTCTTGACTTTGTGCTAGGCTGGTTTG |
| | | | CCAAACCCATATTTATTGATGGCGACTACCCAGAGAGTATG |
| | | | AAGAACAACCTCTCGTCTCTTCTGCCTGATTTTACTGAATCT |
| | | | GAGAAGAGGCTCATCAGAGGAACTGCTGACTTTTTTGCTCT |
| | | | CTCCTTCGGACCAACCTTGAGCTTTCAGCTATTGGACCCTAA |
| | | | CATGAAGTTCCGCCAATTGGAGTCTCCCAACCTGAGGCAGC |
| | | | TTCTGTCTTGGATAGATCTGGAATATAACCACCCTCCAATAT |
| | | | TTATTGTGGAAAATGGCTGGTTTGTCTCGGGAACCACCAAA |
| | | | AGGGATGATGCCAAATATATGTATTATCTCAAGAAGTTCAT |
| | | | AATGGAAACCTTAAAAGCAATCAGACTGGATGGGGTCGAC |
| | | | GTCATTGGGTACACCGCGTGGTCGCTCATGGACGGTTTCGA |
| | | | GTGGCATAGGGGCTACAGCATCCGGCGAGGACTCTTCTACG |
| | | | TTGACTTTCTGAGTCAGGACAAGGAGCTGTTGCCAAAGTCT |
| | | | TCGGCCTTGTTCTACCAAAAGCTGATAGAGGACAATGGCTT |
| | | | TCCTCCTTTACCTGAAAACCAGCCCCTTGAAGGGACATTTCC |
| | | | CTGTGACTTTGCTTGGGGAGTTGTTGACAACTACGTTCAAGT |
| | | | GGACACTACTCTCTCTCAGTTTACTGACCCGAATGTCTATCT |
| | | | GTGGGATGTGCATCACAGTAAGAGGCTTATTAAAGTAGACG |
| | | | GGGTTGTAGCCAAGAAGAGAAAACCTTACTGTGTTGATTTC |
| | | | TCTGCCATCCGGCCTCAGATAACCTTACTTCGAGAAATGCG |
| | | | GGTCACCCACTTTCGCTTCTCCCTGGACTGGGCCCTGATCTT |
| | | | GCCTCTGGGTAACCAGACCCAAGTGAACCACACGGTTCTGC |
| | | | ACTTCTACCGCTGCATGATCAGCGAGCTGGTGCACGCCAAC |
| | | | ATCACTCCAGTGGTGGCCCTGTGGCAGCCAGCAGCCCCGCA |
| | | | CCAAGGCCTGCCACATGCCCTTGCAAAACATGGGGCCTGGG |
| | | | AGAACCCGCACACTGCTCTGGCGTTTGCAGACTACGCAAAC |
| | | | CTGTGTTTTAAAGAGTTGGGTCACTGGGTCAATCTCTGGATC |
| | | | ACCATGAACGAGCCAAACACACGGAACATGACCTATCGTGC |
| | | | CGGGCACCACCTCCTGAGAGCCCATGCCTTGGCTTGGCATC |
| | | | TGTACGATGACAAGTTTAGGGCGGCTCAGAAAGGCAAAATA |
| | | | TCCATCGCCTTGCAGGCTGACTGGATAGAACCGGCCTGCCC |
| | | | TTTCTCTCAAAATGACAAAGAAGTGGCCGAGAGAGTTTTGG |
| | | | AATTTGATATAGGCTGGCTGGCAGAGCCTATTTTTGGTTCCG |
| | | | GAGATTATCCACGTGTGATGAGGGACTGGCTGAACCAAAAA |
| | | | AACAATTTTCTTTTGCCCTATTTCACCGAAGATGAAAAAAA |
| | | | GCTAGTCCGGGGTTCCTTTGACTTCCTGGCGGTGAGTCATTA |
| | | | CACCACCATTCTGGTAGACTGGGAAAAGGAGGATCCGATGA |
| | | | AATACAACGATTACTTGGAGGTACAGGAGATGACTGACATC |
| | | | ACATGGCTCAACTCTCCCAGTCAGGTGGCAGTGGTGCCTTG |
| | | | GGGGCTGCGCAAAGTGCTCAACTGGCTAAGGTTCAAGTACG |
| | | | GAGACCTCCCGATGTATGTGACAGCCAATGGAATCGATGAT |
| | | | GACCCCCACGCCGAGCAAGACTCACTGAGGATCTATTATAT |
| | | | TAAGAATTATGTGAATGAGGCTCTGAAAGCCTACGTGTTGG |
| | | | ACGACATCAACCTTTGTGGCTACTTTGCGTATTCACTTAGTG |
| | | | ATCGCTCAGCTCCCAAGTCTGGCTTTTATCGATATGCTGCGA |
| | | | ATCAGTTTGAGCCCAAACCATCTATGAAACATTACAGGAAA |
| | | | ATTATTGACAGCAATGGCTTCCTGGGTTCTGGAACACTGGG |
| | | | AAGGTTTTGTCCAGAAGAATACACTGTGTGCACCGAATGTG |
| | | | GATTTTTTCAAACCCGGAAGTCTTTGCTGGTCTTCATCTCGT |
| | | | TTCTTGTTTTTACTTTTATTATTTCTCTTGCTCTCATTTTTCAC |
| | | | TACTCCAAGAAAGGCCAGAGAAGTTATAAGTAA |
| mKlotho | SEQ ID NO: 54 | AA | MLARAPPRRPPRLVLLRLLLLHLLLLALRARCLSAEPGQGAQT |
| | | | WARFARAPAPEAAGLLHDTFPDGFLWAVGSAAYQTEGGWRQ |
| | | | HGKGASIWDTFTHHSGAAPSDSPIVVAPSGAPSPPLSSTGDVAS |
| | | | DSYNNVYRDTEGLRELGVTHYRFSISWARVLPNGTAGTPNRE |
| | | | GLRYYRRLLERLRELGVQPVVTLYHWDLPQRLQDTYGGWAN |
| | | | RALADHFRDYAELCFRHFGGQVKYWITIDNPYVVAWHGYAT |
| | | | GRLAPGVRGSSRLGYLVAHNLLLAHAKVWHLYNTSFRPTQGG |
| | | | RVSIALSSHWINPRRMTDYNIRECQKSLDFVLGWFAKPIFIDGD |
| | | | YPESMKNNLSSLLPDFTESEKRLIRGTADFFALSFGPTLSFQLLD |
| | | | PNMKFRQLESPNLRQLLSWIDLEYNHPPIFIVENGWFVSGTTKR |
| | | | DDAKYMYYLKKFIMETLKAIRLDGVDVIGYTAWSLMDGFEW |
| | | | HRGYSIRRGLFYVDFLSQDKELLPKSSALFYQKLIEDNGFPPLP |
| | | | ENQPLEGTFPCDFAWGVVDNYVQVDTTLSQFTDPNVYLWDV |
| | | | HHSKRLIKVDGVVAKKRKPYCVDFSAIRPQITLLREMRVTHFR |
| | | | FSLDWALILPLGNQTQVNHTVLHFYRCMISELVHANITPVVAL |
| | | | WQPAAPHQGLPHALAKHGAWENPHTALAFADYANLCFKELG |
| | | | HWVNLWITMNEPNTRNMTYRAGHHLLRAHALAWHLYDDKF |
| | | | RAAQKGKISIALQADWIEPACPFSQNDKEVAERVLEFDIGWLA |
| | | | EPIFGSGDYPRVMRDWLNQKNNFLLPYFTEDEKKLVRGSFDFL |
| | | | AVSHYTTILVDWEKEDPMKYNDYLEVQEMTDITWLNSPSQVA |
| | | | VVPWGLRKVLNWLRFKYGDLPMYVTANGIDDDPHAEQDSLRI |
| | | | YYIKNYVNEALKAYVLDDINLCGYFAYSLSDRSAPKSGFYRY |
| | | | AANQFEPKPSMKHYRKIIDSNGFLGSGTLGRFCPEEYTVCTEC |
| | | | GFFQTRKSLLVFISFLVFTFIISLALIFHYSKKGQRSYK |
| mMt1 | SEQ ID NO: 55 | DNA | ATGGACCCCAACTGCTCCTGCTCCACCGGCGGCTCCTGCAC |
| | | | TTGCACCAGCTCCTGCGCCTGCAAGAACTGCAAGTGCACCT |
| | | | CCTGCAAGAAGAGTGAGTTGGGACACCTTGGGTGGCGGCTA |
| | | | AGGCTAGGGGCGGGGAACTCCTACAAAACTGGCTCTGAGA |
| | | | AATGTCCTTTGCTTCCCGGAGGCCATTGTATTGTCTCGGGGA |
| | | | CAGAACTATACAGAGAACTATTTAAAAAAACCGAGGTCTTC |
| | | | TCTGTTGGGGACAGGAAGCAGAGGTCTTCAGCCAGGCTGAC |
| | | | CTCTTCCTCCTCCTTTCTAGGCTGCTGCTCCTGCTGTCCCGTG |
| | | | GGCTGCTCCAAATGTGCCCAGGGCTGTGTCTGCAAAGGCGC |
| | | | CGCGGACAAGTGCACGTGCTGTGCCT |
| mMt1 | SEQ ID NO: 56 | AA | MDPNCSCSTGGSCTCTSSCACKCNCKCTSCKKSELGHLGWRLR |
| | | | LGAGNSYKTGSEKCPLLPGGHCIVSGTELYRELFKKTEVFSVG |
| | | | DRKQRSSARLTSSSSFLGCCSCCPVGCSKCAQGCVCKGAADK |
| | | | CTCCA |
| mNeu1 | SEQ ID NO: 57 | DNA | ATGGTGGGGGCAGACCCGACCAGACCCCGGGGACCGCTGA |
| | | | GCTATTGGGCGGGCCGTCGGGGTCAGGGGCTCGCAGCGATC |
| | | | TTCCTGCTCCTGGTGTCCGCGGCGGAATCCGAGGCCAGGGC |
| | | | AGAGGATGACTTCAGCCTGGTGCAGCCGCTGGTGACCATGG |
| | | | AGCAGCTGCTGTGGGTGAGCGGGAAGCAGATCGGCTCTGTA |
| | | | GACACTTTCCGCATCCCGCTCATCACAGCCACCCCTCGGGG |
| | | | CACGCTCCTGGCCTTCGCTGAGGCCAGGAAAAAATCTGCAT |
| | | | CCGATGAGGGGGCCAAGTTCATCGCCATGAGGAGGTCCACG |
| | | | GACCAGGGTAGCACGTGGTCCTCTACAGCCTTCATCGTAGA |
| | | | CGATGGGGAGGCCTCCGATGGCCTGAACCTGGGCGCTGTGG |
| | | | TGAACGATGTAGACACAGGGATAGTGTTCCTTATCTATACC |
| | | | CTCTGTGCTCACAAGGTCAACTGCCAGGTGGCCTCTACCAT |
| | | | GTTGGTTTGGAGTAAGGACGACGGCATTTCCTGGAGCCCAC |
| | | | CCCGGAATCTCTCTGTGGATATTGGCACAGAGATGTTTGCCC |
| | | | CTGGACCTGGCTCAGGCATTCAGAAACAGCGGGAGCCTGGG |
| | | | AAGGGCCGGCTCATTGTGTGTGGACACGGGACGCTGGAGCG |
| | | | AGATGGGGTCTTCTGTCTCCTCAGTGATGACCACGGTGCCTC |
| | | | CTGGCACTACGGCACTGGAGTGAGCGGCATTCCCTTTGGCC |
| | | | AGCCCAAACACGATCACGATTTCAACCCCGACGAGTGCCAG |
| | | | CCCTACGAGCTTCCAGATGGCTCGGTCATCATCAACGCCCG |
| | | | GAACCAGAATAACTACCATTGCCGCTGCAGGATCGTCCTCC |
| | | | GCAGCTATGACGCCTGTGACACCCTCAGGCCCCGGGATGTG |
| | | | ACCTTCGACCCTGAGCTCGTGGACCCTGTGGTAGCTGCAGG |
| | | | AGCACTAGCCACCAGCTCCGGCATTGTCTTCTTCTCCAATCC |
| | | | AGCCCACCCTGAGTTCCGAGTGAACCTGACCCTGCGCTGGA |
| | | | GTTTCAGCAATGGTACATCCTGGCTGAAGGAGAGGGTCCAG |
| | | | GTGTGGCCGGGACCCAGCGGCTACTCGTCCCTGACAGCCCT |
| | | | GGAAAACAGCACGGATGGAAAGAAGCAGCCCCCGCAGCTG |
| | | | TTCGTTCTGTACGAGAAAGGCCTGAACCGGTACACCGAGAG |
| | | | CATCTCCATGGTCAAAATCAGCGTCTACGGCACGCTCTGA |
| mNeu1 | SEQ ID NO: 58 | AA | MVGADPTRPRGPLSYWAGRRGQGLAAIFLLLVSAAESEARAE |
| | | | DDFSLVQPLVTMEQLLWVSGKQIGSVDTFRIPLITATPRGTLLA |
| | | | FAEARKKSASDEGAKFIAMRRSTDQGSTWSSTAFIVDDGEASD |
| | | | GLNLGAVVNDVDTGIVFLIYTLCAHKVNCQVASTMLVWSKD |
| | | | DGISWSPPRNLSVDIGTEMFAPGPGSGIQKQREPGKGRLIVCGH |
| | | | GTLERDGVFCLLSDDHGASWHYGTGVSGIPFGQPKHDHDFNP |
| | | | DECQPYELPDGSVIINARNQNNYHCRCRIVLRSYDACDTLRPR |
| | | | DVTFDPELVDPVVAAGALATSSGIVFFSNPAHPEFRVNLTLRW |
| | | | SFSNGTSWLKERVQVWPGPSGYSSLTALENSTDGKKQPPQLFV |
| | | | LYEKGLNRYTESISMVKISVYGTL |
| mNudt1 | SEQ ID NO: 59 | DNA | ATGAGCACCTCCAGGCTTTATACCCTTGTGCTAGTGCTACAG |
| | | | CCTCAGCGAGTTCTCCTGGGCATGAAGAAGAGGGGCTTTGG |
| | | | TGCTGGCCGCTGGAATGGCTTCGGGGGCAAGGTGCAGGAAG |
| | | | GAGAGACCATTGAGGATGGGGCTAAGAGAGAGCTGCTGGA |
| | | | AGAAAGTGGTCTGAGCGTGGATACACTGCACAAGGTAGGCC |
| | | | ACATCTCGTTTGAATTTGTGGGCTCCCCTGAGCTGATGGACG |
| | | | TGCATATCTTCTCGGCTGACCATGTGCACGGGACGCCCACA |
| | | | GAGAGTGAAGAAATGCGCCCTCAGTGGTTCCAACTGGACCA |
| | | | GATCCCCTTTGCCGACATGIGGCCGGATGACAGCTACTGGT |
| | | | TCCCACTCCTGCTTCAGAAGAAGAAGTTCTGTGGGCACTTC |
| | | | AAGTTCCAGGATCAGGACACGATCCTCAGTTACTCGCTGCG |
| | | | AGAGGTGGACTCATTCTAA |
| mNudt1 | SEQ ID NO: 60 | AA | MSTSRLYTLVLVLQPQRVLLGMKKRGFGAGRWNGFGGKVQE |
| | | | GETIEDGAKRELLEESGLSVDTLHKVGHISFEFVGSPELMDVHI |
| | | | FSADHVHGTPTESEEMRPQWFQLDQIPFADMVVPDDSYWFPLL |
| | | | LQKKKFCGHFKFQDQDTILSYSLREVDSF |
| mPck1 | SEQ ID NO: 61 | DNA | ATGCCTCCTCAGCTGCATAACGGTCTGGACTTCTCTGCCAAG |
| | | | GTCATCCAGGGCAGCCTCGACAGCCTGCCCCAGGCAGTGAG |
| | | | GAAGTTCGTGGAAGGCAATGCTCAGCTGTGCCAGCCGGAGT |
| | | | ATATCCACATCTGCGATGGCTCCGAGGAGGAGTACGGGCAG |
| | | | TTGCTGACCCACATGCAGGAGGAGGGTGTCATCCGCAAGCT |
| | | | GAAGAAATATGACAACTGTTGGCTGGCTCTCACTGACCCTC |
| | | | GAGATGTGGCCAGGATCGAAAGCAAGACAGTCATCATCACC |
| | | | CAAGAGCAGAGAGACACAGTGCCCATCCCCAAAACTGGCC |
| | | | TCAGCCAGCTGGGCCGCTGGATGTCGGAAGAGGACTTTGAG |
| | | | AAAGCATTCAACGCCAGGTTCCCAGGGTGCATGAAAGGCCG |
| | | | CACCATGTATGTCATCCCATTCAGCATGGGGCCACTGGGCT |
| | | | CGCCGCTGGCCAAGATTGGTATTGAACTGACAGACTCGCCC |
| | | | TATGTGGTGGCCAGCATGCGGATCATGACTCGGATGGGCAT |
| | | | ATCTGTGCTGGAGGCCCTGGGAGATGGGGAGTTCATCAAGT |
| | | | GCCTGCACTCTGTGGGGTGCCCTCTCCCCTTAAAAAAGCCTT |
| | | | TGGTCAACAACTGGGCCTGCAACCCTGAGCTGACCCTGATC |
| | | | GCCCACCTCCCGGACCGCAGAGAGATCATCTCCTTTGGAAG |
| | | | CGGATATGGTGGGAACTCACTACTCGGGAAGAAATGCTTTG |
| | | | CGTTGCGGATCGCCAGCCGTCTGGCTAAGGAGGAAGGGTGG |
| | | | CTGGCGGAGCATATGCTGATCCTGGGCATAACTAACCCCGA |
| | | | AGGCAAGAAGAAATACCTGGCCGCAGCCTTCCCTAGTGCCT |
| | | | GTGGGAAGACCAACTTGGCCATGATGAACCCCAGCCTGCCC |
| | | | GGGTGGAAGGTCGAATGTGTGGGCGATGACATCGCCTGGAT |
| | | | GAAGTTTGATGCCCAAGGCAACTTAAGGGCTATCAACCCAG |
| | | | AAAACGGGTTTTTTGGAGTTGCTCCTGGCACCTCAGTGAAG |
| | | | ACAAATCCAAATGCCATTAAAACCATCCAGAAAAACACCAT |
| | | | CTTCACCAACGTGGCTGAGACTAGCGATGGGGGTGTTTACT |
| | | | GGGAAGGCATCGATGAGCCGCTGGCCCCGGGAGTCACCATC |
| | | | ACCTCCTGGAAGAACAAGGAGTGGAGACCGCAGGACGCGG |
| | | | AACCATGTGCCCATCCCAACTCGAGATTCTGCACCCCTGCC |
| | | | AGCCAGTGCCCCATTATTGACCCTGCCTGGGAATCTCCAGA |
| | | | AGGAGTACCCATTGAGGGTATCATCTTTGGTGGCCGTAGAC |
| | | | CTGAAGGTGTCCCCCTTGTCTATGAAGCCCTCAGCTGGCAG |
| | | | CATGGGGTGTTTGTAGGAGCAGCCATGAGATCTGAGGCCAC |
| | | | AGCTGCTGCAGAACACAAGGGCAAGATCATCATGCACGAC |
| | | | CCCTTTGCCATGCGACCCTTCTTCGGCTACAACTTCGGCAAA |
| | | | TACCTGGCCCACTGGCTGAGCATGGCCCACCGCCCAGCAGC |
| | | | CAAGTTGCCCAAGATCTTCCATGTCAACTGGTTCCGGAAGG |
| | | | ACAAAGATGGCAAGTTCCTCTGGCCAGGCTTTGGCGAGAAC |
| | | | TCCCGGGTGCTGGAGTGGATGTTCGGGCGGATTGAAGGGGA |
| | | | AGACAGCGCCAAGCTCACGCCCATCGGCTACATCCCTAAGG |
| | | | AAAACGCCTTGAACCTGAAAGGCCTGGGGGGCGTCAACGTG |
| | | | GAGGAGCTGTTTGGGATCTCTAAGGAGTTCTGGGAGAAGGA |
| | | | GGTGGAGGAGATCGACAGGTATCTGGAGGACCAGGTCAAC |
| | | | ACCGACCTCCCTTACGAAATTGAGAGGGAGCTCCGAGCCCT |
| | | | GAAACAGAGAATCAGCCAGATGTAA |
| mPck1 | SEQ ID NO: 62 | AA | MPPQLHNGLDFSAKVIQGSLDSLPQAVRKFVEGNAQLCQPEYI |
| | | | HICDGSEEEYGQLLTHMQEEGVIRKLKKYDNCWLALTDPRDV |
| | | | ARIESKTVIITQEQRDTVPIPKTGLSQLGRWMSEEDFEKAFNAR |
| | | | FPGCMKGRTMYVIPFSMGPLGSPLAKIGIELTDSPYVVASMRI |
| | | | MTRMGISVLEALGDGEFIKCLHSVGCPLPLKKPLVNNWACNPE |
| | | | LTLIAHLPDRREIISFGSGYGGNSLLGKKCFALRIASRLAKEEG |
| | | | WLAEHMLILGITNPEGKKKYLAAAFPSACGKTNLAMMNPSLP |
| | | | GWKVECVGDDIAWMKFDAQGNLRAINPENGFFGVAPGTSVK |
| | | | TNPNAIKTIQKNTIFTNVAETSDGGVYWEGIDEPLAPGVTITSW |
| | | | KNKEWRPQDAEPCAHPNSRFCTPASQCPIIDPAWESPEGVPIEG |
| | | | IIFGGRRPEGVPLVYEALSWQHGVFVGAAMRSEATAAAEHKG |
| | | | KIIMHDPFAMRPFFGYNFGKYLAHWLSMAHRPAAKLPKIFHV |
| | | | NWFRKDKDGKFLWPGFGENSRVLEWMFGRIEGEDSAKLTPIG |
| | | | YIPKENALNLKGLGGVNVEELFGISKEFWEKEVEEIDRYLEDQ |
| | | | VNTDLPYEIERELRALKQRISQM |
| mSirt6 | SEQ ID NO: 63 | DNA | ATGTGGCAGTCCTCCAGCGTGGTTTTCCACACGGGCGCCGG |
| | | | CATCAGCACCGCCTCTGGCATCCCCGACTTCAGAGGCCCCC |
| | | | ATGGCGTGTGGACCATGGAGGAACGCGGCCTGGCCCCCAA |
| | | | GTTTGACACCACCTTCGAGAATGCTCGGCCCTCGAAGACCC |
| | | | ACATGGCCCTGGTTCAGCTAGAACGCATGGGCTTCCTCAGC |
| | | | TTCCTGGTCAGCCAGAACGTAGACGGGCTGCACGTGCGCTC |
| | | | GGGCTTCCCCAGGGACAAGCTGGCAGAGCTGCACGGAAAC |
| | | | ATGTTTGTAGAGGAATGTCCCAAGTGTAAGACGCAGTACGT |
| | | | CAGAGACACGGTTGTGGGCACCATGGGCCTCAAGGCCACA |
| | | | GGCCGGCTCTGCACCGTGGCCAAGACCAGGGGACTTCGGGC |
| | | | CTGTAGAGGGGAGCTGAGAGACACCATTCTGGACTGGGAG |
| | | | GACTCGTTGCCTGACCGGGACCTGATGCTCGCTGATGAGGC |
| | | | CAGCAGGACCGCAGACCTGTCTGTCACCCTGGGTACCTCGC |
| | | | TGCAGATCCGCCCCAGTGGGAACCTGCCCCTTGCCACTAAG |
| | | | CGCCGAGGAGGCCGTCTGGTCATTGTCAACCTGCAACCCAC |
| | | | AAAACATGACCGCCAGGCTGACCTGCGCATCCACGGCTACG |
| | | | TGGATGAGGTGATGTGCAGACTCATGAAGCATCTGGGGCTG |
| | | | GAGATTCCAGCCTGGGATGGACCCTGCGTGCTAGACAAAGC |
| | | | CCTGCCACCTCTGCCTCGCCCAGTAGCACTCAAGGCTGAGC |
| | | | CCCCCGTGCATCTCAATGGTGCAGTGCATGTTTCGTATAAGT |
| | | | CCAAGCCCAACAGCCCTATACTCCACAGGCCCCCCAAAAGA |
| | | | GTGAAGACCGAGGCTGCCCCCAGCTGA |
| mSirt6 | SEQ ID NO: 64 | AA | MWQSSSVVFHTGAGISTASGIPDFRGPHGVWTMEERGLAPKF |
| | | | DTTFENARPSKTHMALVQLERMGFLSFLVSQNVDGLHVRSGF |
| | | | PRDKLAELHGNMFVEECPKCKTQYVRDTVVGTMGLKATGRL |
| | | | CTVAKTRGLRACRGELRDTILDWEDSLPDRDLMLADEASRTA |
| | | | DLSVTLGTSLQIRPSGNLPLATKRRGGRLVIVNLQPTKHDRQA |
| | | | DLRIHGYVDEVMCRLMKHLGLEIPAWDGPCVLDKALPPLPRP |
| | | | VALKAEPPVHLNGAVHVSYKSKPNSPILHRPPKRVKTEAAPS |
| mTERT | SEQ ID NO: 65 | DNA | ATGACCCGCGCTCCTCGTTGCCCCGCGGTGCGCTCTCTGCTG |
| | | | CGCAGCCGATACCGGGAGGTGTGGCCGCTGGCAACCTTTGT |
| | | | GCGGCGCCTGGGGCCCGAGGGCAGGCGGCTTGTGCAACCC |
| | | | GGGGACCCGAAGATCTACCGCACTTTGGTTGCCCAATGCCT |
| | | | AGTGTGCATGCACTGGGGCTCACAGCCTCCACCTGCCGACC |
| | | | TTTCCTTCCACCAGGTGTCATCCCTGAAAGAGCTGGTGGCCA |
| | | | GGGTTGTGCAGAGACTCTGCGAGCGCAACGAGAGAAACGT |
| | | | GCTGGCTTTTGGCTTTGAGCTGCTTAACGAGGCCAGAGGCG |
| | | | GGCCTCCCATGGCCTTCACTAGTAGCGTGCGTAGCTACTTGC |
| | | | CCAACACTGTTATTGAGACCCTGCGTGTCAGTGGTGCATGG |
| | | | ATGCTACTGTTGAGCCGAGTGGGCGACGACCTGCTGGTCTA |
| | | | CCTGCTGGCACACTGTGCTCTTTATCTTCTGGTGCCCCCCAG |
| | | | CTGTGCCTACCAGGTGTGTGGGTCTCCCCTGTACCAAATTTG |
| | | | TGCCACCACGGATATCTGGCCCTCTGTGTCCGCTAGTTACAG |
| | | | GCCCACCCGACCCGTGGGCAGGAATTTCACTAACCTTAGGT |
| | | | TCTTACAACAGATCAAGAGCAGTAGTCGCCAGGAAGCACCG |
| | | | AAACCCCTGGCCTTGCCATCTCGAGGTACAAAGAGGCATCT |
| | | | GAGTCTCACCAGTACAAGTGTGCCTTCAGCTAAGAAGGCCA |
| | | | GATGCTATCCTGTCCCGAGAGTGGAGGAGGGACCCCACAGG |
| | | | CAGGTGCTACCAACCCCATCAGGCAAATCATGGGTGCCAAG |
| | | | TCCTGCTCGGTCCCCCGAGGTGCCTACTGCAGAGAAAGATT |
| | | | TGTCTTCTAAAGGAAAGGTGTCTGACCTGAGTCTCTCTGGGT |
| | | | CGGTGTGCTGTAAACACAAGCCCAGCTCCACATCTCTGCTG |
| | | | TCACCACCCCGCCAAAATGCCTTTCAGCTCAGGCCATTTATT |
| | | | GAGACCAGACATTTCCTTTACTCCAGGGGAGATGGCCAAGA |
| | | | GCGTCTAAACCCCTCATTCCTACTCAGCAACCTCCAGCCTAA |
| | | | CTTGACTGGGGCCAGGAGACTGGTGGAGATCATCTTTCTGG |
| | | | GCTCAAGGCCTAGGACATCAGGACCACTCTGCAGGACACAC |
| | | | CGTCTATCGCGTCGATACTGGCAGATGCGGCCCCTGTTCCA |
| | | | ACAGCTGCTGGTGAACCATGCAGAGTGCCAATATGTCAGAC |
| | | | TCCTCAGGTCACATTGCAGGTTTCGAACAGCAAACCAACAG |
| | | | GTGACAGATGCCTTGAACACCAGCCCACCGCACCTCATGGA |
| | | | TTTGCTCCGCCTGCACAGCAGTCCCTGGCAGGTATATGGTTT |
| | | | TCTTCGGGCCTGTCTCTGCAAGGTGGTGTCTGCTAGTCTCTG |
| | | | GGGTACCAGGCACAATGAGCGCCGCTTCTTTAAGAACTTAA |
| | | | AGAAGTTCATCTCGTTGGGGAAATACGGCAAGCTATCACTG |
| | | | CAGGAACTGATGTGGAAGATGAAAGTAGAGGATTGCCACT |
| | | | GGCTCCGCAGCAGCCCGGGGAAGGACCGTGTCCCCGCTGCA |
| | | | GAGCACCGTCTGAGGGAGAGGATCCTGGCTACGTTCCTGTT |
| | | | CTGGCTGATGGACACATACGTGGTACAGCTGCTTAGGTCAT |
| | | | TCTTTTACATCACAGAGAGCACATTCCAGAAGAACAGGCTC |
| | | | TTCTTCTACCGTAAGAGTGTGTGGAGCAAGCTGCAGAGCAT |
| | | | TGGAGTCAGGCAACACCTTGAGAGAGTGCGGCTACGGGAG |
| | | | CTGTCACAAGAGGAGGTCAGGCATCACCAGGACACCTGGCT |
| | | | AGCCATGCCCATCTGCAGACTGCGCTTCATCCCCAAGCCCA |
| | | | ACGGCCTGCGGCCCATTGTGAACATGAGTTATAGCATGGGT |
| | | | ACCAGAGCTTTGGGCAGAAGGAAGCAGGCCCAGCATTTCAC |
| | | | CCAGCGTCTCAAGACTCTCTTCAGCATGCTCAACTATGAGC |
| | | | GGACAAAACATCCTCACCTTATGGGGTCTTCTGTACTGGGT |
| | | | ATGAATGACATCTACAGGACCTGGCGGGCCTTTGTGCTGCG |
| | | | TGTGCGTGCTCTGGACCAGACACCCAGGATGTACTTTGTTA |
| | | | AGGCAGATGTGACCGGGGCCTATGATGCCATCCCCCAGGGT |
| | | | AAGCTGGTGGAGGTTGTTGCCAATATGATCAGGCACTCGGA |
| | | | GAGCACGTACTGTATCCGCCAGTATGCAGTGGTCCGGAGAG |
| | | | ATAGCCAAGGCCAAGTCCACAAGTCCTTTAGGAGACAGGTC |
| | | | ACCACCCTCTCTGACCTCCAGCCATACATGGGCCAGTTCCTT |
| | | | AAGCATCTGCAGGATTCAGATGCCAGTGCACTGAGGAACTC |
| | | | CGTTGTCATCGAGCAGAGCATCTCTATGAATGAGAGCAGCA |
| | | | GCAGCCTGTTTGACTTCTTCCTGCACTTCCTGCGTCACAGTG |
| | | | TCGTAAAGATTGGTGACAGGTGCTATACGCAGTGCCAGGGC |
| | | | ATCCCCCAGGGCTCCAGCCTATCCACCCTGCTCTGCAGTCTG |
| | | | TGTTTCGGAGACATGGAGAACAAGCTGTTTGCTGAGGTGCA |
| | | | GCGGGATGGGTTGCTTTTACGTTTTGTTGATGACTTTCTGTT |
| | | | GGTGACGCCTCACTTGGACCAAGCAAAAACCTTCCTCAGCA |
| | | | CCCTGGTCCATGGCGTTCCTGAGTATGGGTGCATGATAAAC |
| | | | TTGCAGAAGACAGTGGTGAACTTCCCTGTGGAGCCTGGTAC |
| | | | CCTGGGTGGTGCAGCTCCATACCAGCTGCCTGCTCACTGCCT |
| | | | GTTTCCCTGGTGTGGCTTGCTGCTGGACACTCAGACTTTGGA |
| | | | GGTGTTCTGTGACTACTCAGGTTATGCCCAGACCTCAATTAA |
| | | | GACGAGCCTCACCTTCCAGAGTGTCTTCAAAGCTGGGAAGA |
| | | | CCATGCGGAACAAGCTCCTGTCGGTCTTGCGGTTGAAGTGT |
| | | | CACGGTCTATTTCTAGACTTGCAGGTGAACAGCCTCCAGAC |
| | | | AGTCTGCATCAATATATACAAGATCTTCCTGCTTCAGGCCTA |
| | | | CAGGTTCCATGCATGTGTGATTCAGCTTCCCTTTGACCAGCG |
| | | | TGTTAGGAAGAACCTCACATTCTTTCTGGGCATCATCTCCAG |
| | | | CCAAGCATCCTGCTGCTATGCTATCCTGAAGGTCAAGAATC |
| | | | CAGGAATGACACTAAAGGCCTCTGGCTCCTTTCCTCCTGAA |
| | | | GCCGCACATTGGCTCTGCTACCAGGCCTTCCTGCTCAAGCTG |
| | | | GCTGCTCATTCTGTCATCTACAAATGTCTCCTGGGACCTCTG |
| | | | AGGACAGCCCAAAAACTGCTGTGCCGGAAGCTCCCAGAGG |
| | | | CGACAATGACCATCCTTAAAGCTGCAGCTGACCCAGCCCTA |
| | | | AGCACAGACTTTCAGACCATTTTGGACTAA |
| mTERT | SEQ ID NO: 66 | AA | MTRAPRCPAVRSLLRSRYREVWPLATFVRRLGPEGRRLVQPG |
| | | | DPKIYRTLVAQCLVCMHWGSQPPPADLSFHQVSSLKELVARV |
| | | | VQRLCERNERNVLAFGFELLNEARGGPPMAFTSSVRSYLPNTV |
| | | | IETLRVSGAWMLLLSRVGDDLLVYLLAHCALYLLVPPSCAYQ |
| | | | VCGSPLYQICATTDIWPSVSASYRPTRPVGRNFTNLRFLQQIKS |
| | | | SSRQEAPKPLALPSRGTKRHLSLTSTSVPSAKKARCYPVPRVEE |
| | | | GPHRQVLPTPSGKSWVPSPARSPEVPTAEKDLSSKGKVSDLSLS |
| | | | GSVCCKHKPSSTSLLSPPRQNAFQLRPFIETRHFLYSRGDGQER |
| | | | LNPSFLLSNLQPNLTGARRLVEIIFLGSRPRTSGPLCRTHRLSRR |
| | | | YWQMRPLFQQLLVNHAECQYVRLLRSHCRFRTANQQVTDAL |
| | | | NTSPPHLMDLLRLHSSPWQVYGFLRACLCKVVSASLWGTRHN |
| | | | ERRFFKNLKKFISLGKYGKLSLQELMWKMKVEDCHWLRSSPG |
| | | | KDRVPAAEHRLRERILATFLFWLMDTYVVQLLRSFFYITESTFQ |
| | | | KNRLFFYRKSVWSKLQSIGVRQHLERVRLRELSQEEVRHHQD |
| | | | TWLAMPICRLRFTPKPNGLRPIVNMSYSMGTRALGRRKQAQHF |
| | | | TQRLKTLFSMLNYERTKHPHLMGSSVLGMNDIYRTWRAFVLR |
| | | | VRALDQTPRMYFVKADVTGAYDAIPQGKLVEVVANMIRHSES |
| | | | TYCIRQYAVVRRDSQGQVHKSFRRQVTTLSDLQPYMGQFLKH |
| | | | LQDSDASALRNSVVIEQSISMNESSSSLFDFFLHFLRHSVVKIGD |
| | | | RCYTQCQGIPQGSSLSTLLCSLCFGDMENKLFAEVQRDGLLLR |
| | | | FVDDFLLVTPHLDQAKTFLSTLVHGVPEYGCMINLQKTVVNFP |
| | | | VEPGTLGGAAPYQLPAHCLFPWCGLLLDTQTLEVFCDYSGYA |
| | | | QTSIKTSLTFQSVFKAGKTMRNKLLSVLRLKCHGLFLDLQVNS |
| | | | LQTVCINIYKIFLLQAYRFHACVIQLPFDQRVRKNLTFFLGIISS |
| | | | QASCCYAILKVKNPGMTLKASGSFPPEAAHWLCYQAFLLKLA |
| | | | AHSVIYKCLLGPLRTAQKLLCRKLPEATMTILKAAADPALSTD |
| | | | FQTILD |
| mTfeb | SEQ ID NO: 67 | DNA | ATGGCGTCACGCATCGGGCTGCGCATGCAGCTCATGCGGGA |
| | | | GCAGGCCCAGCAGGAGGAGCAGCGAGAGCGCATGCAGCAG |
| | | | CAGGCTGTCATGCATTATATGCAACAGCAGCAGCAGCAGCA |
| | | | GCAGCAGCTGGGTGGGCCCCCCACCCCAGCCATCAACACCC |
| | | | CTGTCCACTTCCAGTCGCCCCCGCCTGTGCCCGGGGAGGTG |
| | | | CTGAAGGTGCAGTCCTACCTGGAGAACCCCACCTCCTACCA |
| | | | CCTGCAACAGTCCCAGCATCAGAAGGTTCGGAAGTATCTGT |
| | | | CTGAGACCTATGGGAACAAGTTTGCTGCCCACGTGAGCCCA |
| | | | GCCCAAGGTTCCCCGAAGCCTGCCCCAGCAGCATCCCCAGG |
| | | | GGTGCGGGCTGGACACGTACTGTCCACCTCGGCCGGCAACA |
| | | | GTGCTCCCAACAGTCCCATGGCCATGCTACATATCAGCTCC |
| | | | AACCCCGAGAAAGAGTTTGATGATGTCATTGACAACATTAT |
| | | | GCGCCTGGACAGCGTGCTGGGCTACATCAACCCTGAGATGC |
| | | | AGATGCCTAACACGCTGCCCCTGTCTAGCAGCCACCTGAAC |
| | | | GTGTACAGCGGTGACCCCCAGGTCACAGCCTCCATGGTGGG |
| | | | TGTCACCAGCAGCTCCTGCCCTGCCGACCTGACTCAGAAGC |
| | | | GAGAGCTAACAGATGCTGAGAGCAGAGCCCTGGCCAAGGA |
| | | | GCGGCAGAAGAAAGACAATCACAACCTAATTGAGAGAAGA |
| | | | CGCAGGTTCAACATCAATGACCGGATCAAGGAGCTGGGAAT |
| | | | GCTGATCCCCAAGGCCAACGACCTGGACGTGCGCTGGAACA |
| | | | AAGGCACCATCCTCAAGGCCTCTGTGGATTACATCCGGAGG |
| | | | ATGCAGAAGGACCTGCAGAAGTCCCGGGAGCTGGAGAACC |
| | | | ACTCCCGGCGCCTGGAGATGACTAACAAGCAGCTCTGGCTC |
| | | | CGCATCCAGGAGCTGGAGATGCAGGCACGCGTGCACGGCCT |
| | | | CCCCACCACCTCGCCGTCGGGTGTGAATATGGCCGAGCTGG |
| | | | CCCAGCAGGTGGTGAAGCAAGAGTTGCCCAGTGAGGATGG |
| | | | CCCAGGGGAGGCGCTGATGCTGGGGCCTGAGGTCCCTGAGC |
| | | | CTGAGCAAATGCCGGCTCTTCCTCCCCAGGCTCCGCTGCCCT |
| | | | CGGCCGCCCAGCCACAGTCTCCGTTCCATCACCTGGACTTC |
| | | | AGCCATGGCCTGAGCTTTGGGGGTGGGGGCGACGAGGGGC |
| | | | CCACAGGTTACCCCGATACCCTGGGGACAGAGCACGGCTCC |
| | | | CCATTCCCCAACCTGTCCAAGAAGGATCTGGACTTAATGCT |
| | | | CCTAGATGACTCCCTGCTCCCCCTGGCCTCTGACCCCCTCTT |
| | | | TTCTACCATGTCTCCTGAGGCCTCCAAGGCCAGCAGCCGCC |
| | | | GGAGCAGCTTCAGCATGGAGGAGGGTGATGTTCTGTGA |
| mTfeb | SEQ ID NO: 68 | AA | MASRIGLRMQLMREQAQQEEQRERMQQQAVMHYMQQQQQQ |
| | | | QQQLGGPPTPAINTPVHFQSPPPVPGEVLKVQSYLENPTSYHLQ |
| | | | QSQHQKVRKYLSETYGNKFAAHVSPAQGSPKPAPAASPGVRA |
| | | | GHVLSTSAGNSAPNSPMAMLHISSNPEKEFDDVIDNIMRLDSV |
| | | | LGYINPEMQMPNTLPLSSSHLNVYSGDPQVTASMVGVTSSSCP |
| | | | ADLTQKRELTDAESRALAKERQKKDNHNLIERRRRFNINDRIK |
| | | | ELGMLIPKANDLDVRWNKGTILKASVDYIRRMQKDLQKSREL |
| | | | ENHSRRLEMTNKQLWLRIQELEMQARVHGLPTTSPSGVNMAE |
| | | | LAQQVVKQELPSEDGPGEALMLGPEVPEPEQMPALPPQAPLPS |
| | | | AAQPQSPFHHLDFSHGLSFGGGGDEGPTGYPDTLGTEHGSPFP |
| | | | NLSKKDLDLMLLDDSLLPLASDPLFSTMSPEASKASSRRSSFSM |
| | | | EEGDVL |
| mTxn1 | SEQ ID NO: 69 | DNA | ATGGTGAAGCTGATCGAGAGCAAGGAAGCTTTTCAGGAGGC |
| | | | CCTGGCCGCCGCGGGAGACAAGCTTGTCGTGGTGGACTTCT |
| | | | CTGCTACGTGGTGTGGACCTTGCAAAATGATCAAGCCCTTCT |
| | | | TCCATTCCCTCTGTGACAAGTATTCCAATGTGGTGTTCCTTG |
| | | | AAGTGGATGTGGATGACTGCCAGGATGTTGCTGCAGACTGT |
| | | | GAAGTCAAATGCATGCCGACCTTCCAGTTTTATAAAAAGGG |
| | | | TCAAAAGGTGGGGGAGTTCTCCGGTGCTAACAAGGAAAAG |
| | | | CTTGAAGCCTCTATTACTGAATATGCCTAA |
| mTxn1 | SEQ ID NO: 70 | AA | MVKLIESKEAFQEALAAAGDKLVVVDFSATWCGPCKMIKPFF |
| | | | HSLCDKYSNVVFLEVDVDDCQDVAADCEVKCMPTFQFYKKG |
| | | | QKVGEFSGANKEKLEASITEYA |
| mUcp1 | SEQ ID NO: 71 | DNA | ATGGTGAACCCGACAACTTCCGAAGTGCAACCCACCATGGG |
| | | | GGTCAAGATCTTCTCAGCCGGAGTTTCAGCTTGCCTGGCAG |
| | | | ATATCATCACCTTCCCGCTGGACACTGCCAAAGTCCGCCTTC |
| | | | AGATCCAAGGTGAAGGCCAGGCTTCCAGTACCATTAGGTAT |
| | | | AAAGGTGTCCTAGGGACCATCACCACCCTGGCAAAAACAG |
| | | | AAGGATTGCCGAAACTGTACAGCGGTCTGCCTGCGGGCATT |
| | | | CAGAGGCAAATCAGCTTTGCCTCACTCAGGATTGGCCTCTA |
| | | | CGACTCAGTCCAAGAGTACTTCTCTTCAGGGAGAGAAACAC |
| | | | CTGCCTCTCTCGGAAACAAGATCTCAGCCGGCTTAATGACT |
| | | | GGAGGTGTGGCAGTGTTCATTGGGCAGCCTACAGAGGTCGT |
| | | | GAAGGTCAGAATGCAAGCCCAGAGCCATCTGCATGGGATCA |
| | | | AACCCCGCTACACGGGGACCTACAATGCTTACAGAGTTATA |
| | | | GCCACCACAGAAAGCTTGTCAACACTTTGGAAAGGGACGAC |
| | | | CCCTAATCTAATGAGAAATGTCATCATCAATTGTACAGAGC |
| | | | TGGTAACATATGACCTCATGAAGGGGGCCCTTGTAAACAAC |
| | | | AAAATACTGGCAGATGACGTCCCCTGCCATTTACTGTCAGC |
| | | | TCTTGTTGCCGGGTTTTGCACCACACTCCTGGCCTCTCCAGT |
| | | | GGATGTGGTAAAAACAAGATTCATCAACTCTCTGCCAGGAC |
| | | | AGTACCCAAGCGTACCAAGCTGTGCGATGTCCATGTACACC |
| | | | AAGGAAGGACCGACGGCCTTTTTCAAAGGGTTTGTGGCTTC |
| | | | TTTTCTGCGACTCGGGTCCTGGAACGTCATCATGTTTGTGTG |
| | | | CTTTGAACAGCTGAAAAAAGAGCTGATGAAGTCCAGACAG |
| | | | ACAGTGGATTGTACCACATAA |
| mUcp1 | SEQ ID NO: 72 | AA | MVNPTTSEVQPTMGVKIFSAGVSACLADIITFPLDTAKVRLQIQ |
| | | | GEGQASSTIRYKGVLGTITTLAKTEGLPKLYSGLPAGIQRQISFA |
| | | | SLRIGLYDSVQEYFSSGRETPASLGNKISAGLMTGGVAVFIGQP |
| | | | TEVVKVRMQAQSHLHGIKPRYTGTYNAYRVIATTESLSTLWK |
| | | | GTTPNLMRNVIINCTELVTYDLMKGALVNNKILADDVPCHLLS |
| | | | ALVAGFCTTLLASPVDVVKTRFINSLPGQYPSVPSCAMSMYTK |
| | | | EGPTAFFKGFVASFLRLGSWNVIMFVCFEQLKKELMKSRQTV |
| | | | DCTT |
| nmr Has2 | SEQ ID NO: 73 | DNA | ATGCATTGTGAGAGGTTTCTATGTGTCCTGAGAATAATTGG |
| | | | AACTACACTTTTTGGAGTGTCTCTCCTCCTCGGAATCACAGC |
| | | | TGCTTATATTGTTGGCTACCAGTTTATCCAAACAGATAATTA |
| | | | CTACTTCTCATTTGGACTGTACGGTGCCTTTTTAGCCTCGCA |
| | | | TCTCATCATCCAAAGCCTCTTTGCCTTTTTGGAACACCGGAA |
| | | | AATGAAGAAGTCCCTTGAAACCCCGATTAAATTGAACAAAA |
| | | | CGGTAGCACTCTGCATCGCTGCGTACCAAGAGGACCCTGAC |
| | | | TACTTACGGAAATGTTTGCAATCTGTGAAAAGGCTGACCTA |
| | | | CCCTGGGATTAAAGTCGTGATGGTCATCGATGGGAACTCAG |
| | | | ACGACGACCTTTACATGATGGACATATTCAGCGAAGTTATG |
| | | | GGCAGGGACAAATCGGCCACGTACATCTGGAAGAACAACTT |
| | | | TCATGAAAAGGGACCTGGTGAGACAGAAGAGTCCCATAAA |
| | | | GAAAGTTCACAACATGTCACCCAATTGGTCTTGTCTAGCAA |
| | | | AAGTGTTTGCATCATGCAAAAATGGGGTGGAAAGAGAGAA |
| | | | GTCATGTACACAGCCTTCAGAGCACTGGGGCGAAGCGTGGA |
| | | | TTATGTACAGGTGTGTGACTCAGATACTATGCTTGACCCTGC |
| | | | CTCATCTGTGGAGATGGTGAAGGTCTTAGAGGAAGACCCTA |
| | | | TGGTTGGAGGTGTTGGAGGAGATGTCCAGATTTTAAACAAG |
| | | | TATGATTCCTGGATCTCCTTCCTCAGCAGCGTGAGATACTGG |
| | | | ATGGCTTTTAATATAGAAAGGGCCTGCCAGTCTTATTTTGGC |
| | | | TGTGTCCAGTGCATAAGCGGTCCTCTGGGAATGTACAGAAA |
| | | | CTCCTTGCTGCATGAATTTGTGGAAGACTGGTACAGTCAGG |
| | | | AATTCATGGGTAACCAATGCAGTTTTGGTGACGACAGGCAC |
| | | | CTTACCAACAGGGTGTTGAGTCTGGGCTATGCAACTAAATA |
| | | | CACGGCTCGGTCCAAGTGCCTTACTGAAACTCCCATAGAAT |
| | | | ATCTGAGATGGCTGAACCAGCAGACCCGTTGGAGCAAGTCC |
| | | | TACTTCCGAGAGTGGCTGTACAATGCCATGTGGTTTCACAA |
| | | | GCATCACTTGTGGATGACCTATGAAGCTGTTATCACTGGATT |
| | | | CTTTCCTTTCTTTCTCATTGCCACAGTCATCCAGCTCTTCTAC |
| | | | AGGGGTAAAATCTGGAACATCCTCCTCTTCCTGTTAACTGTC |
| | | | CAGCTAGTGGGTCTCATCAAGTCATCTTTTGCCAGCTGCCTT |
| | | | AGAGGAAATATCGTCATGGTATTCATGTCTCTGTATTCAGTG |
| | | | TTATACATGTCAAGTCTACTTCCTGCCAAGATGTTTGCAATT |
| | | | GCAACCATAAACAAAGCTGGGTGGGGCACATCTGGAAGGA |
| | | | AGACCATTGTTGTTAATTTCATAGGACTTATTCCAGTGTCCG |
| | | | TGTGGTTTACAATCCTTCTAGGTGGTGTAATTTTCACCATTT |
| | | | ATAAGGAATCTAAAAAGCCATTTTCCGAATCCAAACAGACT |
| | | | GTTCTCATCGTGGGAACTTTGATCTATGCATGCTACTGGGTC |
| | | | ATGCTTTTGACTCTCTATGTGGTTCTCATCAATAAGTGTGGC |
| | | | AGGCGGAAGAAGGGACAACAGTATGACATGGTGCTTGATG |
| | | | TATGA |
| nmr Has2 | SEQ ID NO: 74 | AA | MHCERFLCVLRIIGTTLFGVSLLLGITAAYIVGYQFIQTDNYYFS |
| | | | FGLYGAFLASHLIIQSLFAFLEHRKMKKSLETPIKLNKTVALCIA |
| | | | AYQEDPDYLRKCLQSVKRLTYPGIKVVMVIDGNSDDDLYMM |
| | | | DIFSEVMGRDKSATYIWKNNFHEKGPGETEESHKESSQHVTQL |
| | | | VLSSKSVCIMQKWGGKREVMYTAFRALGRSVDYVQVCDSDT |
| | | | MLDPASSVEMVKVLEEDPMVGGVGGDVQILNKYDSWISFLSS |
| | | | VRYWMAFNIERACQSYFGCVQCISGPLGMYRNSLLHEFVEDW |
| | | | YSQEFMGNQCSFGDDRHLTNRVLSLGYATKYTARSKCLTETPI |
| | | | EYLRWLNQQTRWSKSYFREWLYNAMWFHKHHLWMTYEAVI |
| | | | TGFFPFFLIATVIQLFYRGKIWNILLFLLTVQLVGLIKSSFASCLR |
| | | | GNIVMVFMSLYSVLYMSSLLPAKMFAIATINKAGWGTSGRKTI |
| | | | VVNFIGLIPVSVWFTILLGGVIFTIYKESKKPFSESKQTVLIVGTL |
| | | | IYACYWVMLLTLYVVLINKCGRRKKGQQYDMVLDV |
| Adcy5- | SEQ ID NO: 75 | DNA | CCAAGGTATATTGCTGTTGACAGTGAGCGACGCTGCAGATA |
| Coq7 | | | TTCCGCTCTAAGTGAAGCCACAGATGTTAGAGCGGAAAATC |
| | | | TGCAGAGCTGCCTACTGCCTCGGACTTCAAGGGGCTTGCGG |
| | | | CCGCCATCTCCATGGCTGTACCACCTTGTCGGCCAGGTTACT |
| | | | ACAGATATGTATGTTGAATCTCATTACATATCTGTTGTAACC |
| | | | TGCTCTGACATTTTGGTATCTTTCATCTGACCACGTACTACC |
| | | | TTCTATCTGATGTGACAGCTTCTGTAGCACCAGATGAAGATT |
| | | | GGGCTCAATGTTTAGTTATTTGAGCCCAAGCTTCATCTGTGT |
| | | | ACTGCTAGCTGTAGAACTCCAGCTTCGGCCTGTAACTTATGA |
| | | | TAGCAATGTCAGCAGTGCCTGGCAGCCGTGGATCGAATAAT |
| | | | TTAAGATTCTAAAATTATAGTATTCGATCAACGGCTGCAAA |
| | | | GTAAGGTTGACCATACTCTACAGTTGTTGATTTCGTGGCTAC |
| | | | AGAGTTTCCTTAGCAGAGCTGGATGCAGTGCAGCCATATAT |
| | | | TTGTCTAAACTATAATATATGGCTGCACTGCATATAGCTACT |
| | | | GCTAGGCAATCCTTCCCTCGATAAGATGCAGCGGCGGCTCC |
| | | | TCTCCCCATGGCCCTGGCCTTGTTGAAGAGGATTATCCTGGG |
| | | | CTCAGAGATAATCCTCTACAACAAGGGCAGGGACCTGGGGA |
| | | | CCCCGGCACCGGCAGGCTAGC |
| Agtr1a- | SEQ ID NO: 76 | DNA | GGCCAAGGTATATTGCTGTTGACAGTGAGCGACGCTGCAGA |
| Adcy5- | | | TATTCCGCTCTAAGTGAAGCCACAGATGTTAGAGCGGAAAA |
| akt1-ikbkb | | | TCTGCAGAGCTGCCTACTGCCTCGGACTTCAAGGGGCTTGC |
| | | | GGCCGCCATCTCCATGGCTGTACCACCTTGTCGGCCAGGTT |
| | | | ACTACAGATATGTATGTTGAATCTCATTACATATCTGTTGTA |
| | | | ACCTGCTCTGACATTTTGGTATCTTTCATCTGACCACGTACT |
| | | | ACCTTCTATCTGATGTGACAGCTTCTGTAGCACCCTGTTACT |
| | | | ACACATACTTTTGTTTAGTTATAAAGTATGTGGAGTAACAG |
| | | | GTGTACTGCTAGCTGTAGAACTCCAGCTTCGGCCTGTAACTT |
| | | | ATGATAGCAATGTCAGCAGTGCCTCCTGTTCCCTTTCCTAAT |
| | | | CATTTAAGATTCTAAAATTATAGGATTAGGAATGGGAACAG |
| | | | TAAGTAAGGTTGACCATACTCTACAGTTGTTGATTTCGTGGC |
| | | | TACAGAGTTTCCTTAGCAGAGCTGTGGCACCTTTATTGGCTA |
| | | | CAATGTCTAAACTATTTGTAGCCAATAAAGGTGCCCTAGCT |
| | | | ACTGCTAGGCAATCCTTCCCTCGATAAGTATGGGGCCTGGC |
| | | | TCGAGCAGGGGGCGAGGGATGCATCTAGTAGAGCGGATGA |
| | | | TTGGTCCCCTCCCTTAACAAGTCGAACTGTCTTGTCCTTCCC |
| | | | TCCCAATGACCGCGTCTTCGTCACAGTCAGCGGCGGCTCCT |
| | | | CTCCCCATGGCCCTGATGCTGTCCCTGGTCCTTATGCTGGGC |
| | | | TCAGAATACCTGGTGTGAGTCTCAGTCAGGGACCTGGGGAC |
| | | | CCCGGCACCGGCAGGCTA |
| Agtr1a- | SEQ ID NO: 77 | DNA | CCAAGGTATATTGCTGTTGACAGTGAGCGACGCTGCAGATA |
| Adcy5- | | | TTCCGCTCTAAGTGAAGCCACAGATGTTAGAGCGGAAAATC |
| Coq7 | | | TGCAGAGCTGCCTACTGCCTCGGACTTCAAGGGGCTTGCGG |
| | | | CCGCCATCTCCATGGCTGTACCACCTTGTCGGCCAGGTTACT |
| | | | ACAGATATGTATGTTGAATCTCATTACATATCTGTTGTAACC |
| | | | TGCTCTGACATTTTGGTATCTTTCATCTGACCACGTACTACC |
| | | | TTCTATCTGATGTGACAGCTTCTGTAGCACCCTGTTACTACA |
| | | | CATACTTTTGTTTAGTTATAAAGTATGTGGAGTAACAGGTGT |
| | | | ACTGCTAGCTGTAGAACTCCAGCTTCGGCCTGTAACTTATGA |
| | | | TAGCAATGTCAGCAGTGCCTCCTGTTCCCTTTCCTAATCATT |
| | | | TAAGATTCTAAAATTATAGGATTAGGAATGGGAACAGTAAG |
| | | | TAAGGTTGACCATACTCTACAGTTGTTGATTTCGTGGCTACA |
| | | | GAGTTTCCTTAGCAGAGCTGGATGCAGTGCAGCCATATATT |
| | | | TGTCTAAACTATAATATATGGCTGCACTGCATATAGCTACTG |
| | | | CTAGGCAATCCTTCCCTCGATAAGATGCAGCGGCGGCTCCT |
| | | | CTCCCCATGGCCCTGGCCTTGTTGAAGAGGATTATCCTGGGC |
| | | | TCAGAGATAATCCTCTACAACAAGGGCAGGGACCTGGGGAC |
| | | | CCCGGCACCGGCAGGCTAGC |
| Agtr1a- | SEQ ID NO: 78 | DNA | GGCCAAGGTATATTGCTGTTGACAGTGAGCGACGCTGCAGA |
| Adcy5- | | | TATTCCGCTCTAAGTGAAGCCACAGATGTTAGAGCGGAAAA |
| mTOR | | | TCTGCAGAGCTGCCTACTGCCTCGGACTTCAAGGGGCTTGC |
| | | | GGCCGCCATCTCCATGGCTGTACCACCTTGTCGGCCAGGTT |
| | | | ACTACAGATATGTATGTTGAATCTCATTACATATCTGTTGTA |
| | | | ACCTGCTCTGACATTTTGGTATCTTTCATCTGACCACGTACT |
| | | | ACCTTCTATCTGATGTGACAGCTTCTGTAGCACCCTGTTACT |
| | | | ACACATACTTTTGTTTAGTTATAAAGTATGTGGAGTAACAG |
| | | | GTGTACTGCTAGCTGTAGAACTCCAGCTTCGGCCTGTAACTT |
| | | | ATGATAGCAATGTCAGCAGTGCCTCCTGTTCCCTTTCCTAAT |
| | | | CATTTAAGATTCTAAAATTATAGGATTAGGAATGGGAACAG |
| | | | TAAGTAAGGTTGACCATACTCTACAGTTGTTGATTTCGTGGC |
| | | | TACAGAGTTTCCTTAGCAGAGCTGCTGGATGCAGTGGCGAC |
| | | | ATTTTGTCTAAACTATAAATGTCGCCACTGCATCCATTAGCT |
| | | | ACTGCTAGGCAATCCTTCCCTCGATAAGTATGGGGCCTGGC |
| | | | TCGAGCAGGGGGCGAGGGATCAGACAGTTGGACTTGTTAAA |
| | | | TGGTCCCCTCCCTCTTGTCTGAATCAGGTAATGTCCTTCCCT |
| | | | CCCAATGACCGCGTCTTCGTCACAGTCAGCGGCGGCTCCTC |
| | | | TCCCCATGGCCCTGATGCTGTCCCTGGTCCTTATGCTGGGCT |
| | | | CAGACATAAGGACCACGGACAGCAACAGGGACCTGGGGAC |
| | | | CCCGGCACCGGCAGGCTA |
| Agtr1a- | SEQ ID NO: 79 | DNA | GGCCAAGGTATATTGCTGTTGACAGTGAGCGACCTTACCTG |
| Ikbkb- | | | AATCAGACAAGAAGTGAAGCCACAGATGTTCTTGTCTGAAT |
| mTOR | | | CAGGTAATGCTGCCTACTGCCTCGGACTTCAAGGGGCTTGC |
| | | | GGCCGCCATCTCCATGGCTGTACCACCTTGTCGGGCATCTA |
| | | | GTAGAGCGGATGATTGTTGAATCTCATTTCATCCGCTCAACT |
| | | | AGATGGTCTGACATTTTGGTATCTTTCATCTGACCACGTACT |
| | | | ACCTTCTATCTGATGTGACAGCTTCTGTAGCACCCTGTTACT |
| | | | ACACATACTTTTGTTTAGTTATAAAGTATGTGGAGTAACAG |
| | | | GTGTACTGCTAGCTGTAGAACTCCAGCTTCGGCCTGTAACTT |
| | | | ATGATAGCAATGTCAGCAGTGCCTCCTGTTCCCTTTCCTAAT |
| | | | CATTTAAGATTCTAAAATTATAGGATTAGGAATGGGAACAG |
| | | | TAAGTAAGGTTGACCATACTCTACAGTTGTTGATTTCGTGGC |
| | | | TACAGAGTTTCCTTAGCAGAGCTGCTGGATGCAGTGGCGAC |
| | | | ATTTTGTCTAAACTATAAATGTCGCCACTGCATCCATTAGCT |
| | | | ACTGCTAGGCAATCCTTCCCTCGATAAGTATGGGGCCTGGC |
| | | | TCGAGCAGGGGGCGAGGGATCAGACAGTTGGACTTGTTAAA |
| | | | TGGTCCCCTCCCTATGAACGGTCTTTCCCTCTGTCCTTCCCTC |
| | | | CCAATGACCGCGTCTTCGTCACAGTCAGCGGCGGCTCCTCT |
| | | | CCCCATGGCCCTGGCCTTGTTGAAGAGGATTATCCTGGGCTC |
| | | | AGACATAAGGACCACGGACAGCAACAGTGACCTGGGGACC |
| | | | CCGGCACCGGCAGGCTA |
| Agtr1a- | SEQ ID NO: 80 | DNA | CCAAGGTATATTGCTGTTGACAGTGAGCGACCTTACCTGAA |
| Ikbkb | | | TCAGACAAGAAGTGAAGCCACAGATGTTCTTGTCTGAATCA |
| | | | GGTAATGCTGCCTACTGCCTCGGACTTCAAGGGGCTTGCGG |
| | | | CCGCCATCTCCATGGCTGTACCACCTTGTCGGGCATCTAGTA |
| | | | GAGCGGATGATTGTTGAATCTCATTTCATCCGCTCAACTAGA |
| | | | TGGTCTGACATTTTGGTATCTTTCATCTGACCACGTACTACC |
| | | | TTCTATCTGATGTGACAGCTTCTGTAGCACCCTGTTACTACA |
| | | | CATACTTTTGTTTAGTTATAAAGTATGTGGAGTAACAGGTGT |
| | | | ACTGCTAGCTGTAGAACTCCAGCTTCGGCCTGTAACTTATGA |
| | | | TAGCAATGTCAGCAGTGCCTCCTGTTCCCTTTCCTAATCATT |
| | | | TAAGATTCTAAAATTATAGGATTAGGAATGGGAACAGTAAG |
| | | | TAAGGTTGACCATACTCTACAGTTGTTGATTTCGTGGCTACA |
| | | | GAGTTTCCTTAGCAGAGCTGGCTCTAAAGAAGGCTTATGAA |
| | | | TGTCTAAACTATTTCATAAGCCTACTTTAGAGATAGCTACTG |
| | | | CTAGGCAATCCTTCCCTCGATAAGTATGGGGCCTGGCTCGA |
| | | | GCAGGGGGCGAGGGATGCATCTAGTAGAGCGGATGATTGG |
| | | | TCCCCTCCCTCATCCGCTCAACTAGATGAGTCCTTCCCTCCC |
| | | | AATGACCGCGTCTTGGCTAGC |
| Ctf1-Akt1 | SEQ ID NO: 81 | DNA | CCAAGGTATATTGCTGTTGACAGTGAGCGACTCTGAGACTG |
| | | | ACACCAGGTATGTGAAGCCACAGATGATACCTGGTGTGAGT |
| | | | CTCAGCGCTGCCTACTGCCTCGGACTTCAAGGGGCTTGCGG |
| | | | CCGCCATCTCCATGGCTGTACCACCTTGTCGGTGGCACCTTT |
| | | | ATTGGCTACAATGTTGAATCTCATTTGTAGCCAATTAAGGTG |
| | | | CCTTCTGACATTTTGGTATCTTTCATCTGACCACGTACTACC |
| | | | TTCTATCTGATGTGACAGCTTCTGTAGCACGCCACCCTCTTC |
| | | | ACGGCCAATGTTTAGTTATTTGGCCGTGACGAGGGTGGCTG |
| | | | TACTGCTAGCTGTAGAACTCCAGCTTCGGCCTGTAACTTATG |
| | | | ATAGCAATGTCAGCAGTGCCTCGCCACCCTCTTCACGGCCA |
| | | | ATTAAGATTCTAAAATTATTGGGCCGTGAACAGGGTGGCTA |
| | | | AGTAAGGTTGACCATACTCTACAGTTGTTGATTTCGTGGCTA |
| | | | CAGAGTTTCCTTAGCAGAGCTGTGGCACCTTTATTGGCTACA |
| | | | ATGTCTAAACTATTTGTAGCCAATAAAGGTGCCCTAGCTACT |
| | | | GCTAGGCAATCCTTCCCTCGATAAGATGCAGCGGCGGCTCC |
| | | | TCTCCCCATGGCCCTGCTTGGGCTGTCCCTATCTTTCCTGGG |
| | | | CTCAGAGAAAGATAGGGTCAGCCCAACCAGGGACCTGGGG |
| | | | ACCCCGGCACCGGCAGGCTAGC |
| Ctf1-Coq7 | SEQ ID NO: 82 | DNA | CCAAGGTATATTGCTGTTGACAGTGAGCGACCTTGGGCTGT |
| | | | CCCTATCTTTCGTGAAGCCACAGATGGAAAGATAGGGTCAG |
| | | | CCCAATGCTGCCTACTGCCTCGGACTTCAAGGGGCTTGCGG |
| | | | CCGCCATCTCCATGGCTGTACCACCTTGTCGGGGCAGCCGT |
| | | | GGATCGAATAATTGTTGAATCTCATTATATTCGATCAACGGC |
| | | | TGCGTCTGACATTTTGGTATCTTTCATCTGACCACGTACTAC |
| | | | CTTCTATCTGATGTGACAGCTTCTGTAGCACGCCACCCTCTT |
| | | | CACGGCCAATGTTTAGTTATTTGGCCGTGACGAGGGTGGCT |
| | | | GTACTGCTAGCTGTAGAACTCCAGCTTCGGCCTGTAACTTAT |
| | | | GATAGCAATGTCAGCAGTGCCTCGCCACCCTCTTCACGGCC |
| | | | AATTAAGATTCTAAAATTATTGGGCCGTGAACAGGGTGGCT |
| | | | AAGTAAGGTTGACCATACTCTACAGTTGTTGATTTCGTGGCT |
| | | | ACAGAGTTTCCTTAGCAGAGCTGGATGCAGTGCAGCCATAT |
| | | | ATTTGTCTAAACTATAATATATGGCTGCACTGCATATAGCTA |
| | | | CTGCTAGGCAATCCTTCCCTCGATAAGATGCAGCGGCGGCT |
| | | | CCTCTCCCCATGGCCCTGGCCTTGTTGAAGAGGATTATCCTG |
| | | | GGCTCAGAGATAATCCTCTACAACAAGGGCAGGGACCTGGG |
| | | | GACCCCGGCACCGGCAGGCTAGC |
| Ctf1- | SEQ ID NO: 83 | DNA | GGCCAAGGTATATTGCTGTTGACAGTGAGCGACCTTACCTG |
| ikbkb- | | | AATCAGACAAGAAGTGAAGCCACAGATGTTCTTGTCTGAAT |
| Coq7 | | | CAGGTAATGCTGCCTACTGCCTCGGACTTCAAGGGGCTTGC |
| | | | GGCCGCCATCTCCATGGCTGTACCACCTTGTCGGGCATCTA |
| | | | GTAGAGCGGATGATTGTTGAATCTCATTTCATCCGCTCAACT |
| | | | AGATGGTCTGACATTTTGGTATCTTTCATCTGACCACGTACT |
| | | | ACCTTCTATCTGATGTGACAGCTTCTGTAGCACGCCACCCTC |
| | | | TTCACGGCCAATGTTTAGTTATTTGGCCGTGACGAGGGTGG |
| | | | CTGTACTGCTAGCTGTAGAACTCCAGCTTCGGCCTGTAACTT |
| | | | ATGATAGCAATGTCAGCAGTGCCTCGCCACCCTCTTCACGG |
| | | | CCAATTAAGATTCTAAAATTATTGGGCCGTGAACAGGGTGG |
| | | | CTAAGTAAGGTTGACCATACTCTACAGTTGTTGATTTCGTGG |
| | | | CTACAGAGTTTCCTTAGCAGAGCTGGATGCAGTGCAGCCAT |
| | | | ATATTTGTCTAAACTATAATATATGGCTGCACTGCATATAGC |
| | | | TACTGCTAGGCAATCCTTCCCTCGATAAGTATGGGGCCTGG |
| | | | CTCGAGCAGGGGGCGAGGGATGCATCTAGTAGAGCGGATG |
| | | | ATTGGTCCCCTCCCAACTGCCACTGTTGATGTTTGTCCTTCC |
| | | | CTCCCAATGACCGCGTCTTCGTCACAGTCAGCGGCGGCTCC |
| | | | TCTCCCCATGGCCCTGCGATGCAGAGTCCAGGATAATCTGG |
| | | | GCTCAGAGATAATCCTCTACAACAAGGGCAGGGACCTGGGG |
| | | | ACCCCGGCACCGGCAGGCTA |
| Ctf1- | SEQ ID NO: 84 | DNA | GGCCAAGGTATATTGCTGTTGACAGTGAGCGACATGCTGTC |
| mTOR- | | | CCTGGTCCTTATGGTGAAGCCACAGATGCATAAGGACCACG |
| Coq7- | | | GACAGCAGGCTGCCTACTGCCTCGGACTTCAAGGGGCTTGC |
| Slc 13a5 | | | GGCCGCCATCTCCATGGCTGTACCACCTTGTCGGCTGGATG |
| | | | CAGTGGCGACATTTTGTTGAATCTCATTAATGTCGCCAGTGC |
| | | | ATCCACTCTGACATTTTGGTATCTTTCATCTGACCACGTACT |
| | | | ACCTTCTATCTGATGTGACAGCTTCTGTAGCACGCCACCCTC |
| | | | TTCACGGCCAATGTTTAGTTATTTGGCCGTGACGAGGGTGG |
| | | | CTGTACTGCTAGCTGTAGAACTCCAGCTTCGGCCTGTAACTT |
| | | | ATGATAGCAATGTCAGCAGTGCCTCGCCACCCTCTTCACGG |
| | | | CCAATTAAGATTCTAAAATTATTGGGCCGTGAACAGGGTGG |
| | | | CTAAGTAAGGTTGACCATACTCTACAGTTGTTGATTTCGTGG |
| | | | CTACAGAGTTTCCTTAGCAGAGCTGGATGCAGTGCAGCCAT |
| | | | ATATTTGTCTAAACTATAATATATGGCTGCACTGCATATAGC |
| | | | TACTGCTAGGCAATCCTTCCCTCGATAAGTATGGGGCCTGG |
| | | | CTCGAGCAGGGGGCGAGGGATCGAGGGAAACACCGTTCAT |
| | | | ATTGGTCCCCTCCCTTAACAAGTCGAACTGTCTTGTCCTTCC |
| | | | CTCCCAATGACCGCGTCTTCGTCACAGTCAGCGGCGGCTCC |
| | | | TCTCCCCATGGCCCTGGCCTTGTTGAAGAGGATTATCCTGGG |
| | | | CTCAGAGATAATCCTCTACAACAAGGGCAGGGACCTGGGGA |
| | | | CCCCGGCACCGGCAGGCTA |
| Ctf1- | SEQ ID NO: 85 | DNA | GGCCAAGGTATATTGCTGTTGACAGTGAGCGACATGCTGTC |
| mTOR- | | | CCTGGTCCTTATGGTGAAGCCACAGATGCATAAGGACCACG |
| Coq7 | | | GACAGCAGGCTGCCTACTGCCTCGGACTTCAAGGGGCTTGC |
| | | | GGCCGCCATCTCCATGGCTGTACCACCTTGTCGGCTGGATG |
| | | | CAGTGGCGACATTTTGTTGAATCTCATTAATGTCGCCAGTGC |
| | | | ATCCACTCTGACATTTTGGTATCTTTCATCTGACCACGTACT |
| | | | ACCTTCTATCTGATGTGACAGCTTCTGTAGCACGCCACCCTC |
| | | | TTCACGGCCAATGTTTAGTTATTTGGCCGTGACGAGGGTGG |
| | | | CTGTACTGCTAGCTGTAGAACTCCAGCTTCGGCCTGTAACTT |
| | | | ATGATAGCAATGTCAGCAGTGCCTCGCCACCCTCTTCACGG |
| | | | CCAATTAAGATTCTAAAATTATTGGGCCGTGAACAGGGTGG |
| | | | CTAAGTAAGGTTGACCATACTCTACAGTTGTTGATTTCGTGG |
| | | | CTACAGAGTTTCCTTAGCAGAGCTGGATGCAGTGCAGCCAT |
| | | | ATATTTGTCTAAACTATAATATATGGCTGCACTGCATATAGC |
| | | | TACTGCTAGGCAATCCTTCCCTCGATAAGTATGGGGCCTGG |
| | | | CTCGAGCAGGGGGCGAGGGATCAGACAGTTGGACTTGTTAA |
| | | | ATGGTCCCCTCCCTATGAACGGTCTTTCCCTCTGTCCTTCCC |
| | | | TCCCAATGACCGCGTCTTCGTCACAGTCAGCGGCGGCTCCT |
| | | | CTCCCCATGGCCCTGGCCTTGTTGAAGAGGATTATCCTGGGC |
| | | | TCAGAGATAATCCTCTACAACAAGGGCAGGGACCTGGGGAC |
| | | | CCCGGCACCGGCAGGCTA |
| Ctf1- | SEQ ID NO: 86 | DNA | CCAAGGTATATTGCTGTTGACAGTGAGCGACCCTGGGCTTC |
| Slc13a1- | | | TAACTTTGTTAGTGAAGCCACAGATGTAACAAAGTTACAAG |
| pappa | | | CCCAGTGCTGCCTACTGCCTCGGACTTCAAGGGGCTTGCGG |
| | | | CCGCCATCTCCATGGCTGTACCACCTTGTCGGCCGCATCTTA |
| | | | AACTTGGAGTTTGTTGAATCTCATTACTCCAAGTTAAAGATG |
| | | | CGCTCTGACATTTTGGTATCTTTCATCTGACCACGTACTACC |
| | | | TTCTATCTGATGTGACAGCTTCTGTAGCACGCCACCCTCTTC |
| | | | ACGGCCAATGTTTAGTTATTTGGCCGTGACGAGGGTGGCTG |
| | | | TACTGCTAGCTGTAGAACTCCAGCTTCGGCCTGTAACTTATG |
| | | | ATAGCAATGTCAGCAGTGCCTCGCCACCCTCTTCACGGCCA |
| | | | ATTAAGATTCTAAAATTATTGGGCCGTGAACAGGGTGGCTA |
| | | | AGTAAGGTTGACCATACTCTACAGTTGTTGATTTCGTGGCTA |
| | | | CAGAGTTTCCTTAGCAGAGCTGCCTGGAGATTGATGCAGCA |
| | | | ATTGTCTAAACTATATTGCTGCATCAATCTCCAGTTAGCTAC |
| | | | TGCTAGGCAATCCTTCCCTCGATAAGATGCAGCGGCGGCTC |
| | | | CTCTCCCCATGGCCCTGGCGGCTGATGAAGCTCTATATCTGG |
| | | | GCTCAGAATATAGAGCTTGATCAGCCGGCAGGGACCTGGGG |
| | | | ACCCCGGCACCGGCAGGCTAGC |
| Ctf1- | SEQ ID NO: 87 | DNA | CCAAGGTATATTGCTGTTGACAGTGAGCGACGCAGGTATGT |
| Slc13a5 | | | ATCCAATACATGTGAAGCCACAGATGATGTATTGGATTCAT |
| | | | ACCTGAGCTGCCTACTGCCTCGGACTTCAAGGGGCTTGCGG |
| | | | CCGCCATCTCCATGGCTGTACCACCTTGTCGGCGAGGGAAA |
| | | | CACCGTTCATATTGTTGAATCTCATTTATGAACGGTCTTTCC |
| | | | CTCCTCTGACATTTTGGTATCTTTCATCTGACCACGTACTAC |
| | | | CTTCTATCTGATGTGACAGCTTCTGTAGCACGCCACCCTCTT |
| | | | CACGGCCAATGTTTAGTTATTTGGCCGTGACGAGGGTGGCT |
| | | | GTACTGCTAGCTGTAGAACTCCAGCTTCGGCCTGTAACTTAT |
| | | | GATAGCAATGTCAGCAGTGCCTCGCCACCCTCTTCACGGCC |
| | | | AATTAAGATTCTAAAATTATTGGGCCGTGAACAGGGTGGCT |
| | | | AAGTAAGGTTGACCATACTCTACAGTTGTTGAGACTACGCC |
| | | | TGGCTCGAGCAGGGGGCGAGGGATCGAGGGAAACACCGTT |
| | | | CATATTGGTCCCCTCCCTATGAACGGTCTTTCCCTCTGTCCTT |
| | | | CCCTCCCAATGACCGCGTCTTCGTCACAGTCAGCGGCGGCT |
| | | | CCTCTCCCCATGGCCCTGCTTGGGCTGTCCCTATCTTTCCTG |
| | | | GGCTCAGAGAAAGATAGGGTCAGCCCAACCAGGGACCTGG |
| | | | GGACCCCGGCACCGGCAGGCTAGC |
| Dgat1- | SEQ ID NO: 88 | DNA | CCAAGGTATATTGCTGTTGACAGTGAGCGACGCGGCTGATG |
| Pappa | | | AAGCTCTATATGTGAAGCCACAGATGATATAGAGCTTGATC |
| | | | AGCCGAGCTGCCTACTGCCTCGGACTTCAAGGGGCTTGCGG |
| | | | CCGCCATCTCCATGGCTGTACCACCTTGTCGGCCTGGAGATT |
| | | | GATGCAGCAATTGTTGAATCTCATTTTGCTGCATCTATCTCC |
| | | | AGCTCTGACATTTTGGTATCTTTCATCTGACCACGTACTACC |
| | | | TTCTATCTGATGTGACAGCTTCTGTAGCAGCCCTTCAAGGAT |
| | | | ATGGACTATGTTTAGTTATTAGTCCATATACTTGAAGGGAGT |
| | | | ACTGCTAGCTGTAGAACTCCAGCTTCGGCCTGTAACTTATGA |
| | | | TAGCAATGTCAGCAGTGCCTCGGATCATTGAGCGTCTCTTAT |
| | | | TAAGATTCTAAAATTATTGAGAGACGCTGAATGATCCTAAG |
| | | | TAAGGTTGACCATACTCTACAGTTGTTGATTTCGTGGCTACA |
| | | | GAGTTTCCTTAGCAGAGCTGCCTGGAGATTGATGCAGCAAT |
| | | | TGTCTAAACTATATTGCTGCATCAATCTCCAGTTAGCTACTG |
| | | | CTAGGCAATCCTTCCCTCGATAAGATGCAGCGGCGGCTCCT |
| | | | CTCCCCATGGCCCTGCCTGAGTAATGCAAGGTTATTCTGGGC |
| | | | TCAGAAATAACCTTGCTTTACTCAGCCAGGGACCTGGGGAC |
| | | | CCCGGCACCGGCAGCTAGC |
| Pcsk9- | SEQ ID NO: 89 | DNA | CCAAGGTATATTGCTGTTGACAGTGAGCGACCAGAGGCTAC |
| Rps6kb1 | | | AGATTGAACAAGTGAAGCCACAGATGTTGTTCAATCTCTAG |
| | | | CCTCTTGCTGCCTACTGCCTCGGACTTCAAGGGGCTTGCGGC |
| | | | CGCCATCTCCATGGCTGTACCACCTTGTCGGCCCTTTCATTG |
| | | | TGGACCTGATTGTTGAATCTCATTACAGGTCCACCATGAAA |
| | | | GGCTCTGACATTTTGGTATCTTTCATCTGACCACGTACTACC |
| | | | TTCTATCTGATGTGACAGCTTCTGTAGCACCACTCGAACAGC |
| | | | TACAGCTATGTTTAGTTATTAGCTGTAGCGGTTCGAGTGTGT |
| | | | ACTGCTAGCTGTAGAACTCCAGCTTCGGCCTGTAACTTATGA |
| | | | TAGCAATGTCAGCAGTGCCTGCTGATCCACTTCTCTACCAAT |
| | | | TAAGATTCTAAAATTATTGGGTAGAGAACTGGATCAGAAAG |
| | | | TAAGGTTGACCATACTCTACAGTTGTTGATTTCGTGGCTACA |
| | | | GAGTTTCCTTAGCAGAGCTGACATTGTTACACAGCCAGTATT |
| | | | GTCTAAACTATATACTGGCTGTGTAACAATGGTAGCTACTG |
| | | | CTAGGCAATCCTTCCCTCGATAAGATGCAGCGGCGGCTCCT |
| | | | CTCCCCATGGCCCTGGCATGGAACATTGTGAGAAATCTGGG |
| | | | CTCAGAATTTCTCACAAAGTTCCATGGCAGGGACCTGGGGA |
| | | | CCCCGGCACCGGCAGGCTAGC |
| Slc13a1- | SEQ ID NO: 90 | DNA | CCAAGGTATATTGCTGTTGACAGTGAGCGACCTTACCTGAA |
| ikbkb | | | TCAGACAAGAAGTGAAGCCACAGATGTTCTTGTCTGAATCA |
| | | | GGTAATGCTGCCTACTGCCTCGGACTTCAAGGGGCTTGCGG |
| | | | CCGCCATCTCCATGGCTGTACCACCTTGTCGGGCATCTAGTA |
| | | | GAGCGGATGATTGTTGAATCTCATTTCATCCGCTCAACTAGA |
| | | | TGGTCTGACATTTTGGTATCTTTCATCTGACCACGTACTACC |
| | | | TTCTATCTGATGTGACAGCTTCTGTAGCACCTGGGCTTCTAA |
| | | | CTTTGTTATGTTTAGTTATTAACAAAGTTCGAAGCCCAGTGT |
| | | | ACTGCTAGCTGTAGAACTCCAGCTTCGGCCTGTAACTTATGA |
| | | | TAGCAATGTCAGCAGTGCCTCCGCATCTTAAACTTGGAGTTT |
| | | | TAAGATTCTAAAATTATACCTCCAAGTTAAAGATGCGAAAG |
| | | | TAAGGTTGACCATACTCTACAGTTGTTGATTTCGTGGCTACA |
| | | | GAGTTTCCTTAGCAGAGCTGGCCTACATCCTCTTTGTTATTT |
| | | | GTCTAAACTATAATAACAAAGACGATGTAGGATAGCTACTG |
| | | | CTAGGCAATCCTTCCCTCGATAAGTATGGGGCCTGGCTCGA |
| | | | GCAGGGGGCGAGGGATGCATCTAGTAGAGCGGATGATTGG |
| | | | TCCCCTCCCTCATCCGCTCAACTAGATGAGTCCTTCCCTCCC |
| | | | AATGACCGCGTCTTGGCTAGC |
| Slc13a1- | SEQ ID NO: 91 | DNA | CCAAGGTATATTGCTGTTGACAGTGAGCGACCCTGGGCTTC |
| mTOR | | | TAACTTTGTTAGTGAAGCCACAGATGTAACAAAGTTACAAG |
| | | | CCCAGTGCTGCCTACTGCCTCGGACTTCAAGGGGCTTGCGG |
| | | | CCGCCATCTCCATGGCTGTACCACCTTGTCGGCCGCATCTTA |
| | | | AACTTGGAGTTTGTTGAATCTCATTACTCCAAGTTAAAGATG |
| | | | CGCTCTGACATTTTGGTATCTTTCATCTGACCACGTACTACC |
| | | | TTCTATCTGATGTGACAGCTTCTGTAGCACCTGGGCTTCTAA |
| | | | CTTTGTTATGTTTAGTTATTAACAAAGTTCGAAGCCCAGTGT |
| | | | ACTGCTAGCTGTAGAACTCCAGCTTCGGCCTTCACGTGGCTA |
| | | | CAGAGTTTCCTTAGCAGAGCTGCTGGATGCAGTGGCGACAT |
| | | | TTGTCTAAACTATAAATGTCGCCACTGCATCCATTAGCTACT |
| | | | GCTAGGCAATCCTTCCCTCGATAAATGGATGGCCTGGCTCG |
| | | | AGCAGGGGGCGAGGGATCAGACAGTTGGACTTGTTAAATG |
| | | | GTCCCCTCCCTTAACAAGTCGAACTGTCTTGTCCTTCCCTCC |
| | | | CAATGACCGCGTCTTCGTCACAGTCAGCGGCGGCTCCTCTC |
| | | | CCCATGGCCCTGATGCTGTCCCTGGTCCTTATGCTGGGCTCA |
| | | | GACATAAGGACCACGGACAGCAACAGGGACCTGGGGACCC |
| | | | CGGCACCGGCAGGCTAGC |
| Slc13a5- | SEQ ID NO: 92 | DNA | CCGGCCAAGGTATATTGCTGTTGACAGTGAGCGACGCGGCT |
| Pappa | | | GATGAAGCTCTATATGTGAAGCCACAGATGATATAGAGCTT |
| | | | GATCAGCCGAGCTGCCTACTGCCTCGGACTTCAAGGGGCTT |
| | | | GCGGCCGCCATCTCCATGGCTGTACCACCTTGTCGGCCTGG |
| | | | AGATTGATGCAGCAATTGTTGAATCTCATTTTGCTGCATCTA |
| | | | TCTCCAGCTCTGACATTTTGGTATCTTTCATCTGACCACGTA |
| | | | CTACCTTCTATCTGATGTGACAGCTTCTGTAGCAGCAGGTAT |
| | | | GTATCCAATACATTGTTTAGTTATATGTATTGGAGACATACC |
| | | | TGAGTACTGCTAGCTGTAGAACTCCAGCTTCGGCCTGTAACT |
| | | | TATGATAGCAATGTCAGCAGTGCCTCGAGGGAAACACCGTT |
| | | | CATATTTAAGATTCTAAAATTATAGATGAACGGTCTTTCCCT |
| | | | CAAAGTAAGGTTGACCATACTCTACAGTTGTTGATTTCGTGG |
| | | | CTACAGAGTTTCCTTAGCAGAGCTGCCTGGAGATTGATGCA |
| | | | GCAATTGTCTAAACTATATTGCTGCATCAATCTCCAGTTAGC |
| | | | TACTGCTAGGCAATCCTTCCCTCGATAAGTATGGGGCCTGG |
| | | | CTCGAGCAGGGGGCGAGGGATCGAGGGAAACACCGTTCAT |
| | | | ATTGGTCCCCTCCCTATGAACGGTCTTTCCCTCTGTCCTTCC |
| | | | CTCCCAATGACCGCGTCTTGGCTAGC |
| Slc13a5- | SEQ ID NO: 93 | DNA | GGCCAAGGTATATTGCTGTTGACAGTGAGCGACGCCGTGAA |
| PDE4b- | | | GCAAATAGCAGTTGTGAAGCCACAGATGAACTGCTATTTCC |
| mTOR | | | TTCACGGAGCTGCCTACTGCCTCGGACTTCAAGGGGCTTGC |
| | | | GGCCGCCATCTCCATGGCTGTACCACCTTGTCGGCAACCGG |
| | | | ATGCTCAAGATATTTGTTGAATCTCATTATATCTTGAGGATC |
| | | | CGGTTCTCTGACATTTTGGTATCTTTCATCTGACCACGTACT |
| | | | ACCTTCTATCTGATGTGACAGCTTCTGTAGCAGCAGGTATGT |
| | | | ATCCAATACATTGTTTAGTTATATGTATTGGAGACATACCTG |
| | | | AGTACTGCTAGCTGTAGAACTCCAGCTTCGGCCTGTAACTTA |
| | | | TGATAGCAATGTCAGCAGTGCCTCGAGGGAAACACCGTTCA |
| | | | TATTTAAGATTCTAAAATATAGATGAACGGTCTTTCCCTCA |
| | | | AAGTAAGGTTGACCATACTCTACAGTTGTTGATTTCGTGGCT |
| | | | ACAGAGTTTCCTTAGCAGAGCTGCTGGATGCAGTGGCGACA |
| | | | TTTTGTCTAAACTATAAATGTCGCCACTGCATCCATTAGCTA |
| | | | CTGCTAGGCAATCCTTCCCTCGATAAGTATGGGGCCTGGCTC |
| | | | GAGCAGGGGGCGAGGGATCAGACAGTTGGACTTGTTAAAT |
| | | | GGTCCCCTCCCTCATCCGCTCAACTAGATGAGTCCTTCCCTC |
| | | | CCAATGACCGCGTCTTCGTCGTTTCAGCGGCGGCTCCTCTCC |
| | | | CCATGGCCCTGTCTGAGACTGACACCAGGTATCTGGGCTCA |
| | | | GACATAAGGACCACGGACAGCAACAGGGACCTGGGGACCC |
| | | | CGGCACCGGCAGGCTAG |
| miR16-1-5′ | SEQ ID NO: 94 | DNA | TGTCAGCAGTGCCT |
| miR16-1-3′ | SEQ ID NO: 95 | DNA | AGTAAGGTTGACCA |
| mir16-1- | SEQ ID NO: 96 | DNA | TTAAGATTCTAAAATTAT |
| stem-loop | | | |
| miR30a-5′ | SEQ ID NO: 97 | DNA | TGTTGACAGTGAGCGAC |
| miR20a-3′ | SEQ ID NO: 98 | DNA | GTACTGCTAGCTGTAG |
| mir20a-5′ | SEQ ID NO: 99 | DNA | GACAGCTTCTGTAGCA |
| mir20a- | SEQ ID NO: 100 | DNA | TGTTTAGTTAT |
| stem-loop | | | |
| mir21-3′ | SEQ ID NO: 101 | DNA | CTGACATTTTGGTATCT |
| miR21-5′ | SEQ ID NO: 102 | DNA | TGTACCACCTTGTCGG |
| mir21-stem- | SEQ ID NO: 103 | DNA | TGTTGAATCTCATT |
| loop | | | |
| mir30a- | SEQ ID NO: 104 | DNA | GTGAAGCCACAGATG |
| stem-loop | | | |
| mir122- | SEQ ID NO: 105 | DNA | TGTCTAAACTAT |
| stem-loop | | | |
| mir150-3′ | SEQ ID NO: 106 | DNA | CAGGGACCTGGGGAC |
| mir150- | SEQ ID NO: 107 | DNA | CTGGGCTCAGA |
| stem-loop | | | |
| miR-30a | SEQ ID NO: 108 | DNA | GCTGCCTACTGCCTCGG |
| miR-122 | SEQ ID NO: 109 | DNA | TTCCTTCAGCAGAGCTG |
| miR-122 | SEQ ID NO: 110 | DNA | TAGCTACTGCTAGGCA |
| miR-150 | SEQ ID NO: 111 | DNA | CTCCCCATGGCCCTG |
| miR16-1-5′ | SEQ ID NO: 112 | DNA | TGTCAGCAGTGCCT |
| shEf1a | SEQ ID NO: 117 | DNA | AGGCTCCGGTGCCCGTCAGTGGGCAGAGCGCACATCGCCCA |
| | | | CAGTCCCCGAGAAGTTGGGGGGAGGGGTCGGCAATTGAAC |
| | | | CGGTGCCTAGAGAAGGTGGCGCGGGGTAAACTGGGAAAGT |
| | | | GATGTCGTGTACTGGCTCCGCCTTTTTCCCGAGGGTGGGGG |
| | | | AGAACCGTATATAAGTGCAGTAGTCGCCGTGAACGTTCTTT |
| | | | TTCGCAACGGGTTTGCCGCCAGAACACA |
| WPRE3 | SEQ ID NO: 113 | DNA | AATCAACCTCTGGATTACAAAATTTGTGAAAGATTGACTGG |
| | | | TATTCTTAACTATGTTGCTCCTTTTACGCTATGTGGATACGC |
| | | | TGCTTTAATGCCTTTGTATCATGCTATTGCTTCCCGTATGGCT |
| | | | TTCATTTTCTCCTCCTTGTATAAATCCTGGTTAGTTCTTGCCA |
| | | | CGGCGGAACTCATCGCCGCCTGCCTTGCCCGCTGCTGGACA |
| | | | GGGGCTCGGCTGTTGGGCACTGACAATTCCGTGGTGTT |
| SV40 late | SEQ ID NO: 114 | DNA | GCTTTATTTGTGAAATTTGTGATGCTATTGCTTTATTTGTAAC |
| Poly | | | CATTATAAGCTGCAATAAACAAGTTAACAACAACAATTGCA |
| Adenylation | | | TTCATTTTATGTTTCAGGTTCAGGGGGAGATGTGGGAGGTTT |
| | | | TTTAAAGC |
| full ITR-ITR | SEQ ID NO: 115 | DNA | CCTTAATTAGGCTGCGCGCTCGCTCGCTCACTGAGGCCGCC |
| sTgfbR2-Fc | | | CGGGCAAAGCCCGGGCGTCGGGCGACCTTTGGTCGCCCGGC |
| | | | CTCAGTGAGCGAGCGAGCGCGCAGAGAGGGAGTGGCCAAC |
| | | | TCCATCACTAGGGGTTCCTTGTAGTTAATGATTAACCCGCCA |
| | | | TGCTACTTATCTACGTAGCCATGCTCTAGGAAGATCGGAATT |
| | | | CCTTGGCTCCGGTGCCCGTCAGTGGGCAGAGCGCACATCGC |
| | | | CCACAGTCCCCGAGAAGTTGTGGGGAGGGGTCGGCAATTGA |
| | | | ACCGGTGCCTAGAGAAGGTGGCGCGGGGTAAACTGGGAAA |
| | | | GTGATGTCGTGTACTGGCTCCGCCTTTTTCCCGAGGGTGGGG |
| | | | GAGAACCGTATATAAGTGCAGTAGTCGCCGTGAACGTTCTT |
| | | | TTTCGCAACGGGTTTGCCGCCAGAACACAGGTAAGTGCCGT |
| | | | GTGTGGTTCCCGCGGGCCTGGCCTCTTTACGGGTTATGGCCC |
| | | | TTGCGTGCCTTGAATTACTTCCACCTGGCTGCAGTACGTGAT |
| | | | TCTTGATCCCGAGCTTCGGGTTGGAAGTGGGTGGGAGAGTT |
| | | | CGAGGCCTTGCGCTTAAGGAGCCCCTTCGCCTCGTGCTTGA |
| | | | GTTGAGGCCTGGCCTGGGCGCTGGGGCCGCCGCGTGCGAAT |
| | | | CTGGTGGCACCTTCGCGCCTGTCTCGCTGCTTTCGATAAGTC |
| | | | TCTAGCCATTTAAAATTTTTGATGACCTGCTGCGACGCTTTT |
| | | | TTTCTGGCAAGATAGTCTTGTAAATGCGGGCCAAGATCTGC |
| | | | ACACTGGTATTTCGGTTTTTGGGGCCGCGGGCGGCGACGGG |
| | | | GCCCGTGCGTCCCAGCGCACATGTTCGGCGAGGCGGGGCCT |
| | | | GCGAGCGCGGCCACCGAGAATCGGACGGGGGTAGTCTCAA |
| | | | GCTGGCCGGCCTGCTCTGGTGCCTGGCCTCGCGCCGCCGTG |
| | | | TATCGCCCCGCCCTGGGCGGCAAGGCTGGCCCGGTCGGCAC |
| | | | CAGTTGCGTGAGCGGAAAGATGGCCGCTTCCCGGCCCTGCT |
| | | | GCAGGGAGCTCAAAATGGAGGACGCGGCGCTCGGGAGAGC |
| | | | GGGCGGGTGAGTCACCCACACAAAGGAAAAGGGCCTTTCC |
| | | | GTCCTCAGCCGTCGCTTCATGTGACTCCACGGAGTACCGGG |
| | | | CGCCGTCCAGGCACCTCGATTAGTTCTCGAGCTTTTGGAGTA |
| | | | CGTCGTCTTTAGGTTGGGGGGAGGGGTTTTATGCGATGGAG |
| | | | TTTCCCCACACTGAGTGGGTGGAGACTGAAGTTAGGCCAGC |
| | | | TTGGCACTTGATGTAATTCTCCTTGGAATTTGCCCTTTTTGA |
| | | | GTTTGGATCTTGGTTCATTCTCAAGCCTCAGACAGTGGTTCA |
| | | | AAGTTTTTTTCTTCCATTTCAGGTGCGGCCTGCCACCATGGG |
| | | | TCGGGGGCTGCTCCGGGGCCTGTGGCCGCTGCATATCGTCC |
| | | | TGTGGACGCGCATCGCCAGCACGATCCCGCCGCACGTTCCC |
| | | | AAGTCGGATGTGGAAATGGAAGCCCAGAAAGATGCATCCA |
| | | | TCCACCTAAGCTGTAATAGGACCATCCATCCACTGAAACAT |
| | | | TTTAACAGTGATGTCATGGCCAGCGACAATGGCGGTGCGGT |
| | | | CAAGCTTCCACAGCTGTGCAAGTTTTGCGATGTGAGACTGT |
| | | | CCACTTGCGACAACCAGAAGTCCTGCATGAGCAACTGCAGC |
| | | | ATCACGGCCATCTGTGAGAAGCCGCATGAAGTCTGCGTGGC |
| | | | CGTGTGGAGGAAGAACGACAAGAACATTACTCTGGAGACG |
| | | | GTTTGCCACGACCCCAAGCTCACCTACCACGGCTTCACTCTG |
| | | | GAAGATGCCGCTTCTCCCAAGTGTGTCATGAAGGAAAAGAA |
| | | | AAGGGCGGGCGAGACTTTCTTCATGTGTGCCTGTAACATGG |
| | | | AAGAGTGCAACGATTACATCATCTTTTCGGAAGAATACACC |
| | | | ACCAGCAGTCCCGACCCCAGAGGGCCCACAATCAAGCCCTG |
| | | | TCCTCCATGCAAATGCCCAGCACCTAACCTCGAGGGTGGAC |
| | | | CATCCGTCTTCATCTTCCCTCCAAAGATCAAGGATGTACTCA |
| | | | TGATCTCCCTGAGCCCCATAGTCACATGTGTGGTGGTGGAT |
| | | | GTGAGCGAGGATGACCCAGATGTCCAGATCAGCTGGTTTGT |
| | | | GAACAACGTGGAAGTACACACAGCTCAGACACAAACCCAT |
| | | | AGAGAGGATTACAACAGTACTCTCCGGGTGGTCAGTGCCCT |
| | | | CCCCATCCAGCACCAGGACTGGATGAGTGGCAAGGCGTTCG |
| | | | CATGCGCGGTCAACAACAAAGACCTCCCAGCGCCCATCGAG |
| | | | AGAACCATCTCAAAACCCAAAGGGTCAGTAAGAGCTCCAC |
| | | | AGGTATATGTCTTGCCTCCACCAGAAGAAGAGATGACTAAG |
| | | | AAACAGGTCACTCTGACCTGCATGGTCACAGACTTCATGCC |
| | | | TGAAGACATTTACGTGGAGTGGACCAACAACGGGAAAACA |
| | | | GAGCTAAACTACAAGAACACTGAACCAGTCCTGGACTCTGA |
| | | | TGGTTCTTACTTCATGTACAGCAAGCTGAGAGTGGAAAAGA |
| | | | AGAACTGGGTGGAAAGAAATAGCTACTCCTGTTCAGTGGTC |
| | | | CACGAGGGTCTGCACAATCACCACACGACTAAGAGCTTCTC |
| | | | CCGGACTCCGGGTAAATGAGCTAGCAATCAACCTCTGGATT |
| | | | ACAAAATTTGTGAAAGATTGACTGGTATTCTTAACTATGTTG |
| | | | CTCCTTTTACGCTATGTGGATACGCTGCTTTAATGCCTTTGT |
| | | | ATCATGCTATTGCTTCCCGTATGGCTTTCATTTTCTCCTCCTT |
| | | | GTATAAATCCTGGTTAGTTCTTGCCACGGCGGAACTCATCGC |
| | | | CGCCTGCCTTGCCCGCTGCTGGACAGGGGCTCGGCTGTTGG |
| | | | GCACTGACAATTCCGTGGTGTTTATTTGTGAAATTTGTGATG |
| | | | CTATTGCTTTATTTGTAACCATTCTAGCTTTATTTGTGAAATT |
| | | | TGTGATGCTATTGCTTTATTTGTAACCATTATAAGCTGCAAT |
| | | | AAACAAGTTAACAACAACAATTGCATTCATTTTATGTTTCAG |
| | | | GTTCAGGGGGAGATGTGGGAGGTTTTTTAAAGCGGGGGATC |
| | | | CAAATTCCCGATAAGGATCTTCCTAGAGCATGGCTACGTAG |
| | | | ATAAGTAGCATGGCGGGTTAATCATTAACTACAAGGAACCC |
| | | | CTAGTGATGGAGTTGGCCACTCCCTCTCTGCGCGCTCGCTCG |
| | | | CTCACTGAGGCCGGGCGACCAAAGGTCGCCCGACGCCCGG |
| | | | GCTTTGCCCGGGCGGCCTCAGTGAGCGAGCGAGCGCGCAGC |
| | | | CTTAATTAA |
| full ITR-ITR | SEQ ID NO: 116 | DNA | CCTTAATTAGGCTGCGCGCTCGCTCGCTCACTGAGGCCGCC |
| Nrf2 | | | CGGGCAAAGCCCGGGCGTCGGGCGACCTTTGGTCGCCCGGC |
| | | | CTCAGTGAGCGAGCGAGCGCGCAGAGAGGGAGTGGCCAAC |
| | | | TCCATCACTAGGGGTTCCTTGTAGTTAATGATTAACCCGCCA |
| | | | TGCTACTTATCTACGTAGCCATGCTCTAGGAAGATCGGAATT |
| | | | CCTTGGCTCCGGTGCCCGTCAGTGGGCAGAGCGCACATCGC |
| | | | CCACAGTCCCCGAGAAGTTGTGGGGAGGGGTCGGCAATTGA |
| | | | ACCGGTGCCTAGAGAAGGTGGCGCGGGGTAAACTGGGAAA |
| | | | GTGATGTCGTGTACTGGCTCCGCCTTTTTCCCGAGGGTGGGG |
| | | | GAGAACCGTATATAAGTGCAGTAGTCGCCGTGAACGTTCTT |
| | | | TTTCGCAACGGGTTTGCCGCCAGAACACAGGTAAGTGCCGT |
| | | | GTGTGGTTCCCGCGGGCCTGGCCTCTTTACGGGTTATGGCCC |
| | | | TTGCGTGCCTTGAATTACTTCCACCTGGCTGCAGTACGTGAT |
| | | | TCTTGATCCCGAGCTTCGGGTTGGAAGTGGGTGGGAGAGTT |
| | | | CGAGGCCTTGCGCTTAAGGAGCCCCTTCGCCTCGTGCTTGA |
| | | | GTTGAGGCCTGGCCTGGGCGCTGGGGCCGCCGCGTGCGAAT |
| | | | CTGGTGGCACCTTCGCGCCTGTCTCGCTGCTTTCGATAAGTC |
| | | | TCTAGCCATTTAAAATTTTTGATGACCTGCTGCGACGCTTTT |
| | | | TTTCTGGCAAGATAGTCTTGTAAATGCGGGCCAAGATCTGC |
| | | | ACACTGGTATTTCGGTTTTTGGGGCCGCGGGCGGCGACGGG |
| | | | GCCCGTGCGTCCCAGCGCACATGTTCGGCGAGGCGGGGCCT |
| | | | GCGAGCGCGGCCACCGAGAATCGGACGGGGGTAGTCTCAA |
| | | | GCTGGCCGGCCTGCTCTGGTGCCTGGCCTCGCGCCGCCGTG |
| | | | TATCGCCCCGCCCTGGGCGGCAAGGCTGGCCCGGTCGGCAC |
| | | | CAGTTGCGTGAGCGGAAAGATGGCCGCTTCCCGGCCCTGCT |
| | | | GCAGGGAGCTCAAAATGGAGGACGCGGCGCTCGGGAGAGC |
| | | | GGGCGGGTGAGTCACCCACACAAAGGAAAAGGGCCTTTCC |
| | | | GTCCTCAGCCGTCGCTTCATGTGACTCCACGGAGTACCGGG |
| | | | CGCCGTCCAGGCACCTCGATTAGTTCTCGAGCTTTTGGAGTA |
| | | | CGTCGTCTTTAGGTTGGGGGGAGGGGTTTTATGCGATGGAG |
| | | | TTTCCCCACACTGAGTGGGTGGAGACTGAAGTTAGGCCAGC |
| | | | TTGGCACTTGATGTAATTCTCCTTGGAATTTGCCCTTTTTGA |
| | | | GTTTGGATCTTGGTTCATTCTCAAGCCTCAGACAGTGGTTCA |
| | | | AAGTTTTTTTCTTCCATTTCAGGTGCGGCCGCTCCGCCACCA |
| | | | TGATGGACTTGGAGTTGCCACCGCCAGGACTACAGTCCCAG |
| | | | CAGGACATGGATTTGATTGACATCCTTTGGAGGCAAGACAT |
| | | | AGATCTTGGAGTAAGTCGAGAAGTGTTTGACTTTAGTCAGC |
| | | | GACAGAAGGACTATGAGTTGGAAAAACAGAAAAAACTCGA |
| | | | AAAGGAAAGACAAGAGCAACTCCAGAAGGAACAGGAGAA |
| | | | GGCCTTTTTCGCTCAGTTTCAACTGGATGAAGAAACAGGAG |
| | | | AATTCCTCCCAATTCAGCCGGCCCAGCACATCCAGACAGAC |
| | | | ACTAGTGGATCCGCCAGCTACTCCCAGGTTGCCCACATTCC |
| | | | CAAACAAGATGCCTTGTACTTTGAAGACTGTATGCAGCTTTT |
| | | | GGCAGAGACATTCCCATTTGTTGATGACCATGAGTCGCTTG |
| | | | CCCTGGATATCCCCAGCCACGCTGAAAGTTCAGTCTTCACT |
| | | | GCCCCTCATCAGGCCCAGTCCCTCAATAGCTCTCTGGAGGC |
| | | | AGCCATGACTGATTTAAGCAGCATAGAGCAGGACATGGAGC |
| | | | AAGTTTGGCAGGAGCTATTTTCCATTCCCGAATTACAGTGTC |
| | | | TTAATACCGAAAACAAGCAGCTGGCTGATACTACCGCTGTT |
| | | | CCCAGCCCAGAAGCCACACTGACAGAAATGGACAGCAATT |
| | | | ACCATTTTTACTCATCGATCTCCTCGCTGGAAAAAGAAGTG |
| | | | GGCAACTGTGGTCCACATTTCCTTCATGGTTTTGAGGATTCT |
| | | | TTCAGCAGCATCCTCTCCACTGATGATGCCAGCCAGCTGAC |
| | | | CTCCTTAGACTCAAATCCCACCTTAAACACAGATTTTGGCGA |
| | | | TGAATTTTATTCTGCTTTCATAGCAGAGCCCAGTGACGGTGG |
| | | | CAGCATGCCTTCCTCCGCTGCCATCAGTCAGTCACTCTCTGA |
| | | | ACTCCTGGACGGGACTATTGAAGGCTGTGACCTGTCACTGT |
| | | | GTAAAGCTTTCAACCCGAAGCACGCTGAAGGCACAATGGAA |
| | | | TTCAATGACTCTGACTCTGGCATTTCACTGAACACAAGTCCC |
| | | | AGCCGAGCGTCCCCAGAGCACTCCGTGGAGTCTTCCATTTA |
| | | | CGGAGACCCACCGCCTGGGTTCAGTGACTCGGAAATGGAGG |
| | | | AGCTAGATAGTGCCCCTGGAAGTGTCAAACAGAACGGCCCT |
| | | | AAAGCACAGCCAGCACATTCTCCTGGAGACACAGTACAGCC |
| | | | TCTGTCACCAGCTCAAGGGCACAGTGCTCCTATGCGTGAAT |
| | | | CCCAATGTGAAAATACAACAAAAAAAGAAGTTCCCGTGAGT |
| | | | CCTGGTCATCAAAAAGCCCCATTCACAAAAGACAAACATTC |
| | | | AAGCCGCTTAGAGGCTCATCTCACACGAGATGAGCTTAGGG |
| | | | CAAAAGCTCTCCATATTCCATTCCCTGTCGAAAAAATCATTA |
| | | | ACCTCCCTGTTGATGACTTCAATGAAATGATGTCCAAGGAG |
| | | | CAATTCAATGAAGCTCAGCTCGCATTGATCCGAGATATACG |
| | | | CAGGAGAGGTAAGAATAAAGTCGCCGCCCAGAACTGTAGG |
| | | | AAAAGGAAGCTGGAGAACATTGTCGAGCTGGAGCAAGACT |
| | | | TGGGCCACTTAAAAGACGAGAGAGAAAAACTACTCAGAGA |
| | | | AAAGGGAGAAAACGACAGAAACCTCCATCTACTGAAAAGG |
| | | | CGGCTCAGCACCTTGTATCTTGAAGTCTTCAGCATGTTACGT |
| | | | GATGAGGATGGAAAGCCTTACTCTCCCAGTGAATACTCTCT |
| | | | GCAGCAAACCAGAGATGGCAATGTGTTCCTTGTTCCCAAAA |
| | | | GCAAGAAGCCAGATACAAAGAAAAACTAGAGCGGGCTAGC |
| | | | AATCAACCTCTGGATTACAAAATTTGTGAAAGATTGACTGG |
| | | | TATTCTTAACTATGTTGCTCCTTTTACGCTATGTGGATACGC |
| | | | TGCTTTAATGCCTTTGTATCATGCTATTGCTTCCCGTATGGCT |
| | | | TTCATTTTCTCCTCCTTGTATAAATCCTGGTTAGTTCTTGCCA |
| | | | CGGCGGAACTCATCGCCGCCTGCCTTGCCCGCTGCTGGACA |
| | | | GGGGCTCGGCTGTTGGGCACTGACAATTCCGTGGTGTTTATT |
| | | | TGTGAAATTTGTGATGCTATTGCTTTATTTGTAACCATTCTA |
| | | | GCTTTATTTGTGAAATTTGTGATGCTATTGCTTTATTTGTAAC |
| | | | CATTATAAGCTGCAATAAACAAGTTAACAACAACAATTGCA |
| | | | TTCATTTTATGTTTCAGGTTCAGGGGGAGATGTGGGAGGTTT |
| | | | TTTAAAGCGGGGGATCCAAATTCCCGATAAGGATCTTCCTA |
| | | | GAGCATGGCTACGTAGATAAGTAGCATGGCGGGTTAATCAT |
| | | | TAACTACAAGGAACCCCTAGTGATGGAGTTGGCCACTCCCT |
| | | | CTCTGCGCGCTCGCTCGCTCACTGAGGCCGGGCGACCAAAG |
| | | | GTCGCCCGACGCCCGGGCTTTGCCCGGGCGGCCTCAGTGAG |
| | | | CGAGCGAGCGCGCAGCCTTAATTAA |

1.-185. (canceled)

186. A composition comprising:
(1) a viral vector comprising a first nucleic acid sequence encoding a soluble Transforming Growth Factor Beta Receptor II (sTGFβ-R2) polypeptide and a second nucleic acid sequence encoding a Fibroblast growth factor 21 (FGF21) polypeptide; or
(2) a first viral vector comprising a first nucleic acid sequence encoding an sTGFβR2 polypeptide and a second viral vector comprising a second nucleic acid sequence encoding an FGF21 polypeptide.

187. The composition of claim 186, wherein the sTGFβR2 polypeptide has at least 95% sequence identity to amino acids 33-159 of the amino acid sequence set forth in SEQ ID NO: 8.

188. The composition of claim 187, wherein the sTGFβR2 polypeptide comprises amino acids 33-159 of the amino acid sequence set forth in SEQ ID NO: 8.

189. The composition of claim 186, wherein the first nucleic acid sequence encoding the sTGFβR2 polypeptide has at least 85% sequence identity to the nucleic acid sequence set forth in SEQ ID NO: 5.

190. The composition of claim 186, wherein the first nucleic acid sequence encoding the sTGFβR2 polypeptide has at least 90% sequence identity to nucleotides 70 to 471 of the nucleic acid sequence set forth in SEQ ID NO: 5.

191. The composition of claim 186, wherein the sTGFβR2 polypeptide or the FGF21 polypeptide is a fusion polypeptide comprising an Ig Fc domain, wherein the Ig Fc domain is selected from a human, a canine, a feline, a bovine, an ovine, a caprine, an equine, a murine, or a porcine Fc domain.

192. The composition of claim 191, wherein the Fc domain subtype comprises IgG1, IgG2a, IgG2b, IgG3, or IgG4.

193. The composition of claim 192, wherein the Ig Fc domain has at least 90% sequence identity to the amino acid sequence set forth in SEQ ID NO: 11 or 13.

194. The composition of claim 186, wherein the first nucleic acid sequence and/or second nucleic acid is operably linked to a 3′ untranslated region for RNA stability and expression in mammalian cells.

195. The composition of claim 194, wherein the 3′ untranslated region comprises a woodchuck hepatitis posttranscriptional regulatory element (WPRE), a WPRE3, an SV40 late poly adenylation signal, an HBG poly adenylation signal, a Rabbit beta globin poly A, Bovine bgpA, an ETC poly adenylation signal, or a hybrid thereof.

196. The composition of claim 195, wherein the SV40 late poly adenylation signal comprises a truncated SEQ ID NO:114.

197. The composition of claim 195, wherein the WPRE comprises a truncated version of the WPRE.

198. The composition of claim 186, wherein the viral vector further comprises a promoter; or the first viral vector and the second viral vector each comprise a promoter.

199. The composition of claim 198, wherein the first nucleic acid sequence or the second nucleic acid sequence is operatively linked to the promoter.

200. The composition of claim 198, wherein the promoter is a constitutive promoter or an inducible promoter.

201. The composition of claim 198, wherein the promoter comprises an heF1a promoter, CAGGS (cytomegalovirus, chicken beta-actin intron, splice acceptor of the rabbit beta-globin gene), CMV, shEf1a (truncated hEf1a), an AAT promoter, a thyroid hormone-binding globulin promoter, an albumin promoter, a thyroxin-binding globulin (TBG) promoter, a hepatic control region (HCR)-ApoCII hybrid promoter, CASI, a HCR-hAAT hybrid promoter, an AAT promoter combined with mouse albumin gene enhancer (Ealb) element, or an apolipoprotein E promoter.

202. The composition of claim 198, wherein the promoter comprises:
(i) a liver tissue specific promoter for expression of the sTGFβR2 polypeptide or the FGF21 polypeptide in liver cells,
(ii) a heart tissue specific promoter for expression of the sTGFβR2 polypeptide or the FGF21 polypeptide in heart cells, or
(iii) a muscle tissue specific promoter for expression of the sTGFβR2 polypeptide or the FGF21 polypeptide in muscle cells.

203. The composition of claim 186, wherein the viral vector further comprises a polycistronic element.

204. The composition of claim 203, wherein the polycistronic element is an internal ribosome entry site (IRES) or a 2A sequence.

205. The composition of claim 203, wherein the first nucleic acid sequence and second nucleic acid sequence are operably linked via the polycistronic element.

206. The composition of any one of claim 203, wherein the first nucleic acid sequence and second nucleic acid sequence are operably linked via the polycistronic element for expression of the sTGFβR2 polypeptide and the FGF21 polypeptide from a polycistronic transcript.

207. The composition of claim 186, wherein the viral vector comprises:
(i) an apolipoprotein E promoter;
(ii) the first nucleic acid sequence and/or the second nucleic acid sequence;
(iii) a WPRE; and
(iv) an SV40 late polyadenylation signal.

208. The composition of claim 186, wherein the viral vector is an adeno-associated virus (AAV) vector.

209. The composition of claim 208, wherein the AAV vector is derived from an AAV serotype selected from AAV1, AAV2, AAV3, AAV4, AAV5, AAV6, AAV7, AAV8, AAV9, AAV10, AAV11, AAV12, AAV2.5, or AAVrh10.XX viral vectors, where XX represents a variant.

210. The composition of claim 186, wherein the composition is a pharmaceutical composition.

211. The composition of claim 210, further comprising a pharmaceutically acceptable carrier.

212. A method of treating a subject having an age-related disorder, comprising administering to the subject the composition of claim 186.

213. A method of reducing fibrotic tissue development in a subject, comprising administering to the subject the composition of claim 186.

214. A method of treating fibrosis in heart, liver, lung, or kidney in a subject, comprising administering to the subject the composition of claim 186.

215. A method of increasing expression of an sTGFβR2 polypeptide and/or an FGF21 polypeptide in a mammalian cell, the method comprising introducing the composition of claim 186 into the mammalian cell.

216. An AAV particle comprising a viral vector comprising a first nucleic acid sequence encoding an sTGFβR2 polypeptide and a second nucleic acid sequence encoding an FGF21 polypeptide.

217. A population of AAV particles comprising:
(1) a viral vector comprising a first nucleic acid sequence encoding an sTGFβ-R2 polypeptide and a second nucleic acid sequence encoding a FGF21 polypeptide; or
(2) a first viral vector comprising a first nucleic acid sequence encoding an sTGFβR2 polypeptide and a second viral vector comprising a second nucleic acid sequence encoding an FGF21 polypeptide.

218. A kit comprising the composition of claim 186.


SEQUENCE LISTING (151 sequences)

SEQ ID NO: 1 - DNA, 58 bp, synthetic construct
  misc_feature 1..58: note = Description of Artificial Sequence: Synthetic primer
  source 1..58: mol_type = other DNA
cggaagaatacaccaccagcagtcccgaccccagagggcccacaatcaagccctgtcc

SEQ ID NO: 2 - DNA, 28 bp, synthetic construct
  misc_feature 1..28: note = Description of Artificial Sequence: Synthetic primer
  source 1..28: mol_type = other DNA
tcatttacccggagtccgggagaagctc

SEQ ID NO: 3 - DNA, 552 bp, Homo sapiens
  source 1..552: mol_type = other DNA
atgggtcgggggctgctcaggggcctgtggccgctgcacatcgtcctgtggacgcgtatcgccagcacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgac

SEQ ID NO: 4 - DNA, 498 bp, Canis sp.
  source 1..498: mol_type = other DNA
atgcacagtcaagggcggggttgcaacaacacaaaacaaaacaaaacttccggacttcgacctgcagctgagaagaacatctcgcaaagcggcgttaataatgacatgatggtcactgacagcaatggtgtcatcaaatttccacaattgtgtaaattttgtgatgtgagatcttccacctgtgacaaccagaaatcttgcatgagcaactgcagcattacatccatctgtgagaagccacatgaagtctgtctggctgtctggagaaagaatgatgagaacataacactagagactctctgccatgaccccaaggatacctaccatggaattgttctcgaagatgctgcctcttcgaagtgcattatgaaagaaaagaaggtgctgggggagactttctttatgtgttcctgtagctccgacgagtgcaacgactacatcatcttctctgaagaatatgccaccaacaaccctgacttgttgttagtcatattccaa

SEQ ID NO: 5 - DNA, 471 bp, unidentified
  misc_feature 1..471: note = Description of Unknown: sTGFBr2 with mouse/human secretion signal sequence
  source 1..471: mol_type = other DNA
atgggtcgggggctgctccggggcctgtggccgctgcatatcgtcctgtggacgcgcatcgccagcacgaataatgacatgatggtcactgacagcaatggtgtcatcaaatttccacaattgtgtaaattttgtgatgtgagatcttccacctgtgacaaccagaaatcttgcatgagcaactgcagcattacatccatctgtgagaagccacatgaagtctgtctggctgtctggagaaagaatgatgagaacataacactagagactctctgccatgaccccaaggatacctaccatggaattgttctcgaagatgctgcctcttcgaagtgcattatgaaagaaaagaaggtgctgggggagactttctttatgtgttcctgtagctccgacgagtgcaacgactacatcatcttctctgaagaatatgccaccaacaaccctgacttgttgttagtcatattccaa

SEQ ID NO: 6 - DNA, 552 bp, Mus musculus
  source 1..552: mol_type = other DNA
atgggtcgggggctgctccggggcctgtggccgctgcatatcgtcctgtggacgcgcatcgccagcacgatcccgccgcacgttcccaagtcggatgtggaaatggaagcccagaaagatgcatccatccacctaagctgtaataggaccatccatccactgaaacattttaacagtgatgtcatggccagcgacaatggcggtgcggtcaagcttccacagctgtgcaagttttgcgatgtgagactgtccacttgcgacaaccagaagtcctgcatgagcaactgcagcatcacggccatctgtgagaagccgcatgaagtctgcgtggccgtgtggaggaagaacgacaagaacattactctggagacggtttgccacgaccccaagctcacctaccacggcttcactctggaagatgccgcttctcccaagtgtgtcatgaaggaaaagaaaagggcgggcgagactttcttcatgtgtgcctgtaacatggaagagtgcaacgattacatcatcttttcggaagaatacaccaccagcagtcccgac

SEQ ID NO: 7 - protein, 184 aa, Homo sapiens
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 8 - protein, 159 aa, Canis sp.
MHSQGRGCNNTKQNKTSGLRPAAEKNISQSGVNNDMMVTDSNGVIKFPQLCKFCDVRSSTCDNQKSCMSNCSITSICEKPHEVCLAVWRKNDENITLETLCHDPKDTYHGIVLEDAASSKCIMKEKKVLGETFFMCSCSSDECNDYIIFSEEYATNNPD

SEQ ID NO: 9 - protein, 184 aa, Mus musculus
MGRGLLRGLWPLHIVLWTRIASTIPPHVPKSDVEMEAQKDASIHLSCNRTIHPLKHFNSDVMASDNGGAVKLPQLCKFCDVRLSTCDNQKSCMSNCSITAICEKPHEVCVAVWRKNDKNITLETVCHDPKLTYHGFTLEDAASPKCVMKEKKRAGETFFMCACNMEECNDYIIFSEEYTTSSPD

SEQ ID NO: 10 - DNA, 714 bp, Canis sp.
  source 1..714: mol_type = other DNA
cccaaaagagaaaatggaagagttcctcgcccacctgattgtcccaaatgcccagcccctgaaatgctgggagggccttcggtcttcatctttcccccgaaacccaaggacaccctcttgattgcccgaacacctgaggtcacatgtgtggtggtggatctggacccagaagaccctgaggtgcagatcagctggttcgtggacggtaagcagatgcaaacagccaagactcagcctcgtgaggagcagttcaatggcacctaccgtgtggtcagtgtcctccccattgggcaccaggactggctcaaggggaagcagttcacgtgcaaagtcaacaacaaagccctcccatccccgatcgagaggaccatctccaaggccagagggcaagcccatcagcccagtgtgtatgtcctgccgccatcccgggaggagttgagcaagaacacagtcagcttgacatgcctgatcaaagacttcttcccacctgacattgatgtggagtggcagagcaatggacagcaggagcctgagagcaagtaccgcacgaccccgccccagctggacgaggacgggtcctacttcctgtacagcaagctctctgtggacaagagccgctggcagcggggagacaccttcatatgtgcggtgatgcatgaagctctacacaaccactacacacaggaatccctctcccattctccgggtaaatga

SEQ ID NO: 11 - protein, 237 aa, Canis sp.
PKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 12 - DNA, 699 bp, Mus musculus
  source 1..699: mol_type = other DNA
cccagagggcccacaatcaagccctgtcctccatgcaaatgcccagcacctaacctcgagggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggcgttcgcatgcgcggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 13 - protein, 232 aa, Mus musculus
PRGPTIKPCPPCKCPAPNLEGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 14 - DNA, 1794 bp, Mus musculus
  source 1..1794: mol_type = other DNA
atgatggacttggagttgccaccgccaggactacagtcccagcaggacatggatttgattgacatcctttggaggcaagacatagatcttggagtaagtcgagaagtgtttgactttagtcagcgacagaaggactatgagttggaaaaacagaaaaaactcgaaaaggaaagacaagagcaactccagaaggaacaggagaaggcctttttcgctcagtttcaactggatgaagaaacaggagaattcctcccaattcagccggcccagcacatccagacagacactagtggatccgccagctactcccaggttgcccacattcccaaacaagatgccttgtactttgaagactgtatgcagcttttggcagagacattcccatttgttgatgaccatgagtcgcttgccctggatatccccagccacgctgaaagttcagtcttcactgcccctcatcaggcccagtccctcaatagctctctggaggcagccatgactgatttaagcagcatagagcaggacatggagcaagtttggcaggagctattttccattcccgaattacagtgtcttaataccgaaaacaagcagctggctgatactaccgctgttcccagcccagaagccacactgacagaaatggacagcaattaccatttttactcatcgatctcctcgctggaaaaagaagtgggcaactgtggtccacatttccttcatggttttgaggattctttcagcagcatcctctccactgatgatgccagccagctgacctccttagactcaaatcccaccttaaacacagattttggcgatgaattttattctgctttcatagcagagcccagtgacggtggcagcatgccttcctccgctgccatcagtcagtcactctctgaactcctggacgggactattgaaggctgtgacctgtcactgtgtaaagctttcaacccgaagcacgctgaaggcacaatggaattcaatgactctgactctggcatttcactgaacacaagtcccagccgagcgtccccagagcactccgtggagtcttccatttacggagacccaccgcctgggttcagtgactcggaaatggaggagctagatagtgcccctggaagtgtcaaacagaacggccctaaagcacagccagcacattctcctggagacacagtacagcctctgtcaccagctcaagggcacagtgctcctatgcgtgaatcccaatgtgaaaatacaacaaaaaaagaagttcccgtgagtcctggtcatcaaaaagccccattcacaaaagacaaacattcaagccgcttagaggctcatctcacacgagatgagcttagggcaaaagctctccatattccattccctgtcgaaaaaatcattaacctccctgttgatgacttcaatgaaatgatgtccaaggagcaattcaatgaagctcagctcgcattgatccgagatatacgcaggagaggtaagaataaagtcgccgcccagaactgtaggaaaaggaagctggagaacattgtcgagctggagcaagacttgggccacttaaaagacgagagagaaaaactactcagagaaaagggagaaaacgacagaaacctccatctactgaaaaggcggctcagcaccttgtatcttgaagtcttcagcatgttacgtgatgaggatggaaagccttactctcccagtgaatactctctgcagcaaaccagagatggcaatgtgttccttgttcccaaaagcaagaagccagatacaaagaaaaactag

SEQ ID NO: 15 - DNA, 732 bp, Mus musculus
  source 1..732: mol_type = other DNA
atggcgctgttccggggaatgtggagcgtgctaaaagcactggggcgcacgggggtcgagatgtgcgcgggctgcgggggtcgcatcccctcgtctatcagtcttgtctgtattccgaagtgtttttccagcatgggtagctatccaaagaaacctatgagttcataccttcgattttccacagaacagctacccaaatttaaagctaaacacccagatgcaaaactttcagaattggttaggaaaattgcagccctgtggagggagctaccagaagcagaaaaaaaggtttatgaagctgattttaaagctgagtggaaagcatacaaagaagctgtgagcaagtataaagagcagctaactccaagtcagctgatgggtatggagaaggaggcccggcagagacggttaaaaaagaaagcactggtaaagagaagagaattaattttgcttggaaaaccaaaaagacctcgttcagcatataacatttatgtatctgaaagcttccaggaggcaaaggatgattcggctcagggaaaattgaagcttgtaaatgaggcttggaaaaatctgtctcctgaggaaaagcaggcatatattcagcttgctaaagatgataggattcgttacgacaatgaaatgaagtcttgggaagagcagatggctgaagttggacgaagtgatctcatccgtcgaagtgtgaaacgatccggagacatctctgagcattaa

SEQ ID NO: 16 - DNA, 747 bp, unidentified
  misc_feature 1..747: note = Description of Unknown: Adiponectin sequence
  source 1..747: mol_type = other DNA
atgctactgttgcaagctctcctgttcctcttaatcctgcccagtcatgccgaagatgacgttactacaactgaagagctagctcctgctttggtccctccacccaagggaacttgtgcaggttggatggcaggcatcccaggacatcctggccacaatggcacaccaggccgtgatggcagagatggcactcctggagagaagggagagaaaggagatgcaggtcttcttggtcctaagggtgagacaggagatgttggaatgacaggagctgaagggccacggggcttccccggaacccctggcaggaaaggagagcctggagaagccgcttatgtgtatcgctcagcgttcagtgtggggctggagacccgcgtcactgttcccaatgtacccattcgctttactaagatcttctacaaccaacagaatcattatgacggcagcactggcaagttctactgcaacattccgggactctactacttctcttaccacatcacggtgtacatgaaagatgtgaaggtgagcctcttcaagaaggacaaggccgttctcttcacctacgaccagtatcaggaaaagaatgtggaccaggcctctggctctgtgctcctccatctggaggtgggagaccaagtctggctccaggtgtatggggatggggaccacaatggactctatgcagataacgtcaacgactctacatttactggctttcttctctaccatgataccaactgataa

SEQ ID NO: 17 - DNA, 1174 bp, Homo sapiens
  source 1..1174: mol_type = other DNA
ttggctccggtgcccgtcagtgggcagagcgcacatcgcccacagtccccgagaagttgtggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggt

SEQ ID NO: 18 - DNA, 231 bp, Homo sapiens
  source 1..231: mol_type = other DNA
aggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacaca

SEQ ID NO: 19 - DNA, 544 bp, unidentified
  misc_feature 1..544: note = Description of Unknown: rheF1a promoter sequence
  source 1..544: mol_type = other DNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 20 - DNA, 66 bp, Porcine teschovirus-1
  source 1..66: mol_type = other DNA
ggatctggcgccaccaacttctctctgctgaagcaggccggcgacgtggaggagaacccaggccca

SEQ ID NO: 21 - DNA, 54 bp, Thosea asigna virus
  source 1..54: mol_type = other DNA
gagggccgcggcagcctgctgacctgcggcgacgtggaggaaaaccccggcccc

SEQ ID NO: 22 - DNA, 581 bp, synthetic construct
  misc_feature 1..581: note = Description of Artificial Sequence: Synthetic polynucleotide
  source 1..581: mol_type = other DNA
gcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagccacc

SEQ ID NO: 23 - DNA, 130 bp, Adeno-associated virus 2
  source 1..130: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 24 - DNA, 141 bp, Adeno-associated virus 2
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 25 - DNA, 2214 bp, Adeno-associated virus 8
  source 1..2214: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctg

SEQ ID NO: 26 - DNA, 2229 bp, Adeno-associated virus
  source 1..2229: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctctagaactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggtaccggcagagacaacgtggatgcggacaaagtcatgataaccaacgaagaagaaattaaaactactaacccggtagcaacggagtcctatggacaagtggccacaaaccaccagagtgcccaaactttggcggtgccttttaaggcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctg

SEQ ID NO: 27 - DNA, 592 bp, Woodchuck hepatitis virus
  source 1..592: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 28 - DNA, 248 bp, Woodchuck hepatitis virus
  source 1..248: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtt

SEQ ID NO: 29 - DNA, 134 bp, Simian virus 40
  source 1..134: mol_type = other DNA
gctttatttgtgaaatttgtgatgctattgctttatttgtaaccattataagctgcaataaacaagttaacaacaacaattgcattcattttatgtttcaggttcagggggagatgtgggaggttttttaaagc

SEQ ID NO: 30 - DNA, 510 bp, Bos sp.
  source 1..510: mol_type = other DNA
acgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccga

SEQ ID NO: 31 - DNA, 387 bp, Oryctolagus sp.
  source 1..387: mol_type = other DNA
tggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 32 - DNA, 479 bp, Homo sapiens
  source 1..479: mol_type = other DNA
acgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 33 - DNA, 744 bp, Mus musculus
  source 1..744: mol_type = other DNA
atgctactgttgcaagctctcctgttcctcttaatcctgcccagtcatgccgaagatgacgttactacaactgaagagctagctcctgctttggtccctccacccaagggaacttgtgcaggttggatggcaggcatcccaggacatcctggccacaatggcacaccaggccgtgatggcagagatggcactcctggagagaagggagagaaaggagatgcaggtcttcttggtcctaagggtgagacaggagatgttggaatgacaggagctgaagggccacggggcttccccggaacccctggcaggaaaggagagcctggagaagccgcttatgtgtatcgctcagcgttcagtgtggggctggagacccgcgtcactgttcccaatgtacccattcgctttactaagatcttctacaaccaacagaatcattatgacggcagcactggcaagttctactgcaacattccgggactctactacttctcttaccacatcacggtgtacatgaaagatgtgaaggtgagcctcttcaagaaggacaaggccgttctcttcacctacgaccagtatcaggaaaagaatgtggaccaggcctctggctctgtgctcctccatctggaggtgggagaccaagtctggctccaggtgtatggggatggggaccacaatggactctatgcagataacgtcaacgactctacatttactggctttcttctctaccatgataccaactga

SEQ ID NO: 34 - protein, 247 aa, Mus musculus
MLLLQALLFLLILPSHAEDDVTTTEELAPALVPPPKGTCAGWMAGIPGHPGHNGTPGRDGRDGTPGEKGEKGDAGLLGPKGETGDVGMTGAEGPRGFPGTPGRKGEPGEAAYVYRSAFSVGLETRVTVPNVPIRFTKIFYNQQNHYDGSTGKFYCNIPGLYYFSYHITVYMKDVKVSLFKKDKAVLFTYDQYQEKNVDQASGSVLLHLEVGDQVWLQVYGDGDHNGLYADNVNDSTFTGFLLYHDTN

SEQ ID NO: 35 - DNA, 1794 bp, Mus musculus; identical to SEQ ID NO: 14
  source 1..1794: mol_type = other DNA

SEQ ID NO: 36 - protein, 597 aa, Mus musculus
MMDLELPPPGLQSQQDMDLIDILWRQDIDLGVSREVFDFSQRQKDYELEKQKKLEKERQEQLQKEQEKAFFAQFQLDEETGEFLPIQPAQHIQTDTSGSASYSQVAHIPKQDALYFEDCMQLLAETFPFVDDHESLALDIPSHAESSVFTAPHQAQSLNSSLEAAMTDLSSIEQDMEQVWQELFSIPELQCLNTENKQLADTTAVPSPEATLTEMDSNYHFYSSISSLEKEVGNCGPHFLHGFEDSFSSILSTDDASQLTSLDSNPTLNTDFGDEFYSAFIAEPSDGGSMPSSAAISQSLSELLDGTIEGCDLSLCKAFNPKHAEGTMEFNDSDSGISLNTSPSRASPEHSVESSIYGDPPPGFSDSEMEELDSAPGSVKQNGPKAQPAHSPGDTVQPLSPAQGHSAPMRESQCENTTKKEVPVSPGHQKAPFTKDKHSSRLEAHLTRDELRAKALHIPFPVEKIINLPVDDFNEMMSKEQFNEAQLALIRDIRRRGKNKVAAQNCRKRKLENIVELEQDLGHLKDEREKLLREKGENDRNLHLLKRRLSTLYLEVFSMLRDEDGKPYSPSEYSLQQTRDGNVFLVPKSKKPDTKKN

SEQ ID NO: 37 - DNA, 1251 bp, unidentified
  misc_feature 1..1251: note = Description of Unknown: sTGFBRII sequence
  source 1..1251: mol_type = other DNA
atgggtcgggggctgctccggggcctgtggccgctgcatatcgtcctgtggacgcgcatcgccagcacgatcccgccgcacgttcccaagtcggatgtggaaatggaagcccagaaagatgcatccatccacctaagctgtaataggaccatccatccactgaaacattttaacagtgatgtcatggccagcgacaatggcggtgcggtcaagcttccacagctgtgcaagttttgcgatgtgagactgtccacttgcgacaaccagaagtcctgcatgagcaactgcagcatcacggccatctgtgagaagccgcatgaagtctgcgtggccgtgtggaggaagaacgacaagaacattactctggagacggtttgccacgaccccaagctcacctaccacggcttcactctggaagatgccgcttctcccaagtgtgtcatgaaggaaaagaaaagggcgggcgagactttcttcatgtgtgcctgtaacatggaagagtgcaacgattacatcatcttttcggaagaatacaccaccagcagtcccgaccccagagggcccacaatcaagccctgtcctccatgcaaatgcccagcacctaacctcgagggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggcgttcgcatgcgcggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 38 - protein, 416 aa, unidentified
  REGION 1..416: note = Description of Unknown: sTGFBRII sequence
MGRGLLRGLWPLHIVLWTRIASTIPPHVPKSDVEMEAQKDASIHLSCNRTIHPLKHFNSDVMASDNGGAVKLPQLCKFCDVRLSTCDNQKSCMSNCSITAICEKPHEVCVAVWRKNDKNITLETVCHDPKLTYHGFTLEDAASPKCVMKEKKRAGETFFMCACNMEECNDYIIFSEEYTTSSPDPRGPTIKPCPPCKCPAPNLEGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 39 - DNA, 912 bp, unidentified
  misc_feature 1..912: note = Description of Unknown: GDF15 sequence
  source 1..912: mol_type = other DNA
atggccccgcccgcgctccaggcccagcctccaggcggctctcaactgaggttcctgctgttcctgctgctgttgctgctgctgctgtcatggccatcgcagggggacgccctggcaatgcctgaacagcgaccctccggccctgagtcccaactcaacgccgacgagctacggggtcgcttccaggacctgctgagccggctgcatgccaaccagagccgagaggactcgaactcagaaccaagtcctgacccagctgtccggatactcagtccagaggtgagattggggtcccacggccagctgctactccgcgtcaaccgggcgtcgctgagtcagggtctccccgaagcctaccgcgtgcaccgagcgctgctcctgctgacgccgacggcccgcccctgggacatcactaggcccctgaagcgtgcgctcagcctccggggaccccgtgctcccgcattacgcctgcgcctgacgccgcctccggacctggctatgctgccctctggcggcacgcagctggaactgcgcttacgggtagccgccggcagggggcgccgaagcgcgcatgcgcacccaagagactcgtgcccactgggtccagggcgctgctgtcacttggagactgtgcaggcaactcttgaagacttgggctggagcgactgggtgctgtccccgcgccagctgcagctgagcatgtgcgtgggcgagtgtccccacctgtatcgctccgcgaacacgcatgcgcagatcaaagcacgcctgcatggcctgcagcctgacaaggtgcctgccccgtgctgtgtcccctccagctacaccccggtggttcttatgcacaggacagacagtggtgtgtcactgcagacttatgatgacctggtggcccggggctgccactgcgcttga

SEQ ID NO: 40 - protein, 303 aa, unidentified
  REGION 1..303: note = Description of Unknown: GDF15 sequence
MAPPALQAQPPGGSQLRFLLFLLLLLLLLSWPSQGDALAMPEQRPSGPESQLNADELRGRFQDLLSRLHANQSREDSNSEPSPDPAVRILSPEVRLGSHGQLLLRVNRASLSQGLPEAYRVHRALLLLTPTARPWDITRPLKRALSLRGPRAPALRLRLTPPPDLAMLPSGGTQLELRLRVAAGRGRRSAHAHPRDSCPLGPGRCCHLETVQATLEDLGWSDWVLSPRQLQLSMCVGECPHLYRSANTHAQIKARLHGLQPDKVPAPCCVPSSYTPVVLMHRTDSGVSLQTYDDLVARGCHCA

SEQ ID NO: 41 - DNA, 2145 bp, Homo sapiens
  source 1..2145: mol_type = other DNA
atgatgcaggaatctgcgacagagacaataagcaacagttcaatgaatcaaaatggaatgagcactctaagcagccaattagatgctggcagcagagatggaagatcaagtggtgacaccagctctgaagtaagcacagtagaactgctgcatctgcaacaacagcaggctctccaggcagcaagacaacttcttttacagcagcaaacaagtggattgaaatctcctaagagcagtgataaacagagaccactgcaggtgcctgtgtcagtggccatgatgactccccaggtgatcacccctcagcaaatgcagcagatccttcagcaacaagtcctgtctcctcagcagctacaagcccttctccaacaacagcaggctgtcatgctgcagcagcaacaactacaagagttttacaagaaacagcaagagcagttacatcttcagcttttgcagcagcagcagcaacagcagcagcagcaacaacagcagcaacaacagcagcagcaacaacaacaacaacagcagcaacaacagcagcagcagcagcaacagcagcagcagcagcaacagcatcctggaaagcaagcgaaagagcagcagcagcagcagcagcaacagcaattggcagcccagcagcttgtcttccagcagcagcttctccatatgcaacaactccagcagcagcagcatctgctcagccttcagcgtcagggactcatctccattccacctggccaggcagcacttcctgtccaatcgctgcctcaagctggcttaagtcctgctgagattcagcagttatggaaagaagtgactggagttcacagtatggaagacaatggcattaaacatggagggctagacctcactactaacaattcctcctcgactacctcctccaacacttccaaagcatcaccaccaataactcatcattccatagtgaatggacagtcttcagttctaagtgcaagacgagacagctcgtcacatgaggagactggggcctctcacactctctatggccatggagtttgcaaatggccaggctgtgaaagcatttgtgaagattttggacagtttttaaagcaccttaacaatgaacacgcattggatgaccgaagcactgctcagtgtcgagtgcaaatgcaggtggtgcaacagttagaaatacagctttctaaagaacgcgaacgtcttcaagcaatgatgacccacttgcacatgcgaccctcagagcccaaaccatctcccaaacctctaaatctggtgtctagtgtcaccatgtcgaagaatatgttggagacatccccacagagcttacctcaaacccctaccacaccaacggccccagtcaccccgattacccagggaccctcagtaatcaccccagccagtgtgcccaatgtgggagccatacgaaggcgacattcagacaaatacaacattcccatgtcatcagaaattgccccaaactatgaattttataaaaatgcagatgtcagacctccatttacttatgcaactctcataaggcaggctatcatggagtcatctgacaggcagttaacacttaatgaaatttacagctggtttacacggacatttgcttacttcaggcgtaatgcagcaacttggaagaatgcagtacgtcataatcttagcctgcacaagtgttttgttcgagtagaaaatgttaaaggagcagtatggactgtggatgaagtagaataccagaagcgaaggtcacaaaagataacaggaagtccaaccttagtaaaaaatatacctaccagtttaggctatggagcagctcttaatgccagtttgcaggctgccttggcagagagcagtttacctttgctaagtaatcctggactgataaataatgcatccagtggcctactgcaggccgtccacgaagacctcaatggttctctggatcacattgacagcaatggaaacagtagtccgggctgctcacctcagccgcacatacattcaatccacgtcaaggaagagccagtgattgcagaggatgaagactgcccaatgtccttagtgacaacagctaatcacagtccagaattagaagacgacagagagattgaagaagagcctttatctgaagatctggaatga

SEQ ID NO: 42 - protein, 714 aa, Homo sapiens
MMQESATETISNSSMNQNGMSTLSSQLDAGSRDGRSSGDTSSEVSTVELLHLQQQQALQAARQLLLQQQTSGLKSPKSSDKQRPLQVPVSVAMMTPQVITPQQMQQILQQQVLSPQQLQALLQQQQAVMLQQQQLQEFYKKQQEQLHLQLLQQQQQQQQQQQQQQQQQQQQQQQQQQQQQQQQQQQQQQQQHPGKQAKEQQQQQQQQQLAAQQLVFQQQLLHMQQLQQQQHLLSLQRQGLISIPPGQAALPVQSLPQAGLSPAEIQQLWKEVTGVHSMEDNGIKHGGLDLTTNNSSSTTSSNTSKASPPITHHSIVNGQSSVLSARRDSSSHEETGASHTLYGHGVCKWPGCESICEDFGQFLKHLNNEHALDDRSTAQCRVQMQVVQQLEIQLSKERERLQAMMTHLHMRPSEPKPSPKPLNLVSSVTMSKNMLETSPQSLPQTPTTPTAPVTPITQGPSVITPASVPNVGAIRRRHSDKYNIPMSSEIAPNYEFYKNADVRPPFTYATLIRQAIMESSDRQLTLNEIYSWFTRTFAYFRRNAATWKNAVRHNLSLHKCFVRVENVKGAVWTVDEVEYQKRRSQKITGSPTLVKNIPTSLGYGAALNASLQAALAESSLPLLSNPGLINNASSGLLQAVHEDLNGSLDHIDSNGNSSPGCSPQPHIHSIHVKEEPVIAEDEDCPMSLVTTANHSPELEDDREIEEEPLSEDLE

SEQ ID NO: 43 - DNA, 828 bp, Mus musculus
  source 1..828: mol_type = other DNA
atgacagatgacaaagatgtgcttcgagatgtgtggtttggacgaattccaacttgctttactctctatcaggatgagataactgaaagagaagcagaaccatactatttgcttttgccaagagtcagctatttgacgttggtaactgacaaagtgaaaaagcactttcagaaggttatgagacaagaagatgttagtgagatatggtttgaatatgaaggcacacccctgaaatggcattatccaattggtttactatttgatcttcttgcatcaagttcagctcttccttggaacatcacagtacatttcaagagttttccagaaaaggaccttctacactgtccatccaaggatgcggttgaggctcactttatgtcgtgtatgaaagaagctgatgctttaaagcataaaagtcaagtgatcaacgaaatgcagaaaaaagaccacaagcagctctggatgggactgcagaatgacagatttgaccagttttgggccatcaaccggaaactcatggaatatcctccagaagaaaatggatttcgttatatcccctttagaatatatcagaccacgacggagcggcctttcatccagaagctgttccggcctgtggccgcagatggacagctgcacacacttggagatctcctcagagaagtctgtccttccgcagtcgcccctgaagatggagagaagaggagccaggtgatgattcacgggatagagccaatgctggaaacccctctgcagtggctgagcgagcatctgagctacccagataactttcttcatattagcattgtcccccagccaacagattaa

SEQ ID NO: 44 - protein, 275 aa, Mus musculus
MTDDKDVLRDVWFGRIPTCFTLYQDEITEREAEPYYLLLPRVSYLTLVTDKVKKHFQKVMRQEDVSEIWFEYEGTPLKWHYPIGLLFDLLASSSALPWNITVHFKSFPEKDLLHCPSKDAVEAHFMSCMKEADALKHKSQVINEMQKKDHKQLWMGLQNDRFDQFWAINRKLMEYPPEENGFRYIPFRIYQTTTERPFIQKLFRPVAADGQLHTLGDLLREVCPSAVAPEDGEKRSQVMIHGIEPMLETPLQWLSEHLSYPDNFLHISIVPQPTD

SEQ ID NO: 45 - DNA, 3159 bp, Mus musculus
  source 1..3159: mol_type = other DNA
atggcggaggcgagtgaagccatgtgcctggagggagcagagtgggagctgagtaaagaaaacatacagcccttacggcacgggcgggtcatgtccacacttcagggagctttggcaaagcaagagtcagctggccacactgctctgcagcagcagaaacgggcatttgaatctgaaatccgcttttactctggagatgaccctctggatgtgtgggacagatatattaattggacagaacagaactaccctcaaggggggaaggagagtaacatgtcagcgttagtggagagagcgatagaagcactccaaggagagacgcgctattataatgacccccgctttctcagtctctggatcaaattgggacatttgtgcaatgaacctttggatatgtacagctatttacaaagccaaggaattggcgtttcccttgcccagttctatatttcatgggctgaagaatacgaagctagagaaaatttcaagaaagcggacataatatttcaggaagggattgaacgcaaggctgagcccctggacagactgcagtcccagcacagacagttccagtctcgagtgtcgcgacaagctttcttggcccttgggaatgaagaggaggaggctttggagccttctgaaccacagagaagctcgctagctgagctgaagagcagagggaagaagatggccagagcgcccatcagccgtgtcggaagtgctctgaaagctccaggtcagagcagaggattcctaaatgcagttccccagccagtacacggtaatcgcaggatcaccgtttttgatgaaaatgccgataccgcgtctagaccggagttatctaagcctgtagcccagccatggatggcaccccctgtgcccagggccaaagagaacgaacttcagccaggcccatggagcacagacaggccggtgggacgcaggcctcatgacaatccagcctctgtgacgtcgatacccagcgtgcttcccagctttacgccgtacgtggaagagagcgcccagcagacagtcatgacaccatgcaagattgagcctagtatcaaccatgttctcagcaccaggaagccaggaagagaagaaggagaccccttgcagagagttcagagtcatcagcaaggctgtgaggagaagaaggagaagatgatgtactgtaaggagaagatctatgccggagttggggagttctcctttgaggagatccgagctgaagtgttccgaaagaagctgaaagaacgaagggaagccgagctgttgaccagtgcaaagaagagggaggagatgcagaagcagatcgaagagatggagaggaggctgaaggcaatgcaggctgttcagcaagaaggagctggtggccagcaagaagagaagatgcctacagaggacccagccagattgcagattgcttcggggcctcaggaaatgtcgggagttcctctgtcctgttccatctgtccactaagctcgaatcctagggaaatttcacctgctgagaacattttgcaagaacagcctgattctaaaggttccagtatgcctttctccatttttgatgagtctctttcagacaaaaaagacaaaagtcctgctacaggtggtccacaggttctcaatgcccagagaagacccctttcagttctcaaaactacagaagtgggcaccacaaatgaggatgtgtctcccgatatttgtgatgaactcacagaacttgagcctctgagtgaagacgccatcatcactggtttcaggaacgtcactctctgtcccaaccctgaggacacttgtgactttgctagagcagctcgtttggcatctactcctttccatgagatactgtcctcgaagggcatcgctgctgatcccgagggactgttgcaggaagaggatctggatgggaaggccgccgaggctcatcacactgttcatcaccaggccctcatcataaagaaactgagcccaattattgaagacagccgtgaggccacccactcatctggcttctccaggtcttcttcctcagctcccagtacatcctccatcaaaggctttcagcttctggaaaagctggagctgactaatgacggggcagaaaatgctattcagtcaccctggtgttcacagtatcgcctacaactgttaaaatccctactagagataagtgcttttgcggagttttctgtggaagaccgaccgatgcctgtgctggaaatagggaaggagattgagttaggtcctgaggattacgtcatcaagcaagagcacctaacatgtgacgattacaggttattctgggtggcaccaagaagctctgcagagctaaccatgataaaggcatcatctcagcctatcccgtgggatttttatatcaacctcaagttgaaggagcgtctgaatgaggactatgaccagctttgcagctgctgtcagtaccaagatggccatgttgtttggtaccagtatataaactgctccacccttcagaatcttctccaacacagcgaatttgttactcatgaaataatagtgttgattatttacaacctcttgacaatcgtggagaagctacacagagctgaaatagtgcacggagacttgagtccacggagtctgatcctacgaaacagaatccacgacccctatgactatgtaaataaggacgatcacgctgtgaggatcatggacttctcctacagtgttgacctgagggtgcagctggatgcgtttgcctatagtggctttcggactgcacagatcctggaaggacaaaagatcctggctaactgttcttctccctaccatgtagatctgttgggtatagcagacctagcgcacttactcctgttcaaggagcacctccatgtcttctgggatggactcctctggaaacttagccagagcacctctgagctaaaagacagtgaattgtggaataaattctttgtgcggattctgaatgccagtgacaagtccacagtgtctgttctgggggagctggcagcagaaatgggtggggcttttgatgccacattccatagccacctgaacagagccctgtggaagctggggaagacaatcagcccggaagctttgctcactcagcaagacaagcagccaggcggctcccagagccctgcctaa

SEQ ID NO: 46 - protein, 1052 aa, Mus musculus
MAEASEAMCLEGAEWELSKENIQPLRHGRVMSTLQGALAKQESAGHTALQQQKRAFESEIRFYSGDDPLDVWDRYINWTEQNYPQGGKESNMSALVERAIEALQGETRYYNDPRFLSLWIKLGHLCNEPLDMYSYLQSQGIGVSLAQFYISWAEEYEARENFKKADIIFQEGIERKAEPLDRLQSQHRQFQSRVSRQAFLALGNEEEEALEPSEPQRSSLAELKSRGKKMARAPISRVGSALKAPGQSRGFLNAVPQPVHGNRRITVFDENADTASRPELSKPVAQPWMAPPVPRAKENELQPGPWSTDRPVGRRPHDNPASVTSIPSVLPSFTPYVEESAQQTVMTPCKIEPSINHVLSTRKPGREEGDPLQRVQSHQQGCEEKKEKMMYCKEKIYAGVGEFSFEEIRAEVFRKKLKERREAELLTSAKKREEMQKQIEEMERRLKAMQAVQQEGAGGQQEEKMPTEDPARLQIASGPQEMSGVPLSCSICPLSSNPREISPAENILQEQPDSKGSSMPFSIFDESLSDKKDKSPATGGPQVLNAQRRPLSVLKTTEVGTTNEDVSPDICDELTELEPLSEDAIITGFRNVTLCPNPEDTCDFARAARLASTPFHEILSSKGIAADPEGLLQEEDLDGKAAEAHHTVHHQALIIKKLSPIIEDSREATHSSGFSRSSSSAPSTSSIKGFQLLEKLELTNDGAENAIQSPWCSQYRLQLLKSLLEISAFAEFSVEDRPMPVLEIGKEIELGPEDYVIKQEHLTCDDYRLFWVAPRSSAELTMIKASSQPIPWDFYINLKLKERLNEDYDQLCSCCQYQDGHVVWYQYINCSTLQNLLQHSEFVTHEIIVLIIYNLLTIVEKLHRAEIVHGDLSPRSLILRNRIHDPYDYVNKDDHAVRIMDFSYSVDLRVQLDAFAYSGFRTAQILEGQKILANCSSPYHVDLLGIADLAHLLLFKEHLHVFWDGLLWKLSQSTSELKDSELWNKFFVRILNASDKSTVSVLGELAAEMGGAFDATFHSHLNRALWKLGKTISPEALLTQQDKQPGGSQSPA

SEQ ID NO: 47 - DNA, 1692 bp, unidentified
  misc_feature 1..1692: note = Description of Unknown: MCat sequence
  source 1..1692: mol_type = other DNA
atgtccgtcctgacgccgctgctgctgcggggcttgacaggctcggcccggcggctcccagtgccgcgcgccaagatccattcgttgggggatccaccggtcgccaccatgtcggacagtcgggacccagccagcgaccagatgaagcagtggaaggagcagcgggcctcgcagagacctgatgtcctgaccaccggaggcgggaacccaataggagataaacttaatatcatgaccgcggggtcccgagggcccctcctcgttcaggatgtggttttcactgacgagatggcacactttgacagagagcggattcctgagagagtggtacacgcaaaaggagcaggtgcttttggatactttgaggtcacccacgatatcaccagatactccaaggcaaaggtgtttgagcatattggaaagaggacccctattgccgttcgattctccacagtcgctggagagtcaggctcagctgacacagttcgtgaccctcgggggtttgcagtgaaattttacactgaagatggtaactgggatcttgtgggaaacaacacccctattttcttcatcagggatgccatattgtttccatcctttatccatagccagaagagaaacccacagactcacctgaaggatcctgacatggtctgggacttctggagtcttcgtcccgagtctctccatcaggtttctttcttgttcagtgaccgagggattcccgatggtcaccggcacatgaatggctatggatcacacaccttcaagttggttaatgcagatggagaggcagtctattgcaagttccattacaagaccgaccagggcatcaaaaacttgcctgttggagaggcaggaaggcttgctcaggaagatccggattatggcctccgagatcttttcaatgccatcgccaatggcaattacccgtcctggacgttttacatccaggtcatgacttttaaggaggcagaaactttcccatttaatccatttgatctgaccaaggtttggcctcacaaggactaccctcttataccagttggcaaactggttttaaacaaaaatccagttaattactttgctgaagttgaacagatggcttttgacccaagcaatatgccccctggcatcgagcccagccctgacaaaatgcttcagggccgcctttttgcctacccggacactcaccgccaccgcctgggacccaactatctgcagatacctgtgaactgtccctaccgcgctcgagtggccaactaccagcgtgatggccccatgtgcatgcatgacaaccagggtggtgcccccaactattaccccaacagcttcagcgcaccagagcagcagcgctcagccctggagcacagcgtccagtgcgctgtagatgtgaaacgcttcaacagtgctaatgaagacaatgtcactcaggtgcggacattctacacaaaggtgttgaacgaggaggagaggaaacgcctgtgtgagaacattgccggccacctgaaggacgctcagcttttcattcagaagaaagcggtcaagaatttcactgacgtccaccctgactatggggcccgcatccaggctcttctggacaagtacaacgctgagaagcctaagaacgcaattcacacctacacgcaggccggctctcacatggctgcgaagggaaaagctaacctgtaa

SEQ ID NO: 48 - protein, 563 aa, unidentified
  REGION 1..563: note = Description of Unknown: MCat sequence
MSVLTPLLLRGLTGSARRLPVPRAKIHSLGDPPVATMSDSRDPASDQMKQWKEQRASQRPDVLTTGGGNPIGDKLNIMTAGSRGPLLVQDVVFTDEMAHFDRERIPERVVHAKGAGAFGYFEVTHDITRYSKAKVFEHIGKRTPIAVRFSTVAGESGSADTVRDPRGFAVKFYTEDGNWDLVGNNTPIFFIRDAILFPSFIHSQKRNPQTHLKDPDMVWDFWSLRPESLHQVSFLFSDRGIPDGHRHMNGYGSHTFKLVNADGEAVYCKFHYKTDQGIKNLPVGEAGRLAQEDPDYGLRDLFNAIANGNYPSWTFYIQVMTFKEAETFPFNPFDLTKVWPHKDYPLIPVGKLVLNKNPVNYFAEVEQMAFDPSNMPPGIEPSPDKMLQGRLFAYPDTHRHRLGPNYLQIPVNCPYRARVANYQRDGPMCMHDNQGGAPNYYPNSFSAPEQQRSALEHSVQCAVDVKRFNSANEDNVTQVRTFYTKVLNEEERKRLCENIAGHLKDAQLFIQKKAVKNFTDVHPDYGARIQALLDKYNAEKPKNAIHTYTQAGSHMAAKGKANL

SEQ ID NO: 49 - DNA, 408 bp, Mus musculus
  source 1..408: mol_type = other DNA
atggtcctggacagcgtggcccgcatcgtgaaggtgcagctgcccgcctacctcaagcagctcccggtccccgacagcatcaccgggttcgcccgcctcacagtttcagactggctccgcctactgcccttcctcggggtacttgcgcttctgggctacctcgcagtgcgcccattcttcccaaagaagaagcaacagaaggatagcttgatcaatcttaagatacaaaaggaaaatcccaaggtggtgaatgagataaacattgaagatctgtgtctcaccaaagcagcttattgtaggtgctggcggtccaagacgtttcctgcctgtgatggatcccataataagcataatgaattgacaggcgataacgtgggtcctctcatcctgaagaagaaagaagtataa

SEQ ID NO: 50 - protein, 135 aa, Mus musculus
MVLDSVARIVKVQLPAYLKQLPVPDSITGFARLTVSDWLRLLPFLGVLALLGYLAVRPFFPKKKQQKDSLINLKIQKENPKVVNEINIEDLCLTKAAYCRCWRSKTFPACDGSHNKHNELTGDNVGPLILKKKEV

SEQ ID NO: 51 - DNA, 633 bp, Mus musculus
  source 1..633: mol_type = other DNA
atggaatggatgagatctagagttgggaccctgggactgtgggtccgactgctgctggctgtcttcctgctgggggtctaccaagcataccccatccctgactccagccccctcctccagtttgggggtcaagtccggcagaggtacctctacacagatgacgaccaagacactgaagcccacctggagatcagggaggatggaacagtggtaggcgcagcacaccgcagtccagaaagtctcctggagctcaaagccttgaagccaggggtcattcaaatcctgggtgtcaaagcctctaggtttctttgccaacagccagatggagctctctatggatcgcctcactttgatcctgaggcctgcagcttcagagaactgctgctggaggacggttacaatgtgtaccagtctgaagcccatggcctgcccctgcgtctgcctcagaaggactccccaaaccaggatgcaacatcctggggacctgtgcgcttcctgcccatgccaggcctgctccacgagccccaagaccaagcaggattcctgcccccagagcccccagatgtgggctcctctgaccccctgagcatggtagagcctttacagggccgaagccccagctatgcgtcctga

SEQ ID NO: 52 - protein, 210 aa, Mus musculus
MEWMRSRVGTLGLWVRLLLAVFLLGVYQAYPIPDSSPLLQFGGQVRQRYLYTDDDQDTEAHLEIREDGTVVGAAHRSPESLLELKALKPGVIQILGVKASRFLCQQPDGALYGSPHFDPEACSFRELLLEDGYNVYQSEAHGLPLRLPQKDSPNQDATSWGPVRFLPMPGLLHEPQDQAGFLPPEPPDVGSSDPLSMVEPLQGRSPSYAS

SEQ ID NO: 53 - DNA, 3045 bp, Mus musculus
  source 1..3045: mol_type = other DNA
atgctagcccgcgcccctcctcgccgcccgccgcggctggtgctgctccgtttgctgttgctgcatctgctgctgctcgccctgcgcgcccgctgcctgagcgctgagccgggtcagggcgcgcagacctgggctcgcttcgcgcgcgctcctgccccagaggccgctggcctcctccacgacaccttccccgacggtttcctctgggcggtaggcagcgccgcctatcagaccgagggcggctggcgacagcacggcaaaggcgcgtccatctgggacactttcacccatcactctggggcggccccgtccgactccccgatcgtcgtggcgccgtcgggtgccccgtcgcctcccctgtcctccactggagatgtggccagcgatagttacaacaacgtctaccgcgacacagaggggctgcgcgaactgggggtcacccactaccgcttctccatatcgtgggcgcgggtgctccccaatggcaccgcgggcactcccaaccgcgaggggctgcgctactaccggcggctgctggagcggctgcgggagctgggcgtgcagccggtggttaccctgtaccattgggacctgccacagcgcctgcaggacacctatggcggatgggccaatcgcgccctggccgaccatttcagggattatgccgagctctgcttccgccacttcggtggtcaggtcaagtactggatcaccattgacaacccctacgtggtggcctggcacgggtatgccaccgggcgcctggccccgggcgtgaggggcagctccaggctcgggtacctggttgcccacaacctacttttggctcatgccaaagtctggcatctctacaacacctctttccgccccacacagggaggccgggtgtctatcgccttaagctcccattggatcaatcctcgaagaatgactgactataatatcagagaatgccagaagtctcttgactttgtgctaggctggtttgccaaacccatatttattgatggcgactacccagagagtatgaagaacaacctctcgtctcttctgcctgattttactgaatctgagaagaggctcatcagaggaactgctgacttttttgctctctccttcggaccaaccttgagctttcagctattggaccctaacatgaagttccgccaattggagtctcccaacctgaggcagcttctgtcttggatagatctggaatataaccaccctccaatatttattgtggaaaatggctggtttgtctcgggaaccaccaaaagggatgatgccaaatatatgtattatctcaagaagttcataatggaaaccttaaaagcaatcagactggatggggtcgacgtcattgggtacaccgcgtggtcgctcatggacggtttcgagtggcataggggctacagcatccggcgaggactcttctacgttgactttctgagtcaggacaaggagctgttgccaaagtcttcggccttgttctaccaaaagctgatagaggacaatggctttcctcctttacctgaaaaccagccccttgaagggacatttccctgtgactttgcttggggagttgttgacaactacgttcaagtggacactactctctctcagtttactgacccgaatgtctatctgtgggatgtgcatcacagtaagaggcttattaaagtagacggggttgtagccaagaagagaaaaccttactgtgttgatttctctgccatccggcctcagataaccttacttcgagaaatgcgggtcacccactttcgcttctccctggactgggccctgatcttgcctctgggtaaccagacccaagtgaaccacacggttctgcacttctaccgctgcatgatcagcgagctggtgcacgccaacatcactccagtggtggccctgtggcagccagcagccccgcaccaaggcctgccacatgcccttgcaaaacatggggcctgggagaacccgcacactgctctggcgtttgcagactacgcaaacctgtgttttaaagagttgggtcactgggtcaatctctggatcaccatgaacgagccaaacacacggaacatgacctatcgtgccgggcaccacctcctgagagcccatgccttggcttggcatctgtacgatgacaagtttagggcggctcagaaaggcaaaatatccatcgccttgcaggctgactggatagaaccggcctgccctttctctcaaaatgacaaagaagtggccgagagagttttggaatttgatataggctggctggcagagcctatttttggttccggagattatccacgtgtgatgagggactggctgaaccaaaaaaacaattttcttttgccctatttcaccgaagatgaaaaaaagctagtccggggttcctttgacttcctggcggtgagtcattacaccaccattctggtagactgggaaaaggaggatccgatgaaatacaacgattacttggaggtacaggagatgactgacatcacatggctcaactctcccagtcaggtggcagtggtgccttgggggctgcgcaaagtgctcaactggctaaggttcaagtacggagacctcccgatgtatgtgacagccaatggaatcgatgatgacccccacgccgagcaagactcactgaggatctattatattaagaattatgtgaatgaggctctgaaagcctacgtgttggacgacatcaacctttgtggctactttgcgtattcacttagtgatcgctcagctcccaagtctggcttttatcgatatgctgcgaatcagtttgagcccaaaccatctatgaaacattacaggaaaattattgacagcaatggcttcctgggttctggaacactgggaaggttttgtccagaagaatacactgtgtgcaccgaatgtggattttttcaaacccggaagtctttgctggtcttcatctcgtttcttgtttttacttttattatttctcttgctctcatttttcactactccaagaaaggccagagaagttataagtaa

SEQ ID NO: 54 - protein, 1014 aa, Mus musculus
MLARAPPRRPPRLVLLRLLLLHLLLLALRARCLSAEPGQGAQTWARFARAPAPEAAGLLHDTFPDGFLWAVGSAAYQTEGGWRQHGKGASIWDTFTHHSGAAPSDSPIVVAPSGAPSPPLSSTGDVASDSYNNVYRDTEGLRELGVTHYRFSISWARVLPNGTAGTPNREGLRYYRRLLERLRELGVQPVVTLYHWDLPQRLQDTYGGWANRALADHFRDYAELCFRHFGGQVKYWITIDNPYVVAWHGYATGRLAPGVRGSSRLGYLVAHNLLLAHAKVWHLYNTSFRPTQGGRVSIALSSHWINPRRMTDYNIRECQKSLDFVLGWFAKPIFIDGDYPESMKNNLSSLLPDFTESEKRLIRGTADFFALSFGPTLSFQLLDPNMKFRQLESPNLRQLLSWIDLEYNHPPIFIVENGWFVSGTTKRDDAKYMYYLKKFIMETLKAIRLDGVDVIGYTAWSLMDGFEWHRGYSIRRGLFYVDFLSQDKELLPKSSALFYQKLIEDNGFPPLPENQPLEGTFPCDFAWGVVDNYVQVDTTLSQFTDPNVYLWDVHHSKRLIKVDGVVAKKRKPYCVDFSAIRPQITLLREMRVTHFRFSLDWALILPLGNQTQVNHTVLHFYRCMISELVHANITPVVALWQPAAPHQGLPHALAKHGAWENPHTALAFADYANLCFKELGHWVNLWITMNEPNTRNMTYRAGHHLLRAHALAWHLYDDKFRAAQKGKISIALQADWIEPACPFSQNDKEVAERVLEFDIGWLAEPIFGSGDYPRVMRDWLNQKNNFLLPYFTEDEKKLVRGSFDFLAVSHYTTILVDWEKEDPMKYNDYLEVQEMTDITWLNSPSQVAVVPWGLRKVLNWLRFKYGDLPMYVTANGIDDDPHAEQDSLRIYYIKNYVNEALKAYVLDDINLCGYFAYSLSDRSAPKSGFYRYAANQFEPKPSMKHYRKIIDSNGFLGSGTLGRFCPEEYTVCTECGFFQTRKSLLVFISFLVFTFIISLALIFHYSKKGQRSYK

SEQ ID NO: 55 - DNA, 397 bp, Mus musculus
  source 1..397: mol_type = other DNA
atggaccccaactgctcctgctccaccggcggctcctgcacttgcaccagctcctgcgcctgcaagaactgcaagtgcacctcctgcaagaagagtgagttgggacaccttgggtggcggctaaggctaggggcggggaactcctacaaaactggctctgagaaatgtcctttgcttcccggaggccattgtattgtctcggggacagaactatacagagaactatttaaaaaaaccgaggtcttctctgttggggacaggaagcagaggtcttcagccaggctgacctcttcctcctcctttctaggctgctgctcctgctgtcccgtgggctgctccaaatgtgcccagggctgtgtctgcaaaggcgccgcggacaagtgcacgtgctgtgcct

SEQ ID NO: 56 - protein, 132 aa, Mus musculus
MDPNCSCSTGGSCTCTSSCACKNCKCTSCKKSELGHLGWRLRLGAGNSYKTGSEKCPLLPGGHCIVSGTELYRELFKKTEVFSVGDRKQRSSARLTSSSSFLGCCSCCPVGCSKCAQGCVCKGAADKCTCCA

SEQ ID NO: 57 - DNA, 1230 bp, Mus musculus
  source 1..1230: mol_type = other DNA
atggtgggggcagacccgaccagaccccggggaccgctgagctattgggcgggccgtcggggtcaggggctcgcagcgatcttcctgctcctggtgtccgcggcggaatccgaggccagggcagaggatgacttcagcctggtgcagccgctggtgaccatggagcagctgctgtgggtgagcgggaagcagatcggctctgtagacactttccgcatcccgctcatcacagccacccctcggggcacgctcctggccttcgctgaggccaggaaaaaatctgcatccgatgagggggccaagttcatcgccatgaggaggtccacggaccagggtagcacgtggtcctctacagccttcatcgtagacgatggggaggcctccgatggcctgaacctgggcgctgtggtgaacgatgtagacacagggatagtgttccttatctataccctctgtgctcacaaggtcaactgccaggtggcctctaccatgttggtttggagtaaggacgacggcatttcctggagcccaccccggaatctctctgtggatattggcacagagatgtttgcccctggacctggctcaggcattcagaaacagcgggagcctgggaagggccggctcattgtgtgtggacacgggacgctggagcgagatggggtcttctgtctcctcagtgatgaccacggtgcctcctggcactacggcactggagtgagcggcattccctttggccagcccaaacacgatcacgatttcaaccccgacgagtgccagccctacgagcttccagatggctcggtcatcatcaacgcccggaaccagaataactaccattgccgctgcaggatcgtcctccgcagctatgacgcctgtgacaccctcaggccccgggatgtgaccttcgaccctgagctcgtggaccctgtggtagctgcaggagcactagccaccagctccggcattgtcttcttctccaatccagcccaccctgagttccgagtgaacctgaccctgcgctggagtttcagcaatggtacatcctggctgaaggagagggtccaggtgtggccgggacccagcggctactcgtccctgacagccctggaaaacagcacggatggaaagaagcagcccccgcagctgttcgttctgtacgagaaaggcctgaaccggtacaccgagagcatctccatggtcaaaatcagcgtctacggcacgctctga

SEQ ID NO: 58 - protein, 409 aa, Mus musculus
MVGADPTRPRGPLSYWAGRRGQGLAAIFLLLVSAAESEARAEDDFSLVQPLVTMEQLLWVSGKQIGSVDTFRIPLITATPRGTLLAFAEARKKSASDEGAKFIAMRRSTDQGSTWSSTAFIVDDGEASDGLNLGAVVNDVDTGIVFLIYTLCAHKVNCQVASTMLVWSKDDGISWSPPRNLSVDIGTEMFAPGPGSGIQKQREPGKGRLIVCGHGTLERDGVFCLLSDDHGASWHYGTGVSGIPFGQPKHDHDFNPDECQPYELPDGSVIINARNQNNYHCRCRIVLRSYDACDTLRPRDVTFDPELVDPVVAAGALATSSGIVFFSNPAHPEFRVNLTLRWSFSNGTSWLKERVQVWPGPSGYSSLTALENSTDGKKQPPQLFVLYEKGLNRYTESISMVKISVYGTL

SEQ ID NO: 59 - DNA, 471 bp, Mus musculus
  source 1..471: mol_type = other DNA
atgagcacctccaggctttatacccttgtgctagtgctacagcctcagcgagttctcctgggcatgaagaagaggggctttggtgctggccgctggaatggcttcgggggcaaggtgcaggaaggagagaccattgaggatggggctaagagagagctgctggaagaaagtggtctgagcgtggatacactgcacaaggtaggccacatctcgtttgaatttgtgggctcccctgagctgatggacgtgcatatcttctcggctgaccatgtgcacgggacgcccacagagagtgaagaaatgcgccctcagtggttccaactggaccagatcccctttgccgacatgtggccggatgacagctactggttcccactcctgcttcagaagaagaagttctgtgggcacttcaagttccaggatcaggacacgatcctcagttactcgctgcgagaggtggactcattctaa

SEQ ID NO: 60 - protein, 156 aa, Mus musculus
MSTSRLYTLVLVLQPQRVLLGMKKRGFGAGRWNGFGGKVQEGETIEDGAKRELLEESGLSVDTLHKVGHISFEFVGSPELMDVHIFSADHVHGTPTESEEMRPQWFQLDQIPFADMWPDDSYWFPLLLQKKKFCGHFKFQDQDTILSYSLREVDSF

SEQ ID NO: 61 - DNA, 1869 bp, Mus musculus
  source 1..1869: mol_type = other DNA
atgcctcctcagctgcataacggtctggacttctctgccaaggtcatccagggcagcctcgacagcctgccccaggcagtgaggaagttcgtggaaggcaatgctcagctgtgccagccggagtatatccacatctgcgatggctccgaggaggagtacgggcagttgctgacccacatgcaggaggagggtgtcatccgcaagctgaagaaatatgacaactgttggctggctctcactgaccctcgagatgtggccaggatcgaaagcaagacagtcatcatcacccaagagcagagagacacagtgcccatccccaaaactggcctcagccagctgggccgctggatgtcggaagaggactttgagaaagcattcaacgccaggttcccagggtgcatgaaaggccgcaccatgtatgtcatcccattcagcatggggccactgggctcgccgctggccaagattggtattgaactgacagactcgccctatgtggtggccagcatgcggatcatgactcggatgggcatatctgtgctggaggccctgggagatggggagttcatcaagtgcctgcactctgtggggtgccctctccccttaaaaaagcctttggtcaacaactgggcctgcaaccctgagctgaccctgatcgcccacctcccggaccgcagagagatcatctcctttggaagcggatatggtgggaactcactactcgggaagaaatgctttgcgttgcggatcgccagccgtctggctaaggaggaagggtggctggcggagcatatgctgatcctgggcataactaaccccgaaggcaagaagaaatacctggccgcagccttccctagtgcctgtgggaagaccaacttggccatgatgaaccccagcctgcccgggtggaaggtcgaatgtgtgggcgatgacatcgcctggatgaagtttgatgcccaaggcaacttaagggctatcaacccagaaaacgggttttttggagttgctcctggcacctcagtgaagacaaatccaaatgccattaaaaccatccagaaaaacaccatcttcaccaacgtggctgagactagcgatgggggtgtttactgggaaggcatcgatgagccgctggccccgggagtcaccatcacctcctggaagaacaaggagtggagaccgcaggacgcggaaccatgtgcccatcccaactcgagattctgcacccctgccagccagtgccccattattgaccctgcctgggaatctccagaaggagtacccattgagggtatcatctttggtggccgtagacctgaaggtgtcccccttgtctatgaagccctcagctggcagcatggggtgtttgtaggagcagccatgagatctgaggccacagctgctgcagaacacaagggcaagatcatcatgcacgacccctttgccatgcgacccttcttcggctacaacttcggcaaatacctggcccactggctgagcatggcccaccgcccagcagccaagttgcccaagatcttccatgtcaactggttccggaaggacaaagatggcaagttcctctggccaggctttggcgagaactcccgggtgctggagtggatgttcgggcggattgaaggggaagacagcgccaagctcacgcccatcggctacatccctaaggaaaacgccttgaacctgaaaggcctggggggcgtcaacgtggaggagctgtttgggatctctaaggagttctgggagaaggaggtggaggagatcgacaggtatctggaggaccaggtcaacaccgacctcccttacgaaattgagagggagctccgagccctgaaacagagaatcagccagatgtaa

SEQ ID NO: 62 - protein, 622 aa, Mus musculus
MPPQLHNGLDFSAKVIQGSLDSLPQAVRKFVEGNAQLCQPEYIHICDGSEEEYGQLLTHMQEEGVIRKLKKYDNCWLALTDPRDVARIESKTVIITQEQRDTVPIPKTGLSQLGRWMSEEDFEKAFNARFPGCMKGRTMYVIPFSMGPLGSPLAKIGIELTDSPYVVASMRIMTRMGISVLEALGDGEFIKCLHSVGCPLPLKKPLVNNWACNPELTLIAHLPDRREIISFGSGYGGNSLLGKKCFALRIASRLAKEEGWLAEHMLILGITNPEGKKKYLAAAFPSACGKTNLAMMNPSLPGWKVECVGDDIAWMKFDAQGNLRAINPENGFFGVAPGTSVKTNPNAIKTIQKNTIFTNVAETSDGGVYWEGIDEPLAPGVTITSWKNKEWRPQDAEPCAHPNSRFCTPASQCPIIDPAWESPEGVPIEGIIFGGRRPEGVPLVYEALSWQHGVFVGAAMRSEATAAAEHKGKIIMHDPFAMRPFFGYNFGKYLAHWLSMAHRPAAKLPKIFHVNWFRKDKDGKFLWPGFGENSRVLEWMFGRIEGEDSAKLTPIGYIPKENALNLKGLGGVNVEELFGISKEFWEKEVEEIDRYLEDQVNTDLPYEIERELRALKQRISQM

SEQ ID NO: 63 - DNA, 885 bp, Mus musculus
  source 1..885: mol_type = other DNA
atgtggcagtcctccagcgtggttttccacacgggcgccggcatcagcaccgcctctggcatccccgacttcagaggcccccatggcgtgtggaccatggaggaacgcggcctggcccccaagtttgacaccaccttcgagaatgctcggccctcgaagacccacatggccctggttcagctagaacgcatgggcttcctcagcttcctggtcagccagaacgtagacgggctgcacgtgcgctcgggcttccccagggacaagctggcagagctgcacggaaacatgtttgtagaggaatgtcccaagtgtaagacgcagtacgtcagagacacggttgtgggcaccatgggcctcaaggccacaggccggctctgcaccgtggccaagaccaggggacttcgggcctgtagaggggagctgagagacaccattctggactgggaggactcgttgcctgaccgggacctgatgctcgctgatgaggccagcaggaccgcagacctgtctgtcaccctgggtacctcgctgcagatccgccccagtgggaacctgccccttgccactaagcgccgaggaggccgtctggtcattgtcaacctgcaacccacaaaacatgaccgccaggctgacctgcgcatccacggctacgtggatgaggtgatgtgcagactcatgaagcatctggggctggagattccagcctgggatggaccctgcgtgctagacaaagccctgccacctctgcctcgcccagtagcactcaaggctgagccccccgtgcatctcaatggtgcagtgcatgtttcgtataagtccaagcccaacagccctatactccacaggccccccaaaagagtgaagaccgaggctgcccccagctga

SEQ ID NO: 64 - protein, 294 aa, Mus musculus
MWQSSSVVFHTGAGISTASGIPDFRGPHGVWTMEERGLAPKFDTTFENARPSKTHMALVQLERMGFLSFLVSQNVDGLHVRSGFPRDKLAELHGNMFVEECPKCKTQYVRDTVVGTMGLKATGRLCTVAKTRGLRACRGELRDTILDWEDSLPDRDLMLADEASRTADLSVTLGTSLQIRPSGNLPLATKRRGGRLVIVNLQPTKHDRQADLRIHGYVDEVMCRLMKHLGLEIPAWDGPCVLDKALPPLPRPVALKAEPPVHLNGAVHVSYKSKPNSPILHRPPKRVKTEAAPS

SEQ ID NO: 65 - DNA, 3369 bp, Mus musculus
  source 1..3369: mol_type = other DNA
atgacccgcgctcctcgttgccccgcggtgcgctctctgctgcgcagccgataccgggaggtgtggccgctggcaacctttgtgcggcgcctggggcccgagggcaggcggcttgtgcaacccggggacccgaagatctaccgcactttggttgcccaatgcctagtgtgcatgcactggggctcacagcctccacctgccgacctttccttccaccaggtgtcatccctgaaagagctggtggccagggttgtgcagagactctgcgagcgcaacgagagaaacgtgctggcttttggctttgagctgcttaacgaggccagaggcgggcctcccatggccttcactagtagcgtgcgtagctacttgcccaacactgttattgagaccctgcgtgtcagtggtgcatggatgctactgttgagccgagtgggcgacgacctgctggtctacctgctggcacactgtgctctttatcttctggtgccccccagctgtgcctaccaggtgtgtgggtctcccctgtaccaaatttgtgccaccacggatatctggccctctgtgtccgctagttacaggcccacccgacccgtgggcaggaatttcactaaccttaggttcttacaacagatcaagagcagtagtcgccaggaagcaccgaaacccctggccttgccatctcgaggtacaaagaggcatctgagtctcaccagtacaagtgtgccttcagctaagaaggccagatgctatcctgtcccgagagtggaggagggaccccacaggcaggtgctaccaaccccatcaggcaaatcatgggtgccaagtcctgctcggtcccccgaggtgcctactgcagagaaagatttgtcttctaaaggaaaggtgtctgacctgagtctctctgggtcggtgtgctgtaaacacaagcccagctccacatctctgctgtcaccaccccgccaaaatgcctttcagctcaggccatttattgagaccagacatttcctttactccaggggagatggccaagagcgtctaaacccctcattcctactcagcaacctccagcctaacttgactggggccaggagactggtggagatcatctttctgggctcaaggcctaggacatcaggaccactctgcaggacacaccgtctatcgcgtcgatactggcagatgcggcccctgttccaacagctgctggtgaaccatgcagagtgccaatatgtcagactcctcaggtcacattgcaggtttcgaacagcaaaccaacaggtgacagatgccttgaacaccagcccaccgcacctcatggatttgctccgcctgcacagcagtccctggcaggtatatggttttcttcgggcctgtctctgcaaggtggtgtctgctagtctctggggtaccaggcacaatgagcgccgcttctttaagaacttaaagaagttcatctcgttggggaaatacggcaagctatcactgcaggaactgatgtggaagatgaaagtagaggattgccactggctccgcagcagcccggggaaggaccgtgtccccgctgcagagcaccgtctgagggagaggatcctggctacgttcctgttctggctgatggacacatacgtggtacagctgcttaggtcattcttttacatcacagagagcacattccagaagaacaggctcttcttctaccgtaagagtgtgtggagcaagctgcagagcattggagtcaggcaacaccttgagagagtgcggctacgggagctgtcacaagaggaggtcaggcatcaccaggacacctggctagccatgcccatctgcagactgcgcttcatccccaagcccaacggcctgcggcccattgtgaacatgagttatagcatgggtaccagagctttgggcagaaggaagcaggcccagcatttcacccagcgtctcaagactctcttcagcatgctcaactatgagcggacaaaacatcctcaccttatggggtcttctgtactgggtatgaatgacatctacaggacctggcgggcctttgtgctgcgtgtgcgtgctctggaccagacacccaggatgtactttgttaaggcagatgtgaccggggcctatgatgccatcccccagggtaagctggtggaggttgttgccaatatgatcaggcactcggagagcacgtactgtatccgccagtatgcagtggtccggagagatagccaaggccaagtccacaagtcctttaggagacaggtcaccaccctctctgacctccagccatacatgggccagttccttaagcatctgcaggattcagatgccagtgcactgaggaactccgttgtcatcgagcagagcatctctatgaatgagagcagcagcagcctgtttgacttcttcctgcacttcctgcgtcacagtgtcgtaaagattggtgacaggtgctatacgcagtgccagggcatcccccagggctccagcctatccaccctgctctgcagtctgtgtttcggagacatggagaacaagctgtttgctgaggtgcagcgggatgggttgcttttacgttttgttgatgactttctgttggtgacgcctcacttggaccaagcaaaaaccttcctcagcaccctggtccatggcgttcctgagtatgggtgcatgataaacttgcagaagacagtggtgaacttccctgtggagcctggtaccctgggtggtgcagctccataccagctgcctgctcactgcctgtttccctggtgtggcttgctgctggacactcagactttggaggtgttctgtgactactcaggttatgcccagacctcaattaagacgagcctcaccttccagagtgtcttcaaagctgggaagaccatgcggaacaagctcctgtcggtcttgcggttgaagtgtcacggtctatttctagacttgcaggtgaacagcctccagacagtctgcatcaatatatacaagatcttcctgcttcaggcctacaggttccatgcatgtgtgattcagcttccctttgaccagcgtgttaggaagaacctcacattctttctgggcatcatctccagccaagcatcctgctgctatgctatcctgaaggtcaagaatccaggaatgacactaaaggcctctggctcctttcctcctgaagccgcacattggctctgctaccaggccttcctgctcaagctggctgctcattctgtcatctacaaatgtctcctgggacctctgaggacagcccaaaaactgctgtgccggaagctcccagaggcgacaatgaccatccttaaagctgcagctgacccagccctaagcacagactttcagaccattttggactaa

SEQ ID NO: 66 - protein, 1122 aa, Mus musculus
MTRAPRCPAVRSLLRSRYREVWPLATFVRRLGPEGRRLVQPGDPKIYRTLVAQCLVCMHWGSQPPPADLSFHQVSSLKELVARVVQRLCERNERNVLAFGFELLNEARGGPPMAFTSSVRSYLPNTVIETLRVSGAWMLLLSRVGDDLLVYLLAHCALYLLVPPSCAYQVCGSPLYQICATTDIWPSVSASYRPTRPVGRNFTNLRFLQQIKSSSRQEAPKPLALPSRGTKRHLSLTSTSVPSAKKARCYPVPRVEEGPHRQVLPTPSGKSWVPSPARSPEVPTAEKDLSSKGKVSDLSLSGSVCCKHKPSSTSLLSPPRQNAFQLRPFIETRHFLYSRGDGQERLNPSFLLSNLQPNLTGARRLVEIIFLGSRPRTSGPLCRTHRLSRRYWQMRPLFQQLLVNHAECQYVRLLRSHCRFRTANQQVTDALNTSPPHLMDLLRLHSSPWQVYGFLRACLCKVVSASLWGTRHNERRFFKNLKKFISLGKYGKLSLQELMWKMKVEDCHWLRSSPGKDRVPAAEHRLRERILATFLFWLMDTYVVQLLRSFFYITESTFQKNRLFFYRKSVWSKLQSIGVRQHLERVRLRELSQEEVRHHQDTWLAMPICRLRFIPKPNGLRPIVNMSYSMGTRALGRRKQAQHFTQRLKTLFSMLNYERTKHPHLMGSSVLGMNDIYRTWRAFVLRVRALDQTPRMYFVKADVTGAYDAIPQGKLVEVVANMIRHSESTYCIRQYAVVRRDSQGQVHKSFRRQVTTLSDLQPYMGQFLKHLQDSDASALRNSVVIEQSISMNESSSSLFDFFLHFLRHSVVKIGDRCYTQCQGIPQGSSLSTLLCSLCFGDMENKLFAEVQRDGLLLRFVDDFLLVTPHLDQAKTFLSTLVHGVPEYGCMINLQKTVVNFPVEPGTLGGAAPYQLPAHCLFPWCGLLLDTQTLEVFCDYSGYAQTSIKTSLTFQSVFKAGKTMRNKLLSVLRLKCHGLFLDLQVNSLQTVCINIYKIFLLQAYRFHACVIQLPFDQRVRKNLTFFLGIISSQASCCYAILKVKNPGMTLKASGSFPPEAAHWLCYQAFLLKLAAHSVIYKCLLGPLRTAQKLLCRKLPEATMTILKAAADPALSTDFQTILD

SEQ ID NO: 67 - DNA, 1428 bp, Mus musculus
  source 1..1428: mol_type = other DNA
atggcgtcacgcatcgggctgcgcatgcagctcatgcgggagcaggcccagcaggaggagcagcgagagcgcatgcagcagcaggctgtcatgcattatatgcaacagcagcagcagcagcagcagcagctgggtgggccccccaccccagccatcaacacccctgtccacttccagtcgcccccgcctgtgcccggggaggtgctgaaggtgcagtcctacctggagaaccccacctcctaccacctgcaacagtcccagcatcagaaggttcggaagtatctgtctgagacctatgggaacaagtttgctgcccacgtgagcccagcccaaggttccccgaagcctgccccagcagcatccccaggggtgcgggctggacacgtactgtccacctcggccggcaacagtgctcccaacagtcccatggccatgctacatatcagctccaaccccgagaaagagtttgatgatgtcattgacaacattatgcgcctggacagcgtgctgggctacatcaaccctgagatgcagatgcctaacacgctgcccctgtctagcagccacctgaacgtgtacagcggtgacccccaggtcacagcctccatggtgggtgtcaccagcagctcctgccctgccgacctgactcagaagcgagagctaacagatgctgagagcagagccctggccaaggagcggcagaagaaagacaatcacaacctaattgagagaagacgcaggttcaacatcaatgaccggatcaaggagctgggaatgctgatccccaaggccaacgacctggacgtgcgctggaacaaaggcaccatcctcaaggcctctgtggattacatccggaggatgcagaaggacctgcagaagtcccgggagctggagaaccactcccggcgcctggagatgactaacaagcagctctggctccgcatccaggagctggagatgcaggcacgcgtgcacggcctccccaccacctcgccgtcgggtgtgaatatggccgagctggcccagcaggtggtgaagcaagagttgcccagtgaggatggcccaggggaggcgctgatgctggggcctgaggtccctgagcctgagcaaatgccggctcttcctccccaggctccgctgccctcggccgcccagccacagtctccgttccatcacctggacttcagccatggcctgagctttgggggtgggggcgacgaggggcccacaggttaccccgataccctggggacagagcacggctccccattccccaacctgtccaagaaggatctggacttaatgctcctagatgactccctgctccccctggcctctgaccccctcttttctaccatgtctcctgaggcctccaaggccagcagccgccggagcagcttcagcatggaggagggtgatgttctgtga

SEQ ID NO: 68 - protein, 475 aa, Mus musculus
MASRIGLRMQLMREQAQQEEQRERMQQQAVMHYMQQQQQQQQQLGGPPTPAINTPVHFQSPPPVPGEVLKVQSYLENPTSYHLQQSQHQKVRKYLSETYGNKFAAHVSPAQGSPKPAPAASPGVRAGHVLSTSAGNSAPNSPMAMLHISSNPEKEFDDVIDNIMRLDSVLGYINPEMQMPNTLPLSSSHLNVYSGDPQVTASMVGVTSSSCPADLTQKRELTDAESRALAKERQKKDNHNLIERRRRFNINDRIKELGMLIPKANDLDVRWNKGTILKASVDYIRRMQKDLQKSRELENHSRRLEMTNKQLWLRIQELEMQARVHGLPTTSPSGVNMAELAQQVVKQELPSEDGPGEALMLGPEVPEPEQMPALPPQAPLPSAAQPQSPFHHLDFSHGLSFGGGGDEGPTGYPDTLGTEHGSPFPNLSKKDLDLMLLDDSLLPLASDPLFSTMSPEASKASSRRSSFSMEEGDVL

SEQ ID NO: 69 - DNA, 318 bp, Mus musculus
  source 1..318: mol_type = other DNA
atggtgaagctgatcgagagcaaggaagcttttcaggaggccctggccgccgcgggagacaagcttgtcgtggtggacttctctgctacgtggtgtggaccttgcaaaatgatcaagcccttcttccattccctctgtgacaagtattccaatgtggtgttccttgaagtggatgtggatgactgccaggatgttgctgcagactgtgaagtcaaatgcatgccgaccttccagttttataaaaagggtcaaaaggtgggggagttctccggtgctaacaaggaaaagcttgaagcctctattactgaatatgcctaa

SEQ ID NO: 70 - protein, 105 aa, Mus musculus
MVKLIESKEAFQEALAAAGDKLVVVDFSATWCGPCKMIKPFFHSLCDKYSNVVFLEVDVDDCQDVAADCEVKCMPTFQFYKKGQKVGEFSGANKEKLEASITEYA

SEQ ID NO: 71 - DNA, 924 bp, Mus musculus
  source 1..924: mol_type = other DNA
atggtgaacccgacaacttccgaagtgcaacccaccatgggggtcaagatcttctcagccggagtttcagcttgcctggcagatatcatcaccttcccgctggacactgccaaagtccgccttcagatccaaggtgaaggccaggcttccagtaccattaggtataaaggtgtcctagggaccatcaccaccctggcaaaaacagaaggattgccgaaactgtacagcggtctgcctgcgggcattcagaggcaaatcagctttgcctcactcaggattggcctctacgactcagtccaagagtacttctcttcagggagagaaacacctgcctctctcggaaacaagatctcagccggcttaatgactggaggtgtggcagtgttcattgggcagcctacagaggtcgtgaaggtcagaatgcaagcccagagccatctgcatgggatcaaaccccgctacacggggacctacaatgcttacagagttatagccaccacagaaagcttgtcaacactttggaaagggacgacccctaatctaatgagaaatgtcatcatcaattgtacagagctggtaacatatgacctcatgaagggggcccttgtaaacaacaaaatactggcagatgacgtcccctgccatttactgtcagctcttgttgccgggttttgcaccacactcctggcctctccagtggatgtggtaaaaacaagattcatcaactctctgccaggacagtacccaagcgtaccaagctgtgcgatgtccatgtacaccaaggaaggaccgacggcctttttcaaagggtttgtggcttcttttctgcgactcgggtcctggaacgtcatcatgtttgtgtgctttgaacagctgaaaaaagagctgatgaagtccagacagacagtggattgtaccacataa

SEQ ID NO: 72 - protein, 307 aa, Mus musculus
MVNPTTSEVQPTMGVKIFSAGVSACLADIITFPLDTAKVRLQIQGEGQASSTIRYKGVLGTITTLAKTEGLPKLYSGLPAGIQRQISFASLRIGLYDSVQEYFSSGRETPASLGNKISAGLMTGGVAVFIGQPTEVVKVRMQAQSHLHGIKPRYTGTYNAYRVIATTESLSTLWKGTTPNLMRNVIINCTELVTYDLMKGALVNNKILADDVPCHLLSALVAGFCTTLLASPVDVVKTRFINSLPGQYPSVPSCAMSMYTKEGPTAFFKGFVASFLRLGSWNVIMFVCFEQLKKELMKSRQTVDCTT

SEQ ID NO: 73 - DNA, 1659 bp, Heterocephalus glaber
  source 1..1659: mol_type = other DNA
atgcattgtgagaggtttctatgtgtcctgagaataattggaactacactttttggagtgtctctcctcctcggaatcacagctgcttatattgttggctaccagtttatccaaacagataattactacttctcatttggactgtacggtgcctttttagcctcgcatctcatcatccaaagcctctttgcctttttggaacaccggaaaatgaagaagtcccttgaaaccccgattaaattgaacaaaacggtagcactctgcatcgctgcgtaccaagaggaccctgactacttacggaaatgtttgcaatctgtgaaaaggctgacctaccctgggattaaagtcgtgatggtcatcgatgggaactcagacgacgacctttacatgatggacatattcagcgaagttatgggcagggacaaatcggccacgtacatctggaagaacaactttcatgaaaagggacctggtgagacagaagagtcccataaagaaagttcacaacatgtcacccaattggtcttgtctagcaaaagtgtttgcatcatgcaaaaatggggtggaaagagagaagtcatgtacacagccttcagagcactggggcgaagcgtggattatgtacaggtgtgtgactcagatactatgcttgaccctgcctcatctgtggagatggtgaaggtcttagaggaagaccctatggttggaggtgttggaggagatgtccagattttaaacaagtatgattcctggatctccttcctcagcagcgtgagatactggatggcttttaatatagaaagggcctgccagtcttattttggctgtgtccagtgcataagcggtcctctgggaatgtacagaaactccttgctgcatgaatttgtggaagactggtacagtcaggaattcatgggtaaccaatgcagttttggtgacgacaggcaccttaccaacagggtgttgagtctgggctatgcaactaaatacacggctcggtccaagtgccttactgaaactcccatagaatatctgagatggctgaaccagcagacccgttggagcaagtcctacttccgagagtggctgtacaatgccatgtggtttcacaagcatcacttgtggatgacctatgaagctgttatcactggattctttcctttctttctcattgccacagtcatccagctcttctacaggggtaaaatctggaacatcctcctcttcctgttaactgtccagctagtgggtctcatcaagtcatcttttgccagctgccttagaggaaatatcgtcatggtattcatgtctctgtattcagtgttatacatgtcaagtctacttcctgccaagatgtttgcaattgcaaccataaacaaagctgggtggggcacatctggaaggaagaccattgttgttaatttcataggacttattccagtgtccgtgtggtttacaatccttctaggtggtgtaattttcaccatttataaggaatctaaaaagccattttccgaatccaaacagactgttctcatcgtgggaactttgatctatgcatgctactgggtcatgcttttgactctctatgtggttctcatcaataagtgtggcaggcggaagaagggacaacagtatgacatggtgcttgatgtatga

SEQ ID NO: 74 - protein, 552 aa, Heterocephalus glaber
MHCERFLCVLRIIGTTLFGVSLLLGITAAYIVGYQFIQTDNYYFSFGLYGAFLASHLIIQSLFAFLEHRKMKKSLETPIKLNKTVALCIAAYQEDPDYLRKCLQSVKRLTYPGIKVVMVIDGNSDDDLYMMDIFSEVMGRDKSATYIWKNNFHEKGPGETEESHKESSQHVTQLVLSSKSVCIMQKWGGKREVMYTAFRALGRSVDYVQVCDSDTMLDPASSVEMVKVLEEDPMVGGVGGDVQILNKYDSWISFLSSVRYWMAFNIERACQSYFGCVQCISGPLGMYRNSLLHEFVEDWYSQEFMGNQCSFGDDRHLTNRVLSLGYATKYTARSKCLTETPIEYLRWLNQQTRWSKSYFREWLYNAMWFHKHHLWMTYEAVITGFFPFFLIATVIQLFYRGKIWNILLFLLTVQLVGLIKSSFASCLRGNIVMVFMSLYSVLYMSSLLPAKMFAIATINKAGWGTSGRKTIVVNFIGLIPVSVWFTILLGGVIFTIYKESKKPFSESKQTVLIVGTLIYACYWVMLLTLYVVLINKCGRRKKGQQYDMVLDV

SEQ ID NO: 75 - DNA, 727 bp, synthetic construct
  misc_feature 1..727: note = Description of Artificial Sequence: Synthetic polynucleotide
  source 1..727: mol_type = other DNA
ccaaggtatattgctgttgacagtgagcgacgctgcagatattccgctctaagtgaagccacagatgttagagcggaaaatctgcagagctgcctactgcctcggacttcaaggggcttgcggccgccatctccatggctgtaccaccttgtcggccaggttactacagatatgtatgttgaatctcattacatatctgttgtaacctgctctgacattttggtatctttcatctgaccacgtactaccttctatctgatgtgacagcttctgtagcaccagatgaagattgggctcaatgtttagttatttgagcccaagcttcatctgtgtactgctagctgtagaactccagcttcggcctgtaacttatgatagcaatgtcagcagtgcctggcagccgtggatcgaataatttaagattctaaaattatagtattcgatcaacggctgcaaagtaaggttgaccatactctacagttgttgatttcgtggctacagagtttccttagcagagctggatgcagtgcagccatatatttgtctaaactataatatatggctgcactgcatatagctactgctaggcaatccttccctcgataagatgcagcggcggctcctctccccatggccctggccttgttgaagaggattatcctgggctcagagataatcctctacaacaagggcagggacctggggaccccggcaccggcaggctagc

SEQ ID NO: 76 - DNA, 846 bp, synthetic construct
  misc_feature 1..846: note = Description of Artificial Sequence: Synthetic polynucleotide
  source 1..846: mol_type = other DNA
ggccaaggtatattgctgttgacagtgagcgacgctgcagatattccgctctaagtgaagccacagatgttagagcggaaaatctgcagagctgcctactgcctcggacttcaaggggcttgcggccgccatctccatggctgtaccaccttgtcggccaggttactacagatatgtatgttgaatctcattacatatctgttgtaacctgctctgacattttggtatctttcatctgaccacgtactaccttctatctgatgtgacagcttctgtagcaccctgttactacacatacttttgtttagttataaagtatgtggagtaacaggtgtactgctagctgtagaactccagcttcggcctgtaacttatgatagcaatgtcagcagtgcctcctgttccctttcctaatcatttaagattctaaaattataggattaggaatgggaacagtaagtaaggttgaccatactctacagttgttgatttcgtggctacagagtttccttagcagagctgtggcacctttattggctacaatgtctaaactatttgtagccaataaaggtgccctagctactgctaggcaatccttccctcgataagtatggggcctggctcgagcagggggcgagggatgcatctagtagagcggatgattggtcccctcccttaacaagtcgaactgtcttgtccttccctcccaatgaccgcgtcttcgtcacagtcagcggcggctcctctccccatggccctgatgctgtccctggtccttatgctgggctcagaatacctggtgtgagtctcagtcagggacctggggaccccggcaccggcaggcta

SEQ ID NO: 77 - DNA, 727 bp, synthetic construct
  misc_feature 1..727: note = Description of Artificial Sequence: Synthetic polynucleotide
  source 1..727: mol_type = other DNA
ccaaggtatattgctgttgacagtgagcgacgctgcagatattccgctctaagtgaagccacagatgttagagcggaaaatctgcagagctgcctactgcctcggacttcaaggggcttgcggccgccatctccatggctgtaccaccttgtcggccaggttactacagatatgtatgttgaatctcattacatatctgttgtaacctgctctgacattttggtatctttcatctgaccacgtactaccttctatctgatgtgacagcttctgtagcaccctgttactacacatacttttgtttagttataaagtatgtggagtaacaggtgtactgctagctgtagaactccagcttcggcctgtaacttatgatagcaatgtcagcagtgcctcctgttccctttcctaatcatttaagattctaaaattataggattaggaatgggaacagtaagtaaggttgaccatactctacagttgttgatttcgtggctacagagtttccttagcagagctggatgcagtgcagccatatatttgtctaaactataatatatggctgcactgcatatagctactgctaggcaatccttccctcgataagatgcagcggcggctcctctccccatggccctggccttgttgaagaggattatcctgggctcagagataatcctctacaacaagggcagggacctggggaccccggcaccggcaggctagc

SEQ ID NO: 78 - DNA, 846 bp, synthetic construct
  misc_feature 1..846: note = Description of Artificial Sequence: Synthetic polynucleotide
  source 1..846: mol_type = other DNA
ggccaaggtatattgctgttgacagtgagcgacgctgcagatattccgctctaagtgaagccacagatgttagagcggaaaatctgcagagctgcctactgcctcggacttcaaggggcttgcggccgccatctccatggctgtaccaccttgtcggccaggttactacagatatgtatgttgaatctcattacatatctgttgtaacctgctctgacattttggtatctttcatctgaccacgtactaccttctatctgatgtgacagcttctgtagcaccctgttactacacatacttttgtttagttataaagtatgtggagtaacaggtgtactgctagctgtagaactccagcttcggcctgtaacttatgatagcaatgtcagcagtgcctcctgttccctttcctaatcatttaagattctaaaattataggattaggaatgggaacagtaagtaaggttgaccatactctacagttgttgatttcgtggctacagagtttccttagcagagctgctggatgcagtggcgacattttgtctaaactataaatgtcgccactgcatccattagctactgctaggcaatccttccctcgataagtatggggcctggctcgagcagggggcgagggatcagacagttggacttgttaaatggtcccctccctcttgtctgaatcaggtaatgtccttccctcccaatgaccgcgtcttcgtcacagtcagcggcggctcctctccccatggccctgatgctgtccctggtccttatgctgggctcagacataaggaccacggacagcaacagggacctggggaccccggcaccggcaggcta

SEQ ID NO: 79 - DNA, 846 bp, synthetic construct
  misc_feature 1..846: note = Description of Artificial Sequence: Synthetic polynucleotide
  source 1..846: mol_type = other DNA
ggccaaggtatattgctgttgacagtgagcgaccttacctgaatcagacaagaagtgaagccacagatgttcttgtctgaatcaggtaatgctgcctactgcctcggacttcaaggggcttgcggccgccatctccatggctgtaccaccttgtcgggcatctagtagagcggatgattgttgaatctcatttcatccgctcaactagatggtctgacattttggtatctttcatctgaccacgtactaccttctatctgatgtgacagcttctgtagcaccctgttactacacatacttttgtttagttataaagtatgtggagtaacaggtgtactgctagctgtagaactccagcttcggcctgtaacttatgatagcaatgtcagcagtgcctcctgttccctttcctaatcatttaagattctaaaattataggattaggaatgggaacagtaagtaaggttgaccatactctacagttgttgatttcgtggctacagagtttccttagcagagctgctggatgcagtggcgacattttgtctaaactataaatgtcgccactgcatccattagctactgctaggcaatccttccctcgataagtatggggcctggctcgagcagggggcgagggatcagacagttggacttgttaaatggtcccctccctatgaacggtctttccctctgtccttccctcccaatgaccgcgtcttcgtcacagtcagcggcggctcctctccccatggccctggccttgttgaagaggattatcctgggctcagacataaggaccacggacagcaacagtgacctggggaccccggcaccggcaggcta

SEQ ID NO: 80 - DNA, 727 bp, synthetic construct
  misc_feature 1..727: note = Description of Artificial Sequence: Synthetic polynucleotide
  source 1..727: mol_type = other DNA
ccaaggtatattgctgttgacagtgagcgaccttacctgaatcagacaagaagtgaagccacagatgttcttgtctgaatcaggtaatgctgcctactgcctcggacttcaaggggcttgcggccgccatctccatggctgtaccaccttgtcgggcatctagtagagcggatgattgttgaatctcatttcatccgctcaactagatggtctgacattttggtatctttcatctgaccacgtactaccttctatctgatgtgacagcttctgtagcaccctgttactacacatacttttgtttagttataaagtatgtggagtaacaggtgtactgctagctgtagaactccagcttcggcctgtaacttatgatagcaatgtcagcagtgcctcctgttccctttcctaatcatttaagattctaaaattataggattaggaatgggaacagtaagtaaggttgaccatactctacagttgttgatttcgtggctacagagtttccttagcagagctggctctaaagaaggcttatgaatgtctaaactatttcataagcctactttagagatagctactgctaggcaatccttccctcgataagtatggggcctggctcgagcagggggcgagggatgcatctagtagagcggatgattggtcccctccctcatccgctcaactagatgagtccttccctcccaatgaccgcgtcttggctagc

SEQ ID NO: 81 - DNA, 727 bp, synthetic construct
  misc_feature 1..727: note = Description of Artificial Sequence: Synthetic polynucleotide
  source 1..727: mol_type = other DNA
ccaaggtatattgctgttgacagtgagcgactctgagactgacaccaggtatgtgaagccacagatgatacctggtgtgagtctcagcgctgcctactgcctcggacttcaaggggcttgcggccgccatctccatggctgtaccaccttgtcggtggcacctttattggctacaatgttgaatctcatttgtagccaattaaggtgccttctgacattttggtatctttcatctgaccacgtactaccttctatctgatgtgacagcttctgtagcacgccaccctcttcacggccaatgtttagttatttggccgtgacgagggtggctgtactgctagctgtagaactccagcttcggcctgtaacttatgatagcaatgtcagcagtgcctcgccaccctcttcacggccaattaagattctaaaattattgggccgtgaacagggtggctaagtaaggttgaccatactctacagttgttgatttcgtggctacagagtttccttagcagagctgtggcacctttattggctacaatgtctaaactatttgtagccaataaaggtgccctagctactgctaggcaatccttccctcgataagatgcagcggcggctcctctccccatggccctgcttgggctgtccctatctttcctgggctcagagaaagatagggtcagcccaaccagggacctggggaccccggcaccggcaggctagc

SEQ ID NO: 82 - DNA, 727 bp, synthetic construct
  misc_feature 1..727: note = Description of Artificial Sequence: Synthetic polynucleotide
  source 1..727: mol_type = other DNA
ccaaggtatattgctgttgacagtgagcgaccttgggctgtccctatctttcgtgaagccacagatggaaagatagggtcagcccaatgctgcctactgcctcggacttcaaggggcttgcggccgccatctccatggctgtaccaccttgtcggggcagccgtggatcgaataattgttgaatctcattatattcgatcaacggctgcgtctgacattttggtatctttcatctgaccacgtactaccttctatctgatgtgacagcttctgtagcacgccaccctcttcacggccaatgtttagttatttggccgtgacgagggtggctgtactgctagctgtagaactccagcttcggcctgtaacttatgatagcaatgtcagcagtgcctcgccaccctcttcacggccaattaagattctaaaattattgggccgtgaacagggtggctaagtaaggttgaccatactctacagttgttgatttcgtggctacagagtttccttagcagagctggatgcagtgcagccatatatttgtctaaactataatatatggctgcactgcatatagctactgctaggcaatccttccctcgataagatgcagcggcggctcctctccccatggccctggccttgttgaagaggattatcctgggctcagagataatcctctacaacaagggcagggacctggggaccccggcaccggcaggctagc

SEQ ID NO: 83 - DNA, 846 bp, synthetic construct
  misc_feature 1..846: note = Description of Artificial Sequence: Synthetic polynucleotide
  source 1..846: mol_type = other DNA
ggccaaggtatattgctgttgacagtgagcgaccttacctgaatcagacaagaagtgaagccacagatgttcttgtctgaatcaggtaatgctgcctactgcctcggacttcaaggggcttgcggccgccatctccatggctgtaccaccttgtcgggcatctagtagagcggatgattgttgaatctcatttcatccgctcaactagatggtctgacattttggtatctttcatctgaccacgtactaccttctatctgatgtgacagcttctgtagcacgccaccctcttcacggccaatgtttagttatttggccgtgacgagggtggctgtactgctagctgtagaactccagcttcggcctgtaacttatgatagcaatgtcagcagtgcctcgccaccctcttcacggccaattaagattctaaaattattgggccgtgaacagggtggctaagtaaggttgaccatactctacagttgttgatttcgtggctacagagtttccttagcagagctggatgcagtgcagccatatatttgtctaaactataatatatggctgcactgcatatagctactgctaggcaatccttccctcgataagtatggggcctggctcgagcagggggcgagggatgcatctagtagagcggatgattggtcccctcccaactgccactgttgatgtttgtccttccctcccaatgaccgcgtcttcgtcacagtcagcggcggctcctctccccatggccctgcgatgcagagtccaggataatctgggctcagagataatcctctacaacaagggcagggacctggggaccccggcaccggcaggcta

SEQ ID NO: 84 - DNA, 846 bp, synthetic construct
  misc_feature 1..846: note = Description of Artificial Sequence: Synthetic polynucleotide
  source 1..846: mol_type = other DNA
ggccaaggtatattgctgttgacagtgagcgacatgctgtccctggtccttatggtgaagccacagatgcataaggaccacggacagcaggctgcctactgcctcggacttcaaggggcttgcggccgccatctccatggctgtaccaccttgtcggctggatgcagtggcgacattttgttgaatctcattaatgtcgccagtgcatccactctgacattttggtatctttcatctgaccacgtactaccttctatctgatgtgacagcttctgtagcacgccaccctcttcacggccaatgtttagttatttggccgtgacgagggtggctgtactgctagctgtagaactccagcttcggcctgtaacttatgatagcaatgtcagcagtgcctcgccaccctcttcacggccaattaagattctaaaattattgggccgtgaacagggtggctaagtaaggttgaccatactctacagttgttgatttcgtggctacagagtttccttagcagagctggatgcagtgcagccatatatttgtctaaactataatatatggctgcactgcatatagctactgctaggcaatccttccctcgataagtatggggcctggctcgagcagggggcgagggatcgagggaaacaccgttcatattggtcccctcccttaacaagtcgaactgtcttgtccttccctcccaatgaccgcgtcttcgtcacagtcagcggcggctcctctccccatggccctggccttgttgaagaggattatcctgggctcagagataatcctctacaacaagggcagggacctggggaccccggcaccggcaggcta

SEQ ID NO: 85 - DNA, 846 bp, synthetic construct
  misc_feature 1..846: note = Description of Artificial Sequence: Synthetic polynucleotide
  source 1..846: mol_type = other DNA
ggccaaggtatattgctgttgacagtgagcgacatgctgtccctggtccttatggtgaagccacagatgcataaggaccacggacagcaggctgcctactgcctcggacttcaaggggcttgcggccgccatctccatggctgtaccaccttgtcggctggatgcagtggcgacattttgttgaatctcattaatgtcgccagtgcatccactctgacattttggtatctttcatctgaccacgtactaccttctatctgatgtgacagcttctgtagcacgccaccctcttcacggccaatgtttagttatttggccgtgacgagggtggctgtactgctagctgtagaactccagcttcggcctgtaacttatgatagcaatgtcagcagtgcctcgccaccctcttcacggccaattaagattctaaaattattgggccgtgaacagggtggctaagtaaggttgaccatactctacagttgttgatttcgtggctacagagtttccttagcagagctggatgcagtgcagccatatatttgtctaaactataatatatggctgcactgcatatagctactgctaggcaatccttccctcgataagtatggggcctggctcgagcagggggcgagggatcagacagttggacttgttaaatggtcccctccctatgaacggtctttccctctgtccttccctcccaatgaccgcgtcttcgtcacagtcagcggcggctcctctccccatggccctggccttgttgaagaggattatcctgggctcagagataatcctctacaacaagggcagggacctggggaccccggcaccggcaggcta

SEQ ID NO: 86 - DNA, 727 bp, synthetic construct
  misc_feature 1..727: note = Description of Artificial Sequence: Synthetic polynucleotide
  source 1..727: mol_type = other DNA
ccaaggtatattgctgttgacagtgagcgaccctgggcttctaactttgttagtgaagccacagatgtaacaaagttacaagcccagtgctgcctactgcctcggacttcaaggggcttgcggccgccatctccatggctgtaccaccttgtcggccgcatcttaaacttggagtttgttgaatctcattactccaagttaaagatgcgctctgacattttggtatctttcatctgaccacgtactaccttctatctgatgtgacagcttctgtagcacgccaccctcttcacggccaatgtttagttatttggccgtgacgagggtggctgtactgctagctgtagaactccagcttcggcctgtaacttatgatagcaatgtcagcagtgcctcgccaccctcttcacggccaattaagattctaaaattattgggccgtgaacagggtggctaagtaaggttgaccatactctacagttgttgatttcgtggctacagagtttccttagcagagctgcctggagattgatgcagcaattgtctaaactatattgctgcatcaatctccagttagctactgctaggcaatccttccctcgataagatgcagcggcggctcctctccccatggccctggcggctgatgaagctctatatctgggctcagaatatagagcttgatcagccggcagggacctggggaccccggcaccggcaggctagc

SEQ ID NO: 87 - DNA, 726 bp, synthetic construct
  misc_feature 1..726: note = Description of Artificial Sequence: Synthetic polynucleotide
  source 1..726: mol_type = other DNA
ccaaggtatattgctgttgacagtgagcgacgcaggtatgtatccaatacatgtgaagccacagatgatgtattggattcatacctgagctgcctactgcctcggacttcaaggggcttgcggccgccatctccatggctgtaccaccttgtcggcgagggaaacaccgttcatattgttgaatctcatttatgaacggtctttccctcctctgacattttggtatctttcatctgaccacgtactaccttctatctgatgtgacagcttctgtagcacgccaccctcttcacggccaatgtttagttatttggccgtgacgagggtggctgtactgctagctgtagaactccagcttcggcctgtaacttatgatagcaatgtcagcagtgcctcgccaccctcttcacggccaattaagattctaaaattattgggccgtgaacagggtggctaagtaaggttgaccatactctacagttgttgagactacgcctggctcgagcagggggcgagggatcgagggaaacaccgttcatattggtcccctccctatgaacggtctttccctctgtccttccctcccaatgaccgcgtcttcgtcacagtcagcggcggctcctctccccatggccctgcttgggctgtccctatctttcctgggctcagagaaagatagggtcagcccaaccagggacctggggaccccggcaccggcaggctagc

SEQ ID NO: 88 - DNA, 726 bp, synthetic construct
  misc_feature 1..726: note = Description of Artificial Sequence: Synthetic polynucleotide
  source 1..726: mol_type = other DNA
ccaaggtatattgctgttgacagtgagcgacgcggctgatgaagctctatatgtgaagccacagatgatatagagcttgatcagccgagctgcctactgcctcggacttcaaggggcttgcggccgccatctccatggctgtaccaccttgtcggcctggagattgatgcagcaattgttgaatctcattttgctgcatctatctccagctctgacattttggtatctttcatctgaccacgtactaccttctatctgatgtgacagcttctgtagcagcccttcaaggatatggactatgtttagttattagtccatatacttgaagggagtactgctagctgtagaactccagcttcggcctgtaacttatgatagcaatgtcagcagtgcctcggatcattgagcgtctcttattaagattctaaaattattgagagacgctgaatgatcctaagtaaggttgaccatactctacagttgttgatttcgtggctacagagtttccttagcagagctgcctggagattgatgcagcaattgtctaaactatattgctgcatcaatctccagttagctactgctaggcaatccttccctcgataagatgcagcggcggctcctctccccatggccctgcctgagtaatgcaaggttattctgggctcagaaataaccttgctttactcagccagggacctggggaccccggcaccggcagctagc

SEQ ID NO: 89 - DNA, 727 bp, synthetic construct
  misc_feature 1..727: note = Description of Artificial Sequence: Synthetic polynucleotide
  source 1..727: mol_type = other DNA
ccaaggtatattgctgttgacagtgagcgaccagaggctacagattgaacaagtgaagccacagatgttgttcaatctctagcctcttgctgcctactgcctcggacttcaaggggcttgcggccgccatctccatggctgtaccaccttgtcggccctttcattgtggacctgattgttgaatctcattacaggtccaccatgaaaggctctgacattttggtatctttcatctgaccacgtactaccttctatctgatgtgacagcttctgtagcaccactcgaacagctacagctatgtttagttattagctgtagcggttcgagtgtgtactgctagctgtagaactccagcttcggcctgtaacttatgatagcaatgtcagcagtgcctgctgatccacttctctaccaattaagattctaaaattattgggtagagaactggatcagaaagtaaggttgaccatactctacagttgttgatttcgtggctacagagtttccttagcagagctgacattgttacacagccagtattgtctaaactatatactggctgtgtaacaatggtagctactgctaggcaatccttccctcgataagatgcagcggcggctcctctccccatggccctggcatggaacattgtgagaaatctgggctcagaatttctcacaaagttccatggcagggacctggggaccccggcaccggcaggctagc

SEQ ID NO: 90 - DNA, 727 bp, synthetic construct
  misc_feature 1..727: note = Description of Artificial Sequence: Synthetic polynucleotide
  source 1..727: mol_type = other DNA
ccaaggtatattgctgttgacagtgagcgaccttacctgaatcagacaagaagtgaagccacagatgttcttgtctgaatcaggtaatgctgcctactgcctcggacttcaaggggcttgcggccgccatctccatggctgtaccaccttgtcgggcatctagtagagcggatgattgttgaatctcatttcatccgctcaactagatggtctgacattttggtatctttcatctgaccacgtactaccttctatctgatgtgacagcttctgtagcacctgggcttctaactttgttatgtttagttattaacaaagttcgaagcccagtgtactgctagctgtagaactccagcttcggcctgtaacttatgatagcaatgtcagcagtgcctccgcatcttaaacttggagttttaagattctaaaattatacctccaagttaaagatgcgaaagtaaggttgaccatactctacagttgttgatttcgtggctacagagtttccttagcagagctggcctacatcctctttgttatttgtctaaactataataacaaagacgatgtaggatagctactgctaggcaatccttccctcgataagtatggggcctggctcgagcagggggcgagggatgcatctagtagagcggatgattggtcccctccctcatccgctcaactagatgagtccttccctcccaatgaccgcgtcttggctagc

SEQ ID NO: 91 - DNA, 722 bp, synthetic construct
  misc_feature 1..722: note = Description of Artificial Sequence: Synthetic polynucleotide
  source 1..722: mol_type = other DNA
ccaaggtatattgctgttgacagtgagcgaccctgggcttctaactttgttagtgaagccacagatgtaacaaagttacaagcccagtgctgcctactgcctcggacttcaaggggcttgcggccgccatctccatggctgtaccaccttgtcggccgcatcttaaacttggagtttgttgaatctcattactccaagttaaagatgcgctctgacattttggtatctttcatctgaccacgtactaccttctatctgatgtgacagcttctgtagcacctgggcttctaactttgttatgtttagttattaacaaagttcgaagcccagtgtactgctagctgtagaactccagcttcggccttcacgtggctacagagtttccttagcagagctgctggatgcagtggcgacatttgtctaaactataaatgtcgccactgcatccattagctactgctaggcaatccttccctcgataaatggatggcctggctcgagcagggggcgagggatcagacagttggacttgttaaatggtcccctcccttaacaagtcgaactgtcttgtccttccctcccaatgaccgcgtcttcgtcacagtcagcggcggctcctctccccatggccctgatgctgtccctggtccttatgctgggctcagacataaggaccacggacagcaacagggacctggggaccccggcaccggcaggctagc

SEQ ID NO: 92 - DNA, 731 bp, synthetic construct
  misc_feature 1..731: note = Description of Artificial Sequence: Synthetic polynucleotide
  source 1..731: mol_type = other DNA
ccggccaaggtatattgctgttgacagtgagcgacgcggctgatgaagctctatatgtgaagccacagatgatatagagcttgatcagccgagctgcctactgcctcggacttcaaggggcttgcggccgccatctccatggctgtaccaccttgtcggcctggagattgatgcagcaattgttgaatctcattttgctgcatctatctccagctctgacattttggtatctttcatctgaccacgtactaccttctatctgatgtgacagcttctgtagcagcaggtatgtatccaatacattgtttagttatatgtattggagacatacctgagtactgctagctgtagaactccagcttcggcctgtaacttatgatagcaatgtcagcagtgcctcgagggaaacaccgttcatatttaagattctaaaattatagatgaacggtctttccctcaaagtaaggttgaccatactctacagttgttgatttcgtggctacagagtttccttagcagagctgcctggagattgatgcagcaattgtctaaactatattgctgcatcaatctccagttagctactgctaggcaatccttccctcgataagtatggggcctggctcgagcagggggcgagggatcgagggaaacaccgttcatattggtcccctccctatgaacggtctttccctctgtccttccctcccaatgaccgcgtcttggctagc

SEQ ID NO: 93 - DNA, 846 bp, synthetic construct
  misc_feature 1..846: note = Description of Artificial Sequence: Synthetic polynucleotide
  source 1..846: mol_type = other DNA
ggccaaggtatattgctgttgacagtgagcgacgccgtgaagcaaatagcagttgtgaagccacagatgaactgctatttccttcacggagctgcctactgcctcggacttcaaggggcttgcggccgccatctccatggctgtaccaccttgtcggcaaccggatgctcaagatatttgttgaatctcattatatcttgaggatccggttctctgacattttggtatctttcatctgaccacgtactaccttctatctgatgtgacagcttctgtagcagcaggtatgtatccaatacattgtttagttatatgtattggagacatacctgagtactgctagctgtagaactccagcttcggcctgtaacttatgatagcaatgtcagcagtgcctcgagggaaacaccgttcatatttaagattctaaaattatagatgaacggtctttccctcaaagtaaggttgaccatactctacagttgttgatttcgtggctacagagtttccttagcagagctgctggatgcagtggcgacattttgtctaaactataaatgtcgccactgcatccattagctactgctaggcaatccttccctcgataagtatggggcctggctcgagcagggggcgagggatcagacagttggacttgttaaatggtcccctccctcatccgctcaactagatgagtccttccctcccaatgaccgcgtcttcgtcgtttcagcggcggctcctctccccatggccctgtctgagactgacaccaggtatctgggctcagacataaggaccacggacagcaacagggacctggggaccccggcaccggcaggctag

SEQ ID NO: 94 - DNA, 14 bp, unidentified
  misc_feature 1..14: note = Description of Unknown: miR16-1-5' sequence
  source 1..14: mol_type = other DNA
tgtcagcagtgcct

SEQ ID NO: 95 - DNA, 14 bp, unidentified
  misc_feature 1..14: note = Description of Unknown: miR16-1-3' sequence
  source 1..14: mol_type = other DNA
agtaaggttgacca

SEQ ID NO: 96 - DNA, 18 bp, unidentified
  misc_feature 1..18: note = Description of Unknown: mir16-1-stem-loop sequence
  source 1..18: mol_type = other DNA
ttaagattctaaaattat

SEQ ID NO: 97 - DNA, 17 bp, unidentified
  misc_feature 1..17: note = Description of Unknown: miR30a-5' sequence
  source 1..17: mol_type = other DNA
tgttgacagtgagcgac

SEQ ID NO: 98 - DNA, 16 bp, unidentified
  misc_feature 1..16: note = Description of Unknown: miR20a-3' sequence
  source 1..16: mol_type = other DNA
gtactgctagctgtag

SEQ ID NO: 99 - DNA, 16 bp, unidentified
  misc_feature 1..16: note = Description of Unknown: mir20a-5' sequence
  source 1..16: mol_type = other DNA
gacagcttctgtagca

SEQ ID NO: 100 - DNA, 11 bp, unidentified
  misc_feature 1..11: note = Description of Unknown: mir20a-stem-loop sequence
  source 1..11: mol_type = other DNA
tgtttagttat

SEQ ID NO: 101 - DNA, 17 bp, unidentified
  misc_feature 1..17: note = Description of Unknown: mir21-3' sequence
  source 1..17: mol_type = other DNA
ctgacattttggtatct

SEQ ID NO: 102 - DNA, 16 bp, unidentified
  misc_feature 1..16: note = Description of Unknown: miR21-5' sequence
  source 1..16: mol_type = other DNA
tgtaccaccttgtcgg

SEQ ID NO: 103 - DNA, 14 bp, unidentified
  misc_feature 1..14: note = Description of Unknown: mir21-stem-loop sequence
  source 1..14: mol_type = other DNA
tgttgaatctcatt

SEQ ID NO: 104 - DNA, 15 bp, unidentified
  misc_feature 1..15: note = Description of Unknown: mir30a-stem-loop sequence
  source 1..15: mol_type = other DNA
gtgaagccacagatg

SEQ ID NO: 105 - DNA, 12 bp, unidentified
  misc_feature 1..12: note = Description of Unknown: mir122-stem-loop sequence
  source 1..12: mol_type = other DNA
tgtctaaactat

SEQ ID NO: 106 - DNA, 15 bp, unidentified
  misc_feature 1..15: note = Description of Unknown: mir150-3' sequence
  source 1..15: mol_type = other DNA
cagggacctggggac

SEQ ID NO: 107 - DNA, 11 bp, unidentified
  misc_feature 1..11: note = Description of Unknown: mir150-stem-loop sequence
  source 1..11: mol_type = other DNA
ctgggctcaga

SEQ ID NO: 108 - DNA, 17 bp, unidentified
  misc_feature 1..17: note = Description of Unknown: miR-30a sequence
  source 1..17: mol_type = other DNA
gctgcctactgcctcgg

SEQ ID NO: 109 - DNA, 16 bp, unidentified
  misc_feature 1..16: note = Description of Unknown: miR-122 sequence
  source 1..16: mol_type = other DNA
ttccttagcagagctg

SEQ ID NO: 110 - DNA, 16 bp, unidentified
  misc_feature 1..16: note = Description of Unknown: miR-122 sequence
  source 1..16: mol_type = other DNA
tagctactgctaggca

SEQ ID NO: 111 - DNA, 15 bp, unidentified
  misc_feature 1..15: note = Description of Unknown: miR-150 sequence
  source 1..15: mol_type = other DNA
ctccccatggccctg

SEQ ID NO: 112 - DNA, 14 bp, unidentified
  misc_feature 1..14: note = Description of Unknown: miR16-1-5' sequence
  source 1..14: mol_type = other DNA
tgtcagcagtgcct

SEQ ID NO: 113 - DNA, 248 bp, Woodchuck hepatitis virus; identical to SEQ ID NO: 28
  source 1..248: mol_type = other DNA

SEQ ID NO: 114 - DNA, 134 bp, Simian virus 40; identical to SEQ ID NO: 29
  source 1..134: mol_type = other DNA

SEQ ID NO: 115 - DNA, 3302 bp, synthetic construct
  misc_feature 1..3302: note = Description of Artificial Sequence: Synthetic polynucleotide
  source 1..3302: mol_type = other DNA
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattccttggctccggtgcccgtcagtgggcagagcgcacatcgcccacagtccccgagaagttgtggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgcggcctgccaccatgggtcgggggctgctccggggcctgtggccgctgcatatcgtcctgtggacgcgcatcgccagcacgatcccgccgcacgttcccaagtcggatgtggaaatggaagcccagaaagatgcatccatccacctaagctgtaataggaccatccatccactgaaacattttaacagtgatgtcatggccagcgacaatggcggtgcggtcaagcttccacagctgtgcaagttttgcgatgtgagactgtccacttgcgacaaccagaagtcctgcatgagcaactgcagcatcacggccatctgtgagaagccgcatgaagtctgcgtggccgtgtggaggaagaacgacaagaacattactctggagacggtttgccacgaccccaagctcacctaccacggcttcactctggaagatgccgcttctcccaagtgtgtcatgaaggaaaagaaaagggcgggcgagactttcttcatgtgtgcctgtaacatggaagagtgcaacgattacatcatcttttcggaagaatacaccaccagcagtcccgaccccagagggcccacaatcaagccctgtcctccatgcaaatgcccagcacctaacctcgagggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggcgttcgcatgcgcggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatgagctagcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccattctagctttatttgtgaaatttgtgatgctattgctttatttgtaaccattataagctgcaataaacaagttaacaacaacaattgcattcattttatgtttcaggttcagggggagatgtgggaggttttttaaagcgggggatccaaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaa

SEQ ID NO: 116 - DNA, 3854 bp, synthetic construct
  misc_feature 1..3854: note = Description of Artificial Sequence: Synthetic polynucleotide
  source 1..3854: mol_type = other DNA
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattccttggctccggtgcccgtcagtgggcagagcgcacatcgcccacagtccccgagaagttgtggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgcggccgctccgccaccatgatggacttggagttgccaccgccaggactacagtcccagcaggacatggatttgattgacatcctttggaggcaagacatagatcttggagtaagtcgagaagtgtttgactttagtcagcgacagaaggactatgagttggaaaaacagaaaaaactcgaaaaggaaagacaagagcaactccagaaggaacaggagaaggcctttttcgctcagtttcaactggatgaagaaacaggagaattcctcccaattcagccggcccagcacatccagacagacactagtggatccgccagctactcccaggttgcccacattcccaaacaagatgccttgtactttgaagactgtatgcagcttttggcagagacattcccatttgttgatgaccatgagtcgcttgccctggatatccccagccacgctgaaagttcagtcttcactgcccctcatcaggcccagtccctcaatagctctctggaggcagccatgactgatttaagcagcatagagcaggacatggagcaagtttggcaggagctattttccattcccgaattacagtgtcttaataccgaaaacaagcagctggctgatactaccgctgttcccagcccagaagccacactgacagaaatggacagcaattaccatttttactcatcgatctcctcgctggaaaaagaagtgggcaactgtggtccacatttccttcatggttttgaggattctttcagcagcatcctctccactgatgatgccagccagctgacctccttagactcaaatcccaccttaaacacagattttggcgatgaattttattctgctttcatagcagagcccagtgacggtggcagcatgccttcctccgctgccatcagtcagtcactctctgaactcctggacgggactattgaaggctgtgacctgtcactgtgtaaagctttcaacccgaagcacgctgaaggcacaatggaattcaatgactctgactctggcatttcactgaacacaagtcccagccgagcgtccccagagcactccgtggagtcttccatttacggagacccaccgcctgggttcagtgactcggaaatggaggagctagatagtgcccctggaagtgtcaaacagaacggccctaaagcacagccagcacattctcctggagacacagtacagcctctgtcaccagctcaagggcacagtgctcctatgcgtgaatcccaatgtgaaaatacaacaaaaaaagaagttcccgtgagtcctggtcatcaaaaagccccattcacaaaagacaaacattcaagccgcttagaggctcatctcacacgagatgagcttagggcaaaagctctccatattccattccctgtcgaaaaaatcattaacctccctgttgatgacttcaatgaaatgatgtccaaggagcaattcaatgaagctcagctcgcattgatccgagatatacgcaggagaggtaagaataaagtcgccgcccagaactgtaggaaaaggaagctggagaacattgtcgagctggagcaagacttgggccacttaaaagacgagagagaaaaactactcagagaaaagggagaaaacgacagaaacctccatctactgaaaaggcggctcagcaccttgtatcttgaagtcttcagcatgttacgtgatgaggatggaaagccttactctcccagtgaatactctctgcagcaaaccagagatggcaatgtgttccttgttcccaaaagcaagaagccagatacaaagaaaaactagagcgggctagcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccattctagctttatttgtgaaatttgtgatgctattgctttatttgtaaccattataagctgcaataaacaagttaacaacaacaattgcattcattttatgtttcaggttcagggggagatgtgggaggttttttaaagcgggggatccaaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaa

SEQ ID NO: 117 - DNA, 231 bp, Homo sapiens; identical to SEQ ID NO: 18
  source 1..231: mol_type = other DNA

SEQ ID NO: 118 - DNA, 22 bp, synthetic construct
  misc_feature 1..22: note = Description of Artificial Sequence: Synthetic primer
  source 1..22: mol_type = other DNA
gccaccatgggtcgggggctgc

SEQ ID NO: 119 - DNA, 58 bp, synthetic construct
  misc_feature 1..58: note = Description of Artificial Sequence: Synthetic primer
  source 1..58: mol_type = other DNA
ggacagggcttgattgtgggccctctggggtcgggactgctggtggtgtattcttccg

SEQ ID NO: 120 - DNA, 96 bp, Canis sp.
  source 1..96: mol_type = other DNA
atgcacagtcaagggcggggttgcaacaacacaaaacaaaacaaaacttccggacttcgacctgcagctgagaagaacatctcgcaaagcggcgtt

SEQ ID NO: 121 - DNA, 69 bp, unidentified
  misc_feature 1..69: note = Description of Unknown: mouse/human secretion signal sequence
  source 1..69: mol_type = other DNA
atgggtcgggggctgctccggggcctgtggccgctgcatatcgtcctgtggacgcgcatcgccagcacg

SEQ ID NO: 122 - DNA, 1269 bp, synthetic construct
  misc_feature 1..1269: note = Description of Artificial Sequence: Synthetic polynucleotide
  source 1..1269: mol_type = other DNA
atgggtcgggggctgctccggggcctgtggccgctgcatatcgtcctgtggacgcgcatcgccagcacgaataatgacatgatggtcactgacagcaatggtgtcatcaaatttccacaattgtgtaaattttgtgatgtgagatcttccacctgtgacaaccagaaatcttgcatgagcaactgcagcattacatccatctgtgagaagccacatgaagtctgtctggctgtctggagaaagaatgatgagaacataacactagagactctctgccatgaccccaaggatacctaccatggaattgttctcgaagatgctgcctcttcgaagtgcattatgaaagaaaagaaggtgctgggggagactttctttatgtgttcctgtagctccgacgagtgcaacgactacatcatcttctctgaagaatatgccaccaacaaccctgacttgttgttagtcatattccaacccaaaagagaaaatggaagagttcctcgcccacctgattgtcccaaatgcccagcccctgaaatgctgggagggccttcggtcttcatctttcccccgaaacccaaggacaccctcttgattgcccgaacacctgaggtcacatgtgtggtggtggatctggacccagaagaccctgaggtgcagatcagctggttcgtggacggtaagcagatgcaaacagccaagactcagcctcgtgaggagcagttcaatggcacctaccgtgtggtcagtgtcctccccattgggcaccaggactggctcaaggggaagcagttcacgtgcaaagtcaacaacaaagccctcccatccccgatcgagaggaccatctccaaggccagagggcaagcccatcagcccagtgtgtatgtcctgccgccatcccgggaggagttgagcaagaacacagtcagcttgacatgcctgatcaaagacttcttcccacctgacattgatgtggagtggcagagcaatggacagcaggagcctgagagcaagtaccgcacgaccccgccccagctggacgaggacgggtcctacttcctgtacagcaagctctctgtggacaagagccgctggcagcggggagacaccttcatatgtgcggtgatgcatgaagctctacacaaccactacacacaggaatccctctcccattctccgggtaaaggagggagtggtgggtccgattacaaagatcacgatggggactataaagatcacgacatcgactataaggatgacgatgataaatga

SEQ ID NO: 123 - DNA, 54 bp, Homo sapiens
  source 1..54: mol_type = other DNA
atggcttttctttggttgctgagctgctgggcactgctgggtactacttttgga

SEQ ID NO: 124 - protein, 18 aa, Homo sapiens
MAFLWLLSCWALLGTTFG

SEQ ID NO: 125 - DNA, 45 bp, unidentified
  misc_feature 1..45: note = Description of Unknown: Trypsinogen sequence
  source 1..45: mol_type = other DNA
atgaacttgcttctcatcctgacttttgttgcagccgccgtggct

SEQ ID NO: 126 - DNA, 57 bp, unidentified
  misc_feature 1..57: note = Description of Unknown: Heavy chain sequence
  source 1..57: mol_type = other DNA
atggagttcgggctttcttgggtgttcttggtcgctttgtttcggggggtccagtgt

SEQ ID NO: 127 - protein, 19 aa, unidentified
  REGION 1..19: note = Description of Unknown: Heavy chain sequence
MEFGLSWVFLVALFRGVQC

SEQ ID NO: 128 - DNA, 57 bp, unidentified
  misc_feature 1..57: note = Description of Unknown: IL2-ILco1 sequence
  source 1..57: mol_type = other DNA
atgaggatgcaacttctcctcttgatagccctttccttggctctggtcaccaacagc

SEQ ID NO: 129 - protein, 19 aa, unidentified
  REGION 1..19: note = Description of Unknown: IL2-ILco1 sequence
MRMQLLLLIALSLALVTNS

SEQ ID NO: 130 - protein, 20 aa, unidentified
  REGION 1..20: note = Description of Unknown: IL2-ILco2 sequence
MRRMQLLLLIALSLALVTNS

SEQ ID NO: 131 - protein, 14 aa, unidentified
  REGION 1..14: note = Description of Unknown: IL2 sequence
MQLLSCIALILALV

SEQ ID NO: 132 - protein, 18 aa, Homo sapiens
MKWVTFISLLFLFSSAYS

SEQ ID NO: 133 - protein, 19 aa, Homo sapiens
MTRLTVLALLAGLLASSRA

SEQ ID NO: 134 - protein, 17 aa, Gaussia sp.
MGVKVLFALICIAVAEA

SEQ ID NO: 135 - DNA, 2679 bp, Canis sp.
  source 1..2679: mol_type = other DNA
atggccacctgcattttacagatgagattcctaaggctggggaagatactgttccactccagcccacaaagcacaggtggcagtggtgggacccggggacctcgagctccggcacagctgcgaacgcagcgtggcacagataagttagttgctaagtcagagctcaaggctaaaacggcccaccgcgcgctggccgaccacttcagggactacgccgagctctgcttccgccacttctgcggccaggtcaagtactggatcaccatcgacaacccctacgtggtggcctggcacggctacgccaccggtcgcctggcacccggagtcagaggcagcccgcggctcgggtacctggtggcgcacaacctcctcctggctcacgccaaaatctggcatctctacaatacttctttccgcccaactcagggaggccaggtatccattgccctaagctcccactggatcaatcctcgaagaatgaccgaccatagcatcaaagaatgtcaaaaatctcttgactttgtactaggctggtttgccaagcccatatttattgatggtgactatcctgagagcatgaagaataacctgtcatctcttctgcctgtttttactgaatctgagaaaaagttcatcaagggaacagctgacttttttgctctttcttttggaccaactttgagttttcaactcttggaccctcatatgaagttccaccaattagaatctcccagcctgaggcaactcctttcttggattgaccttgaatataaccaccctcaaatatttattgtggaaaatggctggtttgtctcagggaccaccaagagagatgatgccaaatatatgtattacctcaaaaaattcataatggaaaccttaaaagccatcaggctggatggggtggatgtcataggatacacagcgtggtcccttatggatggcttcgagtggcacagaggctacagcatcagacgtggactcttctacgtggactttctaagccaggataagaaactgttgccaaagtcttcagccttgttctaccaaaagctgatagagaaaaatggcttccctcctttacctgaaaatcagcccctagaagggacatttccctgtgactttgcttggggaattgttgacaactacattcaagtggacaccactctgtctcagtttaccgacccgaacgtttacctgtgggacgtccatcacagcaagaggctgattaaggtggacgggctgcgggccaagaagaggaagccctactgcgtggactttgccgccatcgggccccaggtggccctgctgcaggagatgcacgtctcgcattttcacttctcgctggactgggccctgctcctgccgctgggcaaccagtcccgggtgaaccacgcggccctgcactactacggctgcgtggccagcgagctcctgcgcgccaacatcaccccggtggtggcgctctggagaccagccgctgcgcaccagggtctgcctggaccgctggcacagcgcggtgcctgggagaacccacgcaccgccctggcgttcgccgagtacgcgcgcctgtgcttccgcgccctgggccgccacgtcaaggtgtggatcacgctgcgcgagccgcccacgcggaacctgacgctccgcgccgggcacaacctgctgcgggcgcacgcgctggcctggcgcgtgtacgacgagcagttccggggctcgcagcaggggaaggtgtccatcgccctgcaggccgactgggtggagcccgcctgcccctcctcccagaaggaccgcgaagtggccgagagggttctggagttcgacgtcggctggctggccgagcccatcttcggctccggggactacccgcggctgatgcgcgactggctcacccggagagaccattccctcctgccctatttcactgacgaagagaagaggctaatccggggttcctttgacttcctggccttgagccattacaccaccatcctcgtggactgggaaaaggaagacccagtcaaatacaatgattacctggaagtgcaggagatgaccgacatcacctggctcaactcccccagtcaggtggccgtagtgccctggggcctgcgcaaagtgctcaactggctcaagttcaagtacggagacctccccatgtatatcgtatccaacggcatagatgacgatccgcgggcagcccaggactcgttgagggtgtattacatgcagaactatgtaaatgaagctctgaaagcctacgtattggatggtatcaatctttgtggatactttgcctactcatttaatgatcgcacagctccgaagtttggcctctatcattatgctgcaaaccagtttgagcccaaaccgtcggtgaagcattacaggaaaattattgacaacaatggcttcccaggccctgaaactttggggcggttttgtccagaggaattcaccctgtgcaccgaatgcagcttttttcacacccgaaagtctttactggctttcatagctttcctactttttgcttttattatttctctttctctgattttctactactctaggaaaggcagaagaagttataaaggagggagtggtgggtccgattacaaagatcacgatggggactataaagatcacgacatcgactataaggatgacgatgataaatga

SEQ ID NO: 136 - DNA, 633 bp, Canis sp.
  source 1..633: mol_type = other DNA
atgggctgggccgaggccgggttcgagcacctgggactgtgggtccctgtgctggctgtgcttttgctggaagcctgccgggcacatccgatccctgactccagccccctcctacaatttggaggtcaagttcgacagcggtacctctacaccgacgatgcccaggagacagaggcccacctagagatcagggccgatggcacagtggtgggggctgcccgccagagccctgaaagtctcctggagctgaaagccctaaagccaggggtcattcaaatcttgggagtcaaaacatccaggttcctgtgccagggcccagatgggacactatatggctcgctccatttcgaccctgtggcctgcagtttccgagaactgcttcttgaggatgggtacaacatctaccactccgagacccttggtctcccgcttcgcctgcgcccccacaactccgcataccgggacttggcaccccgcgggcctgcccgcttcctgccactgccaggcctgcttccagcacccccagagcctccagggatcctggccccggagcctcctgacgtgggctcctcggaccctctgagcatggtggggccttcacagggccggagtcccagctatgcttcctgatag

SEQ ID NO: 137 - DNA, 727 bp, synthetic construct; identical to SEQ ID NO: 77
  misc_feature 1..727: note = Description of Artificial Sequence: Synthetic polynucleotide
  source 1..727: mol_type = other DNA
  CDS 1..234
  CDS 238..261
  CDS 277..303
  CDS 313..372
  CDS 379..417
  CDS 421..552
  CDS 556..603
  CDS 607..723

SEQ ID NO: 138 - protein, 78 aa, synthetic construct
  REGION 1..78: note = Description of Artificial Sequence: Synthetic polypeptide
PRYIAVDSERRCRYSALSEATDVRAENLQSCLLPRTSRGLRPPSPWLYHLVGQVTTDMYVESHYISVVTCSDILVSFI

SEQ ID NO: 139 - protein, 8 aa, synthetic construct
  REGION 1..8: note = Description of Artificial Sequence: Synthetic peptide
PRTTFYLM

SEQ ID NO: 140 - protein, 9 aa, synthetic construct
  REGION 1..9: note = Description of Artificial Sequence: Synthetic peptide
HPVTTHTFV

SEQ ID NO: 141 - protein, 20 aa, synthetic construct
  REGION 1..20: note = Description of Artificial Sequence: Synthetic peptide
SMWSNRCTASCRTPASACNL

SEQ ID NO: 142 - protein, 13 aa, synthetic construct
  REGION 1..13: note = Description of Artificial Sequence: Synthetic peptide
QCQQCLLFPFLII

SEQ ID NO: 143 - protein, 44 aa, synthetic construct
  REGION 1..44: note = Description of Artificial Sequence: Synthetic polypeptide
DSKIIGLGMGTVSKVDHTLQLLISWLQSFLSRAGCSAAIYLSKL

SEQ ID NO: 144 - protein, 16 aa, synthetic construct
  REGION 1..16: note = Description of Artificial Sequence: Synthetic peptide
YMAALHIATARQSFPR

SEQ ID NO: 145 - protein, 39 aa, synthetic construct
  REGION 1..39: note = Description of Artificial Sequence: Synthetic polypeptide
DAAAAPLPMALALLKRIILGSEIILYNKGRDLGTPAPAG

SEQ ID NO: 146 - DNA, 846 bp, synthetic construct
  misc_feature 1..846: note = Description of Artificial Sequence: Synthetic polynucleotide
  source 1..846: mol_type = other DNA
  CDS 1..258
  CDS 262..456
  CDS 460..486
  CDS 490..576
  CDS 580..846
ggccaaggtatattgctgttgacagtgagcgacatgctgtccctggtccttatggtcaagccacagatgcataaggaccacggacagcaggctgcctactgcctcggacttcaaggggcttgcggccgccatctccatggctgtaccaccttgtcggctggatgcagtggcgacattttgttgaatctcattaatgtcgccagtgcatccactctgacattttggtatctttcatctgaccacgtactaccttctatctgatgtgacagcttctgtagcacgccaccctcttcacggccaatgtttagttatttggccgtgacgagggtggctgtactgctagctgtagaactccagcttcggcctgtaacttatgatagcaatgtcagcagtgcctcgccaccctcttcacggccaattaagattctaaaattattgggccgtgaacagggtggctaagtaaggttgaccatactctacagttgttgatttcgtggctacagagtttccttagcagagctggatgcagtgcagccatatatttgtctaaactataatatatggctgcactgcatatagctactgctaggcaatccttccctcgataagtatggggcctggctcgagcagggggcgagggatcagacagttggacttgttaaatggtcccctccctatgaacggtctttccctctgtccttccctcccaatgaccgcgtcttcgtcacagtcagcggcggctcctctccccatggccctggccttgttgaagaggattatcctgggctcagagataatcctctacaacaagggcagggacctggggaccccggcaccggcaggcta

SEQ ID NO: 147 - protein, 86 aa, synthetic construct
  REGION 1..86: note = Description of Artificial Sequence: Synthetic polypeptide
GQGILLLTVSDMLSLVLMVKPQMHKDHGQQAAYCLGLQGACGRHLHGCTTLSAGCSGDILLNLINVASASTLTFWYLSSDHVLPSI

SEQ ID NO: 148 - protein, 65 aa, synthetic construct
  REGION 1..65: note = Description of Artificial Sequence: Synthetic polypeptide
CDSFCSTPPSSRPMFSYLAVTRVAVLLAVELQLRPVTYDSNVSSASPPSSRPIKILKLLGREQGG

SEQ ID NO: 149 - protein, 9 aa, synthetic construct
  REGION 1..9: note = Description of Artificial Sequence: Synthetic peptide
VRLTILYSC

SEQ ID NO: 150 - protein, 29 aa, synthetic construct
  REGION 1..29: note = Description of Artificial Sequence: Synthetic peptide
FRGYRVSLAELDAVQPYICLNYNIWLHCI

SEQ ID NO: 151 - protein, 89 aa, synthetic construct
  REGION 1..89: note = Description of Artificial Sequence: Synthetic polypeptide
LLLGNPSLDKYGAWLEQGARDQTVGLVKWSPPYERSFPLSFPPNDRVFVTVSGGSSPHGPGLVEEDYPGLRDNPLQQGQGPGDPGTGRL